SEQUENCE LISTING (330 sequences)

SEQ ID NO: 1 - DNA, 37 bp, Artificial Sequence
  Description of Artificial Sequence Synthetic primer
gtaaaacgacggccagtggtcctgtctcagtccctca

SEQ ID NO: 2 - DNA, 37 bp, Artificial Sequence
  Description of Artificial Sequence Synthetic primer
gtaaaacgacggccagtccagcggtacctcatagcat

SEQ ID NO: 3 - DNA, 38 bp, Artificial Sequence
  Description of Artificial Sequence Synthetic primer
gtaaaacgacggccagttggatttcgggtatcacataa

SEQ ID NO: 4 - DNA, 40 bp, Artificial Sequence
  Description of Artificial Sequence Synthetic primer
gtaaaacgacggccagttccctctttttcaaaagcataca

SEQ ID NO: 5 - DNA, 37 bp, Artificial Sequence
  Description of Artificial Sequence Synthetic primer
gtaaaacgacggccagtttgctgtcacagaaggatgc

SEQ ID NO: 6 - DNA, 37 bp, Artificial Sequence
  Description of Artificial Sequence Synthetic primer
gtaaaacgacggccagtaatgatgcttggcacagtga

SEQ ID NO: 7 - DNA, 37 bp, Artificial Sequence
  Description of Artificial Sequence Synthetic primer
gtaaaacgacggccagtggttctagtgctgggctctg

SEQ ID NO: 8 - DNA, 37 bp, Artificial Sequence
  Description of Artificial Sequence Synthetic primer
gtaaaacgacggccagtgctttgtggagcctgttctc

SEQ ID NO: 9 - DNA, 37 bp, Artificial Sequence
  Description of Artificial Sequence Synthetic primer
gtaaaacgacggccagtgtcaaatgaagcgcaacaga

SEQ ID NO: 10 - DNA, 37 bp, Artificial Sequence
  Description of Artificial Sequence Synthetic primer
gtaaaacgacggccagtcaaggagttgcttggtctca

SEQ ID NO: 11 - DNA, 37 bp, Artificial Sequence
  Description of Artificial Sequence Synthetic primer
gtaaaacgacggccagtcgacaggaaatggagaagga

SEQ ID NO: 12 - DNA, 37 bp, Artificial Sequence
  Description of Artificial Sequence Synthetic primer
gtaaaacgacggccagtaaaagtggcttgtgtgtccc

SEQ ID NO: 13 - DNA, 37 bp, Artificial Sequence
  Description of Artificial Sequence Synthetic primer
gtaaaacgacggccagtgaggtttgcttgagacagcc

SEQ ID NO: 14 - DNA, 37 bp, Artificial Sequence
  Description of Artificial Sequence Synthetic primer
gtaaaacgacggccagtggaccctcgcagacattaaa

SEQ ID NO: 15 - DNA, 37 bp, Artificial Sequence
  Description of Artificial Sequence Synthetic primer
gtaaaacgacggccagtgccatgtccagagctaggag

SEQ ID NO: 16 - DNA, 37 bp, Artificial Sequence
  Description of Artificial Sequence Synthetic primer
gtaaaacgacggccagtcttgaaaaccaaggctctcg

SEQ ID NO: 17 - DNA, 37 bp, Artificial Sequence
  Description of Artificial Sequence Synthetic primer
gtaaaacgacggccagtcaaggtgaatggtgacatgc

SEQ ID NO: 18 - DNA, 37 bp, Artificial Sequence
  Description of Artificial Sequence Synthetic primer
gtaaaacgacggccagtctgagtaccagccaagagcc

SEQ ID NO: 19 - DNA, 37 bp, Artificial Sequence
  Description of Artificial Sequence Synthetic primer
gtaaaacgacggccagtttttgactccctccatccag

SEQ ID NO: 20 - DNA, 37 bp, Artificial Sequence
  Description of Artificial Sequence Synthetic primer
gtaaaacgacggccagtctggaactggtgggtcactt

SEQ ID NO: 21 - DNA, 37 bp, Artificial Sequence
  Description of Artificial Sequence Synthetic primer
gtaaaacgacggccagtgcaagtatccgagcaaaacc

SEQ ID NO: 22 - DNA, 37 bp, Artificial Sequence
  Description of Artificial Sequence Synthetic primer
gtaaaacgacggccagtccgacggagagtctcatttt

SEQ ID NO: 23 - DNA, 37 bp, Artificial Sequence
  Description of Artificial Sequence Synthetic primer
gtaaaacgacggccagtggagaggcgtggaactagag

SEQ ID NO: 24 - DNA, 37 bp, Artificial Sequence
  Description of Artificial Sequence Synthetic primer
gtaaaacgacggccagttcataactccggtccctctg

SEQ ID NO: 25 - DNA, 37 bp, Artificial Sequence
  Description of Artificial Sequence Synthetic primer
gtaaaacgacggccagtcatttccaaggcacaaggtt

SEQ ID NO: 26 - DNA, 37 bp, Artificial Sequence
  Description of Artificial Sequence Synthetic primer
gtaaaacgacggccagtttgtcactggtcagctccag

SEQ ID NO: 27 - DNA, 37 bp, Artificial Sequence; identical to SEQ ID NO: 26
  Description of Artificial Sequence Synthetic primer

SEQ ID NO: 28 - DNA, 35 bp, Artificial Sequence
  Description of Artificial Sequence Synthetic primer
gtaaaacgacggccagtcaggtgcatggtggtctg

SEQ ID NO: 29 - DNA, 37 bp, Artificial Sequence
  Description of Artificial Sequence Synthetic primer
gtaaaacgacggccagtctcgttgctgttcttgtcca

SEQ ID NO: 30 - DNA, 37 bp, Artificial Sequence; identical to SEQ ID NO: 29
  Description of Artificial Sequence Synthetic primer

SEQ ID NO: 31 - DNA, 36 bp, Artificial Sequence
  Description of Artificial Sequence Synthetic primer
gtaaaacgacggccagtgcttggcttcatcctcctc

SEQ ID NO: 32 - DNA, 37 bp, Artificial Sequence
  Description of Artificial Sequence Synthetic primer
gtaaaacgacggccagtatgtaggcgctgatgtcgat

SEQ ID NO: 33 - DNA, 37 bp, Artificial Sequence
  Description of Artificial Sequence Synthetic primer
gtaaaacgacggccagtcctctctcccacctttcctc

SEQ ID NO: 34 - DNA, 37 bp, Artificial Sequence
  Description of Artificial Sequence Synthetic primer
gtaaaacgacggccagtggaaaacaagtcaggtggga

SEQ ID NO: 35 - DNA, 37 bp, Artificial Sequence
  Description of Artificial Sequence Synthetic primer
gtaaaacgacggccagtccacactagctggagaagca

SEQ ID NO: 36 - DNA, 37 bp, Artificial Sequence
  Description of Artificial Sequence Synthetic primer
gtaaaacgacggccagtcatggcagagcagctagtca

SEQ ID NO: 37 - DNA, 37 bp, Artificial Sequence
  Description of Artificial Sequence Synthetic primer
gtaaaacgacggccagtaatacccaaccccaggagtc

SEQ ID NO: 38 - DNA, 37 bp, Artificial Sequence
  Description of Artificial Sequence Synthetic primer
gtaaaacgacggccagtgaagccattagtgagctggc

SEQ ID NO: 39 - DNA, 37 bp, Artificial Sequence
  Description of Artificial Sequence Synthetic primer
gtaaaacgacggccagtttgccaaaagtattgggagg

SEQ ID NO: 40 - DNA, 37 bp, Artificial Sequence
  Description of Artificial Sequence Synthetic primer
gtaaaacgacggccagtaagcttcccctttgggataa

SEQ ID NO: 41 - DNA, 37 bp, Artificial Sequence
  Description of Artificial Sequence Synthetic primer
gtaaaacgacggccagtagggtcctaagcagtgagca

SEQ ID NO: 42 - DNA, 37 bp, Artificial Sequence
  Description of Artificial Sequence Synthetic primer
gtaaaacgacggccagtaggtgtgctacctggaatgg

SEQ ID NO: 43 - DNA, 37 bp, Artificial Sequence
  Description of Artificial Sequence Synthetic primer
gtaaaacgacggccagtatctggggactaaaatgggg

SEQ ID NO: 44 - DNA, 37 bp, Artificial Sequence
  Description of Artificial Sequence Synthetic primer
gtaaaacgacggccagtagcaaaggtgaaaggctgaa

SEQ ID NO: 45 - DNA, 37 bp, Artificial Sequence
  Description of Artificial Sequence Synthetic primer
gtaaaacgacggccagttcccaggcaacaaacttacc

SEQ ID NO: 46 - DNA, 37 bp, Artificial Sequence
  Description of Artificial Sequence Synthetic primer
gtaaaacgacggccagttcttctggaggaggaaagca

SEQ ID NO: 47 - DNA, 37 bp, Artificial Sequence
  Description of Artificial Sequence Synthetic primer
gtaaaacgacggccagtagtagtgagggtggcacagg

SEQ ID NO: 48 - DNA, 37 bp, Artificial Sequence
  Description of Artificial Sequence Synthetic primer
gtaaaacgacggccagtcttacacttgcaagcaccca

SEQ ID NO: 49 - DNA, 37 bp, Artificial Sequence
  Description of Artificial Sequence Synthetic primer
gtaaaacgacggccagtcatccaccaagacacaatgc

SEQ ID NO: 50 - DNA, 37 bp, Artificial Sequence
  Description of Artificial Sequence Synthetic primer
gtaaaacgacggccagttcttctccacaattcccctg

SEQ ID NO: 51 - DNA, 37 bp, Artificial Sequence
  Description of Artificial Sequence Synthetic primer
gtaaaacgacggccagtcactcttttcaaacccggag

SEQ ID NO: 52 - DNA, 37 bp, Artificial Sequence
  Description of Artificial Sequence Synthetic primer
gtaaaacgacggccagtactgaggcccatcacttctg

SEQ ID NO: 53 - DNA, 37 bp, Artificial Sequence
  Description of Artificial Sequence Synthetic primer
gtaaaacgacggccagtcttcccacagagggatgtgt

SEQ ID NO: 54 - DNA, 37 bp, Artificial Sequence
  Description of Artificial Sequence Synthetic primer
gtaaaacgacggccagttacaatcacccagccctctc

SEQ ID NO: 55 - DNA, 37 bp, Artificial Sequence
  Description of Artificial Sequence Synthetic primer
gtaaaacgacggccagtagccaagtccctgactctca

SEQ ID NO: 56 - DNA, 37 bp, Artificial Sequence
  Description of Artificial Sequence Synthetic primer
gtaaaacgacggccagtttgaagaatggggtacctgc

SEQ ID NO: 57 - DNA, 37 bp, Artificial Sequence
  Description of Artificial Sequence Synthetic primer
gtaaaacgacggccagttgcggtcatgcactcagtat

SEQ ID NO: 58 - DNA, 39 bp, Artificial Sequence
  Description of Artificial Sequence Synthetic primer
gtaaaacgacggccagtcttccacatattcacaggcagt

SEQ ID NO: 59 - DNA, 37 bp, Artificial Sequence
  Description of Artificial Sequence Synthetic primer
gtaaaacgacggccagtgcggcatgatatgagaaggt

SEQ ID NO: 60 - DNA, 38 bp, Artificial Sequence
  Description of Artificial Sequence Synthetic primer
gtaaaacgacggccagttggtgtcagtgagcatgaaga

SEQ ID NO: 61 - DNA, 37 bp, Artificial Sequence
  Description of Artificial Sequence Synthetic primer
gtaaaacgacggccagtcacaagaggtgaggtgagca

SEQ ID NO: 62 - DNA, 37 bp, Artificial Sequence
  Description of Artificial Sequence Synthetic primer
gtaaaacgacggccagtagcatgcaaatccacaaaca

SEQ ID NO: 63 - DNA, 37 bp, Artificial Sequence
  Description of Artificial Sequence Synthetic primer
gtaaaacgacggccagttttgccccagctaaatcatc

SEQ ID NO: 64 - DNA, 37 bp, Artificial Sequence
  Description of Artificial Sequence Synthetic primer
gtaaaacgacggccagtcctgcctcacacacacagac

SEQ ID NO: 65 - DNA, 37 bp, Artificial Sequence
  Description of Artificial Sequence Synthetic primer
gtaaaacgacggccagtcggctacatctcagtcccat

SEQ ID NO: 66 - DNA, 37 bp, Artificial Sequence
  Description of Artificial Sequence Synthetic primer
gtaaaacgacggccagtccaacaacagcccttaggaa

SEQ ID NO: 67 - DNA, 37 bp, Artificial Sequence
  Description of Artificial Sequence Synthetic primer
gtaaaacgacggccagttgacactgctagatgctggg

SEQ ID NO: 68 - DNA, 37 bp, Artificial Sequence
  Description of Artificial Sequence Synthetic primer
gtaaaacgacggccagtacaactcaaatccaatcggc

SEQ ID NO: 69 - DNA, 37 bp, Artificial Sequence
  Description of Artificial Sequence Synthetic primer
gtaaaacgacggccagtgagaggggcttgggatctac

SEQ ID NO: 70 - DNA, 37 bp, Artificial Sequence
  Description of Artificial Sequence Synthetic primer
gtaaaacgacggccagttcagagcaatcctcaagcaa

SEQ ID NO: 71 - DNA, 37 bp, Artificial Sequence
  Description of Artificial Sequence Synthetic primer
gtaaaacgacggccagtaagtgtagtggctcatccgc

SEQ ID NO: 72 - DNA, 37 bp, Artificial Sequence
  Description of Artificial Sequence Synthetic primer
gtaaaacgacggccagtccaccctacctggccataat

SEQ ID NO: 73 - DNA, 37 bp, Artificial Sequence
  Description of Artificial Sequence Synthetic primer
gtaaaacgacggccagtgagcccctatatgccacaga

SEQ ID NO: 74 - DNA, 40 bp, Artificial Sequence
  Description of Artificial Sequence Synthetic primer
gtaaaacgacggccagtctgtcttgattcaccttgacaat

SEQ ID NO: 75 - DNA, 37 bp, Artificial Sequence
  Description of Artificial Sequence Synthetic primer
gtaaaacgacggccagtggtcaatgatttcctcccaa

SEQ ID NO: 76 - DNA, 38 bp, Artificial Sequence
  Description of Artificial Sequence Synthetic primer
caggaaacagctatgaccatggcaatcgtttcctgttc

SEQ ID NO: 77 - DNA, 37 bp, Artificial Sequence
  Description of Artificial Sequence Synthetic primer
gtaaaacgacggccagtcctgaagctgcagaaaaacc

SEQ ID NO: 78 - DNA, 37 bp, Artificial Sequence
  Description of Artificial Sequence Synthetic primer
gtaaaacgacggccagtgttgacaccccaatccactc

SEQ ID NO: 79 - DNA, 37 bp, Artificial Sequence
  Description of Artificial Sequence Synthetic primer
gtaaaacgacggccagttttccaaaagcacctgatcc

SEQ ID NO: 80 - DNA, 37 bp, Artificial Sequence
  Description of Artificial Sequence Synthetic primer
gtaaaacgacggccagtgatctgctccctgagaggtg

SEQ ID NO: 81 - DNA, 37 bp, Artificial Sequence
  Description of Artificial Sequence Synthetic primer
gtaaaacgacggccagtctccctggtacctctcatgc

SEQ ID NO: 82 - DNA, 37 bp, Artificial Sequence
  Description of Artificial Sequence Synthetic primer
gtaaaacgacggccagttgtgttgagatggacgccta

SEQ ID NO: 83 - DNA, 37 bp, Artificial Sequence
  Description of Artificial Sequence Synthetic primer
gtaaaacgacggccagtctgcctctttgtggcctaag

SEQ ID NO: 84 - DNA, 37 bp, Artificial Sequence
  Description of Artificial Sequence Synthetic primer
gtaaaacgacggccagtgggtttcctcagaacgttga

SEQ ID NO: 85 - DNA, 37 bp, Artificial Sequence
  Description of Artificial Sequence Synthetic primer
gtaaaacgacggccagtttctgccctttgaacttgct

SEQ ID NO: 86 - DNA, 37 bp, Artificial Sequence
  Description of Artificial Sequence Synthetic primer
gtaaaacgacggccagtccacaccctgttcactcctt

SEQ ID NO: 87 - DNA, 37 bp, Artificial Sequence
  Description of Artificial Sequence Synthetic primer
gtaaaacgacggccagttgtgaacatcattcaaggcg

SEQ ID NO: 88 - DNA, 37 bp, Artificial Sequence
  Description of Artificial Sequence Synthetic primer
gtaaaacgacggccagttgcatggcattagcaaagac

SEQ ID NO: 89 - DNA, 38 bp, Artificial Sequence
  Description of Artificial Sequence Synthetic primer
gtaaaacgacggccagtccaaggaaagtaaagttccca

SEQ ID NO: 90 - DNA, 37 bp, Artificial Sequence
  Description of Artificial Sequence Synthetic primer
gtaaaacgacggccagtctcgggagatgaagttggaa

SEQ ID NO: 91 - DNA, 37 bp, Artificial Sequence
  Description of Artificial Sequence Synthetic primer
gtaaaacgacggccagtgtggtaacctcatttcccca

SEQ ID NO: 92 - DNA, 40 bp, Artificial Sequence
  Description of Artificial Sequence Synthetic primer
gtaaaacgacggccagtcaggttttagaaacttcagcagc

SEQ ID NO: 93 - DNA, 39 bp, Artificial Sequence
  Description of Artificial Sequence Synthetic primer
gtaaaacgacggccagtggggcttagagtggcttaattt

SEQ ID NO: 94 - DNA, 37 bp, Artificial Sequence
  Description of Artificial Sequence Synthetic primer
gtaaaacgacggccagttctgaaaaccagaaggtggc

SEQ ID NO: 95 - DNA, 40 bp, Artificial Sequence
  Description of Artificial Sequence Synthetic primer
gtaaaacgacggccagtaccaatttgttttccttgacaga

SEQ ID NO: 96 - DNA, 37 bp, Artificial Sequence
  Description of Artificial Sequence Synthetic primer
gtaaaacgacggccagtaccactgtgcattgcatgat

SEQ ID NO: 97 - DNA, 37 bp, Artificial Sequence
  Description of Artificial Sequence Synthetic primer
gtaaaacgacggccagtctgaaacattgggtggcttt

SEQ ID NO: 98 - DNA, 37 bp, Artificial Sequence
  Description of Artificial Sequence Synthetic primer
gtaaaacgacggccagtatgaacatgaactggagccc

SEQ ID NO: 99 - DNA, 38 bp, Artificial Sequence
  Description of Artificial Sequence Synthetic primer
gtaaaacgacggccagtttaatcttggctccacactgg

SEQ ID NO: 100 - DNA, 39 bp, Artificial Sequence
  Description of Artificial Sequence Synthetic primer
gtaaaacgacggccagttttcttgaaatacggcttacga

SEQ ID NO: 101 - DNA, 37 bp, Artificial Sequence
  Description of Artificial Sequence Synthetic primer
gtaaaacgacggccagtcccatgttttaaatgggcac

SEQ ID NO: 102 - DNA, 36 bp, Artificial Sequence
  Description of Artificial Sequence Synthetic primer
gtaaaacgacggccagtatcagctaccgccaagtcc

SEQ ID NO: 103 - DNA, 37 bp, Artificial Sequence
  Description of Artificial Sequence Synthetic primer
gtaaaacgacggccagtctccagctatagtggggaaa

SEQ ID NO: 104 - DNA, 39 bp, Artificial Sequence
  Description of Artificial Sequence Synthetic primer
gtaaaacgacggccagtccatagaaggggtatttgttgg

SEQ ID NO: 105 - DNA, 39 bp, Artificial Sequence
  Description of Artificial Sequence Synthetic primer
gtaaaacgacggccagtaaagattcaggcaatgtttgtt

SEQ ID NO: 106 - DNA, 43 bp, Artificial Sequence
  Description of Artificial Sequence Synthetic primer
gtaaaacgacggccagtggaatccagtgtttcttttaaatacc

SEQ ID NO: 107 - DNA, 37 bp, Artificial Sequence
  Description of Artificial Sequence Synthetic primer
gtaaaacgacggccagtggctacgacccagttaccat

SEQ ID NO: 108 - DNA, 38 bp, Artificial Sequence
  Description of Artificial Sequence Synthetic primer
gtaaaacgacggccagttgcttgagatcaagattgcag

SEQ ID NO: 109 - DNA, 39 bp, Artificial Sequence
  Description of Artificial Sequence Synthetic primer
gtaaaacgacggccagtgcaacagataactcagattgcc

SEQ ID NO: 110 - DNA, 38 bp, Artificial Sequence
  Description of Artificial Sequence Synthetic primer
gtaaaacgacggccagttgttcatctgcaaaatggaat

SEQ ID NO: 111 - DNA, 37 bp, Artificial Sequence
  Description of Artificial Sequence Synthetic primer
gtaaaacgacggccagtcttcctgtttgctttccagc

SEQ ID NO: 112 - DNA, 35 bp, Artificial Sequence
  Description of Artificial Sequence Synthetic primer
gtaaaacgacggccagtaccacgtcgctctggttc

SEQ ID NO: 113 - DNA, 37 bp, Artificial Sequence
  Description of Artificial Sequence Synthetic primer
gtaaaacgacggccagtaagaaaatcagtgcatgggc

SEQ ID NO: 114 - DNA, 37 bp, Artificial Sequence
  Description of Artificial Sequence Synthetic primer
gtaaaacgacggccagttgttacgacggtttgcagag

SEQ ID NO: 115 - DNA, 37 bp, Artificial Sequence
  Description of Artificial Sequence Synthetic primer
gtaaaacgacggccagtagttggtctgggaaggtgtg

SEQ ID NO: 116 - DNA, 37 bp, Artificial Sequence
  Description of Artificial Sequence Synthetic primer
gtaaaacgacggccagtgcaactttttggctttacgg

SEQ ID NO: 117 - DNA, 37 bp, Artificial Sequence
  Description of Artificial Sequence Synthetic primer
gtaaaacgacggccagtccgagtttctagggattcca

SEQ ID NO: 118 - DNA, 37 bp, Artificial Sequence
  Description of Artificial Sequence Synthetic primer
gtaaaacgacggccagtagaaagctgagacgagtgcc

SEQ ID NO: 119 - DNA, 37 bp, Artificial Sequence
  Description of Artificial Sequence Synthetic primer
gtaaaacgacggccagtggaatcagcagaaacagcct

SEQ ID NO: 120 - DNA, 37 bp, Artificial Sequence
  Description of Artificial Sequence Synthetic primer
gtaaaacgacggccagtggtgaaacaagctgccattt

SEQ ID NO: 121 - DNA, 37 bp, Artificial Sequence
  Description of Artificial Sequence Synthetic primer
gtaaaacgacggccagtcacccttgttctccatgacc

SEQ ID NO: 122 - DNA, 37 bp, Artificial Sequence
  Description of Artificial Sequence Synthetic primer
gtaaaacgacggccagtaaatggagacaccaagtggc

SEQ ID NO: 123 - DNA, 37 bp, Artificial Sequence
  Description of Artificial Sequence Synthetic primer
gtaaaacgacggccagtatgagcaggaggggaaaagt

SEQ ID NO: 124 - DNA, 37 bp, Artificial Sequence
  Description of Artificial Sequence Synthetic primer
gtaaaacgacggccagtactcacccatcgcatacctc

SEQ ID NO: 125 - DNA, 37 bp, Artificial Sequence
  Description of Artificial Sequence Synthetic primer
gtaaaacgacggccagtttccacaggttcctcagctt

SEQ ID NO: 126 - DNA, 37 bp, Artificial Sequence
  Description of Artificial Sequence Synthetic primer
gtaaaacgacggccagtaaggcaagcttacacccaga

SEQ ID NO: 127 - DNA, 37 bp, Artificial Sequence
  Description of Artificial Sequence Synthetic primer
gtaaaacgacggccagtaatgtccaaatgggactgga

SEQ ID NO: 128 - DNA, 37 bp, Artificial Sequence
  Description of Artificial Sequence Synthetic primer
gtaaaacgacggccagtccccagaaggacactcaaaa

SEQ ID NO: 129 - DNA, 37 bp, Artificial Sequence
  Description of Artificial Sequence Synthetic primer
gtaaaacgacggccagtacttgatagccacaccccag

SEQ ID NO: 130 - DNA, 38 bp, Artificial Sequence
  Description of Artificial Sequence Synthetic primer
gtaaaacgacggccagttgcacaaaaggtagaatgcaa

SEQ ID NO: 131 - DNA, 37 bp, Artificial Sequence
  Description of Artificial Sequence Synthetic primer
gtaaaacgacggccagttttcccattttcacccacat

SEQ ID NO: 132 - DNA, 37 bp, Artificial Sequence
  Description of Artificial Sequence Synthetic primer
gtaaaacgacggccagtagggtcaaagcccacttttt

SEQ ID NO: 133 - DNA, 37 bp, Artificial Sequence
  Description of Artificial Sequence Synthetic primer
gtaaaacgacggccagtgtgtggttatgccacagctt

SEQ ID NO: 134 - DNA, 37 bp, Artificial Sequence
  Description of Artificial Sequence Synthetic primer
gtaaaacgacggccagtaccatacggcttaattcccc

SEQ ID NO: 135 - DNA, 39 bp, Artificial Sequence
  Description of Artificial Sequence Synthetic primer
gtaaaacgacggccagttgtcattccattttgtttctgg

SEQ ID NO: 136 - DNA, 37 bp, Artificial Sequence
  Description of Artificial Sequence Synthetic primer
gtaaaacgacggccagttctggatcaactaggccacc

SEQ ID NO: 137 - DNA, 37 bp, Artificial Sequence
  Description of Artificial Sequence Synthetic primer
gtaaaacgacggccagttcaagcagaggcatgttcag

SEQ ID NO: 138 - DNA, 37 bp, Artificial Sequence
  Description of Artificial Sequence Synthetic primer
gtaaaacgacggccagtaatcccatgaacccttaccc

SEQ ID NO: 139 - DNA, 37 bp, Artificial Sequence
  Description of Artificial Sequence Synthetic primer
gtaaaacgacggccagtatcagtggacaactgctccc

SEQ ID NO: 140 - DNA, 37 bp, Artificial Sequence
  Description of Artificial Sequence Synthetic primer
gtaaaacgacggccagtattggcactagtccagggtg

SEQ ID NO: 141 - DNA, 37 bp, Artificial Sequence
  Description of Artificial Sequence Synthetic primer
gtaaaacgacggccagtcggaactcctgagctgaaag

SEQ ID NO: 142 - DNA, 37 bp, Artificial Sequence
  Description of Artificial Sequence Synthetic primer
gtaaaacgacggccagtgtgctgtgtgctgggattac

SEQ ID NO: 143 - DNA, 37 bp, Artificial Sequence
  Description of Artificial Sequence Synthetic primer
gtaaaacgacggccagtccacaacaaaacaccagtgc

SEQ ID NO: 144 - DNA, 39 bp, Artificial Sequence
  Description of Artificial Sequence Synthetic primer
gtaaaacgacggccagttcaaccggaggaagactaaaaa

SEQ ID NO: 145 - DNA, 39 bp, Artificial Sequence
  Description of Artificial Sequence Synthetic primer
gtaaaacgacggccagtaagcaggctaggctaagctatg

SEQ ID NO: 146 - DNA, 37 bp, Artificial Sequence
  Description of Artificial Sequence Synthetic primer
gtaaaacgacggccagttgtctttgaggcatcactgc

SEQ ID NO: 147 - DNA, 37 bp, Artificial Sequence
  Description of Artificial Sequence Synthetic primer
gtaaaacgacggccagtgtggtttcttctttggctgg

SEQ ID NO: 148 - DNA, 39 bp, Artificial Sequence
  Description of Artificial Sequence Synthetic primer
gtaaaacgacggccagttggaagaaatcggtaagaggtg

SEQ ID NO: 149 - DNA, 37 bp, Artificial Sequence
  Description of Artificial Sequence Synthetic primer
gtaaaacgacggccagtttgcacatctcatggggtta

SEQ ID NO: 150 - DNA, 37 bp, Artificial Sequence
  Description of Artificial Sequence Synthetic primer
gtaaaacgacggccagtttacctgcaattggggcatt

SEQ ID NO: 151 - DNA, 37 bp, Artificial Sequence
  Description of Artificial Sequence Synthetic primer
gtaaaacgacggccagtgccaaagggtgaagaggaat

SEQ ID NO: 152 - DNA, 37 bp, Artificial Sequence
  Description of Artificial Sequence Synthetic primer
gtaaaacgacggccagttcatctggacctgggtcttc

SEQ ID NO: 153 - DNA, 37 bp, Artificial Sequence
  Description of Artificial Sequence Synthetic primer
gtaaaacgacggccagtagcccaacccttgtccttac

SEQ ID NO: 154 - DNA, 37 bp, Artificial Sequence
  Description of Artificial Sequence Synthetic primer
gtaaaacgacggccagtggggacatgatcagctatgg

SEQ ID NO: 155 - DNA, 38 bp, Artificial Sequence
  Description of Artificial Sequence Synthetic primer
caggaaacagctatgaccgccacgcactattccttctc

SEQ ID NO: 156 - DNA, 38 bp, Artificial Sequence
  Description of Artificial Sequence Synthetic primer
caggaaacagctatgacctgtggggtgtttccttttct

SEQ ID NO: 157 - DNA, 38 bp, Artificial Sequence
  Description of Artificial Sequence Synthetic primer
caggaaacagctatgacctagcagtgtgagagcctgga

SEQ ID NO: 158 - DNA, 38 bp, Artificial Sequence
  Description of Artificial Sequence Synthetic primer
caggaaacagctatgacctaaggaactaaagggccggt

SEQ ID NO: 159 - DNA, 38 bp, Artificial Sequence
  Description of Artificial Sequence Synthetic primer
caggaaacagctatgaccgaataagaagaggtgggggc

SEQ ID NO: 160 - DNA, 38 bp, Artificial Sequence
  Description of Artificial Sequence Synthetic primer
caggaaacagctatgaccaccaactaggggaaggagga

SEQ ID NO: 161 - DNA, 37 bp, Artificial Sequence
  Description of Artificial Sequence Synthetic primer
gtaaaacgacggccagtcagagaccagggagatcagc

SEQ ID NO: 162 - DNA, 37 bp, Artificial Sequence
  Description of Artificial Sequence Synthetic primer
gtaaaacgacggccagttgccattggggtaatgattt

SEQ ID NO: 163 - DNA, 37 bp, Artificial Sequence
  Description of Artificial Sequence Synthetic primer
gtaaaacgacggccagtagtgaaggccgaatttctga

SEQ ID NO: 164 - DNA, 37 bp, Artificial Sequence
  Description of Artificial Sequence Synthetic primer
gtaaaacgacggccagtggtaagagctgcggtcaaaa

SEQ ID NO: 165 - DNA, 37 bp, Artificial Sequence
  Description of Artificial Sequence Synthetic primer
gtaaaacgacggccagtggctcctgtttgatgaagga

SEQ ID NO: 166 - DNA, 38 bp, Artificial Sequence
  Description of Artificial Sequence Synthetic primer
caggaaacagctatgacctcttaaaggaagatggcccc

SEQ ID NO: 167 - DNA, 38 bp, Artificial Sequence
  Description of Artificial Sequence Synthetic primer
caggaaacagctatgaccgcgttaggcacaatagaggc

SEQ ID NO: 168 - DNA, 38 bp, Artificial Sequence
  Description of Artificial Sequence Synthetic primer
caggaaacagctatgacctccaagaatcactgcaccaa

SEQ ID NO: 169 - DNA, 38 bp, Artificial Sequence
  Description of Artificial Sequence Synthetic primer
caggaaacagctatgaccacccatccattaaagggtcc

SEQ ID NO: 170 - DNA, 38 bp, Artificial Sequence
  Description of Artificial Sequence Synthetic primer
caggaaacagctatgacctgtcatcattcatcctccca

SEQ ID NO: 171 - DNA, 38 bp, Artificial Sequence
  Description of Artificial Sequence Synthetic primer
caggaaacagctatgacccagagcccagcactagaacc

SEQ ID NO: 172 - DNA, 38 bp, Artificial Sequence
  Description of Artificial Sequence Synthetic primer
caggaaacagctatgaccaaaatagagggccacccaag

SEQ ID NO: 173 - DNA, 38 bp, Artificial Sequence
  Description of Artificial Sequence Synthetic primer
caggaaacagctatgaccagaaggatcaagggggaaaa

SEQ ID NO: 174 - DNA, 38 bp, Artificial Sequence
  Description of Artificial Sequence Synthetic primer
caggaaacagctatgaccggagacatgcaacaccacac

SEQ ID NO: 175 - DNA, 38 bp, Artificial Sequence
  Description of Artificial Sequence Synthetic primer
caggaaacagctatgacccacgttctgctgcaatgact

SEQ ID NO: 176 - DNA, 38 bp, Artificial Sequence
  Description of Artificial Sequence Synthetic primer
caggaaacagctatgaccttctgatccttgggttcctg

SEQ ID NO: 177 - DNA, 38 bp, Artificial Sequence
  Description of Artificial Sequence Synthetic primer
caggaaacagctatgaccggctgtctcaagcaaacctc

SEQ ID NO: 178 - DNA, 38 bp, Artificial Sequence
  Description of Artificial Sequence Synthetic primer
caggaaacagctatgaccgaaggcaggtcctctctcct

SEQ ID NO: 179 - DNA, 38 bp, Artificial Sequence
  Description of Artificial Sequence Synthetic primer
caggaaacagctatgacctgttctgcaggcaatcagtc

SEQ ID NO: 180 - DNA, 38 bp, Artificial Sequence
  Description of Artificial Sequence Synthetic primer
caggaaacagctatgacctggcacagtccagagtgaag

SEQ ID NO: 181 - DNA, 38 bp, Artificial Sequence
  Description of Artificial Sequence Synthetic primer
caggaaacagctatgacccacaagtgggttagtggcct

SEQ ID NO: 182 - DNA, 38 bp, Artificial Sequence
  Description of Artificial Sequence Synthetic primer
caggaaacagctatgaccctggatggagggagtcaaaa

SEQ ID NO: 183 - DNA, 38 bp, Artificial Sequence
  Description of Artificial Sequence Synthetic primer
caggaaacagctatgaccaagtgacccaccagttccag

SEQ ID NO: 184 - DNA, 38 bp, Artificial Sequence
  Description of Artificial Sequence Synthetic primer
caggaaacagctatgaccacactggagcgagatgcttt

SEQ ID NO: 185 - DNA, 38 bp, Artificial Sequence
  Description of Artificial Sequence Synthetic primer
caggaaacagctatgacctatacccaggaaacccctcc

SEQ ID NO: 186 - DNA, 38 bp, Artificial Sequence
  Description of Artificial Sequence Synthetic primer
caggaaacagctatgaccgaggaggggagaattcttgg

SEQ ID NO: 187 - DNA, 38 bp, Artificial Sequence
  Description of Artificial Sequence Synthetic primer
caggaaacagctatgacccctgctataggctgggcttc

SEQ ID NO: 188 - DNA, 39 bp, Artificial Sequence
  Description of Artificial Sequence Synthetic primer
caggaaacagctatgacccttggtgcgtctaagatgagg

SEQ ID NO: 189 - DNA, 38 bp, Artificial Sequence
  Description of Artificial Sequence Synthetic primer
caggaaacagctatgaccctggagctgaccagtgacaa

SEQ ID NO: 190 - DNA, 38 bp, Artificial Sequence
  Description of Artificial Sequence Synthetic primer
caggaaacagctatgacctggacaagaacagcaacgag

SEQ ID NO: 191 - DNA, 38 bp, Artificial Sequence
  Description of Artificial Sequence Synthetic primer
caggaaacagctatgaccccttcaacgacgagttcctg

SEQ ID NO: 192 - DNA, 37 bp, Artificial Sequence
  Description of Artificial Sequence Synthetic primer
caggaaacagctatgacccacctgcagttccagatcg

SEQ ID NO: 193 - DNA, 38 bp, Artificial Sequence
  Description of Artificial Sequence Synthetic primer
caggaaacagctatgaccatcgacatcagcgcctacat

SEQ ID NO: 194 - DNA, 38 bp, Artificial Sequence
  Description of Artificial Sequence Synthetic primer
caggaaacagctatgacccgggagaactctaactcccc

SEQ ID NO: 195 - DNA, 36 bp, Artificial Sequence
  Description of Artificial Sequence Synthetic primer
caggaaacagctatgacccaggctggagcccctgta

SEQ ID NO: 196 - DNA, 36 bp, Artificial Sequence
  Description of Artificial Sequence Synthetic primer
caggaaacagctatgacctcggccgacttctacgag

SEQ ID NO: 197 - DNA, 38 bp, Artificial Sequence; identical to SEQ ID NO: 194
  Description of Artificial Sequence Synthetic primer

SEQ ID NO: 198 - DNA, 38 bp, Artificial Sequence
  Description of Artificial Sequence Synthetic primer
caggaaacagctatgaccctgagtgccgggttgtttat

SEQ ID NO: 199 - DNA, 38 bp, Artificial Sequence
  Description of Artificial Sequence Synthetic primer
caggaaacagctatgacctggatctaagattggccagg

SEQ ID NO: 200 - DNA, 38 bp, Artificial Sequence
  Description of Artificial Sequence Synthetic primer
caggaaacagctatgaccggggctcttaccctgtgaac

SEQ ID NO: 201 - DNA, 38 bp, Artificial Sequence
  Description of Artificial Sequence Synthetic primer
caggaaacagctatgacctgtgtggctcctgagagaga

SEQ ID NO: 202 - DNA, 38 bp, Artificial Sequence
  Description of Artificial Sequence Synthetic primer
caggaaacagctatgacccttcctgtctgcctctgtcc

SEQ ID NO: 203 - DNA, 38 bp, Artificial Sequence
  Description of Artificial Sequence Synthetic primer
caggaaacagctatgacccaacttggtcccgttcttgt

SEQ ID NO: 204 - DNA, 38 bp, Artificial Sequence
  Description of Artificial Sequence Synthetic primer
caggaaacagctatgaccccagttggatccagaaagga

SEQ ID NO: 205 - DNA, 38 bp, Artificial Sequence
  Description of Artificial Sequence Synthetic primer
caggaaacagctatgacccagggtgtgtgggtctagga

SEQ ID NO: 206 - DNA, 38 bp, Artificial Sequence
  Description of Artificial Sequence Synthetic primer
caggaaacagctatgacccggtctttccattccaggta

SEQ ID NO: 207 - DNA, 38 bp, Artificial Sequence
  Description of Artificial Sequence Synthetic primer
caggaaacagctatgacccagggcttaggctctgtgag

SEQ ID NO: 208 - DNA, 38 bp, Artificial Sequence
  Description of Artificial Sequence Synthetic primer
caggaaacagctatgacccctggactcttttctggctg

SEQ ID NO: 209 - DNA, 38 bp, Artificial Sequence
  Description of Artificial Sequence Synthetic primer
caggaaacagctatgaccagcccaaggtcaaggagatt

SEQ ID NO: 210 - DNA, 38 bp, Artificial Sequence
  Description of Artificial Sequence Synthetic primer
caggaaacagctatgaccgaacaagttggagaccaggc

SEQ ID NO: 211 - DNA, 38 bp, Artificial Sequence
  Description of Artificial Sequence Synthetic primer
caggaaacagctatgacccctgtgccaccctcactact

SEQ ID NO: 212 - DNA, 38 bp, Artificial Sequence
  Description of Artificial Sequence Synthetic primer
caggaaacagctatgaccctcctctttgcatcgggtaa

SEQ ID NO: 213 - DNA, 38 bp, Artificial Sequence
  Description of Artificial Sequence Synthetic primer
caggaaacagctatgaccgcctcgtgaccactgtgtaa

SEQ ID NO: 214 - DNA, 38 bp, Artificial Sequence
  Description of Artificial Sequence Synthetic primer
caggaaacagctatgaccctgtcactgttccgggtttt

SEQ ID NO: 215 - DNA, 38 bp, Artificial Sequence
  Description of Artificial Sequence Synthetic primer
caggaaacagctatgaccagggccgtgtttcctagatt

SEQ ID NO: 216 - DNA, 38 bp, Artificial Sequence
  Description of Artificial Sequence Synthetic primer
caggaaacagctatgaccgcgctaatctcttccagagc

SEQ ID NO: 217 - DNA, 38 bp, Artificial Sequence
  Description of Artificial Sequence Synthetic primer
caggaaacagctatgacccattgtgtttgaggcgagtg

SEQ ID NO: 218 - DNA, 38 bp, Artificial Sequence
  Description of Artificial Sequence Synthetic primer
caggaaacagctatgaccgaacagctaaacggccagag

SEQ ID NO: 219 - DNA, 38 bp, Artificial Sequence
  Description of Artificial Sequence Synthetic primer
caggaaacagctatgaccagcggtcaatgatccaaaac

SEQ ID NO: 220 - DNA, 38 bp, Artificial Sequence; identical to SEQ ID NO: 219
  Description of Artificial Sequence Synthetic primer

SEQ ID NO: 221 - DNA, 38 bp, Artificial Sequence
  Description of Artificial Sequence Synthetic primer
caggaaacagctatgaccggtgggggcatattacacag

SEQ ID NO: 222 - DNA, 38 bp, Artificial Sequence
  Description of Artificial Sequence Synthetic primer
caggaaacagctatgaccgatcctcttctctcccccac

SEQ ID NO: 223 - DNA, 38 bp, Artificial Sequence
  Description of Artificial Sequence Synthetic primer
caggaaacagctatgacccttcagcaggctttgttgtg

SEQ ID NO: 224 - DNA, 38 bp, Artificial Sequence
  Description of Artificial Sequence Synthetic primer
caggaaacagctatgacccgcaagggtaacaaaattcg

SEQ ID NO: 225 - DNA, 40 bp, Artificial Sequence
  Description of Artificial Sequence Synthetic primer
caggaaacagctatgaccttttagattttgtggtggatgc

SEQ ID NO: 226 - DNA, 38 bp, Artificial Sequence
  Description of Artificial Sequence Synthetic primer
caggaaacagctatgaccgtgaccctttttgttgcgtt

SEQ ID NO: 227 - DNA, 38 bp, Artificial Sequence
  Description of Artificial Sequence Synthetic primer
caggaaacagctatgaccgtgtgcccaattactgcctt

SEQ ID NO: 228 - DNA, 38 bp, Artificial Sequence
  Description of Artificial Sequence Synthetic primer
caggaaacagctatgaccgtacagcccttgccacgtat

SEQ ID NO: 229 - DNA, 38 bp, Artificial Sequence
  Description of Artificial Sequence Synthetic primer
caggaaacagctatgacccttgggggtgggagagtatt

SEQ ID NO: 230 - DNA, 38 bp, Artificial Sequence
  Description of Artificial Sequence Synthetic primer
caggaaacagctatgaccatttgtagcttcccgcagaa

SEQ ID NO: 231 - DNA, 38 bp, Artificial Sequence
  Description of Artificial Sequence Synthetic primer
caggaaacagctatgacccccagcatctagcagtgtca

SEQ ID NO: 232 - DNA, 38 bp, Artificial Sequence
  Description of Artificial Sequence Synthetic primer
caggaaacagctatgaccgccgattggatttgagttgt

SEQ ID NO: 233 - DNA, 38 bp, Artificial Sequence
  Description of Artificial Sequence Synthetic primer
caggaaacagctatgacctgccctgatgttgacatttt

SEQ ID NO: 234 - DNA, 38 bp, Artificial Sequence
  Description of Artificial Sequence Synthetic primer
caggaaacagctatgacctgcgcatcagttttacttgc

SEQ ID NO: 235 - DNA, 40 bp, Artificial Sequence
  Description of Artificial Sequence Synthetic primer
caggaaacagctatgaccttcttgataacaccatgcacaa

SEQ ID NO: 236 - DNA, 38 bp, Artificial Sequence
  Description of Artificial Sequence Synthetic primer
caggaaacagctatgaccttctgcttcccagtgctctt

SEQ ID NO: 237 - DNA, 38 bp, Artificial Sequence
  Description of Artificial Sequence Synthetic primer
caggaaacagctatgacctgcttcctttgcctaacacc

SEQ ID NO: 238 - DNA, 38 bp, Artificial Sequence
  Description of Artificial Sequence Synthetic primer
caggaaacagctatgacctgcttattggtgagaggggt

SEQ ID NO: 239 - DNA, 40 bp, Artificial Sequence
  Description of Artificial Sequence Synthetic primer
caggaaacagctatgaccggctacagcttaaggttgtcct

SEQ ID NO: 240 - DNA, 38 bp, Artificial Sequence; identical to SEQ ID NO: 76
  Description of Artificial Sequence Synthetic primer

SEQ ID NO: 241 - DNA, 37 bp, Artificial Sequence
  Description of Artificial Sequence Synthetic primer
caggaaacagctatgaccgcagcacaaatgagcacct

SEQ ID NO: 242 - DNA, 38 bp, Artificial Sequence
  Description of Artificial Sequence Synthetic primer
caggaaacagctatgacctccatcaccggtacctccta

SEQ ID NO: 243 - DNA, 38 bp, Artificial Sequence
  Description of Artificial Sequence Synthetic primer
caggaaacagctatgaccgtgaccggctcctcagataa

SEQ ID NO: 244 - DNA, 39 bp, Artificial Sequence
  Description of Artificial Sequence Synthetic primer
caggaaacagctatgacctcattgtcgttttaaccctgc

SEQ ID NO: 245 - DNA, 38 bp, Artificial Sequence
  Description of Artificial Sequence Synthetic primer
caggaaacagctatgaccagaggctggctgtgtgaact

SEQ ID NO: 246 - DNA, 37 bp, Artificial Sequence
  Description of Artificial Sequence Synthetic primer
caggaaacagctatgaccgtgggtttgcccttcagat

SEQ ID NO: 247 - DNA, 38 bp, Artificial Sequence
  Description of Artificial Sequence Synthetic primer
caggaaacagctatgaccggtgtactcagagccttcgc

SEQ ID NO: 248 - DNA, 38 bp, Artificial Sequence
  Description of Artificial Sequence Synthetic primer
caggaaacagctatgaccattctggttgaaagatggcg

SEQ ID NO: 249 - DNA, 40 bp, Artificial Sequence
  Description of Artificial Sequence Synthetic primer
caggaaacagctatgaccctgacacctagctgtgatcctg

SEQ ID NO: 250 - DNA, 39 bp, Artificial Sequence
  Description of Artificial Sequence Synthetic primer
caggaaacagctatgaccaaagccacatggctagaaaaa

SEQ ID NO: 251 - DNA, 38 bp, Artificial Sequence
  Description of Artificial Sequence Synthetic primer
caggaaacagctatgacctggcaaacctatcaaaaggg

SEQ ID NO: 252 - DNA, 38 bp, Artificial Sequence
  Description of Artificial Sequence Synthetic primer
caggaaacagctatgacctgttcagcataccatgcaaa

SEQ ID NO: 253 - DNA, 38 bp, Artificial Sequence
  Description of Artificial Sequence Synthetic primer
caggaaacagctatgaccggtgcttagtggccatttgt

SEQ ID NO: 254 - DNA, 38 bp, Artificial Sequence
  Description of Artificial Sequence Synthetic primer
caggaaacagctatgacccgtctgcagtcaactggaat

SEQ ID NO: 255 - DNA, 38 bp, Artificial Sequence
  Description of Artificial Sequence Synthetic primer
caggaaacagctatgaccactccagcctaggggaaaaa

SEQ ID NO: 256 - DNA, 38 bp, Artificial Sequence
  Description of Artificial Sequence Synthetic primer
caggaaacagctatgaccgggacaaaccagataggcag

SEQ ID NO: 257 - DNA, 38 bp, Artificial Sequence
  Description of Artificial Sequence Synthetic primer
caggaaacagctatgaccattaatccggtgtttttgcg

SEQ ID NO: 258 - DNA, 38 bp, Artificial Sequence
  Description of Artificial Sequence Synthetic primer
caggaaacagctatgaccgtctctgttgctgccggtat

SEQ ID NO: 259 - DNA, 38 bp, Artificial Sequence
  Description of Artificial Sequence Synthetic primer
caggaaacagctatgaccggattttgctggctcagaga

SEQ ID NO: 260 - DNA, 38 bp, Artificial Sequence
  Description of Artificial Sequence Synthetic primer
caggaaacagctatgacccgagcagtacacttcaccca

SEQ ID NO: 261 - DNA, 38 bp, Artificial Sequence
  Description of Artificial Sequence Synthetic primer
caggaaacagctatgacctgaaaagtggctgaaacgtg

SEQ ID NO: 262 - DNA, 38 bp, Artificial Sequence
  Description of Artificial Sequence Synthetic primer
caggaaacagctatgaccttgggctttagatcacaggg

SEQ ID NO: 263 - DNA, 38 bp, Artificial Sequence; identical to SEQ ID NO: 262
  Description of Artificial Sequence Synthetic primer

SEQ ID NO: 264 - DNA, 38 bp, Artificial Sequence
  Description of Artificial Sequence Synthetic primer
caggaaacagctatgaccgcttgcaaatgccttgaaat

SEQ ID NO: 265 - DNA, 38 bp, Artificial Sequence
  Description of Artificial Sequence Synthetic primer
caggaaacagctatgaccccggcccagtgtatgtagtt

SEQ ID NO: 266 - DNA, 38 bp, Artificial Sequence
  Description of Artificial Sequence Synthetic primer
caggaaacagctatgaccatgatgcttgaggggaacac

SEQ ID NO: 267 - DNA, 38 bp, Artificial Sequence
  Description of Artificial Sequence Synthetic primer
caggaaacagctatgaccgcaacctgaccagggttaaa

SEQ ID NO: 268 - DNA, 38 bp, Artificial Sequence
  Description of Artificial Sequence Synthetic primer
caggaaacagctatgaccctgtatccccctgaagtcca

SEQ ID NO: 269 - DNA, 39 bp, Artificial Sequence
  Description of Artificial Sequence Synthetic primer
caggaaacagctatgacctgccaacaatgttttacctca

SEQ ID NO: 270 - DNA, 41 bp, Artificial Sequence
  Description of Artificial Sequence Synthetic primer
caggaaacagctatgacctctcactcgataatctggatgac

SEQ ID NO: 271 - DNA, 40 bp, Artificial Sequence
  Description of Artificial Sequence Synthetic primer
caggaaacagctatgaccgaaacccaaaatctgttttcca

SEQ ID NO: 272 - DNA, 38 bp, Artificial Sequence
  Description of Artificial Sequence Synthetic primer
caggaaacagctatgacctaaaacccattgcttttggc

SEQ ID NO: 273 - DNA, 38 bp, Artificial Sequence
  Description of Artificial Sequence Synthetic primer
caggaaacagctatgaccgccataaggccttttccttc

SEQ ID NO: 274 - DNA, 39 bp, Artificial Sequence
  Description of Artificial Sequence Synthetic primer
caggaaacagctatgaccttttgacgctgtgtacattgg

SEQ ID NO: 275 - DNA, 38 bp, Artificial Sequence
  Description of Artificial Sequence Synthetic primer
caggaaacagctatgacctaaaacgggaaagtgccatc

SEQ ID NO: 276 - DNA, 37 bp, Artificial Sequence
  Description of Artificial Sequence Synthetic primer
caggaaacagctatgacccacgcgctaccacacctac

SEQ ID NO: 277 - DNA, 38 bp, Artificial Sequence
  Description of Artificial Sequence Synthetic primer
caggaaacagctatgaccatcctcgtcctcttgggagt

SEQ ID NO: 278 - DNA, 38 bp, Artificial Sequence
  Description of Artificial Sequence Synthetic primer
caggaaacagctatgaccaccctggtacataggccaca

SEQ ID NO: 279 - DNA, 38 bp, Artificial Sequence
  Description of Artificial Sequence Synthetic primer
caggaaacagctatgaccggaagggaagggaaatcttg

SEQ ID NO: 280 - DNA, 38 bp, Artificial Sequence
  Description of Artificial Sequence Synthetic primer
caggaaacagctatgaccggaaagacaagaaaagcccc

SEQ ID NO: 281 - DNA, 38 bp, Artificial Sequence
  Description of Artificial Sequence Synthetic primer
caggaaacagctatgaccggtaacttgtgctgaagggc

SEQ ID NO: 282 - DNA, 39 bp, Artificial Sequence
  Description of Artificial Sequence Synthetic primer
caggaaacagctatgacccattgctattcctctgcaacc

SEQ ID NO: 283 - DNA, 38 bp, Artificial Sequence
  Description of Artificial Sequence Synthetic primer
caggaaacagctatgaccgcagaaccagaacgttttcc

SEQ ID NO: 284 - DNA, 38 bp, Artificial Sequence
  Description of Artificial Sequence Synthetic primer
caggaaacagctatgaccaaccacgtgcataaggaaca

SEQ ID NO: 285 - DNA, 38 bp, Artificial Sequence
  Description of Artificial Sequence Synthetic primer
caggaaacagctatgacctttgggcctcataaacaacc

SEQ ID NO: 286 - DNA, 38 bp, Artificial Sequence
  Description of Artificial Sequence Synthetic primer
caggaaacagctatgacctggttgactgctttcacctg

SEQ ID NO: 287 - DNA, 38 bp, Artificial Sequence
  Description of Artificial Sequence Synthetic primer
caggaaacagctatgaccgaggtatgcgatgggtgagt

SEQ ID NO: 288 - DNA, 38 bp, Artificial Sequence
  Description of Artificial Sequence Synthetic primer
caggaaacagctatgacctggtgtggtagtggcagaaa

SEQ ID NO: 289 - DNA, 38 bp, Artificial Sequence
  Description of Artificial Sequence Synthetic primer
caggaaacagctatgaccagatagtgctgtgttggggg

SEQ ID NO: 290 - DNA, 38 bp, Artificial Sequence
  Description of Artificial Sequence Synthetic primer
caggaaacagctatgaccgagaagtgcacctggtgtga

SEQ ID NO: 291 - DNA, 38 bp, Artificial Sequence
  Description of Artificial Sequence Synthetic primer
caggaaacagctatgaccggttccaccttaattggcct

SEQ ID NO: 292 - DNA, 38 bp, Artificial Sequence
  Description of Artificial Sequence Synthetic primer
caggaaacagctatgaccactggccctgacatttcaac

SEQ ID NO: 293 - DNA, 38 bp, Artificial Sequence
  Description of Artificial Sequence Synthetic primer
caggaaacagctatgacccaaattgctgccagactcaa

SEQ ID NO: 294 - DNA, 38 bp, Artificial Sequence
  Description of Artificial Sequence Synthetic primer
caggaaacagctatgaccttcccccaactcatgaagac

SEQ ID NO: 295 - DNA, 38 bp, Artificial Sequence
  Description of Artificial Sequence Synthetic primer
caggaaacagctatgaccacgtgggatttcacacaaca

SEQ ID NO: 296 - DNA, 38 bp, Artificial Sequence
  Description of Artificial Sequence Synthetic primer
caggaaacagctatgaccacccaattctcagggtcaga

SEQ ID NO: 297 - DNA, 38 bp, Artificial Sequence
  Description of Artificial Sequence Synthetic primer
caggaaacagctatgacctgaggccatgtggttacaga

SEQ ID NO: 298 - DNA, 40 bp, Artificial Sequence
  Description of Artificial Sequence Synthetic primer
caggaaacagctatgaccccaaagaggaagtttttgttgc

SEQ ID NO: 299 - DNA, 39 bp, Artificial Sequence
  Description of Artificial Sequence Synthetic primer
caggaaacagctatgacctgttacaattgctgccaatga

SEQ ID NO: 300 - DNA, 38 bp, Artificial Sequence
  Description of Artificial Sequence Synthetic primer
caggaaacagctatgaccctgctaagctgtcctcagcc

SEQ ID NO: 301 - DNA, 38 bp, Artificial Sequence
  Description of Artificial Sequence Synthetic primer
caggaaacagctatgaccgggggcaaaaccaaaataat

SEQ ID NO: 302 - DNA, 38 bp, Artificial Sequence
  Description of Artificial Sequence Synthetic primer
caggaaacagctatgacctatttccaaaccttggctgg

SEQ ID NO: 303 - DNA, 38 bp, Artificial Sequence
  Description of Artificial Sequence Synthetic primer
caggaaacagctatgaccaccagacctcatcgttgtcc

SEQ ID NO: 304 - DNA, 38 bp, Artificial Sequence
  Description of Artificial Sequence Synthetic primer
caggaaacagctatgaccatgaaacgcaggtaagtggg

SEQ ID NO: 305 - DNA, 38 bp, Artificial Sequence
  Description of Artificial Sequence Synthetic primer
caggaaacagctatgaccactgtgacctttccccactg

SEQ ID NO: 306 - DNA, 38 bp, Artificial Sequence
  Description of Artificial Sequence Synthetic primer
caggaaacagctatgaccgcaggagagttgcttgaacc

SEQ ID NO: 307 - DNA, 38 bp, Artificial Sequence
  Description of Artificial Sequence Synthetic primer
caggaaacagctatgaccgtgccaggagctgttctagg

SEQ ID NO: 308 - DNA, 38 bp, Artificial Sequence
  Description of Artificial Sequence Synthetic primer
caggaaacagctatgaccaaagcattggtcagggaaaa

SEQ ID NO: 309 - DNA, 38 bp, Artificial Sequence
  Description of Artificial Sequence Synthetic primer
caggaaacagctatgaccatcagccaagattgcaccat

SEQ ID NO: 310 - DNA, 38 bp, Artificial Sequence
  Description of Artificial Sequence Synthetic primer
caggaaacagctatgaccaaggaccagaccagctttca

SEQ ID NO: 311 - DNA, 38 bp, Artificial Sequence
  Description of Artificial Sequence Synthetic primer
caggaaacagctatgaccgcgcacagaggaagagaatc

SEQ ID NO: 312 - DNA, 38 bp, Artificial Sequence
  Description of Artificial Sequence Synthetic primer
caggaaacagctatgacccaagggtggttgggagtaga

SEQ ID NO: 313 - DNA, 37 bp, Artificial Sequence
  Description of Artificial Sequence Synthetic primer
caggaaacagctatgaccctgcttgccacaggtctcc

SEQ ID NO: 314 - DNA, 38 bp, Artificial Sequence
  Description of Artificial Sequence Synthetic primer
caggaaacagctatgaccagtcacagcacatgacggag

SEQ ID NO: 315 - DNA, 41 bp, Artificial Sequence
  Description of Artificial Sequence Synthetic primer
caggaaacagctatgaccgcaggctaggctaagctatgatg

SEQ ID NO: 316 - DNA, 38 bp, Artificial Sequence
  Description of Artificial Sequence Synthetic primer
caggaaacagctatgaccgtaaggacaagggttgggct

SEQ ID NO: 317 - DNA, 38 bp, Artificial Sequence
  Description of Artificial Sequence Synthetic primer
caggaaacagctatgacccccctctgagtcaggaaaca

SEQ ID NO: 318 - DNA, 38 bp, Artificial Sequence
  Description of Artificial Sequence Synthetic primer
caggaaacagctatgacccagccattcttttcctgctc

SEQ ID NO: 319 - DNA, 38 bp, Artificial Sequence
  Description of Artificial Sequence Synthetic primer
caggaaacagctatgacctccttaaagccccaagaggt

SEQ ID NO: 320 - DNA, 37 bp, Artificial Sequence
  Description of Artificial Sequence Synthetic primer
gtaaaacgacggccagtgggaaatctaagggtgaggc

SEQ ID NO: 321 - DNA, 37 bp, Artificial Sequence
  Description of Artificial Sequence Synthetic primer
gtaaaacgacggccagtgttggggatcatcctaccct

SEQ ID NO: 322 - DNA, 37 bp, Artificial Sequence
  Description of Artificial Sequence Synthetic primer
gtaaaacgacggccagtggagtgtgaatgggagtggt

SEQ ID NO: 323 - DNA, 37 bp, Artificial Sequence
  Description of Artificial Sequence Synthetic primer
gtaaaacgacggccagtccatcattccctcctgattg

SEQ ID NO: 324 - DNA, 37 bp, Artificial Sequence
  Description of Artificial Sequence Synthetic primer
gtaaaacgacggccagtggcttttcactggattctgg

SEQ ID NO: 325 - DNA, 37 bp, Artificial Sequence
  Description of Artificial Sequence Synthetic primer
gtaaaacgacggccagtctgtgcagagatcagtggga

SEQ ID NO: 326 - DNA, 37 bp, Artificial Sequence
  Description of Artificial Sequence Synthetic primer
gtaaaacgacggccagtgactgctaggggaatgcaaa

SEQ ID NO: 327 - DNA, 38 bp, Artificial Sequence
  Description of Artificial Sequence Synthetic primer
caggaaacagctatgacccaaggtcacatccagggact

SEQ ID NO: 328 - DNA, 38 bp, Artificial Sequence
  Description of Artificial Sequence Synthetic primer
caggaaacagctatgacctccaaggcctgtacaaggag

SEQ ID NO: 329 - DNA, 37 bp, Artificial Sequence
  Description of Artificial Sequence Synthetic primer
caggaaacagctatgaccctacagcagccagagcagc

SEQ ID NO: 330 - DNA, 38 bp, Artificial Sequence
  Description of Artificial Sequence Synthetic primer
caggaaacagctatgaccgtaaggagttcaaggcagcg

(19) United States
(12) Patent Application Publication
(10) Pub. No.: US 2015/0031641 A1
(43) Pub. Date: Jan. 29, 2015

(54) METHODS AND COMPOSITIONS FOR THE DIAGNOSIS, PROGNOSIS AND TREATMENT OF ACUTE MYELOID LEUKEMIA

(71) Applicant: MEMORIAL SLOAN-KETTERING CANCER CENTER, New York, NY (US)

(72) Inventors: Ross L. Levine, New York, NY (US); Omar Abdel-Waheb, New York, NY (US); Jay P. Patel, Sayville, NY (US); Mithat Gonen, New York, NY (US)

(73) Assignee: MEMORIAL SLOAN-KETTERING CANCER CENTER, New York, NY (US)

(21) Appl. No.: 14/384,580

(22) PCT Filed: Mar. 11, 2013
(86) PCT No.: PCT/US13/30208

§ 371 Date: Sep. 11, 2014

Related U.S. Application Data
(60) Provisional application No. 61/609,723, filed on Mar. 12, 2012.

(51) Int. Cl. C12Q 1/68; A61K 31/704

(52) U.S. Cl. CPC C12Q 1/6886; A61K 31/704; C12Q 2600/118; C12Q 2600/156
USPC 514/34; 435/6.11

(57) ABSTRACT

Gene mutations are associated with the progression of acute myeloid leukemia (AML). The invention relates to methods and systems for evaluating the progression of AML based on these gene mutations. The present invention also relates to methods and compositions for treating AML patients by modulating the expression or activity of certain genes involved in AML progression and/or their encoded proteins. The invention further relates to methods and compositions for determining the responsiveness of an AML patient to induction chemotherapy therapy.

### CROSS REFERENCE TO RELATED APPLICATION

This application is a national phase filing under 35 U.S.C. §371 of PCT International Application PCT/US2013/030208, filed Mar. 11, 2013, and published under PCT Article 21(2) in English as WO 2013/138237 A1 on Sep. 19, 2013. This application also claims priority to U.S. provisional patent application No. 61/609,723 filed Mar. 12, 2012; the entire contents of these applications are incorporated by reference.

### FEDERALLY-SPONSORED RESEARCH OR DEVELOPMENT

This invention was made with Government support under contract U54CA143798-01 awarded by the National Cancer Institute Physical Sciences Oncology Center. The U.S. Government has certain rights in this invention.

### SEQUENCE LISTING

The instant application contains a Sequence Listing, in computer readable form that is hereby incorporated by reference in its entirety into the present disclosure. The sequence listing file, created on Mar. 7, 2013 and updated on Sep. 10, 2014 is named 3314022A_SequenceListing.txt and is 73.6 KB in size.

### FIELD OF INVENTION

The invention described herein relates to methods useful in the diagnosis, treatment and management of cancers. The field of the present invention is molecular biology, genetics, oncology, clinical diagnostics, bioinformatics. In particular, the field of the present invention relates to the diagnosis, prognosis and treatment of blood cancer.

### BACKGROUND OF THE INVENTION

The following description of the background of the invention is provided simply as an aid in understanding the invention and is not admitted to describe or constitute prior art to the invention.

After cardiovascular disease, cancer is the leading cause of death in the developed world. In the United States alone, over one million people are diagnosed with cancer each year, and over 500,000 people die each year as a result of it. It is estimated that 1 in 3 Americans will develop cancer during their lifetime, and one in five will die from cancer. Further, it is predicted that cancer may surpass cardiovascular diseases as the number one cause of death within 5 years. As such, considerable efforts are directed at improving treatment and diagnosis of this disease.

Most cancer patients are not killed by their primary tumor. They succumb instead to metastases: multiple widespread tumor colonies established by malignant cells that detach themselves from the original tumor and travel through the body, often to distant sites. In the case of blood cancers, there are four types depending upon the origin of the affected cells and the course of the disease. The latter criterion classifies the types into either acute or chronic. The former criterion further divides the types as lymphoblastic or lymphocytic leukemias and myeloid or myelogenous leukemias. These malignancies have varying prognoses, depending on the patient and the specifics of the condition.

Blood primarily consists of red blood cells (RBC), white blood cells (WBC) and platelets. The red blood cells' function is to carry oxygen to the body, the white blood cells protect our body, and platelets help clot the blood after injury. Irrespective of the types of the disease, any abnormality in these cell types leads to blood cancer. The main categories of blood cancer include Acute Lymphocytic or Lymphoblastic Leukemias (ALL), Chronic Lymphocytic or Lymphoblastic Leukemias (CLL), Acute Myelogenous or Myeloid Leukemias (AML), and Chronic Myelogenous or Myeloid Leukemias (CML).

In the case of leukemia, the bone marrow and the blood itself are attacked, such that the cancer interferes with the body's ability to make blood. In the patient, this most commonly manifests itself in the form of fatigue, anemia, weakness, and bone pain. It is diagnosed with a blood test in which specific types of blood cells are counted. Treatment for leukemia usually includes chemotherapy and radiation to kill the cancer, and measures like stem cell transplants are sometimes required. As outlined above, there are several different types of leukemia, with myeloid leukemia being usually subdivided into two groups: Acute Myeloid Leukemia (AML) and Chronic Myeloid Leukemia (CML).

AML is characterized by an increase in the number of myeloid cells in the marrow and an arrest in their maturation, frequently resulting in hematopoietic insufficiency. In the United States, the annual incidence of AML is approximately 2.4 per 100,000 and it increases progressively with age to a peak of 12.6 per 100,000 adults 65 years of age or older. Despite improved therapeutic approaches, prognosis of AML is very poor around the globe. Even in the United States, the five-year survival rate among patients who are less than 65 years of age is less than 40%. During approximately the last decade this value was 15. Similarly, the prognosis of CML is also very poor in spite of advancement of clinical medicine.

Acute myeloid leukemia (AML) is a heterogeneous disorder that includes many entities with diverse genetic abnormalities and clinical features. The pathogenesis has only been fully delineated for relatively few types of leukemia. Patients with intermediate and poor risk cytogenetics represent the majority of AML; chemotherapy based regimens fail to cure most of these patients, and stem cell transplantation is frequently the treatment choice. Since allogeneic stem cell transplantation is not an option for many patients with high risk leukemia, there is a need to improve our understanding of the biology of these leukemias and to develop improved therapies.

Since not enough is known of the etiology, cell physiology and molecular genetics of acute myeloid leukemia, the development of effective new agents and novel treatment and/or prognostic methods against myeloid leukemia, and in particular acute myeloid leukemia, is a major focal point today in translational oncology research. However, there are inherent difficulties in the diagnosis and treatment of cancer including, among other things, the existence of many different subgroups of cancer and the concomitant variation in appropriate treatment strategies to maximize the likelihood of positive patient outcome.

One relatively new approach is to investigate the genetic profile of cancer, an effort aimed at identifying perturbations in genes that lead to the malignant phenotype. These gene profiles, including gene expression and mutations, provide valuable information about biological processes in normal and disease cells. However, cancers differ widely in their genetic “signature,” leading to difficulty in diagnosis and treatment, as well as in the development of effective therapeutics.

Increasingly, genetic signatures are being identified and exploited as tools for disease detection as well as for prognosis and prospective assessment of therapeutic success. Genetic profiling of cancers, including leukemias, may provide a more effective approach to cancer management and/or treatment. In the context of the present invention, specific genes and gene products, and groups of genes and their gene products, involved in progression of meyoloblasts into a malignant phenotype is still largely unknown. As such, there is a great need in the art to better understand the genetic profile of acute myeloid leukemia, in an effort to provide improved therapeutics, and tools for the treatment, therapy and diagnosis of acute myeloid leukemia and other cancers of the blood. There is a great need for improved methods for diagnosing acute myeloid leukemia and for determining the prognosis of patients afflicted by this disease.

### SUMMARY OF THE INVENTION

One aspect of the present disclosure is a method of predicting survival of a patient with acute myeloid leukemia, said method comprising: analyzing a genetic sample isolated from the patient for the presence of cytogenetic abnormalities and a mutation in at least one of FLT3, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2 genes; and (i) predicting poor survival of the patient if a mutation is present in at least one of FLT3, MLL-PTD, ASXL1 and PHF6 genes, or (ii) predicting favorable survival of the patient if a mutation is present in IDH2R140 and/or a mutation is present in CEBPA. In one embodiment, the method further comprises, predicting intermediate survival of the patient with cytogenetically-defined intermediate risk AML if: (i) no mutation is present in any of FLT3-ITD, TET2, MLL-PTD, DNMT3A, ASXL1 or PHF6 genes, (ii) a mutation in CEBPA is present in the presence of a FLT3-ITD mutation, or (iii) a mutation is present in FLT3-ITD but trisomy 8 is absent. In another embodiment, the method further comprises predicting unfavorable survival of the patient if (i) a mutation in TET2, ASXL1, or PHF6 or an MLL-PTD is present in a patient without the FLT3-ITD mutation, or (ii) the patient has a FLT3-ITD mutation and a mutation in TET2, DNMT3A, MLL-PTD or trisomy 8.

Unless context demands otherwise, in this and any other aspect of the invention, the mutation may be any one of those described in the Table below entitled “Specific somatic mutations identified in the sequencing of 18 genes in AML patients, and the nature of these mutations”.

In one embodiment, the sample is DNA and it is extracted from bone marrow or blood from the patient. The extraction may be historical, and in all embodiments herein the sample may be utilized in the invention as a previously provided sample i.e. the extraction or isolation is not part of the method per se. In a related embodiment, the genetic sample is DNA isolated from mononuclear cells (MNC) from the patient. In one embodiment, poor or unfavorable survival of the patient is survival of less than or equal to about 10 months. In another embodiment, intermediate survival the patient is survival of about 18 months to about 30 months. In another embodiment, favorable survival of the patient is survival of about 32 months or more.

In one aspect, the present disclosure is a method of predicting survival of a patient with acute myeloid leukemia, said method comprising, assaying a genetic sample from the patient's blood or bone marrow for the presence of a mutation in at least one of genes FLT3, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2 in said sample; and predicting a poor survival of the patient if a mutation is present in at least one of genes FLT3-ITD, MLL-PTD, ASXL1, PHF6; or predicting a favorable survival of the patient if a mutation is present in CEBPA or a mutation is present in IDH2 at R140. In one embodiment, the patient is characterized as intermediate-risk on the basis of cytogenetic analysis.

In one embodiment, amongst patients with cytogenetically-defined intermediate-risk acute myeloid leukemia who have FLT3-ITD mutation, at least one of the following: trisomy 8 or a mutation in TET2, DNMT3A, or the MLL-PTD are associated with an adverse outcome and poor overall survival of the patient. In another embodiment, amongst patients with cytogenetically-defined intermediate-risk acute myeloid leukemia who have a mutation in FLT3-ITD gene, a mutation in CEBPA gene is associated with improved outcome and overall survival of the patient. In one embodiment, in a cytogenetically-defined intermediate risk AML patient with both IDH1/IDH2 and NPM1 mutations, the overall survival is improved compared to NPM1-mutant patients wild-type for both IDH1 and IDH2. In one embodiment, amongst patients acute myeloid leukemia, IDH2R140 mutations are associated with improved overall survival. Poor or unfavorable survival (adverse risk) of the patient, in one example, is survival of less than or equal to about 10 months. Favorable survival of the patient, in one example, is survival of about 32 months or more.

One aspect of the present disclosure is a method of predicting survival of a patient with acute myeloid leukemia, said method comprising assaying a genetic sample from the patient's blood or bone marrow for the presence of a mutation in genes ASXL1 and WT1; and determining the patient has or will develop primary refractory acute myeloid leukemia if mutated ASXL1 and WT1 genes are detected.

Another aspect of the present disclosure is a method of determining responsiveness of a patient with acute myeloid leukemia to high dose therapy, said method comprising analyzing a genetic sample isolated from the patient for the presence of a mutation in genes DNMT3A, and NPM1, and for the presence of a MLL translocation; and (i) identifying the patient as one who will respond to high dose therapy if a mutation in DNMT3A or NPM1 or an MLL translocation are present, or (ii) identifying the patient as one who will not respond to high dose therapy in the absence of mutations in DNMT3A or NPM1 or an MLL translocation.

In one embodiment, the therapy comprises the administration of anthracycline. In one example, the anthracycline is selected from the group consisting of Daunorubicin, Doxorubicin, Epirubicin, Idarubicin, Mitoxantrone, and Adriamycin. In a particular example, the anthracycline is Daunorubicin. In one embodiment, the high dose administration is Daunorubicin administered at 60 mg per square meter of body-surface area (60 mg/m2), or higher, daily for three days. In a particular embodiment, the high dose administration is Daunorubicin administered at about 90 mg per square meter of body-surface area (90 mg/m2), daily for three days. In one embodiment, the high dose daunorubicin is administered at about 70 mg/m2 to about 140 mg/m2. In a particular embodiment, the high dose daunorubicin is administered at about 70 mg/m2 to about 120 mg/m2. In a related embodiment, this high dose administration is given each day for three days, that is for example a total of about 300 mg/m2 over the three days (3×100 mg/m2). In another example, this high dose is administered daily for 2-6 days. In other clinical situations, an intermediate daunorubicin dose is administered. In one embodiment, the intermediate dose daunorubicin is administered at about 60 mg/m2. In one embodiment, the intermediate dose daunorubicin is administered at about 30 mg/m2 to about 70 mg/m2. Additionally, the related anthracycline idarubicin, in one embodiment, is administered at from about 4 mg/m2 to about 25 mg/m2. In one embodiment, the high dose idarubicin is administered at about 10 mg/m2 to 20 mg/m2. In one embodiment, the intermediate dose idarubicin is administered at about 6 mg/m2 to about 10 mg/m2. In a particular embodiment, idarubicin is administered at a dose of about 8 mg/m2 daily for five days. In another example, this intermediate dose is administered daily for 2-10 days.

In one aspect, the present disclosure is a method of predicting whether a patient suffering from acute myeloid leukemia will respond better to high dose chemotherapy than to standard dose chemotherapy, the method comprising: obtaining a DNA sample obtained from the patient's blood or bone marrow; determining the mutational status of genes DNMT3A and NPM1, and the presence of a MLL translocation; and predicting that the subject will be more responsive to high dose chemotherapy than standard dose chemotherapy where the sample is positive for a mutation in DNMT3A or NPM1 or an MLL translocation, or predicting that the subject will be non-responsive to high dose chemotherapy compared to standard dose chemotherapy where the sample is wild type with no mutations in DNMT3a or NPM1 genes and no translocation in MLL.

One aspect of the present disclosure is a method of screening a patient with acute myeloid leukemia for responsiveness to treatment with high dose of Daunorubicin or a pharmaceutically acceptable salt, solvate, or hydrate thereof, comprising: obtaining a genetic sample comprising an acute myeloid leukemic cell from said individual; and assaying the sample and detecting the presence of a mutation in DNMT3A or NPM1 or an MLL translocation; and correlating a finding of a mutation in DNMT3A or NPM1 or an MLL translocation, as compared to wild type controls where there is no mutation, with said acute myeloid leukemia patient being more sensitive to high dose treatment with Daunorubicin or a pharmaceutically acceptable salt, solvate, or hydrate thereof. In one embodiment, the method further comprises predicting the patient is at a lower risk of relapse of acute myeloid leukemia following chemotherapy if a mutation in DNMT3A or NPM1 or an MLL translocation is detected.

Another aspect of the present disclosure is a method of determining whether a human has an increased genetic risk for developing or developing a relapse of acute myeloid leukemia, comprising, analyzing a genetic sample isolated from the human's blood or bone marrow for the presence of a mutation in at least one gene from FLT3, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2; and determining the individual with cytogenetically-defined intermediate risk AML has an increased genetic risk for developing or developing a relapse of acute myeloid leukemia, relative to a control human with no such gene mutations in said genes, when: (i) a mutation in at least one of TET2, MLL-PTD, ASXL1 and PHF6 genes is detected when the patient has no FLT3-ITD mutation, or (ii) a mutation in at least one of TET2, MLL-PTD, and DNMT3A genes or trisomy 8 is detected when the patient has a FLT3-ITD mutation.

In one aspect, the present disclosure is a method for preparing a personalized genomics profile for a patient with acute myeloid leukemia, comprising: subjecting mononuclear cells extracted from a bone marrow aspirate or blood sample from the patient to gene mutational analysis; assaying the sample and detecting the presence of a cytoegentic abnormality and one or more mutations in a gene selected from the group consisting of FLT3, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2 in said cells; and generating a report of the data obtained by the gene mutation analysis, wherein the report comprises a prediction of the likelihood of survival of the patient or a response to therapy.

In one aspect, the disclosure is a kit for determining treatment of a patient with AML, the kit comprising means for detecting a mutation in at least one gene selected from the group consisting of ASXL1, DNMT3A, NPM1, PHF6, WT1, TP53, EZH2, CEBPA, TET2, RUNX1, PTEN, FLT3, HRAS, KRAS, NRAS, KIT, IDH1, and IDH2; and instructions for recommended treatment based on the presence of a mutation in one or more of said genes. In one example, the instructions for recommended treatment for the patient based on the presence of a DNMT3A or NPM1 mutation or MLL translocation indicate high-dose daunorubicin as the recommended treatment.

One aspect of the present disclosure is a method of treating, preventing or managing acute myeloid leukemia in a patient, comprising, analyzing a genetic sample isolated from the patient for the presence of a mutation in genes DNMT3A, and NPM1, and for the presence of a MLL translocation; identifying the patient as one who will respond to high dose chemotherapy better than standard dose chemotherapy if a mutation in DNMT3A or NPM1 or a MLL translocation are present; and administering high dose therapy to the patient. The patient, in one example, is characterized as intermediate-risk on the basis of cytogenetic analysis. In one example, the therapy comprises the administration of anthracycline. In a related embodiment, administering high dose therapy comprises administering one or more high dose anthracycline antibiotics selected from the group consisting of Daunorubicin, Doxorubicin, Epirubicin, Idarubicin, Mitoxantrone, and Adriamycin.

One aspect of the present disclosure is directed to a method of predicting survival of a patient with acute myeloid leukemia, comprising: (a) analyzing a sample isolated from the patient for the presence of (i) a mutation in at least one of FLT3, MLL-PTD, ASXL1, and PHF6 genes, plus optionally one or more of NPM1, DNMT3A, NRAS, CEBPA, TET2, WT1, IDH1, IDH2, KIT, RUNX1, KRAS, PTEN, P53, HRAS, and EZH2 genes; or (ii) a mutation in IDH2 and/or CEBPA genes, plus optionally one or more of FLT3, MLL-PTD, ASXL1, PHF6, NPM1, DNMT3A, NRAS, TET2, WT1, IDH1, KIT, RUNX1, KRAS, PTEN, P53, HRAS, and EZH2 genes; and (b) (i) predicting poor survival of the patient if a mutation is present in at least one of FLT3, MLL-PTD, ASXL1 and PHF6 genes, or (ii) predicting favorable survival of the patient if a mutation is present in IDH2R140 and/or a mutation is present in CEBPA. The method further comprises analyzing the sample for the presence of cytogenetic abnormalities. The method further comprises predicting favorable survival of the patient if the following mutation is present: IDH2R140Q.

Other aspects of the present disclosure include the chemotherapeutics for use in the methods described herein, or use of those in the preparation of a medicament when used in the methods described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the mutational complexity of AML. Circos diagram depicting relative frequency and pairwise co-occurrence of mutations in de novo AML patients enrolled in the ECOG protocol E1900 (Panel A). The arc length corresponds to the frequency mutations in the first gene and the ribbon width corresponds to the percentage of patients that also have a mutation in the second gene. Pairwise co-occurrence of mutations is denoted only once, beginning with the first gene in the clockwise direction. Since only pairwise mutations are encoded for clarity, the arc length was adjusted to maintain the relative size of the arc and the correct proportion of patients with a single mutant allele is represented by the empty space within each mutational subset. Panel A also contains the mutational frequency in the test cohort. Panels B and C show the mutational events in DNMT3A and FLT3 mutant patients respectively.

FIG. 2 shows multivariate risk classification of intermediate-risk AML. Kaplan-Meier estimates of overall survival (OS) are shown for the risk stratification of intermediate-risk AML (p-values represent a comparison of all curves). For FLT3-ITD negative, intermediate-risk AML (Panel A) there are three genotypes: poor defined by mutant TET2 or ASXL1 or PHF6 or MLL-PTD, good defined by mutant IDH1 or IDH2 and mutant NPM1, and intermediate defined by all other genotypes. For FLT3-ITD positive, intermediate-risk AML (Panel B), there is the mutant CEBPA genotype, poor defined by mutant TET2 or DNMT3A or MLL-PTD or trisomy 8, and all other genotypes.

FIG. 3 shows revised AML risk stratification based on integrated genetic analysis. FIG. 3A shows a revised risk stratification based on integrated cytogenetic and mutational analysis. Final overall risk groups are on the right. FIG. 3B shows the impact of integrated mutational analysis on risk stratification in the test cohort of AML patients (p-values represent a comparison of all curves). The black curves show the patients in the cytogenetic risk groups that remained unchanged. The green curve shows patients that were reclassifed from intermediate-risk to favorable-risk. The red curve shows patients that were reclassified from intermediate-risk to unfavorable-risk. FIG. 3C confirms the reproducibility of the genetic prognostic schema in an independent cohort of 104 samples from the E1900 trial (p-values represent a comparison of all curves).

FIG. 4 shows the molecular determinants of response to high-dose Daunorubicin induction chemotherapy. Kaplan-Meier estimates of OS in the entire cohort according to DNMT3A mutational status (Panel A) and DNMT3A status in patients receiving high-dose or standard-dose daunorubicin (Panel B). OS in patients according to treatment arm is shown in patients with DNMT3A or NPM1 mutations or MLL translocations (Panel C) and patients lacking DNMT3A or NPM1 mutations or MLL translocations (Panel D).

FIG. 5 shows comprehensive mutational profiling improves risk-stratification and clinical management of patients with acute myeloid leukemia (AML). Use of mutational profiling delineates subsets of cytogenetically defined intermediate-risk patients with markedly different prognoses and reallocates a substantial proportion of patients to favorable or unfavorable-risk categories (A). In addition, mutational profiling identifies genetically defined subsets of AML patients with improved outcome with high-dose anthracycline induction chemotherapy (B).

FIG. 6 shows Circos diagrams for each gene.

FIG. 7 shows Circos diagrams for all genes and some relevant cytogenetic abnormalities in patients within cytogenetically-defined favorablerisk (Panel A), intermediate-risk (Panel B), and unfavorable-risk (Panel C) subgroups. The percentage of patients in each cytogenetic risk category with >2 mutations is displayed in Panel D. The proportion of intermediate risk patients with 2 or more somatic mutations was significantly higher than of patients in the other 2 cytogenetic subgroups

FIG. 8 is a Circos diagram, showing the mutual exclusivity of IDH1, IDH2, TET2, and WT1 mutations.

FIG. 9 shows Kaplan-Meier estimates of OS according to mutational status: data are shown for OS in the entire cohort according to the mutational status of PHF6 (Panel A) and ASXL1 (Panel B).

FIG. 10 shows Kaplan-Meier survival estimates shown for IDH2 (Panel A), IDH2 R140 (Panel B), IDH1 (Panel C) and the IDH2 R172 allele (Panel D) in the entire cohort. Panel E shows both IDH2 alleles while Panel F shows all three IDH alleles (pvalue represents comparison of all curves). These data show that the IDH2 R140 allele is the only IDH allele to have prognostic relevance in the entire cohort.

FIG. 11 shows Kaplan-Meier estimates of OS in patients from the test cohort with core-binding factor alterations with mutations in KIT versus those wildtype for KIT. KIT mutations were not associated with a difference in OS when patients with any corebinding factor alteration (i.e. patients with t(8;21), inv(16), or t(16;16)) were studied (A). In contrast, KIT mutations were associated with a significant decrease in OS in patients bearing t(8;21) specifically (B). KIT mutations were not associated with adverse OS in patients with inv(16) or t(16;16) (C).

FIG. 12 shows Kaplan-Meier survival estimates for TET2 in cytogenetically defined intermediate-risk patients in the cohort.

FIG. 13 shows Kaplan-Meier survival estimates for NPM1-mutant patients with cytogenetically-defined intermediate-risk in the cohort. Only those with concomitant IDH mutations have improved survival.

FIG. 14 shows the risk classification schema for FLT3-ITD widltype (A) and mutant (B) intermediate-risk AML shown in FIG. 3 is shown here for normal-karyotype patients only.

FIG. 15 shows that the mutational prognostic schema predicts outcome regardless of post-remission therapy with no transplantation (A), autologous transplantation (B), and allogeneic transplantation (C) (p-value represents comparison of all curves). Note, curves represent overall risk categories integrating cytogenetic and mutational analysis (as shown in final column in FIG. 3A).

FIG. 16 shows Kaplan-Meier estimates of OS in the entire cohort according to DNMT3A mutational status (Panel A and B), MLL translocation status (Panel C and D) or NPM1 mutational status in patients receiving high-dose or standard-dose daunorubicin (Panels E and F). OS in patients according to treatment arm is shown in DNMT3A mutant (Panel A) and wild-type (Panel B) patients. Panel C shows OS in MLL translocated patients receiving high-dose or standard-dose daunorubicin while Panel D shows OS in non-MLL translocated patients depending on daunorubicin dose. OS in patients according to treatment arm is shown in NPM1 mutant (Panel E) and wild-type (Panel F) patients as well.

### DETAILED DESCRIPTION OF THE INVENTION

To facilitate understanding of the invention, the following definitions are provided. It is to be understood that, in general, terms not otherwise defined are to be given their meaning or meanings as generally accepted in the art. The terminology used herein is for the purpose of describing particular embodiments only and is not intended to limit the scope of the present invention which will be limited only by the appended claims.

In practicing the present invention, many conventional techniques in molecular biology are used. These techniques are described in greater detail in, for example, Molecular Cloning: a Laboratory Manual 3^{rd} edition, J. F. Sambrook and D. W. Russell, ed. Cold Spring Harbor Laboratory Press 2001 and DNA Microarrays: A Molecular Cloning Manual. D. Bowtell and J. Sambrook, eds. Cold Spring Harbor Laboratory Press 2002. Additionally, standard protocols, known to and used by those of skill in the art in mutational analysis of mammalian cells, including manufacturers' instruction manuals for preparation of samples and use of microarray platforms are hereby incorporated by reference.

In the description that follows, a number of terms are used extensively. The following definitions are provided to facilitate understanding of the invention. Unless otherwise specified, “a,” “an,” “the,” and “at least one” are used interchangeably and mean one or more than one.

The terms “cancer”, “cancerous”, or “malignant” refer to or describe the physiological condition in mammals that is typically characterized by unregulated growth of tumor cells. Examples of a blood cancer include but are not limited to acute myeloid leukemia.

The term “diagnose” as used herein refers to the act or process of identifying or determining a disease or condition in a mammal or the cause of a disease or condition by the evaluation of the signs and symptoms of the disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to said particular disease; i.e. there may be differential diagnoses that can be inferred from a diagnostic factor or symptom. Likewise, there may be instances where a factor or symptom that is indicative of a particular disease is present in an individual that does not have the particular disease.

“Expression profile” as used herein may mean a genomic expression profile. Profiles may be generated by any convenient means for determining a level of a nucleic acid sequence e.g. quantitative hybridization of microRNA, labeled microRNA, amplified microRNA, cRNA, etc., quantitative PCR, ELISA for quantitation, and the like, and allow the analysis of differential gene expression between two samples. A subject or patient tumor sample, e.g., cells or collections thereof, e.g., tissues, is assayed. Samples are collected by any convenient method, as known in the art.

“Gene” as used herein may be a natural (e.g., genomic) gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. The term “gene” has its meaning as understood in the art. However, it will be appreciated by those of ordinary skill in the art that the term “gene” has a variety of meanings in the art, some of which include gene regulatory sequences (e.g., promoters, enhancers, etc.) and/or intron sequences, and others of which are limited to coding sequences. It will further be appreciated that definitions of “gene” include references to nucleic acids that do not encode proteins but rather encode functional RNA molecules such as tRNAs. For the purpose of clarity we note that, as used in the present application, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid.

“Mammal” for purposes of treatment or therapy refers to any animal classified as a mammal, including humans, domestic and farm animals, and zoo, sports, or pet animals, such as dogs, horses, cats, cows, etc. Preferably, the mammal is human.

“Microarray” refers to an ordered arrangement of hybridizable array elements, preferably polynucleotide probes, on a substrate.

Therapeutic agents for practicing a method of the present invention include, but are not limited to, inhibitors of the expression or activity of genes identified and disclosed herein, or protein translation thereof. An “inhibitor” is any substance which retards or prevents a chemical or physiological reaction or response. Common inhibitors include but are not limited to antisense molecules, antibodies, and antagonists.

The term “poor” as used herein may be used interchangeably with “unfavorable.” The term “good” as used herein may be referred to as “favorable.” The term “poor responder” as used herein refers to an individual whose cancer grows during or shortly thereafter standard therapy, for example radiation-chemotherapy, or who experiences a clinically evident decline attributable to the cancer. The term “respond to therapy” as used herein refers to an individual whose tumor or cancer either remains stable or becomes smaller/reduced during or shortly thereafter standard therapy, for example radiation-chemotherapy.

“Probes” may be derived from naturally occurring or recombinant single- or double-stranded nucleic acids or may be chemically synthesized. They are useful in detecting the presence of identical or similar sequences. Such probes may be labeled with reporter molecules using nick translation, Klenow fill-in reaction, PCR or other methods well known in the art. Nucleic acid probes may be used in southern, northern or in situ hybridizations to determine whether DNA or RNA encoding a certain protein is present in a cell type, tissue, or organ.

“Prognosis” as used herein refers to a forecast as to the probable outcome of cancer, including the prospect of recovery from the cancer. As used herein the terms prognostic information and predictive information are used interchangeably to refer to any information that may be used to foretell any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

The term “prognosis” as used herein refers to a prediction of the probable course and outcome of a clinical condition or disease. A prognosis of a patient is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis may be expressed as the number of months, on average, that a patient can expect to survive as a result of a condition or disease. The prognosis of a patient may be considered as an expression of relativism, with many factors effecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

The terms “favorable prognosis” and “positive prognosis,” or “unfavorable prognosis” and “negative prognosis” as used herein are relative terms for the prediction of the probable course and/or likely outcome of a condition or a disease. A favorable or positive prognosis predicts a better outcome for a condition than an unfavorable or negative or adverse prognosis. In a general sense a “favorable prognosis” is an outcome that is relatively better than many other possible prognoses that could be associated with a particular condition, whereas an “unfavorable prognosis” predicts an outcome that is relatively worse than many other possible prognoses that could be associated with a particular condition. Typical examples of a favorable or positive prognosis include a better than average cure rate, a lower propensity for metastasis, a longer than expected life expectancy, differentiation of a benign process from a cancerous process, and the like. For example, if a prognosis is that a patient has a 50% probability of being cured of a particular cancer after treatment, while the average patient with the same cancer has only a 25% probability of being cured, then that patient exhibits a positive prognosis. A positive prognosis may be diagnosis of a benign tumor if it is distinguished over a cancerous tumor.

The term “relapse” or “recurrence” as used in the context of cancer in the present application refers to the return of signs and symptoms of cancer after a period of remission or improvement.

As used herein a “response” to treatment may refer to any beneficial alteration in a subject's condition that occurs as a result of treatment. Such alteration may include stabilization of the condition (e.g., prevention of deterioration that would have taken place in the absence of the treatment), amelioration of symptoms of the condition, improvement in the prospects for cure of the condition. One may refer to a subject's response or to a tumor's response. In general these concepts are used interchangeably herein.

“Treatment” or “therapy” refer to both therapeutic treatment and prophylactic or preventative measures. The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder.

For the recitation of numeric ranges herein, each intervening number there between with the same degree of precision is explicitly contemplated. For example, for the range of 2-5, the numbers 3 and 4 are contemplated in addition to 2 and 5, and for the range 2.0-3.0, the number 2.0, 2.1, 2.2, 2.3, 2.4, 2.5, 2.6, 2.7, 2.8, 2.9 and 3.0 are explicitly contemplated. As used herein, the term “about” X or “approximately” X refers to +/−10% of the stated value of X.

Inherent difficulties in the diagnosis and treatment of cancer include among other things, the existence of many different subgroups of cancer and the concomitant variation in appropriate treatment strategies to maximize the likelihood of positive patient outcome. Current methods of cancer treatment are relatively non-selective. Typically, surgery is used to remove diseased tissue; radiotherapy is used to shrink solid tumors; and chemotherapy is used to kill rapidly dividing cells.

In the case of blood cancers, it is worthy to begin by noting that blood primarily consists of red blood cells (RBC), white blood cells (WBC) and platelets. Red blood cells carry oxygen to the body, the white blood cells police and protect the body, and platelets help clot the blood when there is injury. Abnormalities in these cell types can lead to blood cancer. The main categories of blood cancer are Acute Lymphocytic or Lymphoblastic Leukemias (ALL), Chronic Lymphocytic or Lymphoblastic Leukemias (CLL), Acute Myelogenous or Myeloid Leukemias (AML), and Chronic Myelogenous or Myeloid Leukemias (CML).

Both leukemia and lymphoma are hematologic malignancies (cancers) of the blood and bone marrow. In the case of leukemia, the cancer is characterized by abnormal proliferation of leukocytes and is one of the four major types of cancer. The cancer interferes with the body's ability to make blood, and the cancer attacks the bone marrow and the blood itself, causing fatigue, anemia, weakness, and bone pain. Leukemia is diagnosed with a blood test in which specific types of blood cells are counted; it accounts for about 29,000 adults and 2,000 children diagnosed each year in the United States. Treatment for leukemia typically includes chemotherapy and radiation to kill the cancer, and may involve bone marrow transplantation in some cases.

Leukemias are classified according to the type of leukocyte most prominently involved. Acute leukemias are predominantly undifferentiated cell populations and chronic leukemias have more mature cell forms. The acute leukemias are divided into lymphoblastic (ALL) and non-lymphoblastic (ANLL) types, with ALL being predominantly a childhood disease while ANLL, also known as acute myeloid leukemia (AML), being a more common acute leukemia among adults.

AML is characterized by an increase in the number of myeloid cells in the marrow and an arrest in their maturation, frequently resulting in hematopoietic insufficiency. In the United States, the annual incidence of AML is approximately 2.4 per 100,000 and it increases progressively with age to a peak of 12.6 per 100,000 adults 65 years of age or older. Despite improved therapeutic approaches, prognosis of AML is very poor around the globe. Even in the United States, five-year survival rate among patients who are less than 65 years of age is less than 40%.

Acute myeloid leukemia (AML) is a heterogeneous disorder that includes many entities with diverse genetic abnormalities and clinical features. The pathogenesis is known for relatively few types of leukemia. Patients with intermediate and poor risk cytogenetics represent the majority of AML; chemotherapy based regimens fail to cure most of these patients and stem cell transplantation is frequently the treatment choice. Since allogeneic stem cell transplantation is not an option for many patients with high risk leukemia, there is a need to improve our understanding of the biology of these leukemias and to develop improved therapies. Despite considerable advances, not enough is known of the etiology, cell physiology and molecular genetics of acute myeloid leukemia. As such, the development of effective new agents and novel treatment and/or prognostic methods against myeloid leukemia, and in particular acute myeloid leukemia, remains a focal point today in translational oncology research.

Significant progress has been made in understanding risk factors, including genetic factors, that may contribute to AML, but the relevance of these factors to clinical outcome remains unclear. In addition, the expression level and antibody staining pattern of several proteins have been shown to be predictive of outcome and of the likelihood of response to therapy. However, the clinical outcome of individual patients remains uncertain, and the ability to predict which patients are likely to benefit from a particular type of therapy (e.g., a certain drug or class of drug) remains elusive.

In the present disclosure, leukemic samples from patients with diagnosed AML were obtained. Bone marrow or peripheral blood samples were collected, prepared by Ficoll-Hypaque (Nygaard) gradient centrifugation. Cytogenetic analyses of the samples were performed at presentation, as previously described (Bloomfield; Leukemia 1992; 6:65-67. 21). The criteria used to describe a cytogenetic clone and karyotype followed the recommendations of the International System for Human Cytogenetic Nomenclature. DNA was extracted from diagnostic bone marrow aspirate samples or peripheral blood samples using method described previously (Zuo et al. Mod Pathol. 2009; 22, 1023-1031).

The present disclosure is based on mutational analysis of 18 genes in 398 patients with AML younger than 60 years of age randomized to receive induction therapy including high-dose or standard dose daunorubicin. Prognostic findings were further validated in an independent set of 104 patients.

The inventors of the instant application have identified ≧1 somatic alteration in 97.3% of patients. These Applicants discovered (1) that FLT3-ITD (p=0.001), MLL-PTD (p=0.009), ASXL1 (p=0.05), and PHF6 (p=0.006) mutations are associated with reduced overall survival (“OS”); and (2) that CEBPA (p=0.05) and IDH2R140Q (p=0.01) mutations were associated with improved OS.

Accordingly, in one aspect of the present disclosure is a method of predicting survival of a patient with acute myeloid leukemia, said method comprising: analyzing a genetic sample isolated from the patient for the presence of cytogenetic abnormalities and a mutation in at least one of FLT3, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2 genes; and (i) predicting poor survival of the patient if a mutation is present in at least one of FLT3, MLL-PTD, ASXL1 and PHF6 genes, or (ii) predicting favorable survival of the patient if a mutation is present in IDH2R140 (e.g. IDH2R140Q) and/or a mutation is present in CEBPA. In one embodiment, the method further comprises, predicting intermediate survival of the patient with cytogenetically-defined intermediate risk AML if: (i) no mutation is present in any of FLT3-ITD, TET2, MLL-PTD, DNMT3A, ASXL1 or PHF6 genes, (ii) a mutation in CEBPA is and the FLT3-ITD is present, or (iii) a mutation is present in FLT3-ITD but trisomy 8 is absent. In another embodiment, the method further comprises predicting unfavorable survival of the patient if (i) a mutation in TET2, ASXL1, or PHF6 or an MLL-PTD is present in a patient without the FLT3-ITD mutation, or (ii) the patient has a FLT3-ITD mutation and a mutation in TET2, DNMT3A, MLL-PTD or trisomy 8.

The genetic sample may be obtained from a bone marrow aspirate or the patient's blood. Once the sample is obtained, in one example, the mononuclear cells are isolated according to known techniques including Ficoll separation and their DNA is extracted. In a particular embodiment, poor survival or adverse risk of the patient is survival of less than or equal to about 10 months. Whereas, in one embodiment, intermediate survival the patient is survival of about 18 months to about 30 months. In another embodiment, favorable survival of the patient is survival of about 32 months or more.

In another aspect, the present disclosure teaches a method of predicting survival of a patient with acute myeloid leukemia, said method comprising, assaying a genetic sample from the patient's blood or bone marrow for the presence of a mutation in at least one of genes FLT3, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2 in said sample; and predicting a poor survival of the patient if a mutation is present in at least one of genes FLT3-ITD, MLL-PTD, ASXL1, PHF6; or predicting a favorable survival of the patient if a mutation is present in CEBPA or a mutation is present in IDH2 at R140. In one embodiment, the patient is characterized as intermediate-risk on the basis of cytogenetic analysis.

In one embodiment, amongst patients with cytogenetically-defined intermediate-risk acute myeloid leukemia who have FLT3-ITD mutation, at least one of the following: trisomy 8 or a mutation in TET2, DNMT3A, or the MLL-PTD are associated with an adverse outcome and poor overall survival of the patient. In another embodiment, amongst patients with cytogenetically-defined intermediate-risk acute myeloid leukemia who have a mutation in FLT3-ITD gene, a mutation in CEBPA gene is associated with improved outcome and overall survival of the patient. In one embodiment, in a cytogenetically-defined intermediate risk AML patient with both IDH1/IDH2 and NPM1 mutations, the overall survival is improved compared to NPM1-mutant patients wild-type for both IDH1 and IDH2. In one embodiment, amongst patients with acute myeloid leukemia, IDH2R140 mutations are associated with improved overall survival. Poor or unfavorable survival (adverse risk) of the patient, in one example, is survival of less than or equal to about 10 months. Favorable survival of the patient, in one example, is survival of about 32 months or more.

In one embodiment, the favorable impact of NPM1 mutations was restricted to patients with co-occurring IDH1/IDH2 and NPM1 mutations. Further, Applicants identified genetic predictors of outcome that improved risk stratification in AML independent of age, WBC count, induction dose, and post-remission therapy and validated their significance in an independent cohort. Applicants discovered that high-dose daunorubicin improved survival in patients with DNMT3A or NPM1 mutations or MLL translocations (p=0.001) relative to treatment with standard dose daunorubicin, but not in patients wild-type for these alterations (p=0.67).

These data provide clinical implications of genetic alterations in AML by delineating mutations that predict outcome in AML and improve AML risk stratification. Applicants have herein discovered and demonstrated the utility of mutational profiling to improve prognostic and therapeutic decisions in AML, and in particular, have shown that DNMT3A or NPM1 mutations or MLL translocations predict for improved outcome with high-dose induction chemotherapy.

Previous studies have highlighted the clinical and biologic heterogeneity of acute myeloid leukemia (AML). However, a relatively small number of cytogenetic and molecular lesions have sufficient relevance to influence clinical practice. The prognostic relevance of cytogenetic abnormalities has led to the widespread adoption of risk stratification into three cytogenetically-defined risk groups with significant differences in OS. Although progress has been made in defining prognostic markers for AML, a significant proportion of patients lack a specific abnormality of prognostic significance. Additionally, there is significant heterogeneity in outcome for individual patients in each risk group.

Recent studies have identified a number of recurrent somatic mutations in patients with AML, however, to date, whether mutational profiling of a larger set of genes would improve prognostication in AML has not been investigated in a clinical trial cohort. Here, Applicants conceived that integrated mutational analysis of all known molecular alterations occurring in >5% of AML patients would allow for the identification of novel molecular markers of outcome in AML and allow for the identification of molecularly defined subsets of patients who benefit from dose-intensified induction chemotherapy.

High-Throughput Mutational Profiling in AML: Comprehensive Genetic Analysis

Clinical studies have demonstrated that acute myeloid leukemia (AML) is heterogeneous with respect to presentation and to clinical outcome, and studies have shown that cytogenetics can be used to improve prognostication and to guide therapeutic decisions. More recently, genetic studies have improved our understanding of the genetic basis of AML. Applicants recognized genetic lesions represent prognostic markers which can be used to risk stratify AML patients and guide therapeutic decisions. However, although a number of gene mutations occur at significant frequency in AML, their prognostic value is not known in large phase III clinical trial cohorts.

Applicants report for the first time in a uniformly treated clinical cohort, the mutational status of all genes known to be significantly (>5%) mutated in AML as well as the effect of mutations in these genes on outcome and response to therapy. Applicants used a high throughput re-sequencing platform to perform full length resequencing of the coding regions of FLT3, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2 in pre-treatment genomic DNA from 398 patients with de novo AML enrolled in the ECOG E1900 Study.

Including both mutations and cytogenetic abnormalities, Applicants were able to identify a clonal alteration in 91.2% of all patients in the E1900 cohort; 42% had 1 somatic alteration, 36.4% had 2 alterations, 11.3% had 3 alterations and 1.5% had 4 alterations. Mutational data from each patient was correlated with overall survival, disease-free survival, and with treatment assignment (standard dose or high dose daunorubicin). Applicants discovered somatic mutations in FLT3 (37% total; 30% ITD, 7% TKD), DNMT3A (23%), NPM1 (14%), CEBPA (10%), TET2 (10%), NRAS (10%), WT1 (10%), KIT (9%), IDH2 (8%), IDH1 (6%), RUNX1 (6%), ASXL1 (4%), PHF6 (3%), KRAS (2.5%), TP53 (2%), PTEN (1.5%); the only genes without mutations in Applicants' screen were HRAS and EZH2.

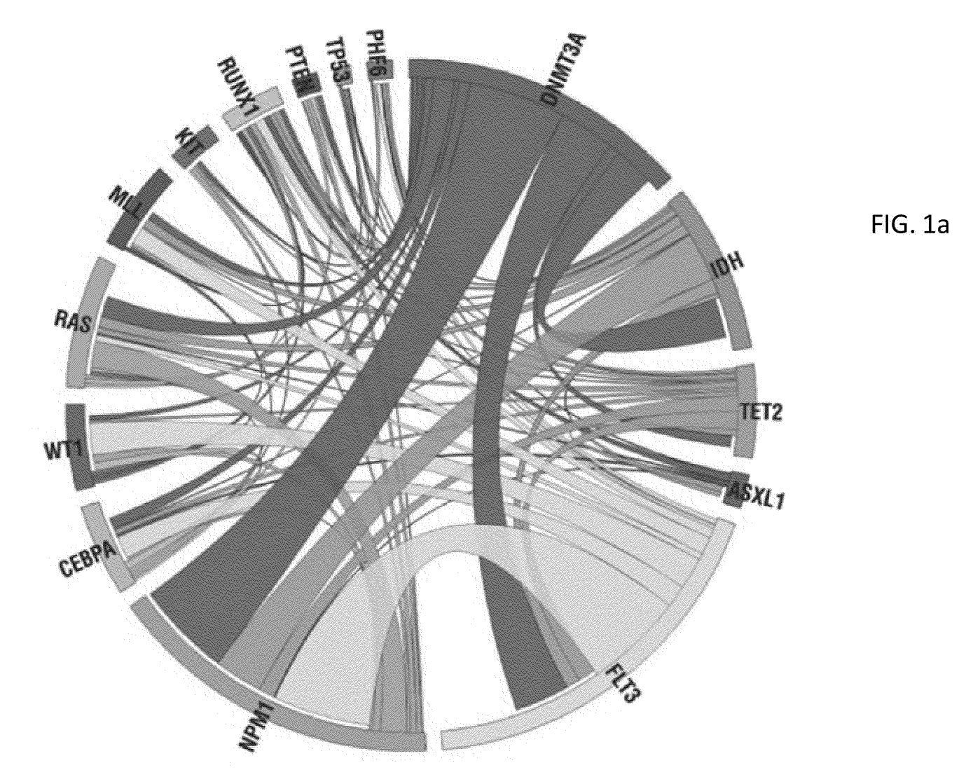

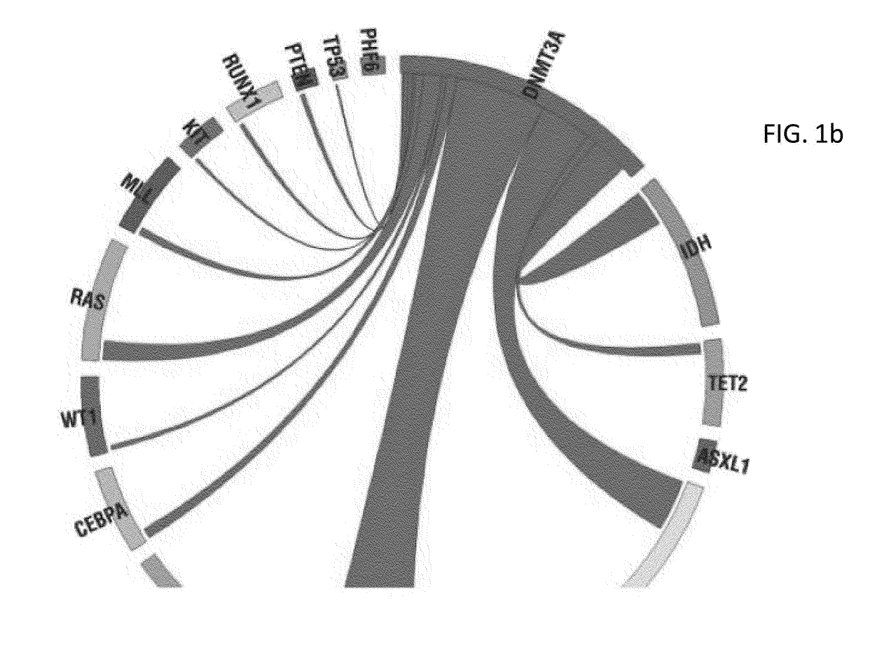

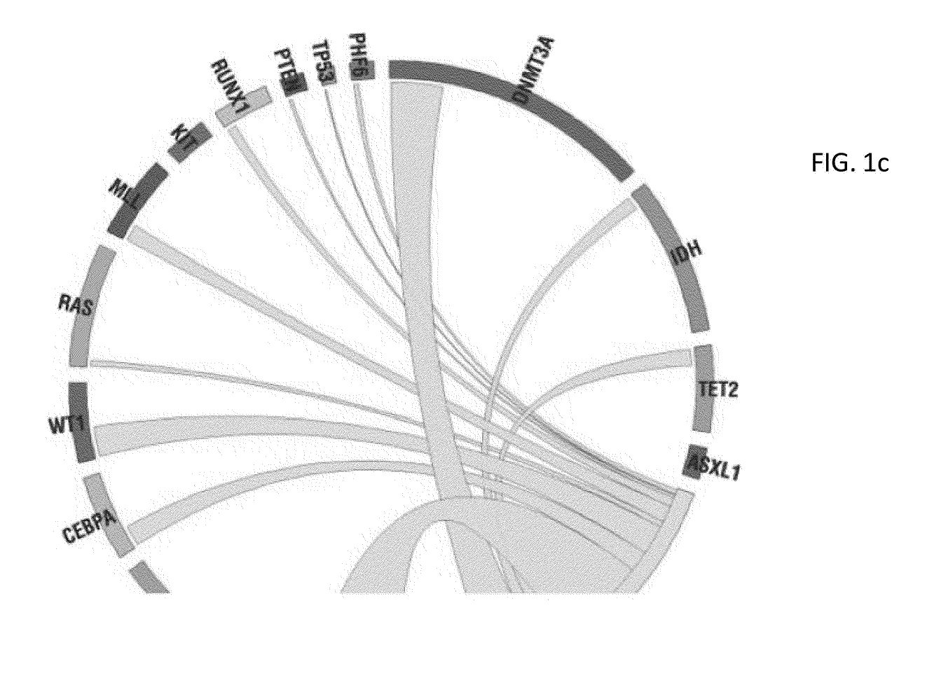

Applicants next used correlation analysis to assess whether mutations were positively or negatively correlated (FIG. 1). In addition to identified mutational correlations (FLT3 and NPM1, KIT and core binding factor leukemia), Applicants found that FLT3 and ASXL1 mutations were mutually exclusive in this large cohort (p=0.0008). Further, Applicants found that IDH1/IDH2 mutations were mutually exclusive of both TET2 (p=0.02), and WT1 (p=0.01) mutations, suggesting these mutations have overlapping roles in AML pathogenesis.

Applicants next set out to investigate if any mutations were associated with lack of response to chemotherapy; notably mutations in ASXL1 (p=0.0002) and WT1 (p=0.03) were enriched in patients with primary refractory-AML. Integration of mutational data with outcome in the ECOG E1900 trial revealed significant effects that mutations in FLT3 (p=0.0005), ASXL1 (p=0.005), and PHF6 (p=0.02) were associated with reduced overall survival. In addition, Applicants found that mutations in CEBPA (p=0.04) and in IDH2 (p=0.003) were associated with improved overall survival; the favorable impact of IDH1 mutations on outcome was exclusively seen in patients with IDH2R140 mutations.

This data represents a comprehensive mutational analysis of 18 genes in a uniformly-treated de novo AML cohort, which allowed Applicants to delineate the mutational frequency of this gene set in de novo AML, the pattern of mutational cooperativity in AML and the clinical effects of gene mutations on survival and response to therapy in AML. In one embodiment, Applicants identified mutations in ASXL1 and WT1 as being significantly enriched in patients who failed to respond to standard induction chemotherapy in AML. This data provides important clinical implications of genetic alterations in AML and provides insight into the multistep pathogenesis of adult AML. In one embodiment, the acute myeloid leukemia is selected from newly diagnosed, relapsed or refractory acute myeloid leukemia.

Accordingly, one aspect of the present disclosure is a method of predicting survival of a patient with acute myeloid leukemia, said method comprising assaying a genetic sample from the patient's blood or bone marrow for the presence of a mutation in genes ASXL1 and WT1; and determining the patient has or will develop primary refractory acute myeloid leukemia if mutated ASXL1 and WT1 genes are detected. The sample can be a bone marrow aspirate or the patient's blood. Further, in one embodiment, the mononuclear cells are isolated for use in the assay.

Applicants have developed a mutational classifier which can be used to predict risk of relapse in adults with acute myeloid leukemia by combining standard analysis of cytogenetics with full length sequencing of FLT3, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2. The teachings of the instant application allow for the development of an integrated mutation classifier which can more accurately predict outcome and relapse risk than currently available techniques. In one embodiment, poor survival is survival of less than or equal to about ten months. In another embodiment, intermediate survival of the patient is survival of about 18 months to about 30 months. In a related embodiment, favorable survival of the patient is survival of about 32 months or more.

In one embodiment, in patients with FLT3-ITD wild-type intermediate-risk acute myeloid leukemia, TET2, ASXL1, PHF6, and MLL-PTD gene mutations were independently shown to be associated with adverse outcome and poor overall survival of the patient. In another embodiment, in patients with FLT3-ITD mutant intermediate-risk acute myeloid leukemia, CEBPA gene mutations were associated with improved outcome and overall survival of the patient. In yet another embodiment, in cytogenetically-defined intermediate risk AML patients with FLT3-ITD mutant intermediate-risk acute myeloid leukemia, trisomy 8 and TET2, DNMT3A, and MLL-PTD mutations were associated with an adverse outcome and poor overall survival of the patient. In one embodiment, cytogenetically-defined intermediate risk AML patients with both IDH1/IDH2 and NPM1 mutations have an improved overall survival compared to NPM1-mutant patients wild-type for both IDH1 and IDH2. In a related embodiment, IDH2 R140Q mutations are associated with improved overall survival in the overall cohort of AML patients.

One aspect of the present disclosure is directed to a method of predicting survival of a patient with acute myeloid leukemia, comprising: (a) analyzing a sample isolated from the patient for the presence of (i) a mutation in at least one of FLT3, MLL-PTD, ASXL1, and PHF6 genes, plus optionally one or more of NPM1, DNMT3A, NRAS, CEBPA, TET2, WT1, IDH1, IDH2, KIT, RUNX1, KRAS, PTEN, P53, HRAS, and EZH2 genes; or (ii) a mutation in IDH2 and/or CEBPA genes, plus optionally one or more of FLT3, MLL-PTD, ASXL1, PHF6, NPM1, DNMT3A, NRAS, TET2, WT1, IDH1, KIT, RUNX1, KRAS, PTEN, P53, HRAS, and EZH2 genes; and (b) (i) predicting poor survival of the patient if a mutation is present in at least one of FLT3, MLL-PTD, ASXL1 and PHF6 genes, or (ii) predicting favorable survival of the patient if a mutation is present in IDH2R140 and/or a mutation is present in CEBPA. The method may further comprise analyzing the sample for the presence of cytogenetic abnormalities. The method may further comprise predicting favorable survival of the patient if the following mutation is present: IDH2R140Q.

Furthermore, Applicants have discovered that DNMT3A mutations, NPM1 mutations or MLL fusions predict for improved outcome with high dose chemotherapy, which includes dose-intensified induction therapy. The teachings of the instant application provide for accurate risk stratification of AML patients and the ability to decide which patients need more agreessive therapy given high risk, and identification of low risk patients less in need of intensive post remission therapy. Moreover, it is possible to identify genotypically defined subsets of patients who would benefit from induction with dose-intensified anthracyclines, for example, daunorubicin. The present disclosure provides for more accurate assessment in risk classification. Presently, there is no effective way to determine which patients suffering from AML benefit from high dose daunorubicin. In one embodiment, the present disclosure provides for a novel classifier as well as a predictor of response.

Accordingly, one aspect of the present disclosure is a method of determining responsiveness of a patient with acute myeloid leukemia to high dose therapy, said method comprising analyzing a genetic sample isolated from the patient for the presence of a mutation in genes DNMT3A, and NPM1, and for the presence of a MLL translocation; and (i) identifying the patient as one who will respond to high dose therapy if a mutation in DNMT3A or NPM1 or an MLL translocation are present, or (ii) identifying the patient as one who will not respond to high dose therapy in the absence of mutations in DNMT3A or NPM1 or an MLL translocation. In one embodiment, the sample is DNA extracted from bone marrow or blood from the patient. The genetic sample may be DNA isolated from mononuclear cells (MNC) from blood or bone marrow of the patient. In one embodiment, the therapy comprises the administration of anthracycline. Examples of anthracyclines include Daunorubicin, Doxorubicin, Epirubicin, Idarubicin, Mitoxantrone, and Adriamycin. In a particular example, the anthracycline is Daunorubicin.

The method may be used to predict a patient's response to therapy before beginning therapy, during therapy, or after therapy is completed. For example, by predicting a patient's response to therapy before beginning therapy, the information may be used in determining the best therapy option for the patient.

One embodiment of the present invention is directed to methods to screen a patient for the prognosis for acute myeloid leukemia. The invention may provide information concerning the survival rate of a patient, the predicted life span of the patient, and/or the predicted likelihood of survival for the patient. In one embodiment, poor survival is referred generally as survival of about 10 months or less, and good prognosis or long-term survival is considered to be more than about 36 months or longer. In one embodiment, poor survival is considered as about one to 16 months, whereas good, favorable or long-term survival is considered to be range of about 30 to 42 months, more than about 46 months, or more than about 60 months. In one embodiment, good survival is considered to be about 30 months or longer.

In any aspect of the invention, unless context demands otherwise, the following combinations of genes and\or cytogenetic defects may be analyzed or assayed: FLT3 and CEBPA; FLT3 and trisomy 8; FLT3 and TET2; FLT3 and DNMT3A; FLT3 and MLL; FLT3, MLL, ASXL1 and PHF6, optionally with TET2 or DNMT3A; IDH2 and CEBPA; IDH1, IDH2 and NPM1; IDH2, ASXL1 and WT1; DNMT3A, NPM1 and MLL. Any of these combinations may be combined with any one or more other genes shown in the Table entitled ‘Genes analyzed for somatic mutations in genomic DNA of patients with AML and their clinical associations’. Optionally at least 2, 3, 4, 5, 6, 7, 8, 9, 10, 11, 12, 13, 14, 15, 16, 17, 18 or 19 genes are analyzed or assayed, which genes are listed in said table.

The present invention is also directed to a method for determining if an individual will respond to one or more therapies for acute myeloid leukemia. The therapy may be of any kind, but in specific embodiments it comprises chemotherapy, such as one or more anthracycline antibiotic agents. In one embodiment, the chemotherapy comprises the antimetabolite cytarabine in combination with an anthracycline.

In certain embodiments of the invention the therapy is chemotherapy, immunotherapy, antibody-based therapy, radiation therapy, or supportive therapy (essentially any implemented for leukemia). In a particular embodiment, the therapy comprises the administration of a chemotherapeutic agent comprising anthracycline antibiotics. Examples of such anthracycline antibiotics include, but are not limited to, Daunorubicin, Doxorubicin, Epirubicin, Idarubicin, Mitoxantrone, and Adriamycin. In some embodiments, the chemotherapy is Gleevac or idarubicin and ara-C. In a particular embodiment, daunorubicin is used.

Often, diagnostic assays are directed by a medical practitioner treating a patient, the diagnostic assays are performed by a technician who reports the results of the assay to the medical practitioner, and the medical practitioner uses the values from the assays as criteria for diagnosing the patient. Accordingly, the component steps of the method of the present invention may be performed by more than one person.

Prognosis may be a prediction of the likelihood that a patient will survive for a particular period of time, or said prognosis is a prediction of how long a patient may live, or the prognosis is the likelihood that a patent will recover from a disease or disorder. There are many ways that prognosis can be expressed. For example prognosis can be expressed in terms of complete remission rates (CR), overall survival (OS) which is the amount of time from entry to death, disease-free survival (DFS) which is the amount of time from CR to relapse or death. In one embodiment, favorable likelihood of survival, or overall survival, of the patient includes survival of the patient for about eighteen months or more.

A prognosis is often determined by examining one or more prognostic factors or indicators. These are markers, the presence or amount of which in a patient (or a sample obtained from the patient) signal a probability that a given course or outcome will occur. The skilled artisan will understand that associating a prognostic indicator with a predisposition to an adverse outcome may involve statistical analysis. Additionally, a change in factor concentration from a baseline level may be reflective of a patient prognosis, and the degree of change in marker level may be related to the severity of adverse events. Statistical significance is often determined by comparing two or more populations, and determining a confidence interval and/or a p value. See, e.g., Dowdy and Wearden, Statistics for Research, John Wiley & Sons, New York, 1983. In one embodiment, confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. Exemplary statistical tests for associating a prognostic indicator with a predisposition to an adverse outcome are described.

One approach to the study of cancer is genetic profiling, an effort aimed at identifying perturbations in gene expression and/or mutation that lead to the malignant phenotype. These gene expression profiles and mutational status provide valuable information about biological processes in normal and disease cells. However, cancers differ widely in their genetic signature, leading to difficulty in diagnosis and treatment, as well as in the development of effective therapeutics. Increasingly, gene mutations are being identified and exploited as tools for disease detection as well as for prognosis and prospective assessment of therapeutic success.

The inventors of the instant application hypothesized that genetic profiling of acute myeloid leukemia would provide a more effective approach to cancer management and/or treatment. The inventors have herein identified that mutations of a panel of genes lead to the malignant phenotype.

The present inventors have used a molecular approach to the problem and have identified a set of gene mutations in acute myeloid leukemia correlates significantly with overall survival. Accordingly, the present invention relates to gene mutation profiles useful in assessing prognosis and/or predicting the recurrence of acute myeloid leukemia. In one aspect, the present invention relates to a set of genes, the mutation of which in bone marrow or blood cells, in particular mononuclear cells, of a patient correlates with the likelihood of poor survival. The present invention relates to the prognosis and/or therapy response outcome of a patient with acute myeloid leukemia. The present invention provides several genes, the mutation of which, alone or in combination, has prognostic value, specifically with respect to survival.

In one example, the disclosure is a method of determining whether a human has an increased genetic risk for developing or developing a relapse of acute myeloid leukemia, comprising, analyzing a genetic sample isolated from the human's blood or bone marrow for the presence of a mutation in at least one gene from FLT3, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2; and determining the individual with cytogenetically-defined intermediate risk AML has an increased genetic risk for developing or developing a relapse of acute myeloid leukemia, relative to a control human with no such gene mutations in said genes, when: (i) a mutation in at least one of TET2, MLL-PTD, ASXL1 and PHF6 genes is detected when the patient has no FLT3-ITD mutation, or (ii) a mutation in at least one of TET2, MLL-PTD, and DNMT3A genes or trisomy 8 is detected when the patient has a FLT3-ITD mutation.

To date, no test exists that predicts outcome in acute myeloid leukemia, where one can stratify AML patients into good versus poor responders, and in particular, identify patients who would respond better to high dose chemotherapy. As a consequence, some individuals may be overtreated, in that they unnecessarily receive treatment that has minimal effect. Alternatively, some individuals may be undertreated, in that additional agents added to standard therapy may improve outcome for these patients who would be refractory to standard treatment alone. As such, it is desirable to prospectively distinguish responders from non-responders to standard therapy prior to the initiation of therapy in order to optimize therapy for individual patients.

Accordingly, one aspect of the present disclosure is a method of predicting whether a patient suffering from acute myeloid leukemia will respond better to high dose chemotherapy than to standard dose chemotherapy, the method comprising, obtaining a DNA sample obtained from the patient's blood or bone marrow; determining the mutational status of genes DNMT3A and NPM1, and the presence of a MLL translocation; and predicting that the subject will be more responsive to high dose chemotherapy than standard dose chemotherapy where the sample is positive for a mutation in DNMT3A or NPM1 or an MLL translocation, or predicting that the subject will be non-responsive to high dose chemotherapy compared to standard dose chemotherapy where the sample is wild type with no mutations in DNMT3A or NPM1 genes and no translocation in MLL.

In one embodiment, the invention provides a clinical test that is useful to predict outcome in acute myeloid leukemia. The mutational status and/or expression of one or more specific genes is measured in the sample. Individuals are stratified into those who are likely to respond well to therapy vs. those who will not. The information from the results of the test is used to help determine the best therapy for the patient in need of therapy. Patients are stratified into those who are likely to have a poor prognosis vs. those who will have a good prognosis with standard therapy. A health care provider uses the results of the test to help determine the course of action, for example the best therapy, for the patient in need of therapy.

Because certain markers from a patient relate to the prognosis of a patient in a continuous fashion, the determination of prognosis can be performed using statistical analyses to relate the determined marker status to the prognosis of the patient. A skilled artisan is capable of designing appropriate statistical methods. For example the methods of the present invention may employ the chi-squared test, the Kaplan-Meier method, the log-rank test, multivariate logistic regression analysis, Cox's proportional-hazard model and the like in determining the prognosis. Computers and computer software programs may be used in organizing data and performing statistical analyses.

In one embodiment, a test is provided whereby a sample, for example a bone marrow or blood sample, is profiled for a gene set and, from the mutation profile results, an estimate of the likelihood of response to standard acute myeloid leukemia therapy is determined. In another embodiment, the invention concerns a method of predicting the prognosis and/or likelihood of response to standard and/or high dose chemotherapy, following treatment, in an individual with acute myeloid leukemia, comprising determining the mutational status of one or more genes, in particular one to DNMT3A or NPM1 genes, or a MLL translocation, in a genetic sample obtained from the patient, normalized against a control gene or genes. A total value is computed for each individual from the mutational status of the individual genes in this gene set.

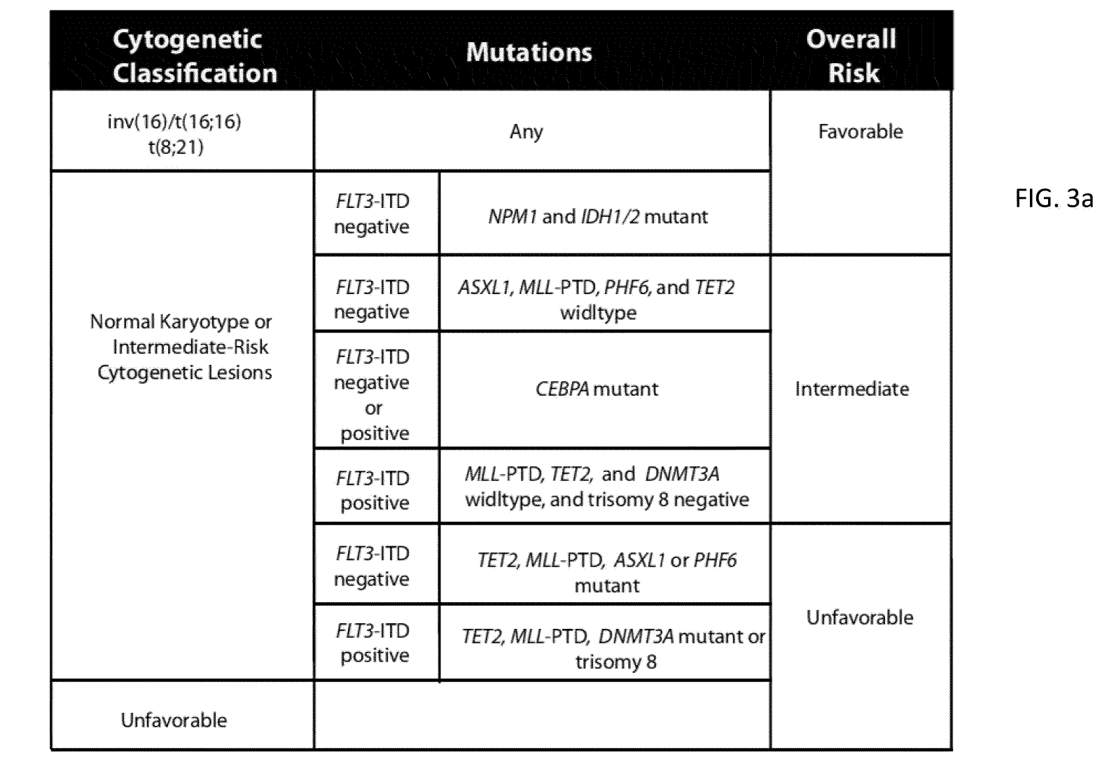

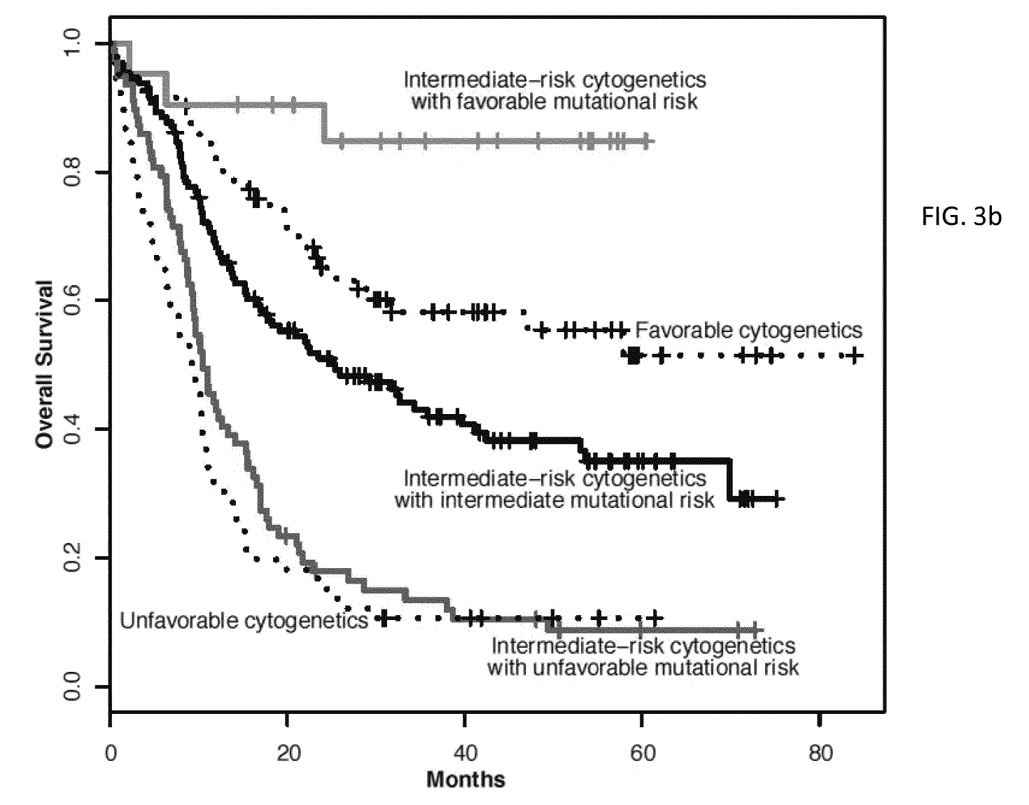

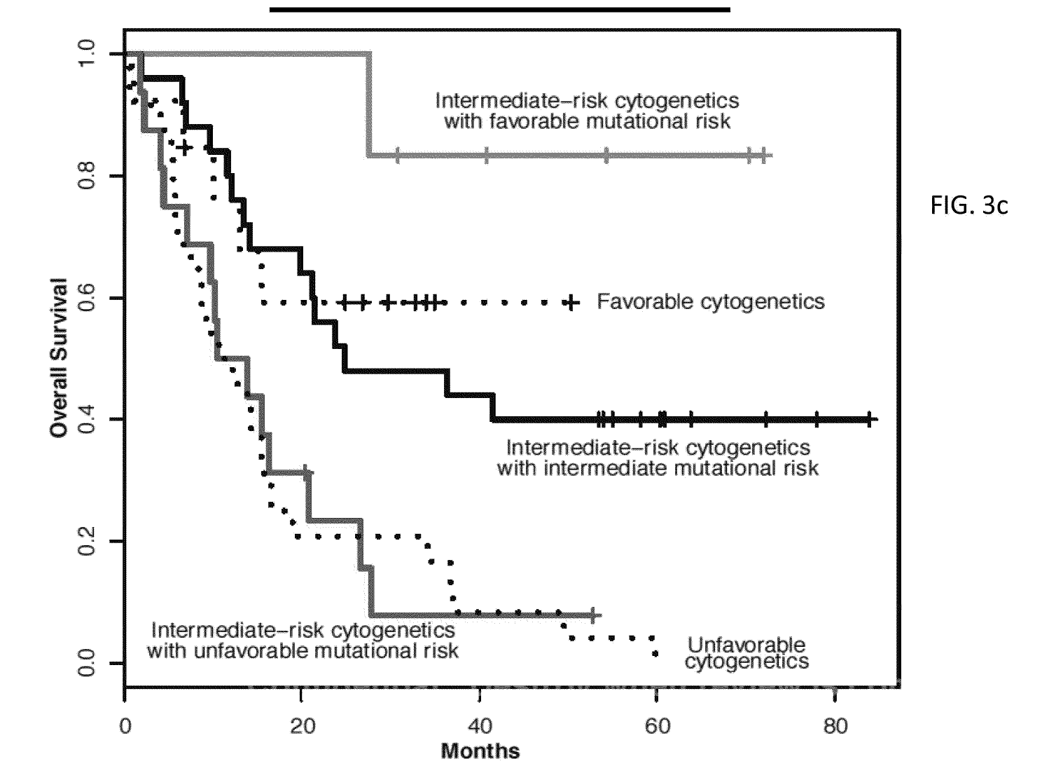

The present invention relates to the diagnosis, prognosis and treatment of blood cancer, including predicting the response to therapy and stratifying patients for therapy. The present disclosure teaches the mutational frequency, prognostic significance, and therapeutic relevance of integrated mutation profiling in 398 patients from the ECOG E1900 phase III clinical trial and validates these data in an independent cohort of 104 patients from the same trial. Previous studies have suggested that mutational analysis of CEBPA, NPM1, and FLT3-ITD can be used to risk stratify intermediate-risk AML patients. By performing comprehensive mutational analysis on a large cohort of patients treated on a single clinical trial, Applicants demonstrate that more extensive mutational analysis can better discriminate AML patients into relevant prognostic groups (FIG. 3). For example, FLT3-ITD-negative NPM1/IDH mutant patients represent a favorable risk AML subset defined by a specific mutational genotype, whereas FLT3-ITD-negative NPM1-mutant patients without concurrent IDH mutations had a much less favorable outcome, particularly in patients with concurrent poor-risk mutations.

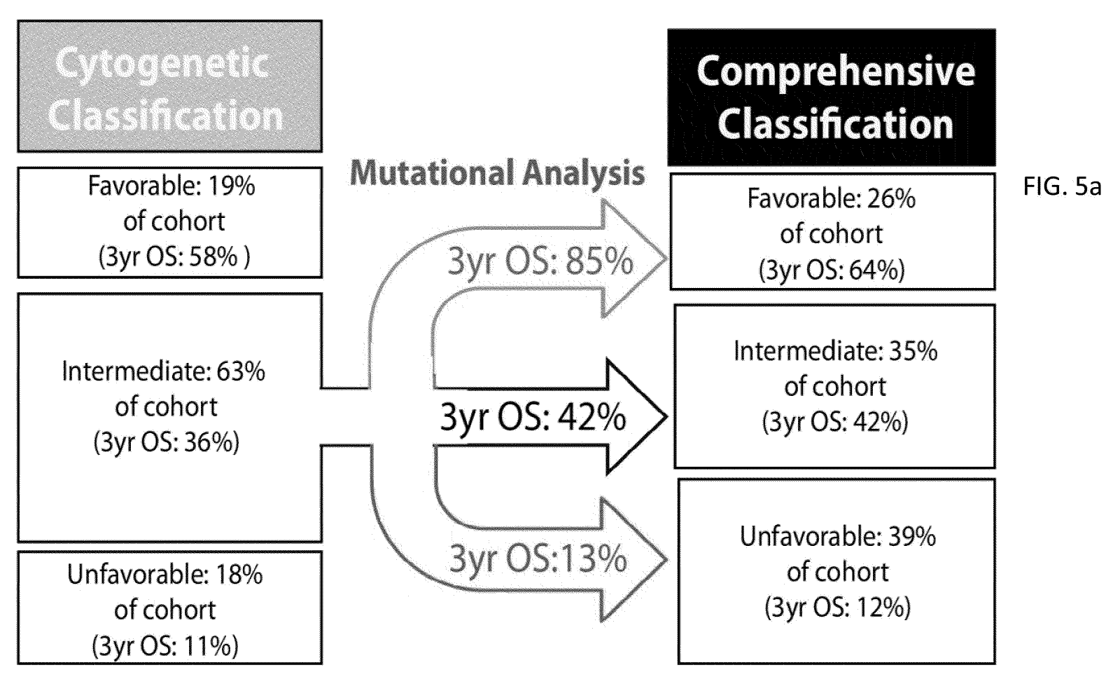

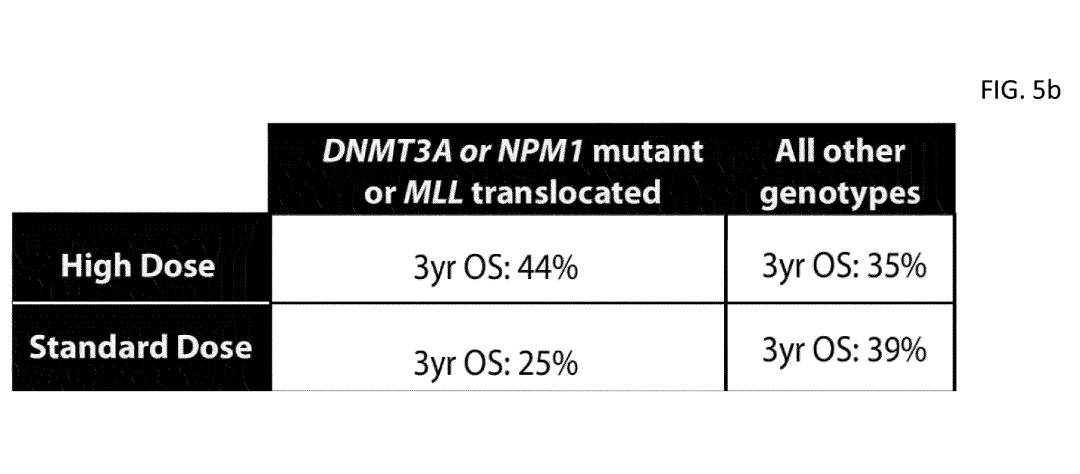

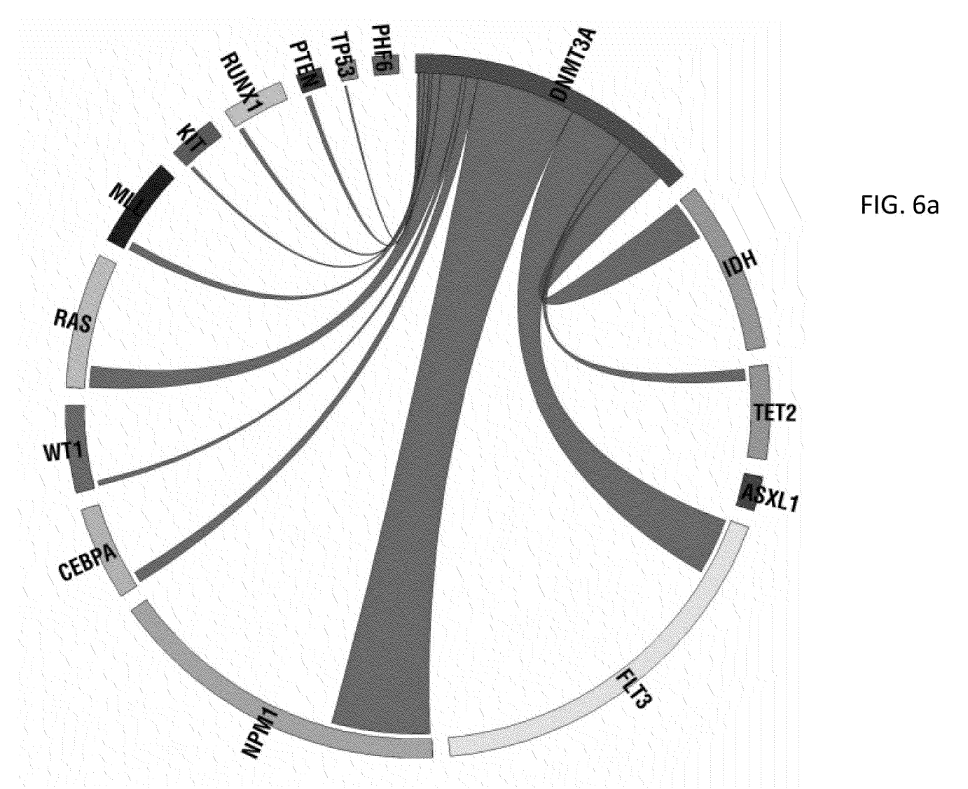

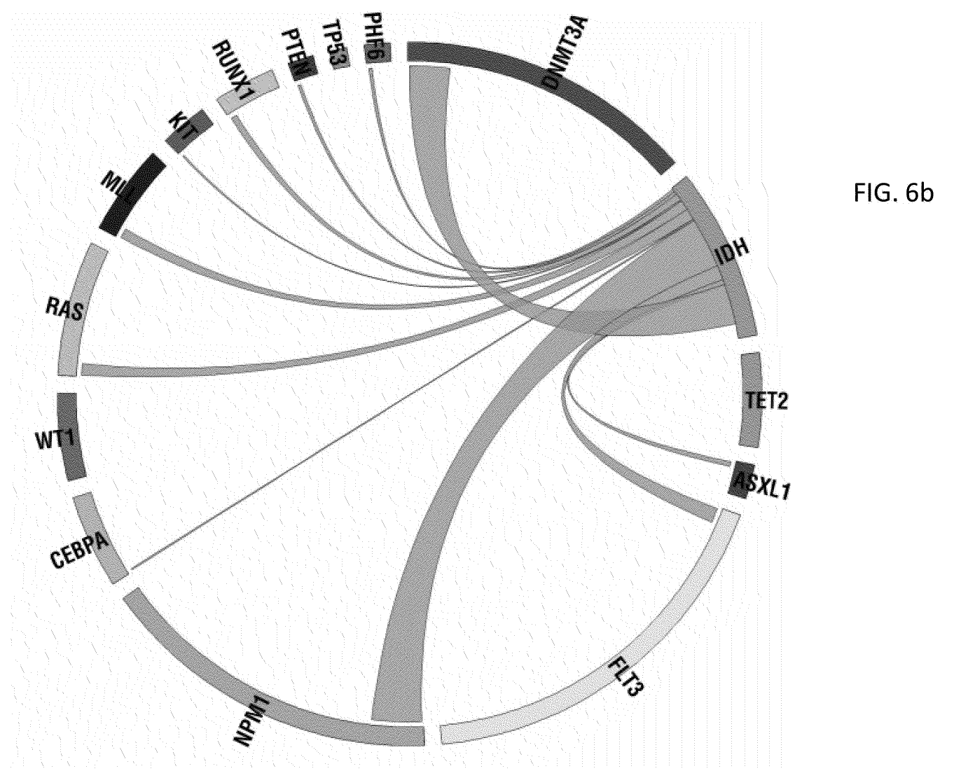

Furthermore, Applicants discovered that TET2, ASXL1, MLL-PTD, PHF6, and DNMT3A mutations can be used to define patients with adverse outcome in cytogenetically-defined intermediate-risk AML patients without the FLT3-ITD. Taken together, these data demonstrate that mutational analysis of a larger set of genetic alterations can be used to discriminate AML patients into more precise subsets with favorable, intermediate, or unfavorable risk with marked differences in overall outcome. This approach can be used to define an additional set of patients with mutationally defined favorable outcome with induction and consolidation therapy alone, and a set of patients with mutationally defined unfavorable risk who are candidates for allogeneic stem cell transplantation or clinical trials given their poor outcome with standard AML therapy (FIG. 5A).

The two recent randomized trials examining the benefits of anthracycline dose-intensification in AML demonstrated that more intensive induction chemotherapy improves outcomes in AML. (Fernandez et al., N Engl J Med, 2009, 361, 1249-59; Lowenberg et al., N Engl J Med, 2009, 361, 1235-48). Notably, re-evaluation of the original E1900 trial using our 502 patient cohort revealed that there was an even distribution of patients within each genetic risk category in both treatment arms of the original trial (p=0.41, Pearson's Chi-squared test). However, the initial reports of these studies did not identify whether dose-intensified induction therapy improved outcomes in different AML subgroups.

Applicants have discovered that anthracycline dose-intensification markedly improves outcomes in patients with mutations in DNMT3A or NPM1 or MLL translocations, suggesting mutational profiling can be used to determine which patients benefit from dose-intensive induction therapy (FIG. 5B).

Applicants also discovered mutational combinations that commonly occur in AML patients and those that rarely, if ever, co-occur consistent with the existence of additional mutational complementation groups. For example, the observation that TET2 and IDH mutations are mutually exclusive in this AML cohort led to functional studies linking IDH mutations and loss-of-function TET2 mutations in a shared mechanism of hematopoietic transformation.

As is true in the case of many treatment regimens, some patients respond to treatment with chemotherapy, for example an anthracycline antibiotic, daunorubicin, and others do not. Prescribing the treatment to a patient who is unlikely to respond to it is not desirable. Thus, it would be useful to know how a patient could be expected to respond to such treatment before a drug is administered so that non-responders would not be unnecessarily treated and so that those with the best chance of benefiting from the drug are properly treated and monitored. Further, of those who respond to treatment, there may be varying degrees of response. Treatment with therapeutics other than anthracycline or treatment with therapeutics in addition to the anthracycline daunorubicin may be beneficial for those patients who would not respond to a particular chemotherapy or in whom response to the particular chemotherapy, e.g. daunorubicin, or a similar anthracycline antibiotic, alone is less than desired.

The present disclosure demonstrates the ability of integrated mutational profiling of a clinical trial cohort to advance our understanding of AML biology, improve current prognostic models, and inform therapeutic decisions. In particular, these data indicate that more detailed genetic analysis can lead to improved risk stratification and identification of patients who benefit from more intensive induction chemotherapy.

In a specific aspect, the present disclosure is a method of screening a patient with acute myeloid leukemia for responsiveness to treatment with high dose of Daunorubicin or a pharmaceutically acceptable salt, solvate, or hydrate thereof, comprising: obtaining a genetic sample comprising an acute myeloid leukemic cell from said individual; and assaying the sample and detecting the presence of a mutation in DNMT3A or NPM1 or an MLL translocation; and correlating a finding of a mutation in DNMT3A or NPM1 or an MLL translocation, as compared to wild type controls where there is no mutation, with said acute myeloid leukemia patient being more sensitive to high dose treatment with Daunorubicin or a pharmaceutically acceptable salt, solvate, or hydrate thereof. In one embodiment, the method further comprises predicting the patient is at a lower risk of relapse of acute myeloid leukemia following chemotherapy if a mutation in DNMT3A or NPM1 or an MLL translocation is detected. In one embodiment, the method further comprises predicting the patient is at a lower risk of relapse of acute myeloid leukemia following chemotherapy if either DNMT3A or NPM1 mutations or an MLL translocation are detected.

Stratification of patient populations to predict therapeutic response is becoming increasingly valuable in the clinical management of cancer patients. For example, companion diagnostics are required for the stratification of patients being treated with targeted therapies such as trastuzumab (Herceptin, Genentech) in metastatic breast cancer, and cetuximab (Erbitux, Merck) in colorectal cancer. Predictive biomarkers are also being utilized for imatinib (Gleevec, Novartis) in gastrointestinal stromal tumors, and for gefitinib (Iressa, Astra-Zeneca) in lung cancer. Currently there is no method available to predict response to an anthracycline antibiotic in acute myeloid leukemia. To identify genes that are associated with greater sensitivity to an anthracycline antibiotic, and in particular to daunorubicine, Applicants assayed for the presence of mutations in certain genes as described above.

Genes Analyzed for Somatic Mutations in Genomic DNA of Patients with AML and their Clinical Associations, as Presently Disclosed

| GENE | CLINICAL ASSOCIATION IN AML |
|---|---|
| FLT3 | Internal tandem duplications or mutations in the tyrosine kinase |
| | domain of the receptor tyrosine kinase FLT3 are important for |
| | predicting survival in the overall cohort of AML patients as well as |
| | those with cytogenetically-defined intermediate-risk AML. |
| DNMT3A | Mutations in DNMT3A were relevant for (a) predicting for adverse |
| | overall survival in the presence of the FLT3-ITD in patients with |
| | cytogenetically-defined intermediate-risk AML and (b) predicting |
| | for responsiveness to high-dose induction chemotherapy with daunorubicin |
| | and cytarabine. |
| NPM1 | Mutations in NPM1 were relevant for (a) predicting for improved |
| | overall survival when they co-occurred with IDH1/2 mutations in |
| | cytogenetically-defined intermediate-risk AML and (b) predicting |
| | for responsiveness to high-dose induction chemotherapy with |
| | daunorubicin and cytarabine. |
| NRAS | Activating mutations in NRAS were seen in 10% of AML patients studied |
| | here. |
| CEBPA | Mutations in CEBPA were relevant for (a) predicting for improved |
| | overall survival in the overall cohort of AML patients regardless of |
| | cytogenetic risk (b) predicting for intermediate overall risk in |
| | patients with cytogenetically-defined intermediate-risk AML and |
| | the presence of the FLT3ITD. |
| TET2 | Mutations in TET2 were relevant for predicting for worsened |
| | overall risk in patients with cytogenetically-defined intermediate- |
| | risk AML regardless of the presence of the FLT3ITD. |
| WT1 | Mutations in WT1 were present in 8% of AML patients here overall |
| | but were enriched amongst patients who were refractory to initial induction |
| | chemotherapy. |
| IDH2 | Mutations in IDH2 were relevant for (a) predicting for improved |
| | overall survival in the overall cohort of AML patients regardless of |
| | cytogenetic risk specifically when mutations were present at |
| | Arginine 140; (b) predicting for favorable overall risk in patients |
| | with cytogenetically-defined intermediate-risk AML and no |
| | FLT3ITD when accompanied by an NPM1 mutation. |
| IDH1 | Mutations in IDH1 were relevant for predicting for favorable |
| | overall risk in patients with cytogenetically-defined intermediate- |
| | risk AML and no FLT3ITD when accompanied by an NPM1 |
| | mutation. |
| KIT | Mutations in KIT were seen in 6% of AML patients overall but |
| | were enriched in patients with core-binding factor translocations. In |
| | the presence of a mutation in KIT, patients with t(8;16) had an |
| | worsened overall survival compared to t(8;16) AML patients who |
| | were KIT wildtype. |
| RUNX1 | Mutations in RUNX1 were present in 5% of AML patients here. |
| MLL | Partial tandem duplications in MLL were relevant for (a) predicting |
| | for improved overall survival in patients receiving high-dose |
| | induction chemotherapy and (b) predicting for adverse overall |
| | survival in patients with cytogenetically-defined intermediate-risk |
| | AML regardless of mutations in FLT3. |
| ASXL1 | Mutations in ASXL1 were relevant for (a) predicting for adverse |
| | overall survival in the entire cohort of AML patients (b) predicting |
| | for adverse overall survival in cytogenetically-defined |
| | intermediate-risk AML patients who did not have the FLT3ITD and |
| | (c) were enriched amongst patients who failed to respond to initial |
| | induction chemotherapy. |
| PHF6 | Mutations in ASXL1 were relevant for (a) predicting for adverse |
| | overall survival in the entire cohort of AML patients and (b) |
| | predicting for adverse overall survival in cytogenetically-defined |
| | intermediate-risk AML patients who did not have the FLT3ITD. |
| KRAS | Mutations in KRAS were present in 2% of AML patients studied here. |
| PTEN | Mutations in PTEN were present in 2% of AML patients studied here. |
| TP53 | Mutations in TP53 were present in 2% of AML patients studied here. |
| HRAS | Mutations in HRAS were found in none of the AML patients studied here. |
| EZH2 | Mutations in EZH2 were found in none of the AML patients studied here. |

Specific Somatic Mutations Identified in the Sequencing of 18 Genes in AML Patients, and the Nature of these Mutations

| | NATURE AND TYPE OF SOMATIC MUTATIONS |
|---|---|
| GENE | IDENTIFIED |
| FLT3 | Numerous somatic internal tandem duplications in FLT3 were identified. |
| | These have been shown to result in constitutive activation of FLT3 |
| | signaling and are listed below. In addition, mutations in the tyrosine |
| | kinase domain of FLT3 were also identified and also shown to result in |
| | hyperactive signaling of FLT3. |
| | The specific internal tandem duplication mutations identified were as |
| | followed, though any in-frame insertion of nucleotides in the |
| | juxtamembrane domain of FLT3 is scored as an internal tandem |
| | duplication. |
| | FLT3 p.Q580_V581ins12; FLT3 p.D586_N587ins15; FLT3 |
| | p.F590_Y591ins14; FLT3 p.Y591_V592ins23; FLT3 |
| | p.D593_F594ins12; FLT3 p.F594_R595ins14; FLT3 p.R595_E596ins12; |
| | FLT3 p.Y597_E598ins17; FLT3 p.E598_Y599ins14; FLT3 |
| | p.Y599_D600ins14; FLT3 p.D600_L601ins21; FLT3 |
| | p.K602_W603ins14; FLT3 p.E604_F605ins15; FLT3 p.L610_E611ins11; |
| | FLT3 p.F612_G613ins30 |
| | Tyrosine kinase domain mutations identified: |
| | FLT3 D835Y; FLT3 D835E; FLT3 D835H; FLT3 D835V |
| DNMT3A | Mutations in DNMT3A were found as (1) out-of-frame insertion/deletions |
| | predicted to result in loss-of-function of the protein, (2) somatic nonsense |
| | mutations also predicted to result in loss-of-function of the protein, and |
| | (3) somatic missense mutations. Any out-of-frame insertion/deletion or |
| | somatic nonsense mutation would be scored as a mutation in the |
| | algorithm. |
| | Insertions/Deletions: |
| | FS at amino acid (AA) 296; FS at AA 458; FS at AA 492; FS at AA |
| | 537; FS at AA 571; FS at AA 592; FS at AA 639; FS at AA 695; FS at |
| | AA 706; FS at AA 731; FS at AA 765; FS at AA 772; FS at AA 804; FS |
| | at AA 902. |
| | Nonsense mutations: |
| | DNMT3A W581C; DNMT3A W581R; DNMT3A Y660X; DNMT3A |
| | Q696X; DNMT3A W753X; DNMT3A Q816X; DNMT3A Q886X; |
| | DNMT3A S892X. |
| | Missense mutations: |
| | DNMT3A E30A; DNMT3A P76Q; DNMT3A S105N; DNMT3A L125V; |
| | DNMT3A W297S; DNMT3A G298W; DNMT3A V328F; DNMT3A |
| | G511E; DNMT3A C537Y; DNMT3A W581C; DNMT3A W581R; |
| | DNMT3A R635W; DNMT3A V636L; DNMT3A S663P; DNMT3A |
| | E664K; DNMT3A R676W; DNMT3A I681T; DNMT3A G699S; |
| | DNMT3A S714C; DNMT3A V716I; DNMT3A T727A; DNMT3A F734L; |
| | DNMT3A T862N; DNMT3A R882C; DNMT3A R882H; DNMT3A |
| | R882S; |
| NPM1 | Insertion/deletion mutations in NPM1 which disrupt the N-terminal |
| | nucleolar localization signal of nueleophosmin and generate a nuclear |
| | export signal in its place were identified. |
| | NPM1 p.W288fs*12; NPM1 p.W288fs*16; NPM1 p.W290fs*8; NPM1 |
| | p.W290fs*10; NPM1 p.W290_K292>CFSK |
| NRAS | Activating mutations in NRAS were identified. |
| | NRas G12A; NRas G12D; NRas G12S, NRas G13D; NRas G13R; NRas |
| | Q61R; NRas Q61E; NRas Q61H; NRas Q61K; NRas Q61R; NRas |
| | Q64D |
| CEBPA | Mutations in CEBPA were identified as (1) out-of-frame |
| | insertions/deletions (2) nonsense mutations and (3) somatic missense |
| | mutations. All of these mutations have been previously identified as |
| | somatic mutations and were shown to either result in a predicted shorter |
| | protein product with altered function or to affect dimerization of CEBPA. |
| | Insertions/deletions: |
| | CEBPA FS at AA 13; CEBPA FS at AA 15; CEBPA FS at AA 20; |
| | CEBPA FS at AA 28; CEBPA FS at AA 35; CEBPA FS at AA 50; |
| | CEBPA FS at AA 93; CEBPA FS at AA 190; CEBPA FS at AA 195; |
| | CEBPA FS at AA 197; CEBPA FS at AA301; CEBPA FS at AA 303; |
| | CEBPA FS at AA 305; CEBPA FS at AA 308; CEBPA FS at AA 309; |
| | CEBPA FS at AA 311; CEBPA FS at AA 312; CEBPA FS at AA 313; |
| | CEBPA FS at AA 315. |
| | Nonsense mutations: |
| | CEBPA K275X; CEBPA E329X |
| | Somatic missense mutations: |
| | CEBPA R291C; CEBPA R300H; CEBPA L335R; CEBPA R339P. |
| TET2 | Mutations in TET2 were found as out-of-frame insertions/deletions |
| | predicted to result in loss of functional protein, nonsense mutations also |
| | predicted to result in loss of functional protein, and somatic missense |
| | mutations. Any out-of-frame insertion/deletion or somatic nonsense |
| | mutation would be scored as a mutation in our algorithm. |
| | Insertions/deletions: |
| | TET2 FS at AA 270; TET2 FS at AA 586; TET2 FS at AA 912; TET2 FS |
| | at AA 921; TET2 FS at AA 958; TET2 FS at AA 966; TET2 FS at AA |
| | 1034; TET2 FS at AA 1114; TET2 FS at AA 1118; TET2 FS at AA |
| | 1299; TET2 FS at AA 1322; TET2 FS at AA 1395; TET2 FS at AA |
| | 1439; TET2 FS at AA1448; TET2 FS at AA 1893; TET2 FS at AA1960. |
| | Nonsense mutations: |
| | TET2 S327X; TET2 K433X; TET2 R544X; TET2 R550X; TET2 Q622X; |
| | TET2 Q891X; TET2 Q916X; TET2 W1003X; TET2 E1405X; TET2 |
| | S1486X; TET2 Q1524X; TET2 Y1902X |
| | Missense mutations: |
| | TET2 P426L; TET2 E452A; TET2 F868L; TET2 Q1021R; TET2 |
| | Q1084P; TET2 E1141K; TET2 H1219Y; TET2 N1260K; TET2 R1261C; |
| | TET2 G1283D; TET2 W1292R; TET2 R1365H; TET2 G1369V; TET2 |
| | R1572W; TET2 H1817N; TET2 E1851K; TET2 I1873T; TET2 R1896M; |
| | TET2 S1898F; TET2 P1962L |
| WT1 | Mutations in WT1 were identified as out-of-frame insertion/deletions as |
| | well as somatic nonsense mutations all of which are predicted to disrupt |
| | function of WT1. Somatic missense mutations were also identified. |
| | Insertions/Deletions: |
| | WT1 FS at AA 95; WT1 FS at AA 123; WT1 FS at AA 303; WT1 FS at |
| | AA 368; WT1 FS at AA 369; WT1 FS at AA 370; WT1 FS at AA 371; |
| | WT1 FS at AA 377; WT1 FS at AA 380; WT1 FS at AA 381; WT1 FS at |
| | AA 390; WT1 FS at AA 395; WT1 FS at AA 409; WT1 FS at AA 420; |
| | WT1 FS at AA 471. |
| | Nonsense mutations: |
| | WT1 E302X; WT1 C350X; WT1 S381X; WT1 K459X |
| | Missense mutations: |
| | WT1 G60R; WT1 M250T; WT1 C350R; WT1 T337R. |
| IDH2 | Gain-of-function point mutations in IDH2 were found. |
| | IDH2 R140Q, IDH2 R172K |
| IDH1 | Gain-of-function point mutations in IDH1 were found. |
| | IDH1 R132C, IDH1 R132G, IDH1 R132H, IDH1 R132S. |
| KIT | Somatic missense mutations in KIT which result in hyperactivation of |
| | KIT signaling were identified. These are found as missense mutations at |
| | amino acid 816 or in-frame deletions in exon 8. |
| | In-frame deletions: |
| | KIT FS at AA 418; KIT FS at AA 530. |
| | Somatic missense mutations: |
| | KIT D816Y; KIT D816V. |
| RUNX1 | Mutations in RUNX1 were found as somatic out-of-frame |
| | insertion/deletion mutations and nonsense mutations which are all |
| | predicted to result in loss-of-function. Somatic missense mutations were |
| | also found. Any out-of-frame insertion/deletion or somatic nonsense |
| | mutation would be scored as a mutation in the algorithm. |
| | Somatic insertions/deletions: |
| | RUNX1 FS at AA 135.; RUNX1 FS at AA 147; RUNX1 FS at AA 183; |
| | RUNX1 FS at AA 185; RUNX1 FS at AA 220; RUNX1 FS at AA 236; |
| | RUNX1 FS at AA 321; RUNX1 FS at AA 340; RUNX1 FS at AA 415. |
| | Somatic nonsense mutations: |
| | RUNX1 Y140X; RUNX1 R204X; RUNX1 Q272X; RUNX1 E316X; |
| | RUNX1 Y414X. |
| | Somatic missense mutations: |
| | RUNX1 E8Q; RUNX1 G24A; RUNX1 V31A; RUNX1 L56S; RUNX1 |
| | W106C; RUNX1 F158S; RUNX1 D160A; RUNX1 D160E; RUNX1 |
| | R166G; RUNX1 S167T; RUNX1 G168E; RUNX1 D198N; RUNX1 |
| | R232W. |
| MLL | Somatic insertions which result in partial tandem duplications in MLL |
| | were identified. |
| ASXL1 | Mutations in ASXL1 were found as somatic out-of-frame |
| | insertion/deletion mutations and nonsense mutations which are all |
| | predicted to result in loss-of-function. Somatic missense mutations were |
| | also found. Any out-of-frame insertion/deletion or somatic nonsense |
| | mutation would be scored as a mutation in the algorithm. |
| | ASXL1 FS at AA 590; ASXL1 FS at AA 630; ASXL1 FS at AA 633; |
| | ASXL1 FS at AA 634; ASXL1 FS at AA 640; ASXL1 FS at AA 685; |
| | ASXL1 FS at AA 890. |
| | Somatic nonsense mutations: |
| | ASXL1 C594X; ASXL1 R693X; ASXL1 R1068X |
| | Somatic missense mutations: |
| | ASXL1 E348Q; ASXL1 M1050V. |
| PHF6 | Somatic out-of-frame insertion/deletion mutations, missense mutations, |
| | and nonsense mutations were seen in PHF6, all of which are predicted |
| | to result in a loss-of-function. Any out-of-frame insertion/deletion or |
| | somatic nonsense mutation would be scored as a mutation in the |
| | algorithm. |
| | Insertion/deletions: |
| | PHF6 FS at AA 176. |
| | Nonsense mutations: |
| | PHF6 R274X; PHF6 G291X; PHF6 Y301X. |
| | Somatic missense mutations: |
| | PHF6 I115K; PHF6 I314T; PHF6 H329L; PHF6 L362P. |
| KRAS | Activating mutations in KRAS were seen. |
| | KRas G12D; KRas G12S; KRas G12V; KRas G13D; KRas I36M; KRas |
| | Q61H. |
| PTEN | Somatic missense mutations in PTEN were identified which result in |
| | loss-of-function of PTEN. Any out-of-frame insertion/deletion or |
| | somatic nonsense mutation would be scored as a mutation in the |
| | algorithm. |
| | PTEN H75L; PTEN N82Y; PTEN R142W; PTEN R308H; PTEN |
| | P339S; PTEN S380C; PTEND386G |
| TP53 | Mutations in TP53 were found as somatic out-of-frame |
| | insertion/deletions, nonsense mutations, and missense mutations all of |
| | which are predicted to result in loss of TP53 function. Any out-of-frame |
| | insertion/deletion or somatic nonsense mutation would be scored as a |
| | mutation in our algorithm. |
| | Insertion/Deletions: |
| | TP53 FS at AA 30; TP53 FS at AA 31; TP53 FS at AA 45; TP53 FS at |
| | AA 93; TP53 FS at AA 337. |
| | Nonsense mutations: |
| | TP53 R213X |
| | Misense mutations: |
| | TP53 S20L; TP53 F54L; TP53 H193R; TP53 R196Q; TP53 C242Y; |
| | TP53 R267Q); TP53 R273H; TP53 T284P; TP53 G356R. |

Based on the present studies, a revised risk stratification for AML patients was devised. First, patients with internal tandem duplications in FLT3, partial tandem duplications in MLL, or mutations in ASXL1 or PHF6 are considered to have adverse overall survival regardless of cytogenetic characteristics. In contrast, patients with mutations in IDH2 at R140 or mutations in CEBPA are predicted to have favorable overall risk. For patients who do not have any of the above molecular alterations, cytogenetic status is then considered in order to determine overall risk. Cytogenetic status is defined in this prediction algorithm based on the study by Slovak, M et al. Blood 2000; 96:4075-83. In this cytogenetic classification, patients with cytogenetic alterations denoted as predicting for favorable cytogenetic risk (t(8;21), inv(16), or t(16;16)) or adverse cytogenetic risk (del(5q)/25, 27/del(7q), abn 3q, 9q, 11q, 20q, 21q, 17p, t(6;9), t(9;22) and complex karyotypes (≧3 unrelated abn)) are predicted to have an overall favorable risk or an overall adverse risk respectively. Patients which do not have any of the aforementioned favorable or adverse cytogenetic alterations, are then considered to have cytogenetically defined intermediate-risk AML. Such patients with cytogenetically defined intermediate-risk AML are further subdivided based on the presence or absence of the FLT3ITD mutation to determine overall risk. Patients with cytogenetically-defined intermediate risk AML and no FLT3ITD mutation are expected to have (1) a favorable overall risk if they have mutations in both NPM1 and IDH1/2, (2) an unfavorable overall risk if they have mutations in any one of TET2, ASXL1, PHF6, or have the MLL-PTD mutation, (3) an intermediate overall risk if they have no mutations in TET2, ASXL1, PHF6, and no MLL-PTD mutation and no NPM1 mutation in the presence of an IDH1 or IDH2 mutation. In contrast, patients with cytogenetically-defined intermediate risk AML and the presence of the FLT3ITD mutation are expected to have (1) an intermediate overall risk if they have a CEBPA mutation as well, (2) an unfavorable overall risk if they have a mutation in TET2 or DNMT3A, or have the MLL-PTD mutation or trisomy 8, (3) an intermediate overall risk if they have no mutations in TET2, DNMT3A, and no MLL-PTD mutation and no trisomy 8. In addition to the above algorithm which serves to predict overall risk at the time of diagnosis of AML patients, the present study also identified molecular predictors for response to high-dose induction chemotherapy for AML. In this part of the study, patients with mutations in any one of DNMT3A or NPM1 or an MLL-translocation/rearrangement were found to have an improved overall survival after induction chemotherapy compared with patients with no mutations in DNMT3A or NPM1 and no MLL-translocation/rearrangement.

In one embodiment, expression of nucleic acid markers is used to select clinical treatment paradigms for acute myeloid leukemia. Treatment options, as described herein, may include but are not limited to chemotherapy, radiotherapy, adjuvant therapy, or any combination of the aforementioned methods. Aspects of treatment that may vary include, but are not limited to: dosages, timing of administration, or duration or therapy; and may or may not be combined with other treatments, which may also vary in dosage, timing, or duration.

One of ordinary skill in the medical arts may determine an appropriate treatment paradigm based on evaluation of differential mutational profile of one or more nucleic acid targets identified. In one embodiment, cancers that express markers that are indicative of acute myeloid leukemia and poor prognosis may be treated with more aggressive therapies, as taught above. In another embodiment, where the gene mutations that are indicative of being a poor responder to one or more therapies may be treated with one or more alternative therapies.

In one embodiment, the sample is obtained from blood by phlebotomy or by any suitable means in the art, for example, by fine needle aspirated cells, e.g. cells from the bone marrow. The sample may comprise one or more mononuclear cells. A sample size required for analysis may range from 1, 100, 500, 1000, 5000, 10,000, to 50,000, 10,000,000 or more cells. The appropriate sample size may be determined based on the cellular composition and condition of the sample and the standard preparative steps for this determination and subsequent isolation of the nucleic acid and/or protein for use in the invention are well known to one of ordinary skill in the art.

Without limiting the scope of the present invention, any number of techniques known in the art can be employed for profiling of acute myeloid leukemia. In one embodiment, the determining step(s) comprises use of a detection assay including, but not limited to, sequencing assays, polymerase chain reaction assays, hybridization assays, hybridization assay employing a probe complementary to a mutation, fluorescent in situ hybridization (FISH), nucleic acid array assays, bead array assays, primer extension assays, enzyme mismatch cleavage assays, branched hybridization assays, NASBA assays, molecular beacon assays, cycling probe assays, ligase chain reaction assays, invasive cleavage structure assays, ARMS assays, and sandwich hybridization assays. In some embodiments, the detecting step is carried out using cell lysates. In some embodiments, the methods may comprise detecting a second nucleic acid target. In one embodiment, the second nucleic acid target is RNA. In one embodiment, the determining step comprises polymerase chain reaction, microarray analysis, immunoassay, or a combination thereof.

In one embodiment of the presently claimed method, mutations in one or more of the FLT3-ITD, DNMT3A, NPM1, IDH1, TET2, KIT, MLL-PTD, ASXL1, WT1, PHF6, CEBPA, IDH2 genes provides information about survival and/or response to therapy, wherein mutations in one or more of said genes is associated with a change in overall survival. One embodiment of the present invention further comprises detecting the mutational status of one or more genes selected from the group consisting of TET2, ASXL1, DNMT3A, PHF6, WT1, TP53, EZH2, RUNX1, PTEN, FLT3, CEBPA, MLL, HRAS, KRAS, NRAS, KIT, IDH1, and IDH2.

Identification of predictors that precisely distinguish individuals who will and will not experience a durable response to standard acute myeloid leukemia therapy is needed. The inventors of the present application identified a need for a consensus gene profile that is reproducibly associated with patient outcome for acute myeloid leukemia. In particular, the inventors of the present application have discovered certain mutations of genes in patients with acute myeloid leukemia correlate with poor survival and patient outcome. In one embodiment, the method is screening an individual for acute myeloid leukemia prognosis. In another embodiment, the method is screening an individual for response to acute myeloid leukemia therapy.

In one embodiment, the coding regions of one or more of the genes from the group consisting of TET2, ASXL1, DNMT3A, PHF6, WT1, TP53, EZH2, NPM1, CEBPA, RUNX1, and PTEN, and coding exons of one or more of the genes from the group consisting of FLT3, HRAS, KRAS, NRAS, KIT, IDH1, and IDH2 were assayed to detect the presence of mutations. In a particular embodiment, the mutational status of one or more of the FLT3-ITD, MLL-PTD, ASXL1, PHF6, DNMT3A, IDH2, and NPM1 genes provides information about survival and/or response to therapy. The acute myeloid leukemia can be newly diagnosed, relapsed or refractory acute myeloid leukemia.

One embodiment of the present invention is directed to a kit for determining treatment of a patient with AML, the kit comprising means for detecting a mutation in at least one gene selected from the group consisting of ASXL1, DNMT3A, NPM 1, PHF6, WT1, TP53, EZH2, CEBPA, TET2, RUNX1, PTEN, FLT3, HRAS, KRAS, NRAS, KIT, IDH1, and IDH2; and instructions for recommended treatment based on the presence of a mutation in one or more of said genes. In one example, the instructions for recommended treatment for the patient based on the presence of a DNMT3A or NPM1 mutation or MLL translocation indicate high-dose daunorubicin as the recommended treatment.

Kits of the invention may comprise any suitable reagents to practice at least part of a method of the invention, and the kit and reagents are housed in one or more suitable containers. For example, the kit may comprise an apparatus for obtaining a sample from an individual, such as a needle, syringe, and/or scalpel. The kit may include other reagents, for example, reagents suitable for polymerase chain reaction, such as nucleotides, thermophilic polymerase, buffer, and/or salt. The kit may comprise a substrate comprising polynucleotides, such as a microarray, wherein the microarray comprises one or more of the genes ASXL1, DNMT3A, PHF6, NPM1, CEBPA, TET2, WT1, TP53, EZH2, RUNX1, PTEN, FLT3, HRAS, KRAS, NRAS, KIT, IDH1, and IDH2.

In another embodiment, an array comprises polynucleotides hybridizing to at least 2, or at least 3, or at least 5, or at least 8, or at least 11, or at least 18 of the genes: TET2, ASXL1, DNMT3A, PHF6, WT1, TP53, EZH2, RUNX1, PTEN, FLT3, HRAS, KRAS, NRAS, NPM1, CEPA, KIT, IDH1, and IDH2. In one embodiment, the arrays comprise polynucleotides hybridizing to all of the listed genes.

As noted, the drugs of the instant invention can be therapeutics directed to gene therapy or antisense therapy. Oligonucleotides with sequences complementary to an mRNA sequence can be introduced into cells to block the translation of the mRNA, thus blocking the function of the gene encoding the mRNA. The use of oligonucleotides to block gene expression is described, for example, in, Strachan and Read, Human Molecular Genetics, 1996. These antisense molecules may be DNA, stable derivatives of DNA such as phosphorothioates or methylphosphonates, RNA, stable derivatives of RNA such as 2′-O-alkylRNA, or other antisense oligonucleotide mimetics. Antisense molecules may be introduced into cells by microinjection, liposome encapsulation or by expression from vectors harboring the antisense sequence.

One aspect of the present disclosure is a method of treating, preventing or managing acute myeloid leukemia in a patient, comprising, analyzing a genetic sample isolated from the patient for the presence of a mutation in genes DNMT3A, and NPM1, and for the presence of a MLL translocation; identifying the patient as one who will respond to high dose chemotherapy better than standard dose chemotherapy if a mutation in DNMT3A or NPM1 or a MLL translocation are present; and administering high dose therapy to the patient. The patient, in one example, is characterized as intermediate-risk on the basis of cytogenetic analysis. In one example, the therapy comprises the administration of anthracycline. In a related embodiment, administering high dose therapy comprises administering one or more high dose anthracycline antibiotics selected from the group consisting of Daunorubicin, Doxorubicin, Epirubicin, Idarubicin, Mitoxantrone, and Adriamycin. In one embodiment, Daunorubicin, Idarubicin and/or Mitoxantrone is used.

In one embodiment, the high dose administration is Daunorubicin administered at 60 mg per square meter of body-surface area (60 mg/m2), or higher, daily for three days. In a particular embodiment, the high dose administration is Daunorubicin administered at about 90 mg per square meter of body-surface area (90 mg/m2), daily for three days. In one embodiment, the high dose daunorubicin is administered at about 70 mg/m2 to about 140 mg/m2. In a particular embodiment, the high dose daunorubicin is administered at about 70 mg/m2 to about 120 mg/m2. In a related embodiment, this high dose administration is given each day for three days, that is for example a total of about 300 mg/m2 over the three days (3×100 mg/m2). In another example, this high dose is administered daily for 2-6 days. In other clinical situations, an intermediate daunorubicin dose is administered. In one embodiment, the intermediate dose daunorubicin is administered at about 60 mg/m2. In one embodiment, the intermediate dose daunorubicin is administered at about 30 mg/m2 to about 70 mg/m2. Additionally, the related anthracycline idarubicin, in one embodiment, is administered at from about 4 mg/m2 to about 25 mg/m2. In one embodiment, the high dose idarubicin is administered at about 10 mg/m2 to 20 mg/m2. In one embodiment, the intermediate dose idarubicin is administered at about 6 mg/m2 to about 10 mg/m2. In a particular embodiment, idarubicin is administered at a dose of about 8 mg/m2 daily for five days. In another example, this intermediate dose is administered daily for 2-10 days.

In another aspect, the present disclosure is a method for preparing a personalized genomics profile for a patient with acute myeloid leukemia, comprising: subjecting mononuclear cells extracted from a bone marrow aspirate or blood sample from the patient to gene mutational analysis; assaying the sample and detecting the presence of trisomy 8 and one or more mutations in a gene selected from the group consisting of FLT3ITD, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2 in said cells; and generating a report of the data obtained by the gene mutation analysis, wherein the report comprises a prediction of the likelihood of survival of the patient or a response to therapy.

Methods of monitoring gene expression by monitoring RNA or protein levels are known in the art. RNA levels can be measured by any methods known to those of skill in the art such as, for example, differential screening, subtractive hybridization, differential display, and microarrays. A variety of protocols for detecting and measuring the expression of proteins, using either polyclonal or monoclonal antibodies specific for the proteins, are known in the art. Examples include Western blotting, enzyme-linked immunosorbent assay (ELISA), radioimmunoassay (RIA), and fluorescence activated cell sorting (FACS).

### Examples

The invention, having been generally described, may be more readily understood by reference to the following examples, which are included merely for purposes of illustration of certain aspects and embodiments of the present invention, and are not intended to limit the invention in any way.

Each of the applications and patents cited in this text, as well as each document or reference cited in each of the applications and patents (“application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List or in the text itself; and, each of these documents or references (“herein-cited references”), as well as each document or reference cited in each of the herein-cited references (including any manufacturer's specifications, instructions, etc.), is hereby expressly incorporated herein by reference.

Patients

Mutational analysis was performed on diagnostic patient samples from the ECOG E1900 trial in the test (n=398) and validation (n=104) cohorts. The test cohort comprised of all E1900 patients for whom viably frozen cells were available for DNA extraction and mutational profiling. The validation cohort comprised of a second set of patients for whom samples were banked in Trizol, which was used to extract DNA for mutational studies.

Clinical characteristics of the patients studied compared to the complete E1900 trial cohort are in Table 1. The median follow-up time of patients included for analysis was 47.4 months from induction randomization. Cytogenetic analysis, fluorescent in situ hybridization, and RT-PCR for recurrent cytogenetic lesions was performed as described initially by Slovak et al. and utilized previously with central review by the ECOG Cytogenetics Committee (see ref. 16 and 17).

Mutational Analysis

Source of the DNA was bone marrow for 55.2% (277/502) and peripheral blood for 44.8% (225/502) of the samples. Applicants sequenced the entire coding regions of TET2, ASXL1, DNMT3A, CEBPA, PHF6, WT1, TP53, EZH2, RUNX1, and PTEN and the regions of previously described mutations for FLT3, NPM1, HRAS, KRAS, NRAS, KIT, IDH1, and IDH2.

The genomic coordinates and sequences of all primers utilized in the instant disclosure are provided for in Table 2. Paired remission DNA was available from 241 of the 398 samples in the initially analyzed cohort and 65 of the 104 in the validation cohort. Variants that could not be validated as bona fide somatic mutations due to unavailable remission DNA and their absence from the published literature of somatic mutations were censored with respect to mutational status for that specific gene. Further details of the sequencing methodology are provided infra.

Statistical Analysis

Mutual exclusivity of pairs of mutations was evaluated by fourfold contingency tables and Fisher's exact test. The association between mutations and cytogenetic risk classification was tested using the chi-square test. Hierarchical clustering was performed using the Lance-Williams dissimilarity formula and complete linkage.

Survival time was measured from date of randomization to date of death for those who died and date of last follow-up for those who were alive at the time of analysis. Survival probabilities were estimated using the Kaplan-Meier method and compared across mutant and wild-type patients using the log-rank test. Multivariate analyses were conducted using the Cox model with forward selection. Proportional hazards assumption was checked by testing for a non-zero slope in a regression of the scaled Schoenfeld residuals on functions of time (Table 3).

When necessary, such as the analyses performed in various subsets, results of the univariate analyses were used to select the variables to be included in the forward variable search. Final multivariate models informed the development of novel risk classification rules. When indicated, p-values were adjusted to control the family wise error rate (FWER) using the complete null distribution approximated by resampling obtained through PROC MULTTEST in SAS or the multtest library in R¹⁹. These adjustements were performed to adjust for the probability of making one or more false discoveries given that multiple pairwise tests were being performed. The only exception is adjustment for tests regarding effect of mutations on response to induction dose where a step-down Holm procedure was used to correct for multiple testing. All analyses were performed using SAS 9.2 (www.sas.com) and R 2.12 (www.r-project.org).

Supplementary Methods

Diagnostic Samples from ECOG 1900 Clinical Trial: DNA was isolated from pretreatment bone marrow samples of 398 patients enrolled in the ECOG E1900 trial; DNA was isolated from mononuclear cells after Ficoll purification. IRB approval was obtained at Weill Cornell Medical College and Memorial Sloan Kettering Cancer Center. All genomic DNA samples were whole genome amplified using 029 polymerase. Remission DNA was available from 241 patients who achieved complete remission after induction chemotherapy. Cytogenetic, fluorescent in situ hybridization, and RT-PCR for recurrent cytogenetic lesions was performed as described previously (Bullinger et al., N Engl J Med 2004, 350, 1605-1616) with central review by the ECOG Cytogenetics Committee.

Integrated Mutational Analysis:

Mutational analysis of the entire coding regions of TET2, ASXL1, DNMT3A, PHF6, WT1, TP53, NPM1, CEBPA, EZH2, RUNX1, and PTEN and of coding exons of FLT3, HRAS, KRAS, NRAS, KIT, IDH1, and IDH2 with known somatic mutations was performed using PCR amplification and bidirectional Sanger sequencing as previously described. 13 Primer sequences and PCR conditions are provided in Table 1.

Target regions in individual patient samples were PCR amplified using standard techniques and sequenced using conventional Sanger sequencing, yielding 93.3% of all trimmed reads with an average quality score of 20 or more. All traces were reviewed manually using Mutation Surveyor (SoftGenetics, State College, Pa.). All variants were validated by repeat PCR amplification and Sanger resequencing of unamplified diagnostic DNA. All mutations which were not previously reported to be either somatic or germline were analyzed in matched remission DNA, when available, to determine somatic status. All patients with variants whose somatic status could not be determined were censored with regard to mutational status for the specific gene.

NPM1/CEBPA Next-Generation Sequencing Analysis:

A mononucleotide tract near the canonical frameshift mutations in NPM1 and the high GC content of the CEBPA gene limited Applicants' ability to obtain sufficiently high quality Sanger sequence traces for primary mutation calling. Applicants therefore performed pooled amplicon resequencing of NPM1 and CEBPA using the SOLiD 4 system. We performed PCR amplification followed by barcoding (20 pools each with 20 samples) and SOLiD sequencing. The data was processed through the Bioscope pipeline: all variants not present in reference sequence were manually inspected and validated by repeat PCR amplification and Sanger sequencing.

Mutational Cooperativity Matrix:

Applicants adapted the Circos graphical algorithm to visualize co-occuring mutations in AML patients. The arc length corresponds to the proportion of patient with mutations in the first gene and the ribbon corresponds to the percentage of patients with a coincident mutation in the second gene. Pairwise cooccurrence of mutations is denoted only once, beginning with the first gene in the clockwise direction. Since only pairwise mutations are encoded for clarity, the arc length was adjusted to maintain the relative size of the arc and the correct proportion of patients with a single mutant allele is represented by the empty space within each mutational subset.

Statistical Analysis:

Mutual exclusivitity of pairs of mutations were evaluated by fourfold contingency tables and Fisher's exact test. The association between mutations and cytogenetic risk classification was tested using the chi-square test. Hierarchical clustering was performed using the Lance-Williams dissimilarity formula and complete linkage. Survival time was measured from date of randomization to date of death for those who died and date of last follow-up for those who were alive at the time of analysis. Survival probabilities were estimated using the Kaplan-Meier method and compared across mutant and wildtype patients using the log-rank test. Multivariate analyses were conducted using the Cox model. Proportional hazards assumption was checked by testing for a non-zero slope in a regression of the scaled Schoenfeld residuals on functions of time. Many of the statistical analyses conducted in this study use Cox regression which depends on the assumption of proportional hazards.

Table 3 shows the results of the checks which were conducted for each mutation to determine whether the resultant survival curves (one curve for mutant and one curve for wildtype for each mutation) satisfy this assumption. A significant p-value indicates a departure from the proposal hazard assumption. Out of the 27 mutations included in this study, only a single one significantly deviated from proportional hazards (MLL-PTD, p=0.04). Considering the possible multiple testing problem, one would have expected 1-2 significances in this table by chance only hence Applicants conclude that it is acceptable to use the Cox regression model for all mutations. Forward model selection was employed. When necessary, such as the analyses performed in various subsets, results of the univariate analyses were used to select the variables to be included in the forward variable search. Final multivariate models informed the development of novel risk classification rules. All analyses were performed using SAS 9.2 (www.sas.com) and R 2.12 (www.r-project.org).

Frequency of Genetic Alterations in De Novo AML.

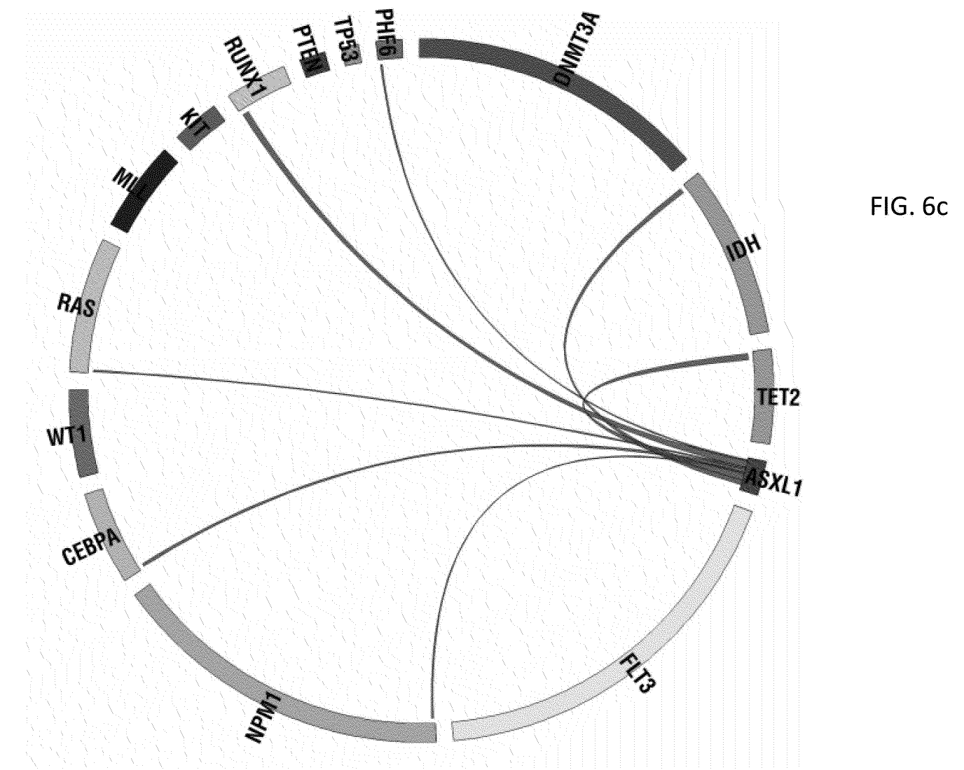

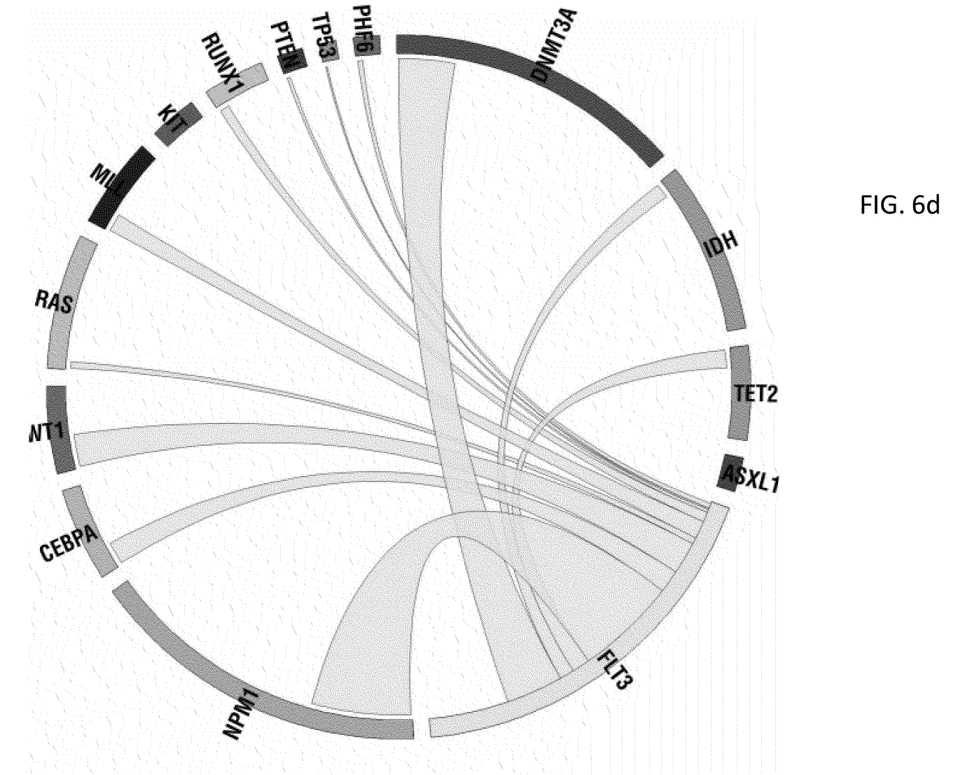

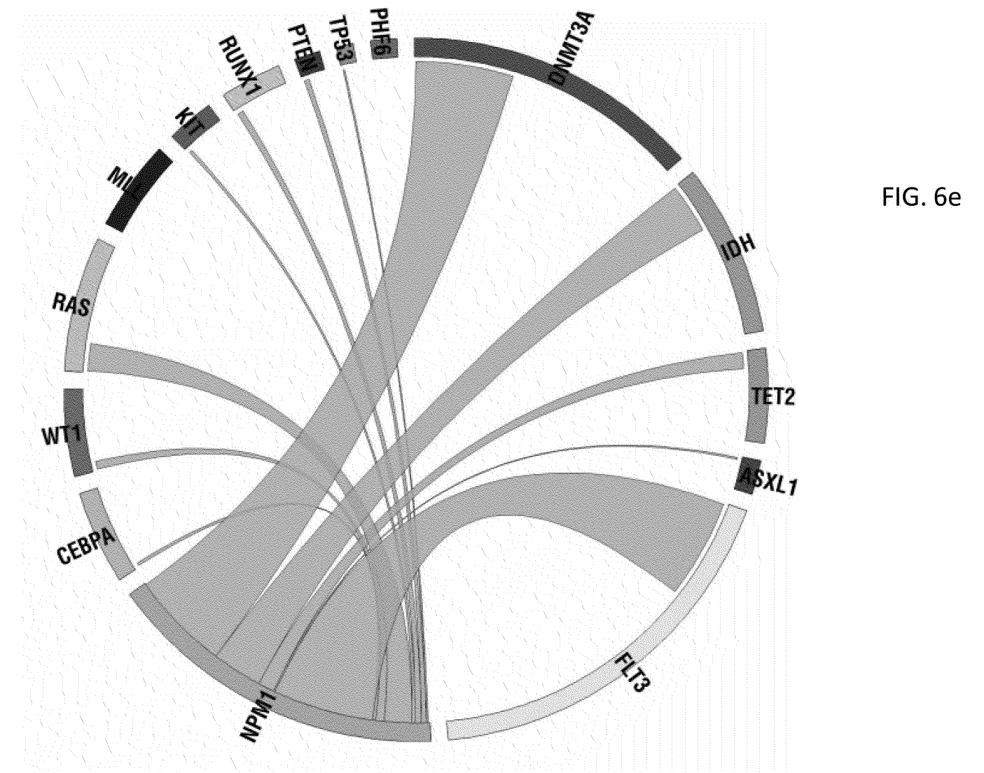

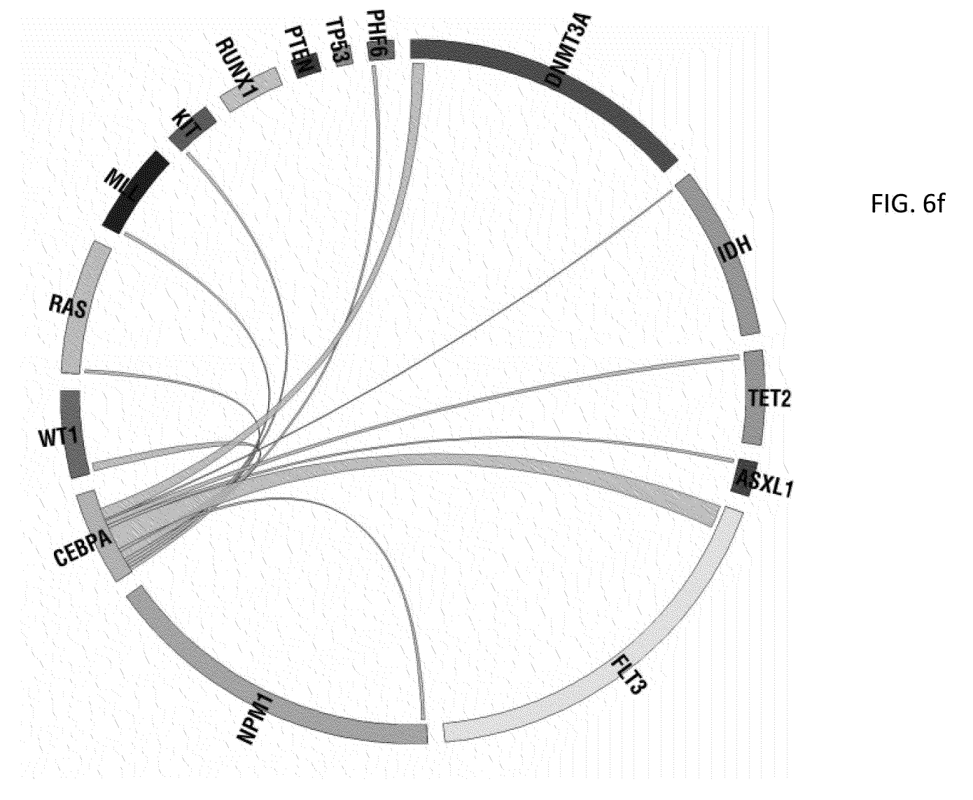

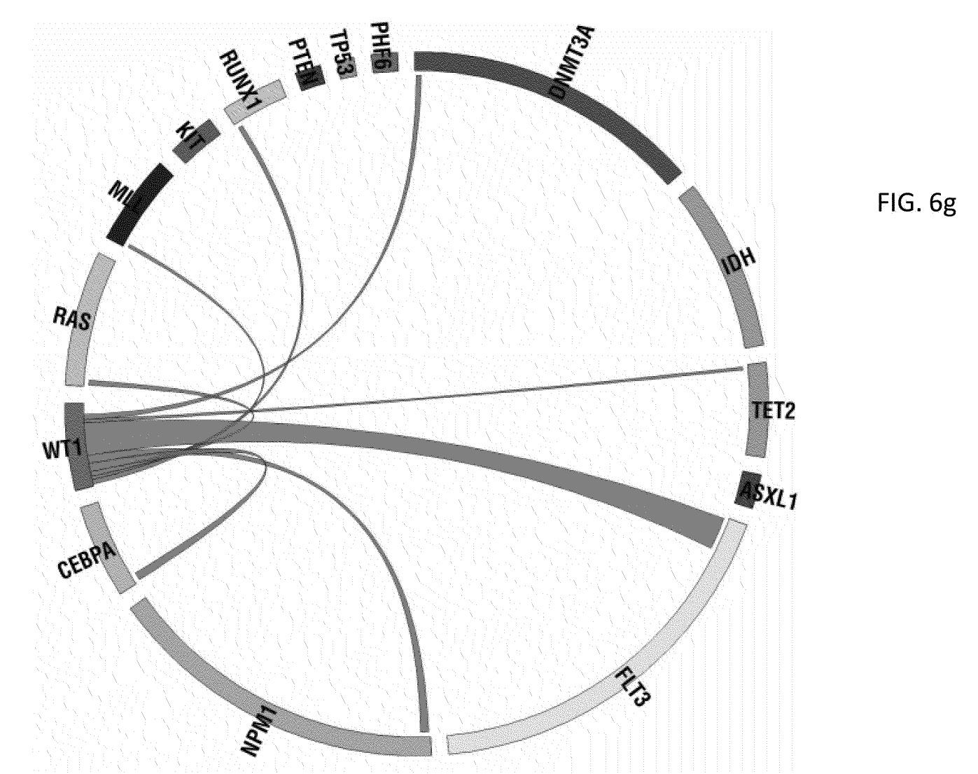

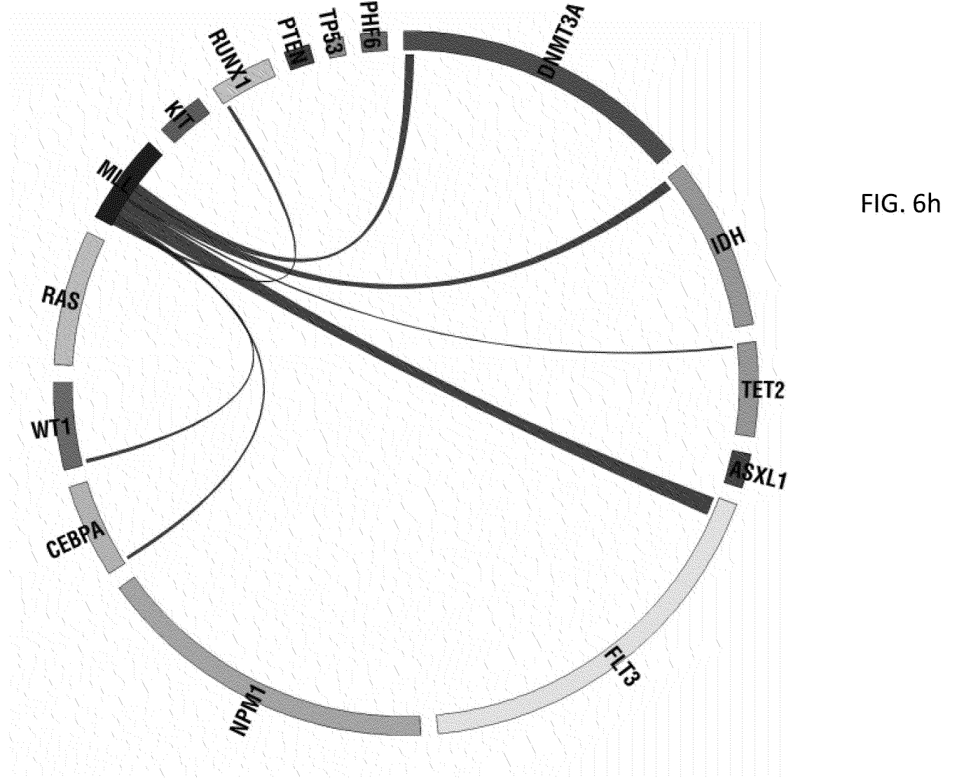

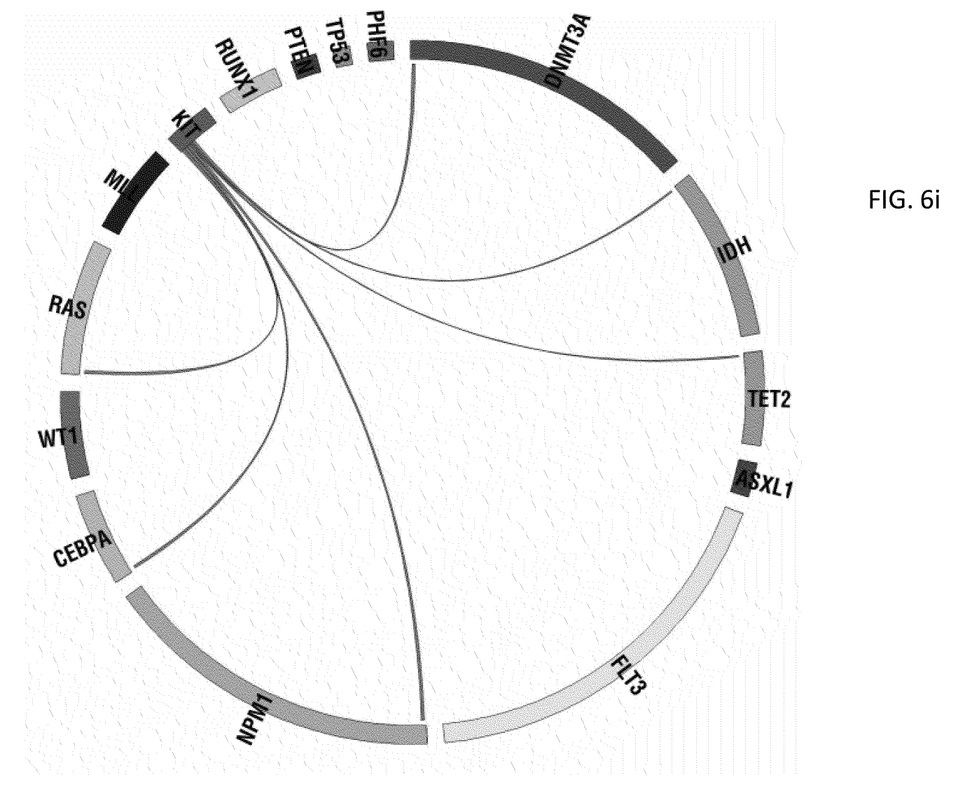

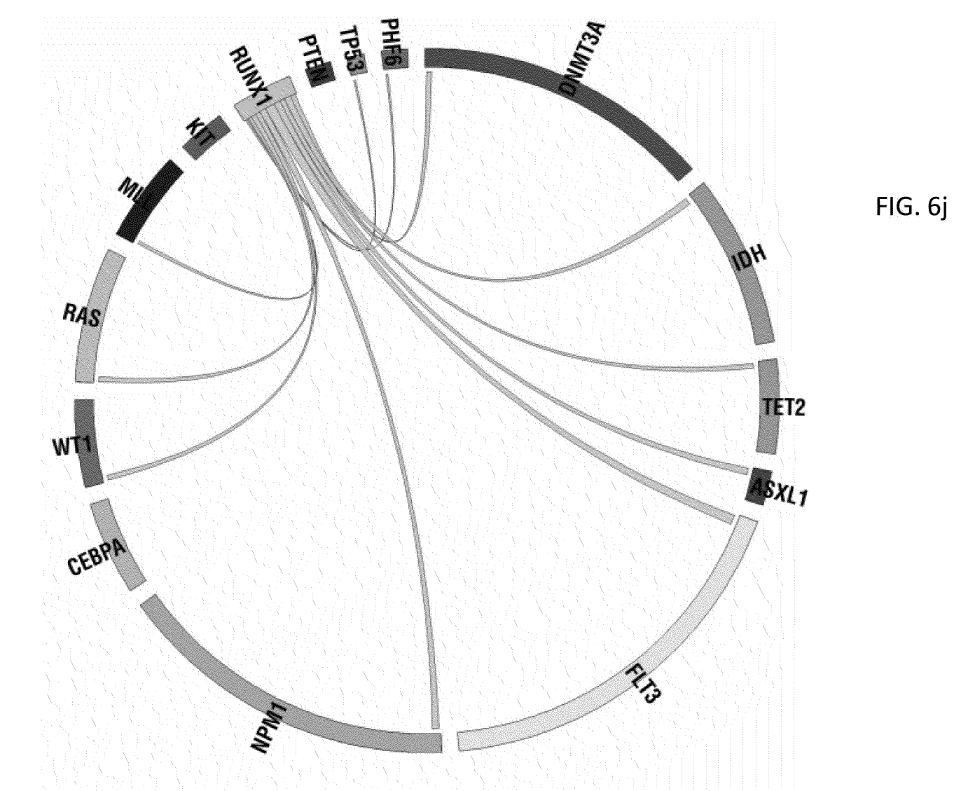

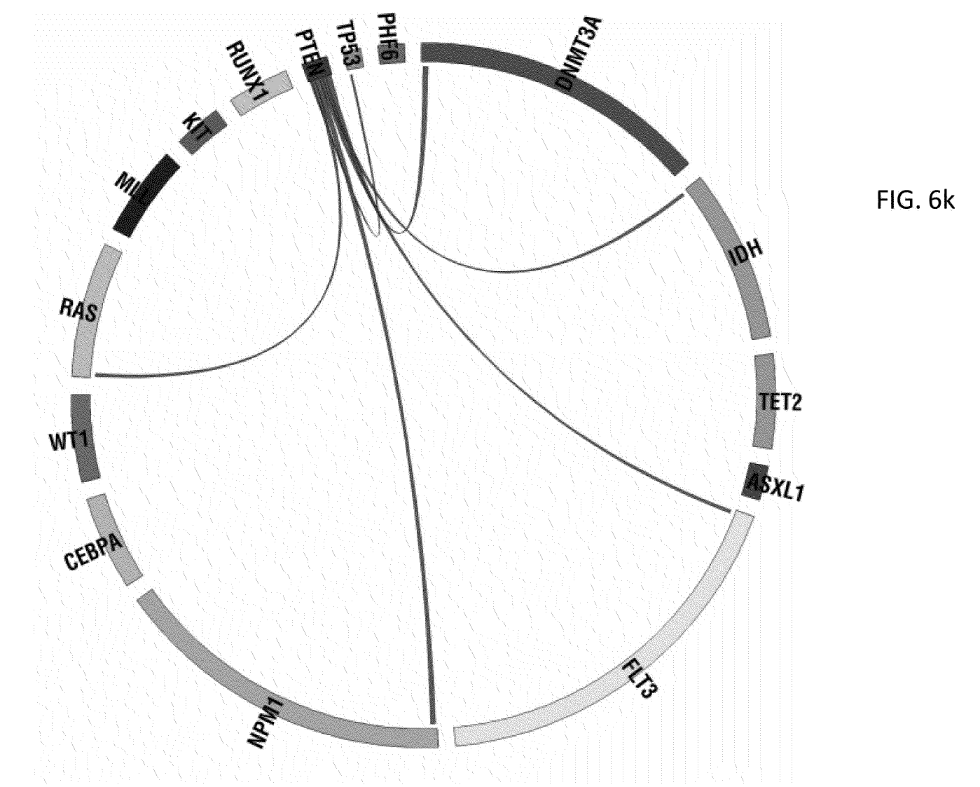

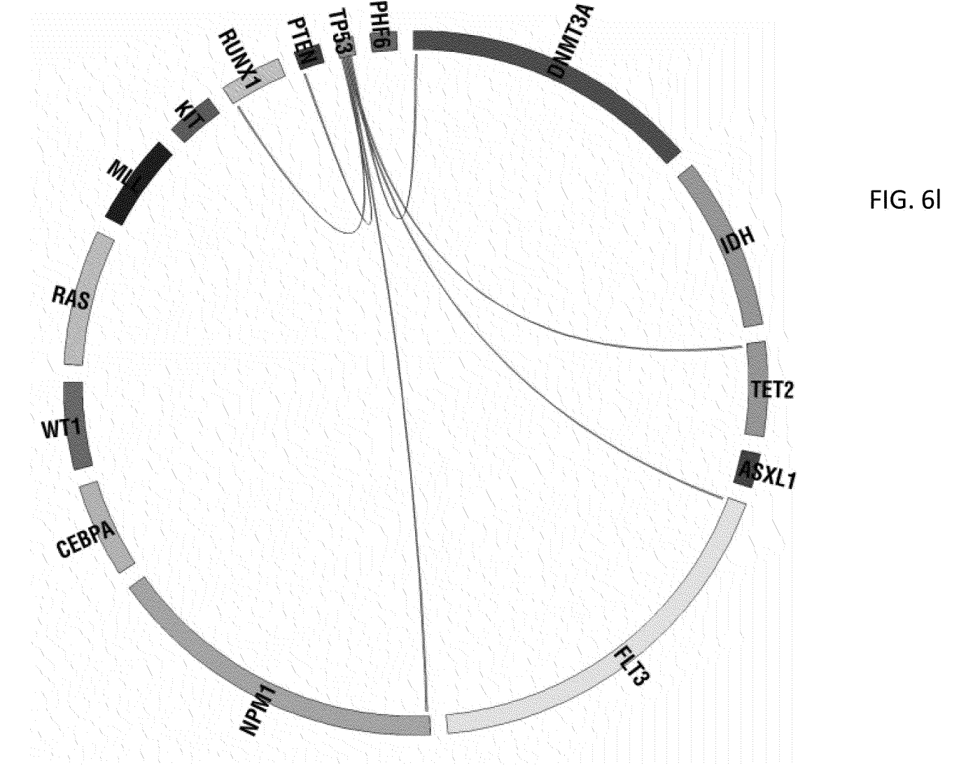

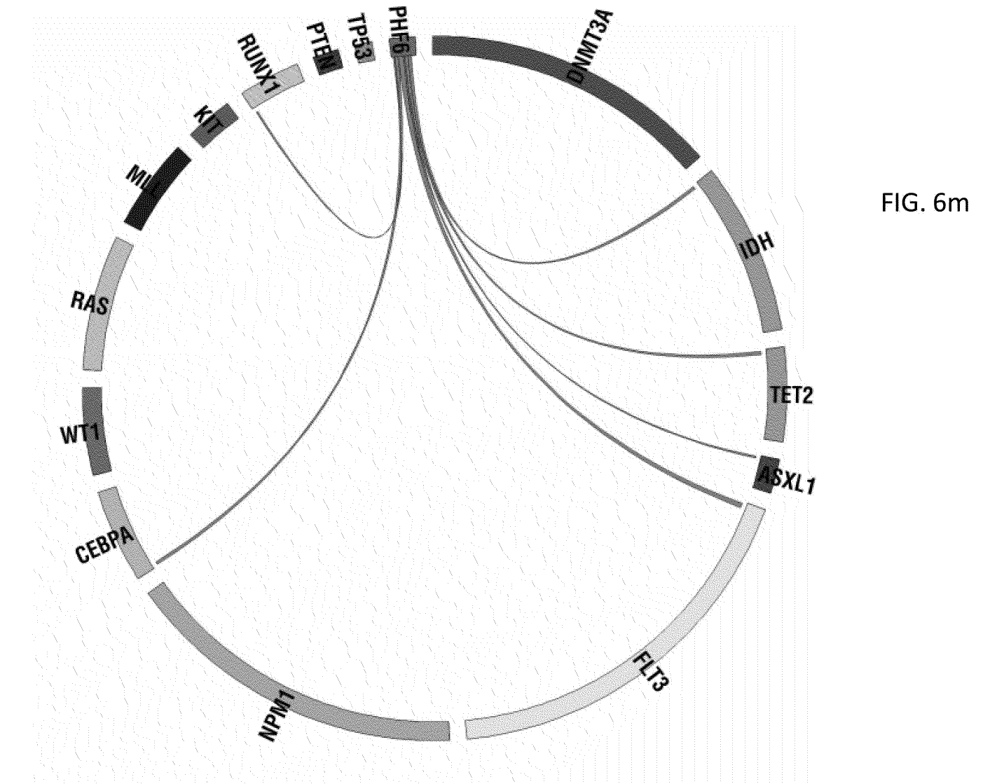

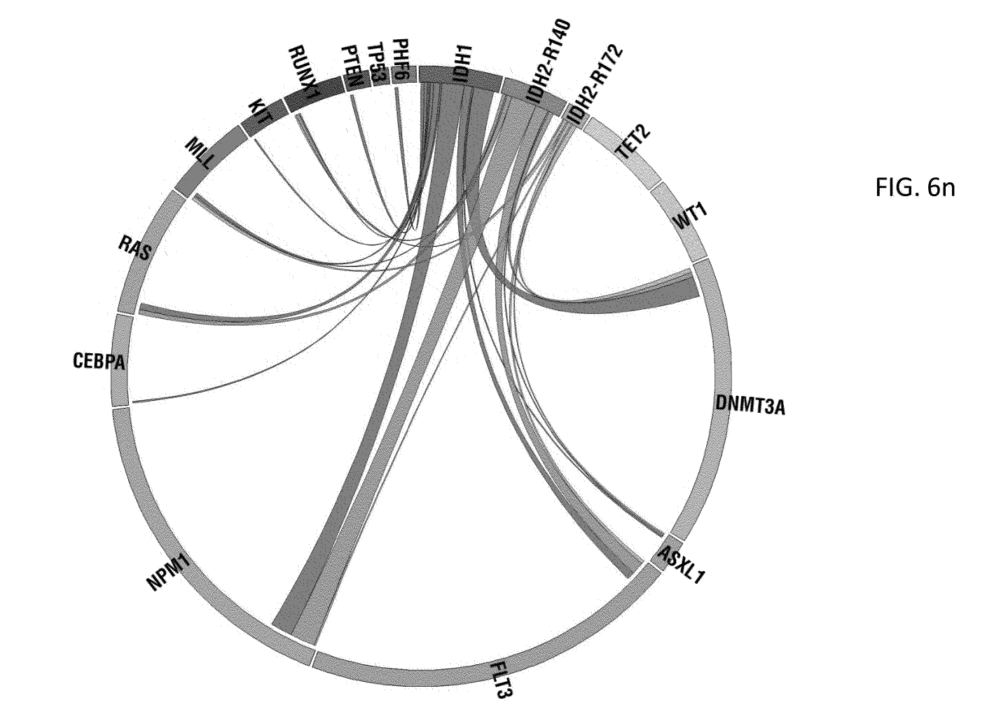

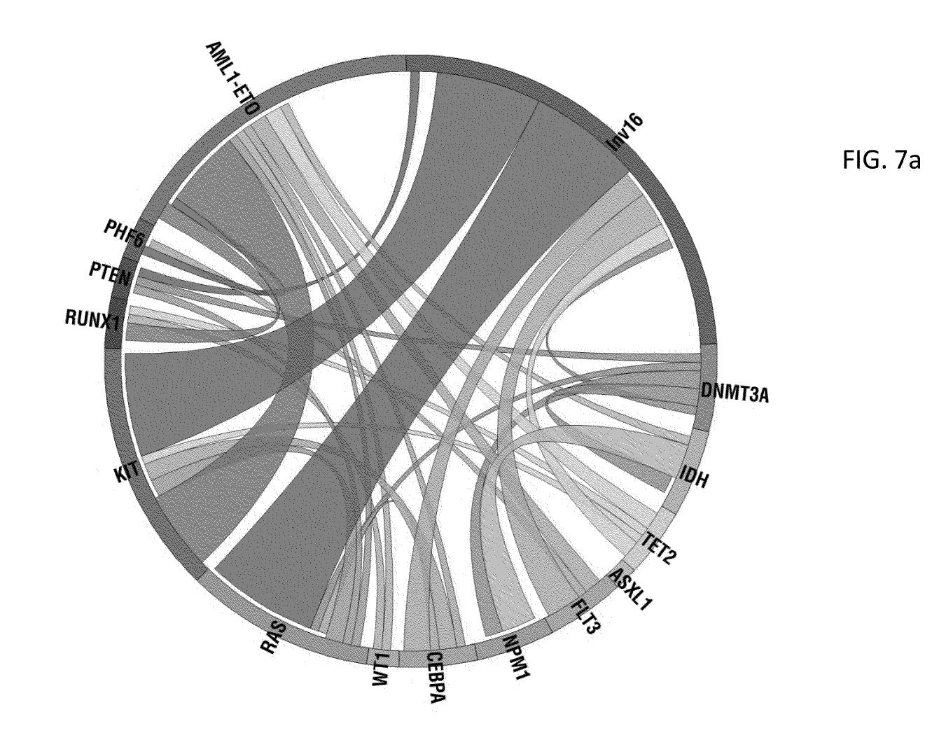

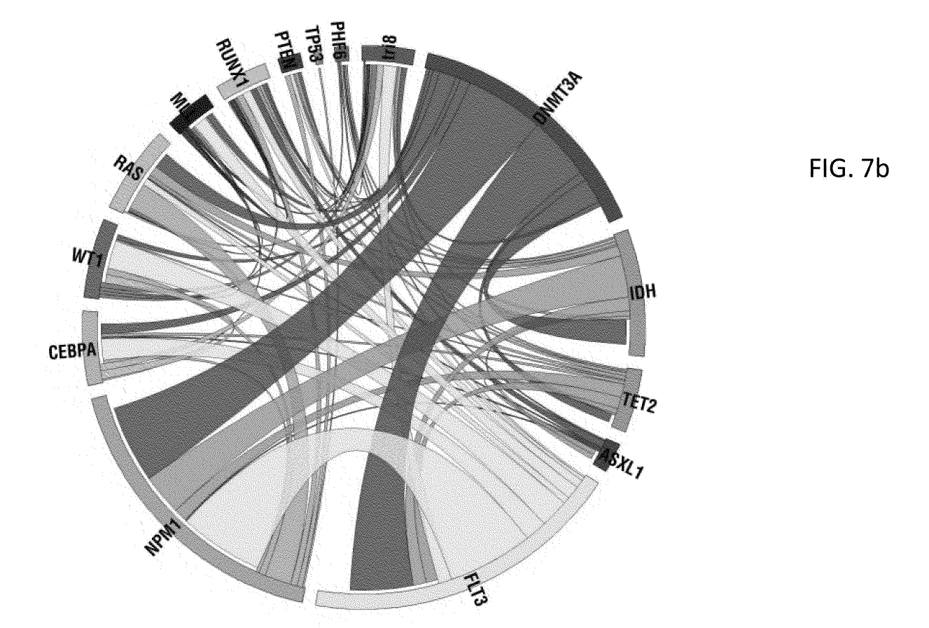

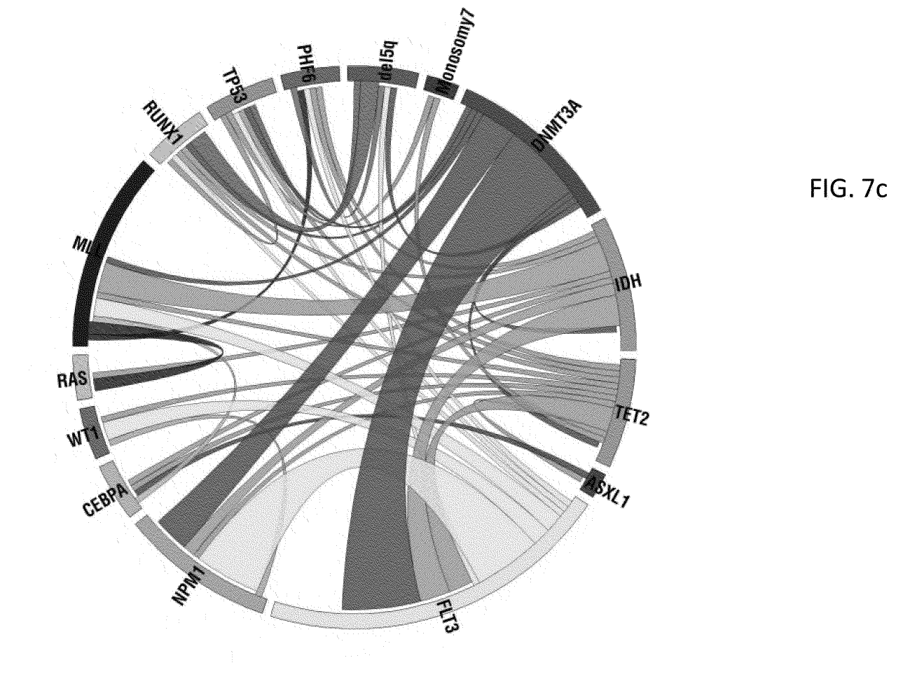

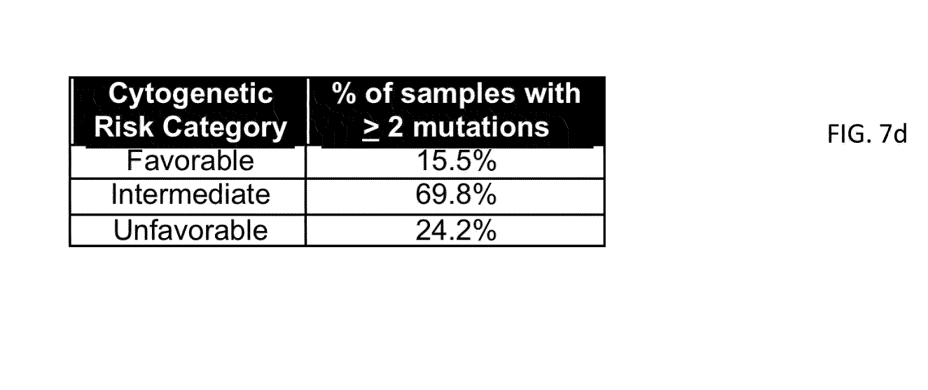

Somatic alterations were identified in 97.3% of patients. FIGS. 1A-C show the frequency of somatic mutations in the entire cohort and the interrelationships between the various mutations visually represented using a Circos plot. Data for all molecular subsets are provided in FIGS. 6 and 7 and Tables 4 and 5. In particular, mutational heterogeneity in patients with intermediate risk AML was higher than in patients with favorable or unfavorable risk AML (p=0.01; FIG. 7D).

Mutational Complementation Groups in AML.

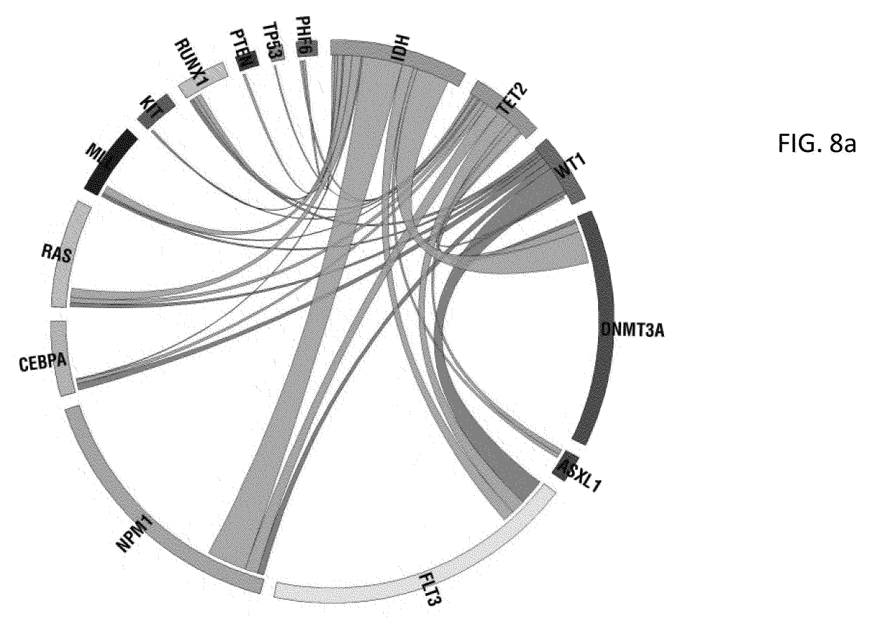

Integrated mutational analysis allowed Applicants to identify frequently co-occurring mutations and mutations that were mutually exclusive in the E1900 patient cohort (Table 6). In addition to noting a frequent co-occurrence between KIT mutations and core-binding factor alterations t(8;21) and inv(16)/t(16;16) (p<0.001), Applicants found significant co-occurrence of IDH1 or IDH2 mutations with NPM1 mutations (p<0.001), and DNMT3A mutations with NPM1, FLT3, and IDH1 alleles (p<0.001 for all) (Table 7). Applicants previously reported IDH1 and IDH2 mutations were mutually exclusive with TET2 mutations; detailed mutational analysis revealed that IDH1/2 mutations were also exclusive with WT1 mutations (p<0.001; FIG. 8 and Table 8). Applicants also observed that DNMT3A mutations and MLL-translocations were mutually exclusive (p<0.01).

Molecular Determinants of Overall Survival in AML.

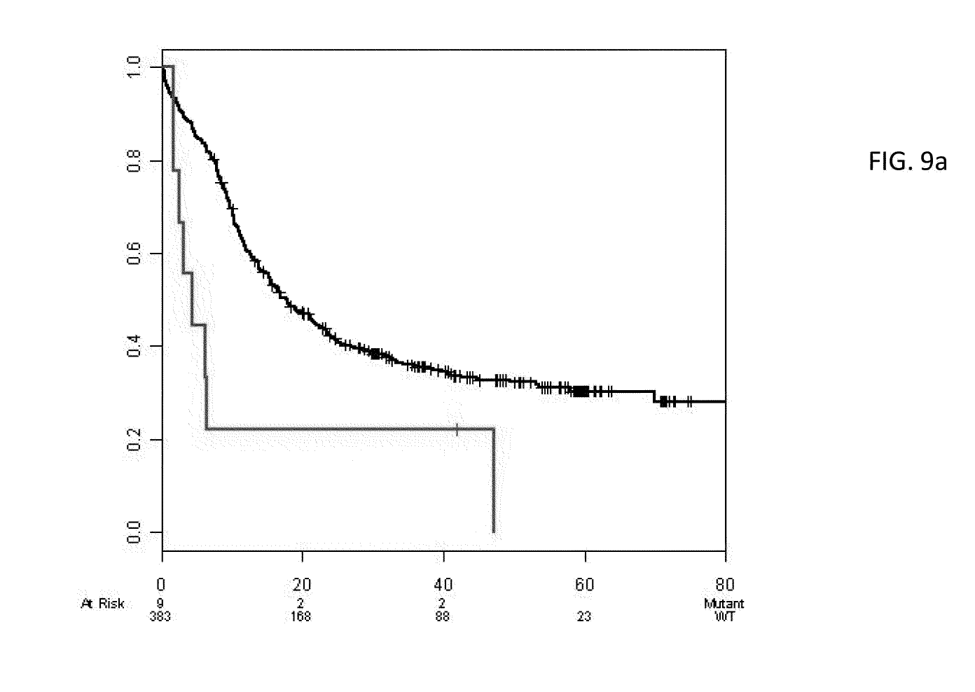

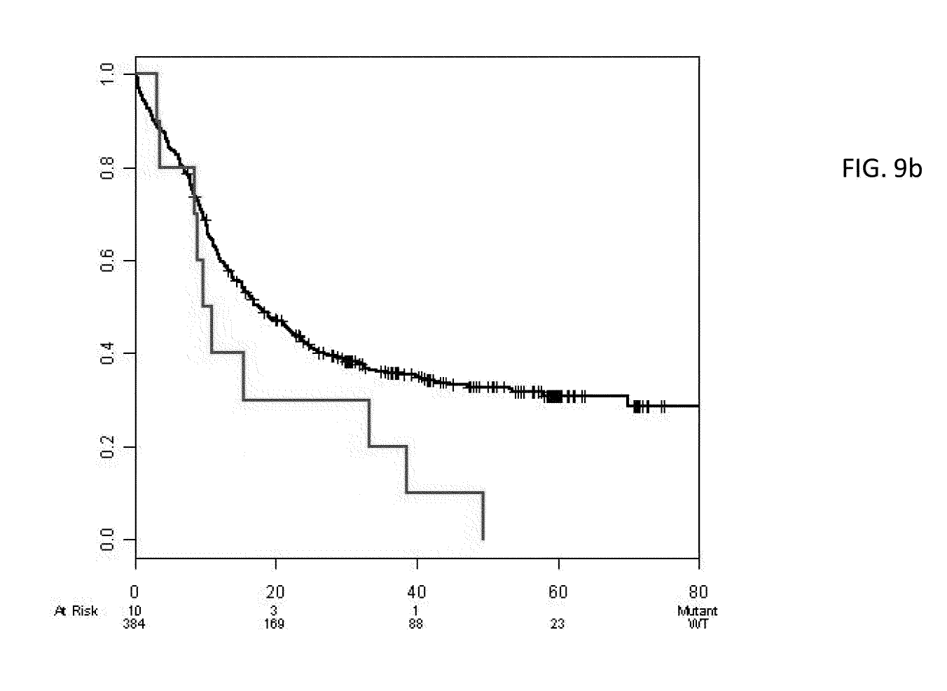

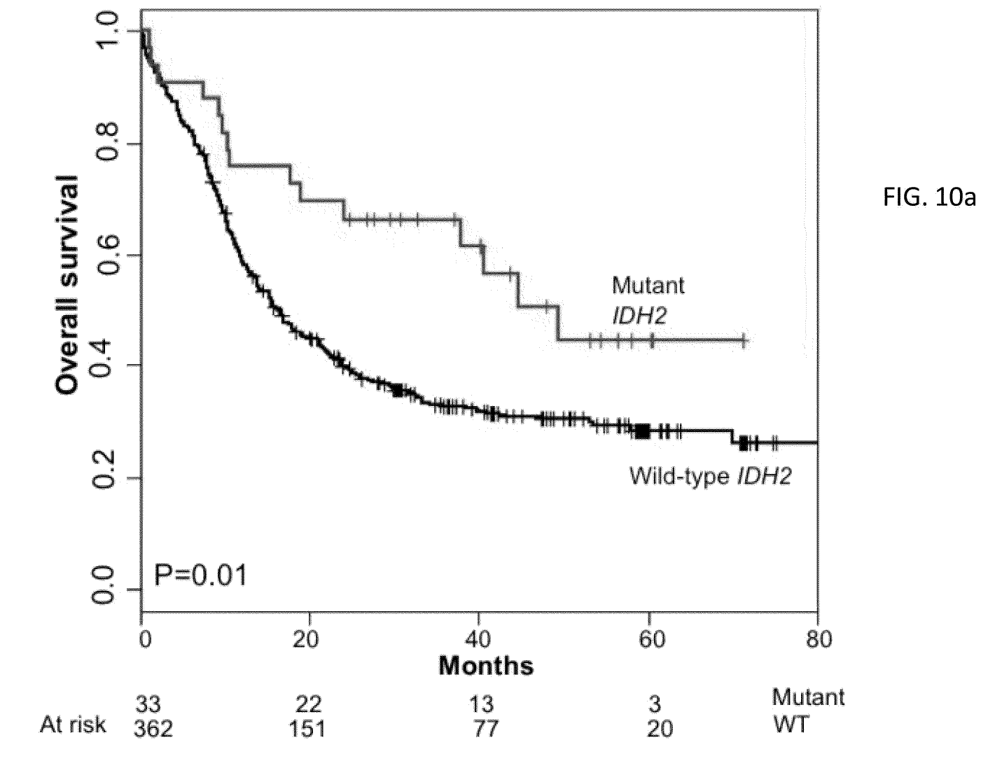

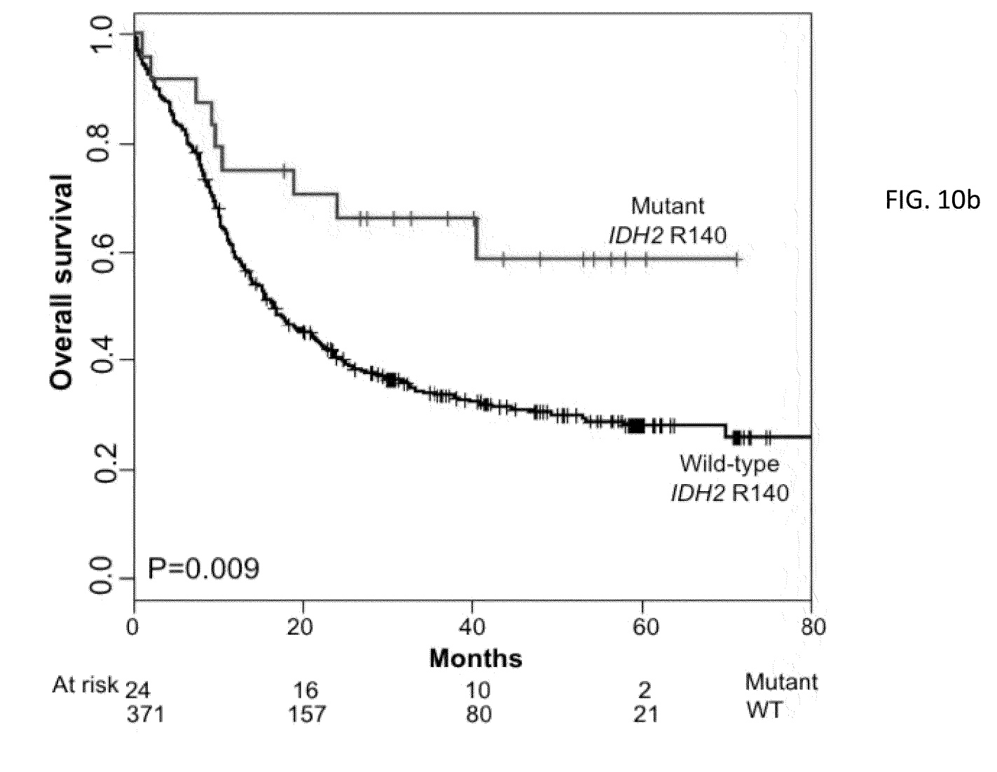

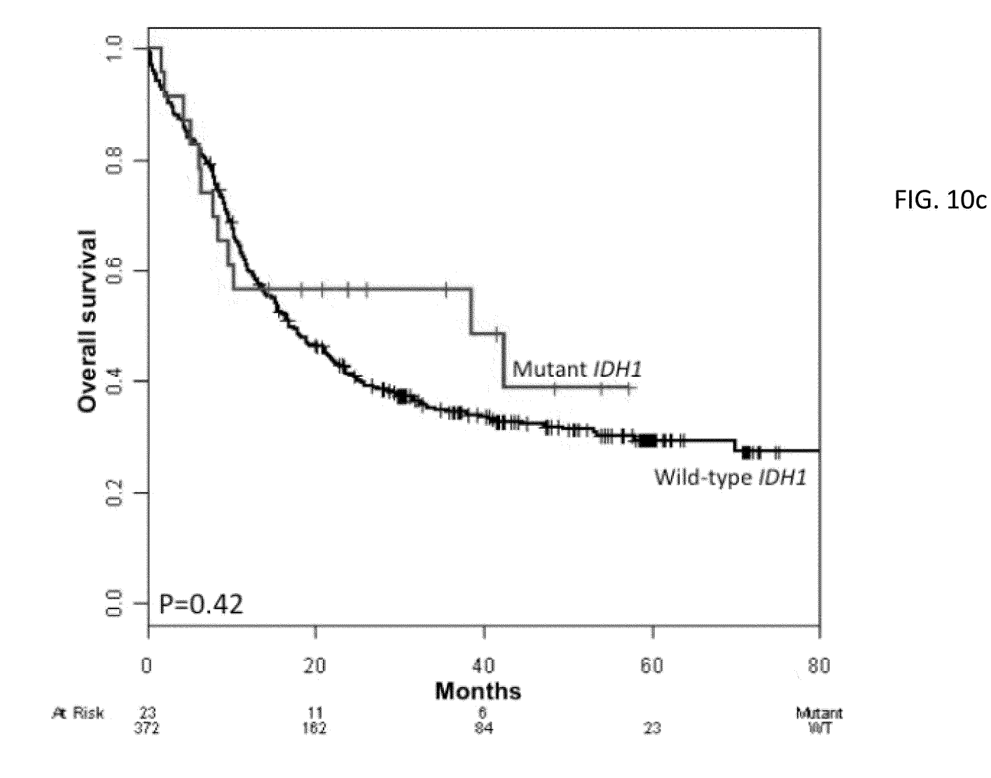

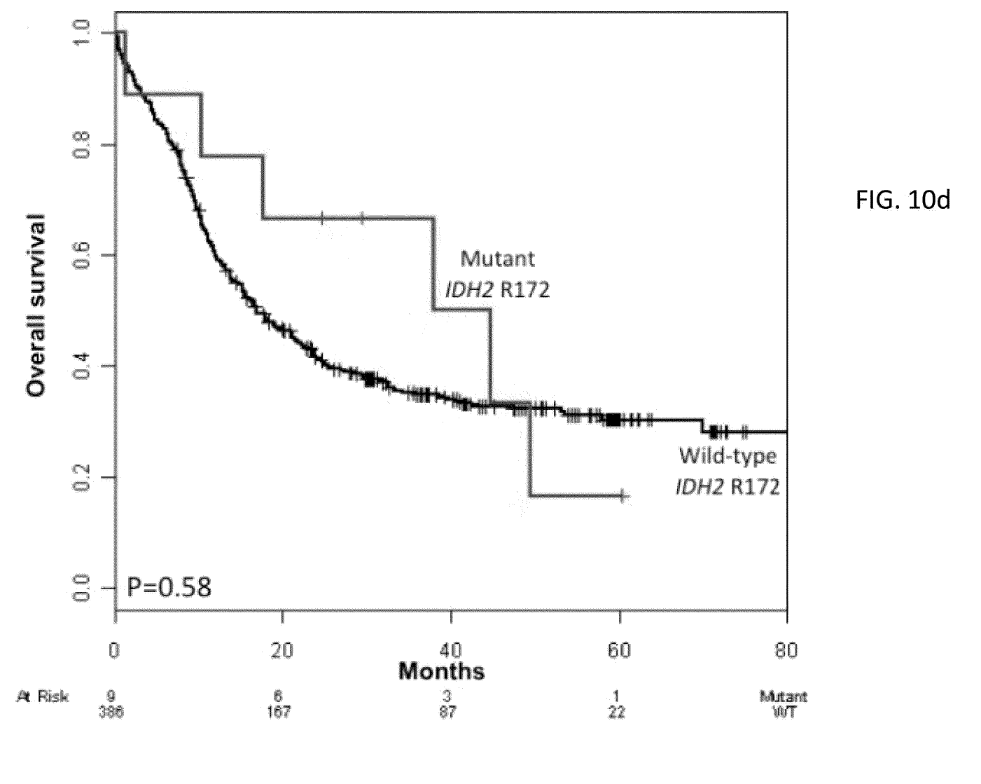

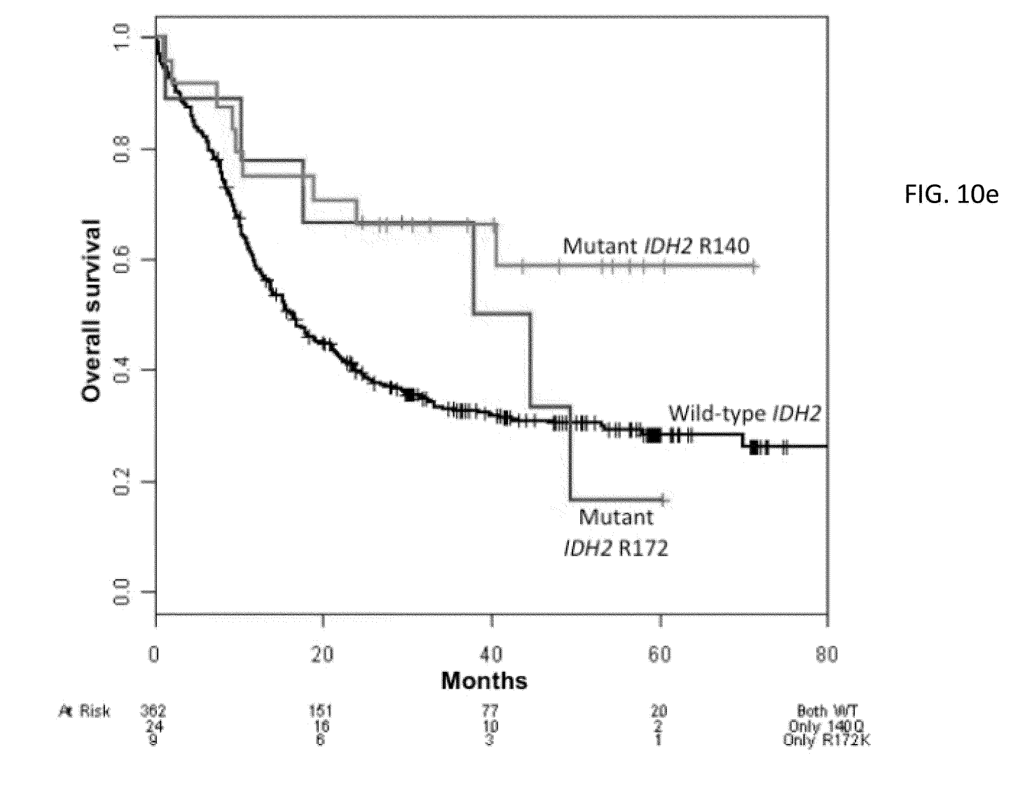

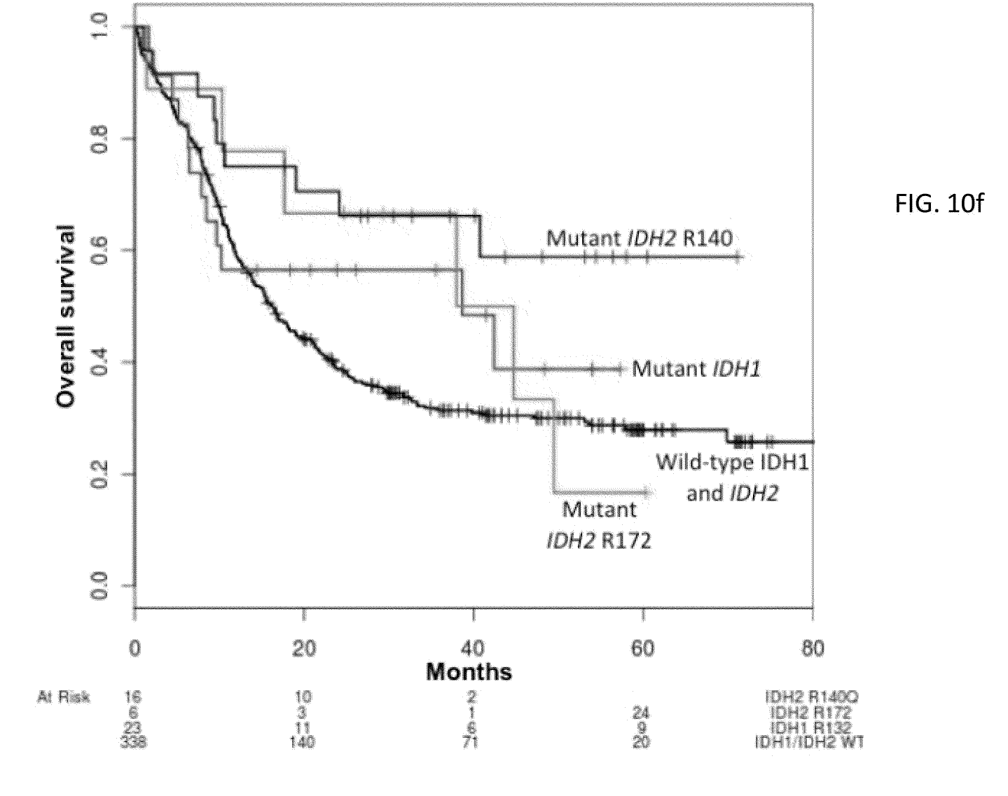

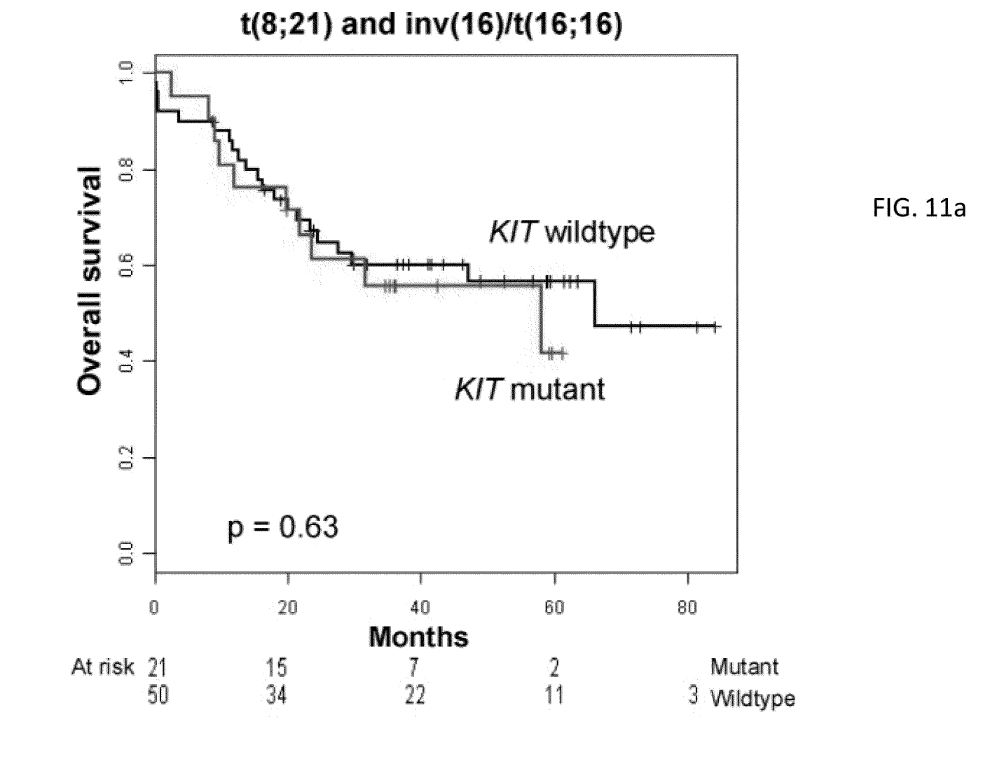

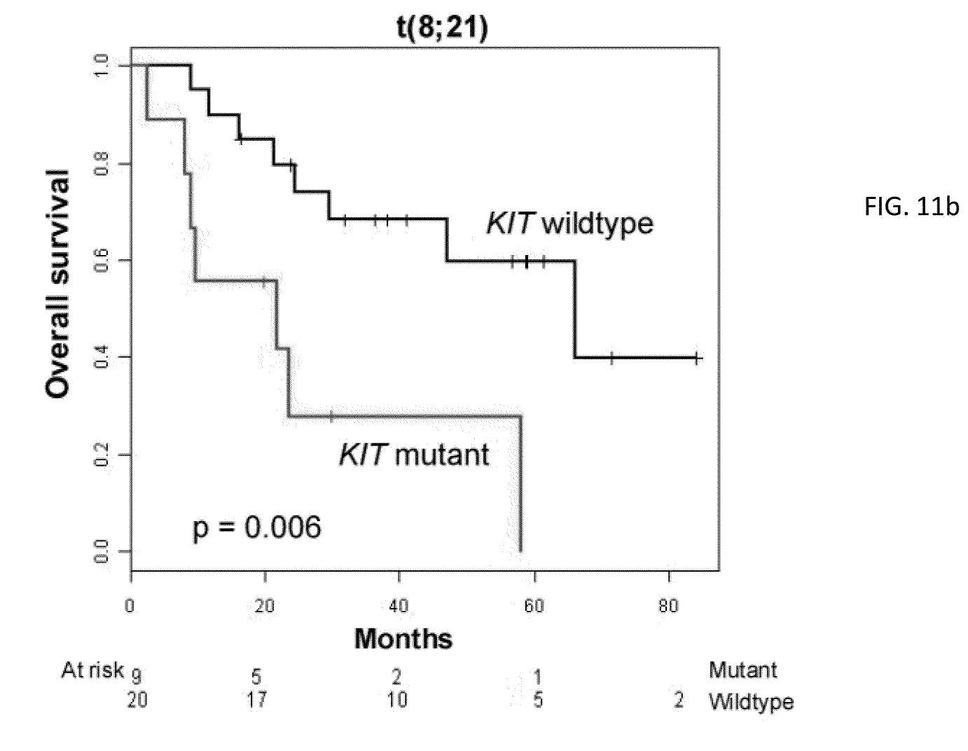

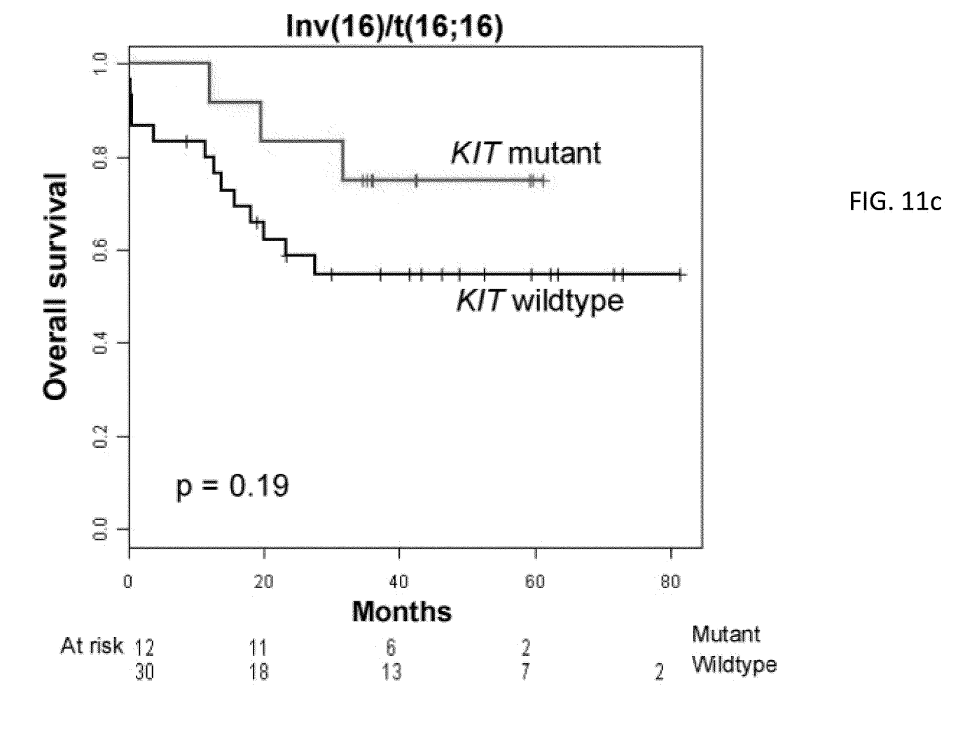

Univariate analysis revealed that FLT3 internal tandem duplication (FLT3-ITD) (p=0.001) and MLL partial tandem duplication (MLL-PTD) (p=0.009) mutations were associated with adverse OS (Table 9), while CEBPA (p=0.05) mutations and patients with core-binding factor alterations t(8;21) and inv(16)/t(16;16) (p<0.001) were associated with improved OS.^{2,23} In addition, PHF6 (p=0.006) and ASXL1 (p=0.05) mutations were associated with reduced OS (FIG. 9). IDH2 mutations were associated with improved OS in the entire cohort (FIG. 10) (p=0.01; 3 year OS=66%). The favorable impact of IDH2 mutations was exclusive to patients with IDH2 R140Q mutations (p=0.009; FIG. 10). All findings in univariate analysis were also statistically significant in multivariate analysis (adjusted p<0.05) (taking into account age, white blood cell count, transplantation and cytogenetics) (Table 9) with the exception of MLL-PTD, PHF6 and ASXL1 mutations. KIT mutations were associated with reduced OS in t(8;21)-positive AML (p=0.006) but not in patients with inv(16)/t(16;16) (p=0.19) (FIG. 11).

Prognostic Value of Molecular Alterations in Intermediate-Risk AML.

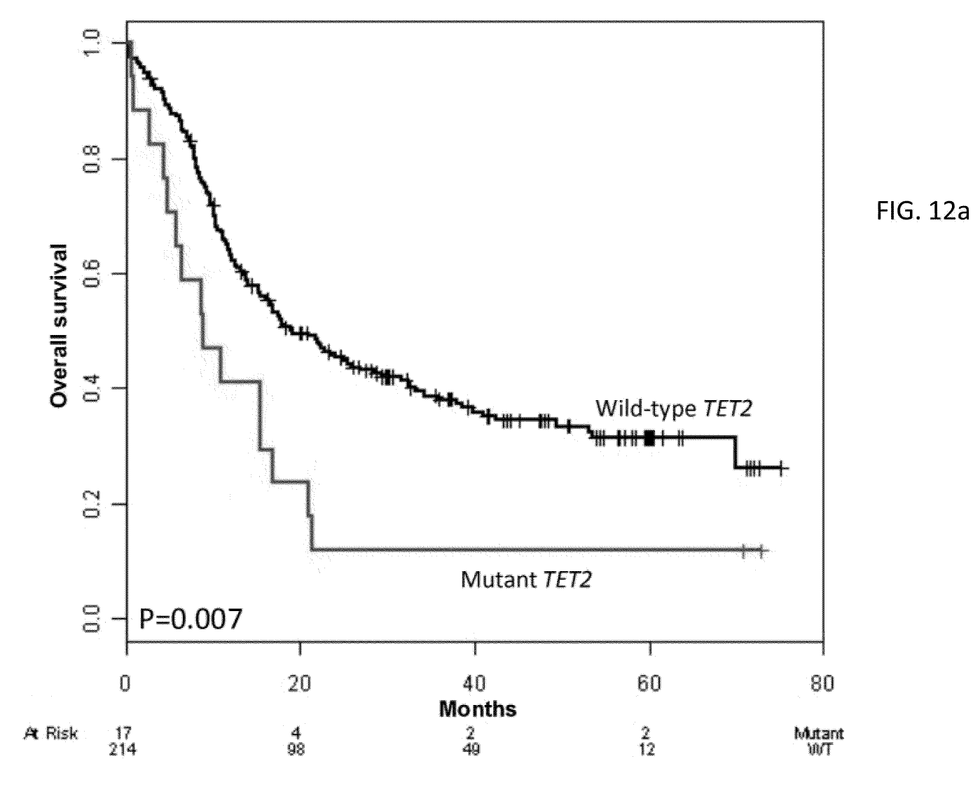

Amongst patients with cytogenetically-defined intermediate-risk AML (Table 10), FLT3-ITD mutations were associated with reduced OS (p=0.008). Similar to their effect on the entire cohort, ASXL1 and PHF6 mutations were associated with reduced survival and IDH2 R140Q mutations were associated with improved survival (Table 10). In addition, Applicants found that TET2 mutations were associated with reduced OS in patients with intermediate-risk AML (p=0.007; FIG. 12).

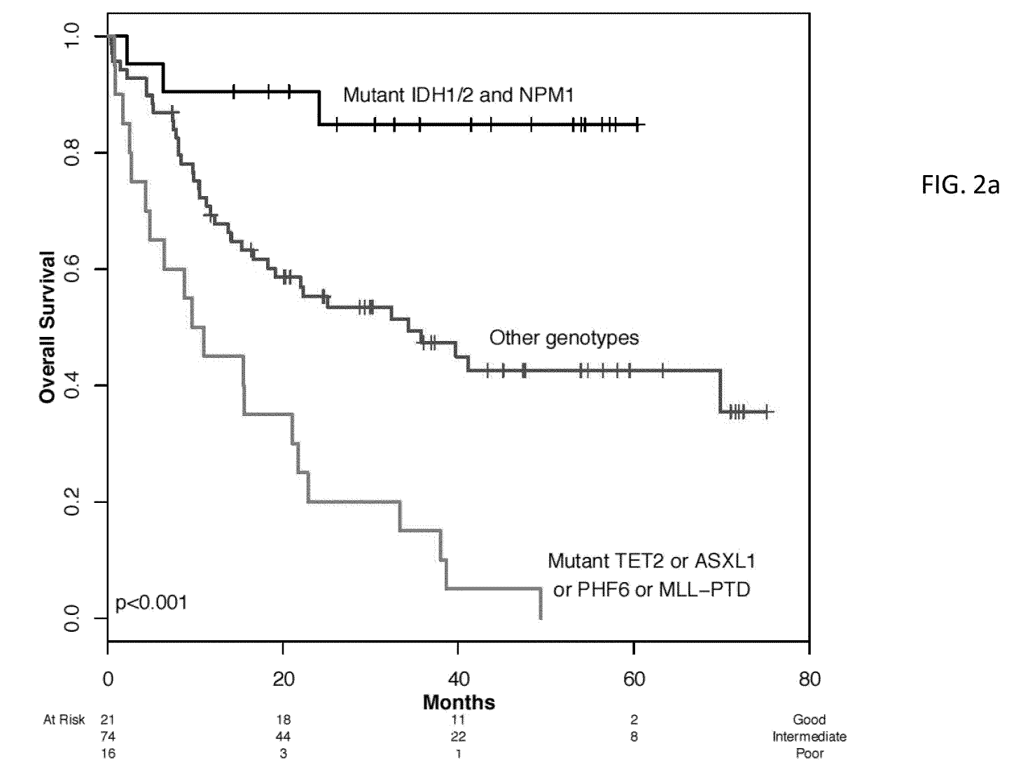

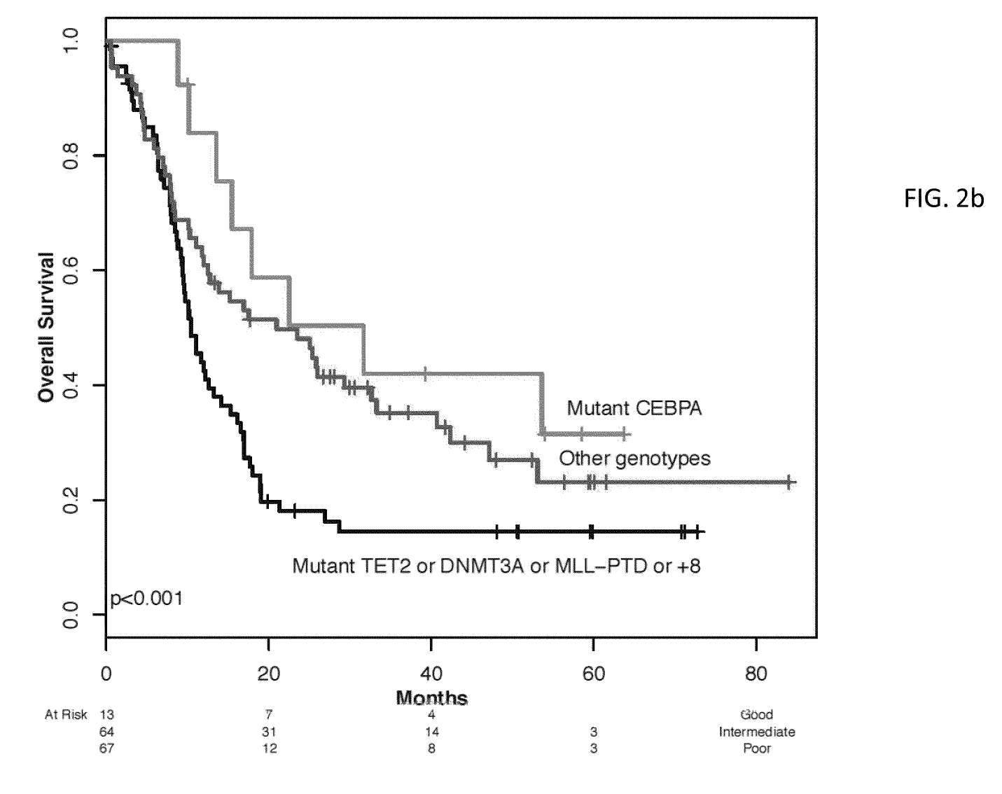

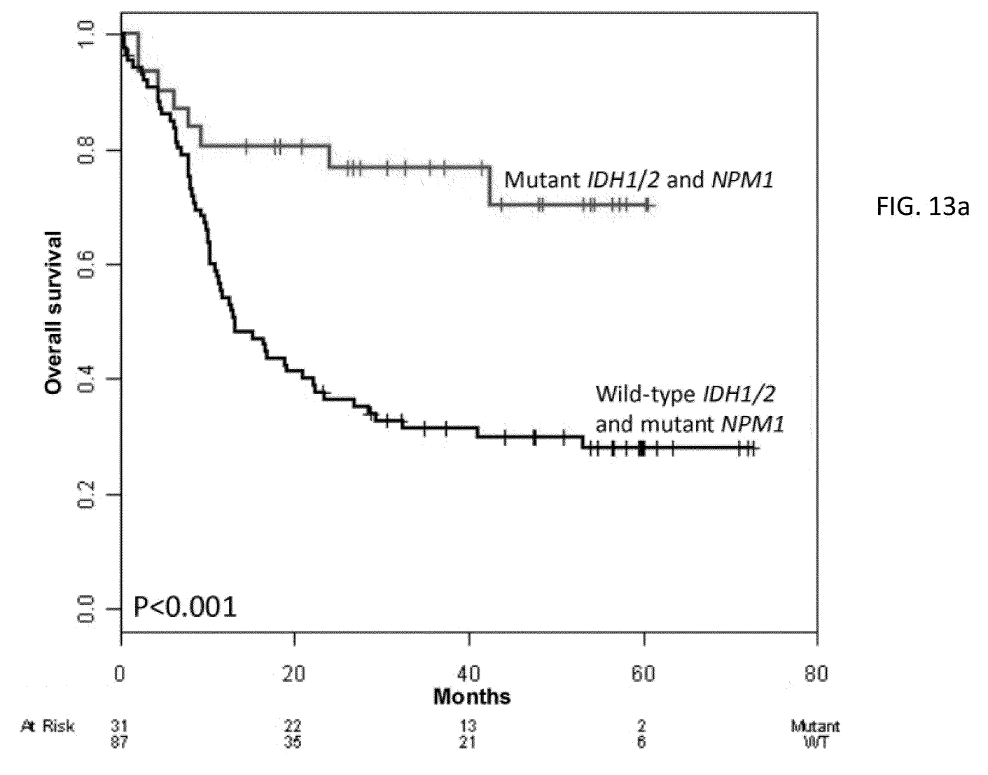

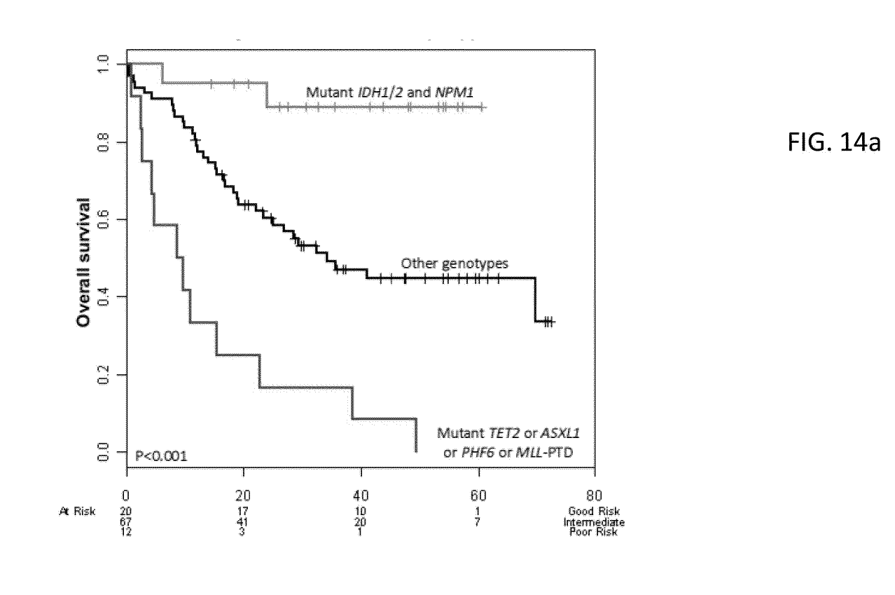

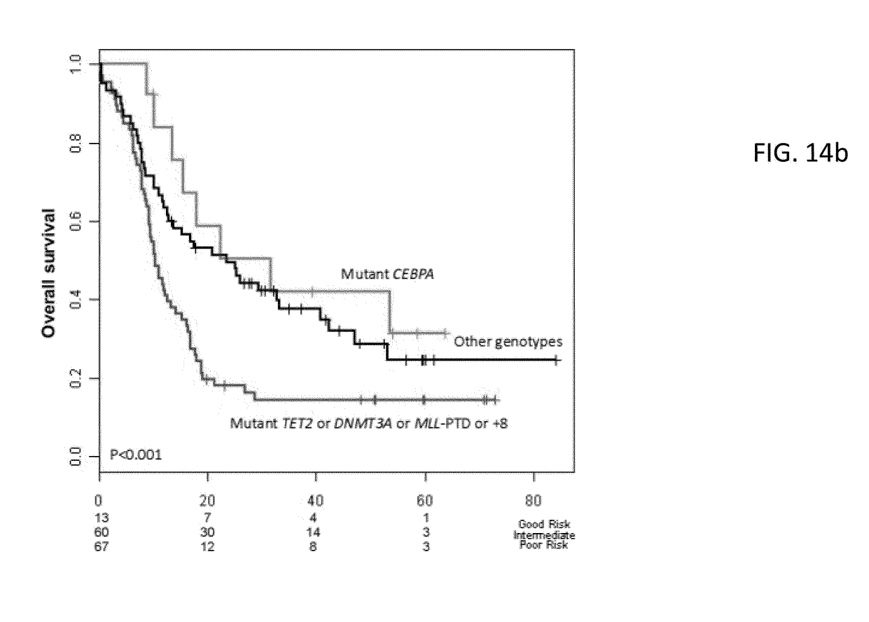

Multivariate statistical analysis revealed that FLT3-ITD mutations represented the primary predictor of outcome in patients with intermediate-risk AML (adjusted p<0.001). Applicants then performed multivariate analysis to identify mutations that affected outcome in patients with FLT3-ITD wild-type and mutant intermediate-risk AML, respectively. In patients with FLT3-ITD wild-type intermediate-risk AML, TET2, ASXL1, PHF6, and MLL-PTD mutations were independently associated with adverse outcome. Importantly, patients with both IDH1/IDH2 and NPM1 mutations (3 year OS=89%) but not NPM1-mutant patients wild-type for both IDH1 and IDH2 (3 year OS=31%), had improved OS within this subset of patients (p<0.001, FIG. 13). We then classified patients with FLT3-ITD wild-type intermediate-risk AML into three categories with marked differences in OS (adjusted p<0.001, FIG. 2A): patients with IDH1/IDH2 and NPM1 mutations (3 year OS=89%), patients with either TET2, ASXL1, PHF6, or MLL-PTD mutations (3 year OS=6.3%), and patients wild-type for TET2, ASXL1, PHF6, and MLL-PTD without co-occurring IDH1/NPM1 mutations (3 year OS=46.2%). Similar results were obtained when analysis was restricted to patients with a normal karyotype (FIG. 14A).

In patients with FLT3-ITD mutant, intermediate-risk AML, Applicants found that CEBPA mutations were associated with improved outcome and that trisomy 8 and TET2, DNMT3A, and MLL-PTD mutations were associated with adverse outcome. We used these data to classify patients with FLT3-ITD mutant intermediate-risk AML into three categories. The first category included patients with trisomy 8 or TET2, DNMT3A, or MLL-PTD mutations, which were associated with adverse outcome (3 year OS=14.5%) significantly worse than for patients wild-type for CEBPA, TET2, DNMT3A, and MLL-PTD (3 year OS=35.2%; p<0.001) or for patients with CEBPA mutations (3 year OS=42%; p<0.001, FIG. 2B). The survival of patients with FLT3-ITD mutant intermediate-risk AML who were wild-type for CEBPA, TET2, DNMT3A, and MLL-PTD did not differ from patients with CEBPA-mutant/FLT3-ITD mutant AML (p=0.34), suggesting that the presence of poor risk mutations more precisely identifies FLT3-ITD mutant AML patients with adverse outcome than the absence of CEBPA mutations alone. These same three risk groups also had significant prognostic value in FLT3-ITD mutant, normal karyotype AML (FIG. 14B).

Prognostic Schema Using Integrated Mutational and Cytogenetic Profiling.

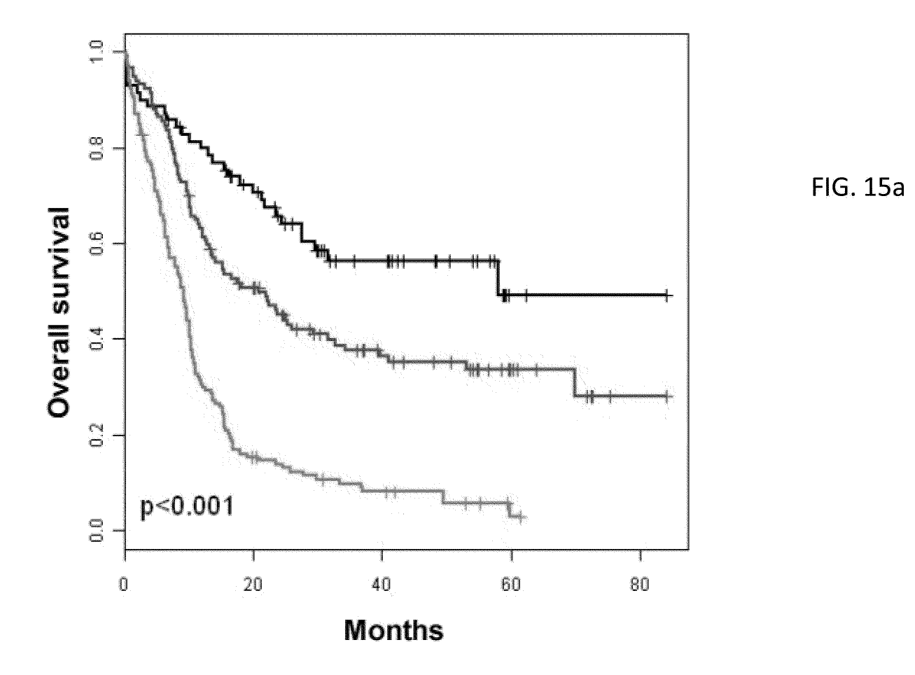

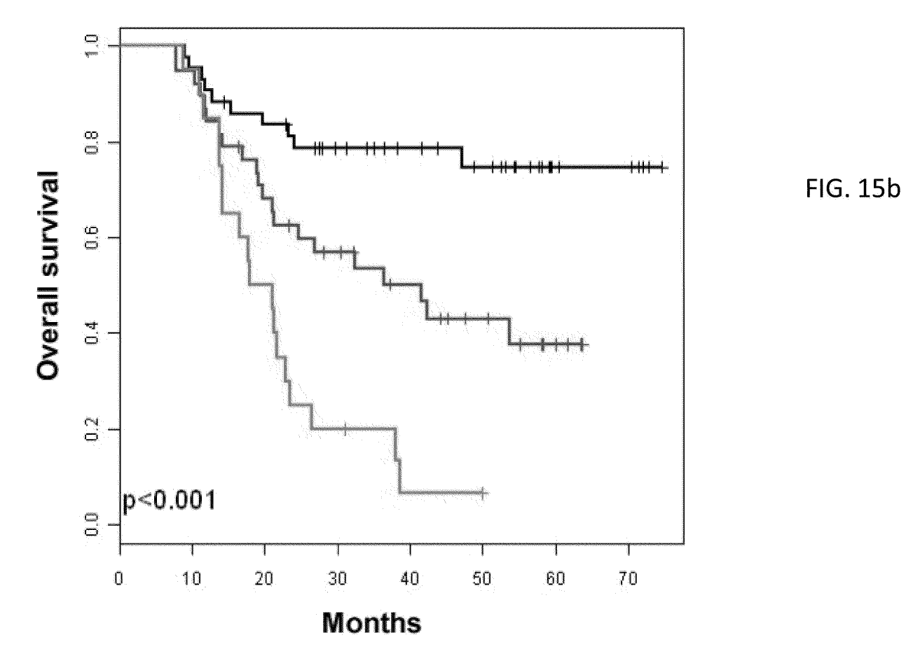

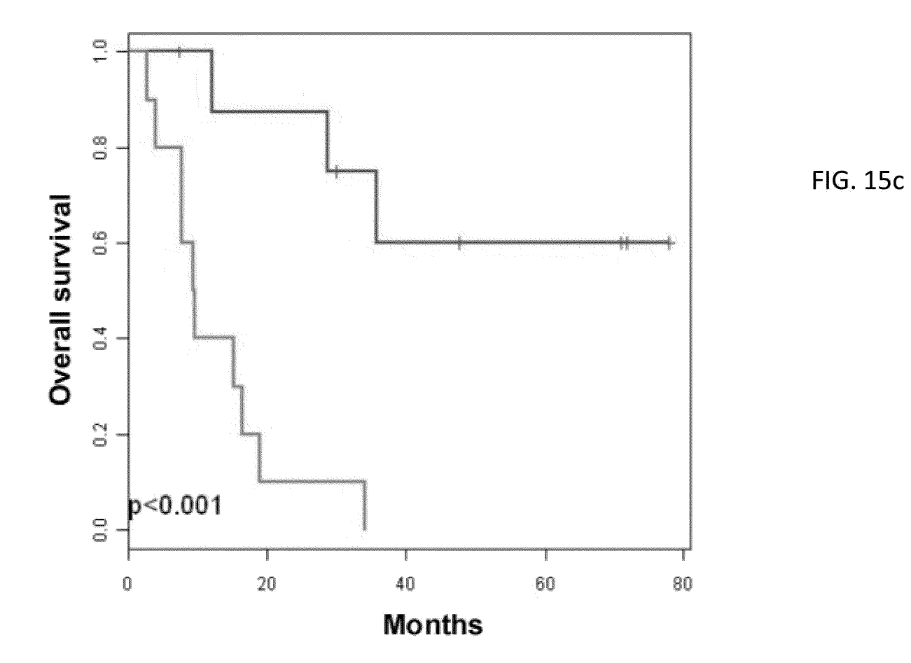

These results allowed us to develop a prognostic schema integrating our findings from comprehensive mutational analysis with cytogenetic data into 3 risk groups with favorable (median: not reached, 3-year: 64%), intermediate (25.4 months, 42%), and adverse risk (10.1 months, 12%) (FIGS. 3A and 3B, Table 11). The mutational prognostic schema predicted for outcome independent age, WBC count, induction dose, and transplantation status in multivariate analysis (adjusted p<0.001). Our classification held true regardless of post-remission therapy with autologous, allogeneic, or consolidation chemotherapy alone (FIG. 15). Given the number of variables on our prognostic classification, we tested the reproducibility of this predictor in an independent cohort of 104 patients from the ECOG E1900 trial. Importantly, mutational analysis of the validation cohort confirmed the reproducibility of our prognostic schema to predict outcome in AML (adjusted p<0.001; FIG. 3C). The mutational prognostic schema was independent of treatment-related mortality (death within 30 days) or lack of response to induction chemotherapy (inability to achieve complete remission) in the test cohort and in the combined test/validation cohorts (Table 12).

Genetic Predictors of Response to Induction Chemotherapy.

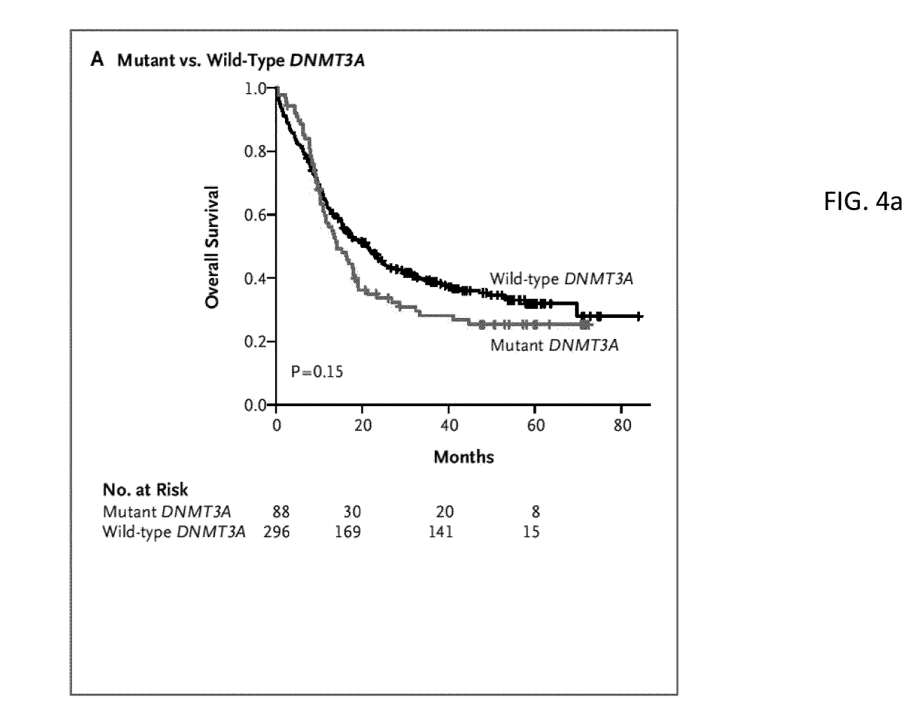

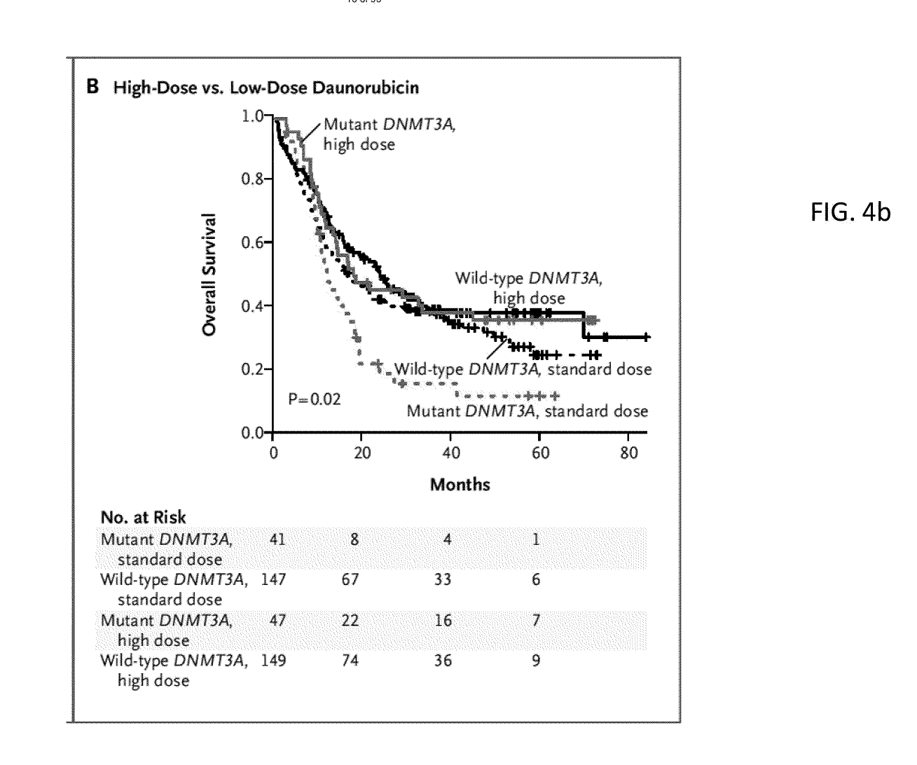

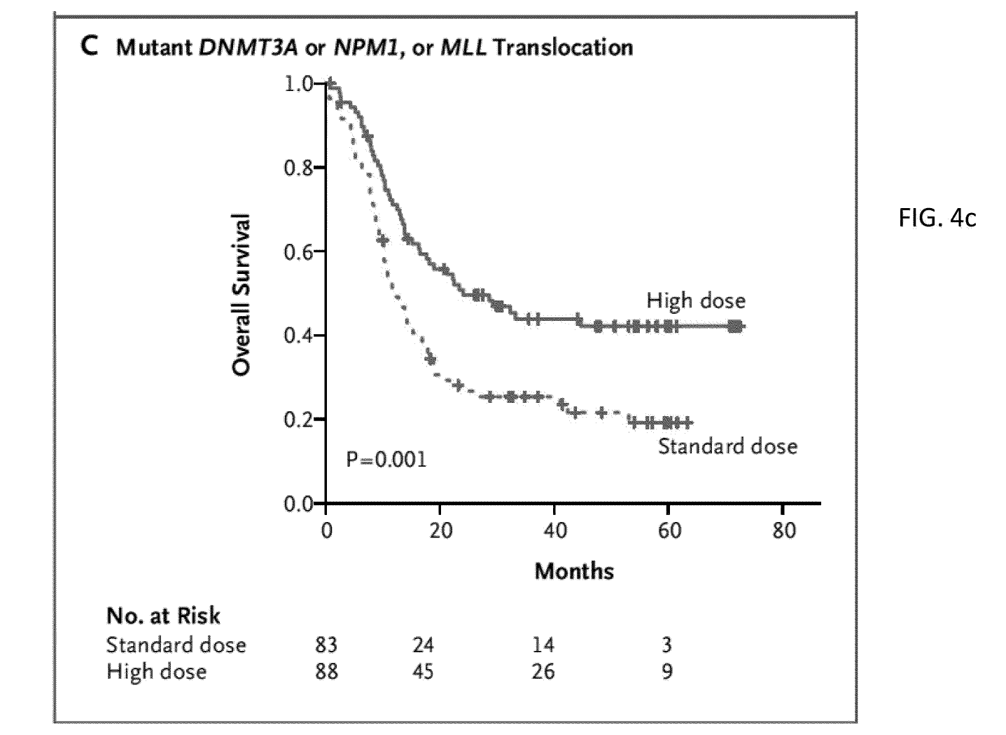

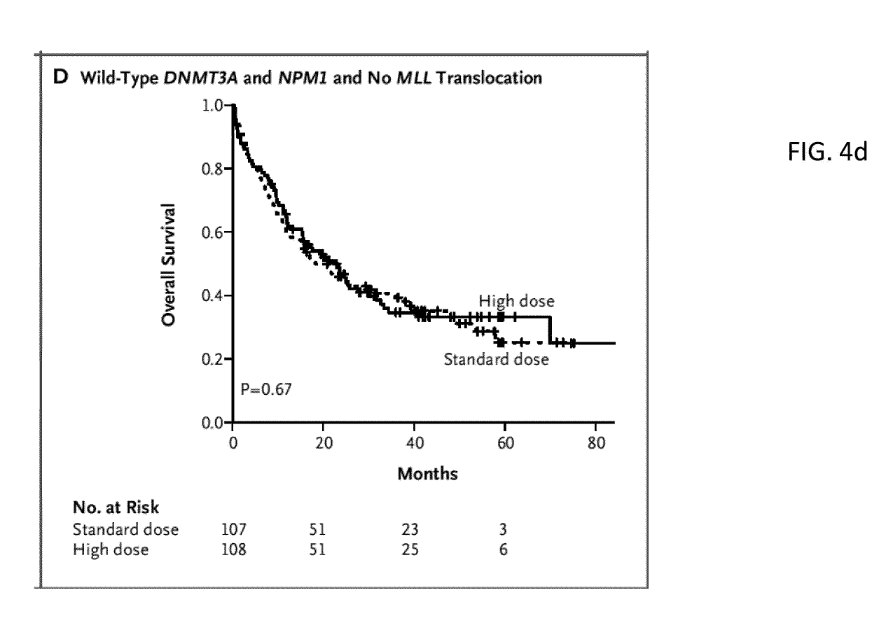

Recent studies noted that DNMT3A-mutant AML is associated with adverse outcome. However, Applicants here found that DNMT3A mutations were not associated with adverse outcome in the ECOG 1900 cohort (FIG. 4A; p=0.15). The ECOG 1900 trial randomized patients to induction therapy with cytarabine plus 45 or 90 mg/m² daunorubicin (Fernandez et al. N Eng J Med 2009, 361: 1249-1259). Applicants therefore conceived that high dose daunorubicin improved outcomes in AML patients with DNMT3A mutations. Indeed Applicants found that DNMT3A mutational status had a significant impact on the outcome with dose-intensive chemotherapy (FIG. 4B; p=0.02).

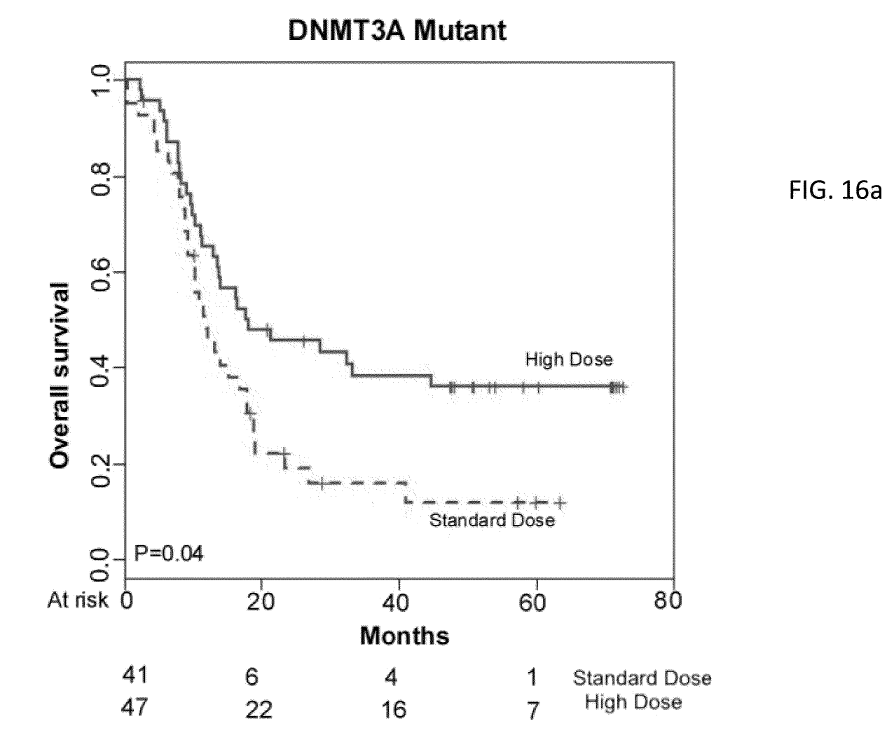

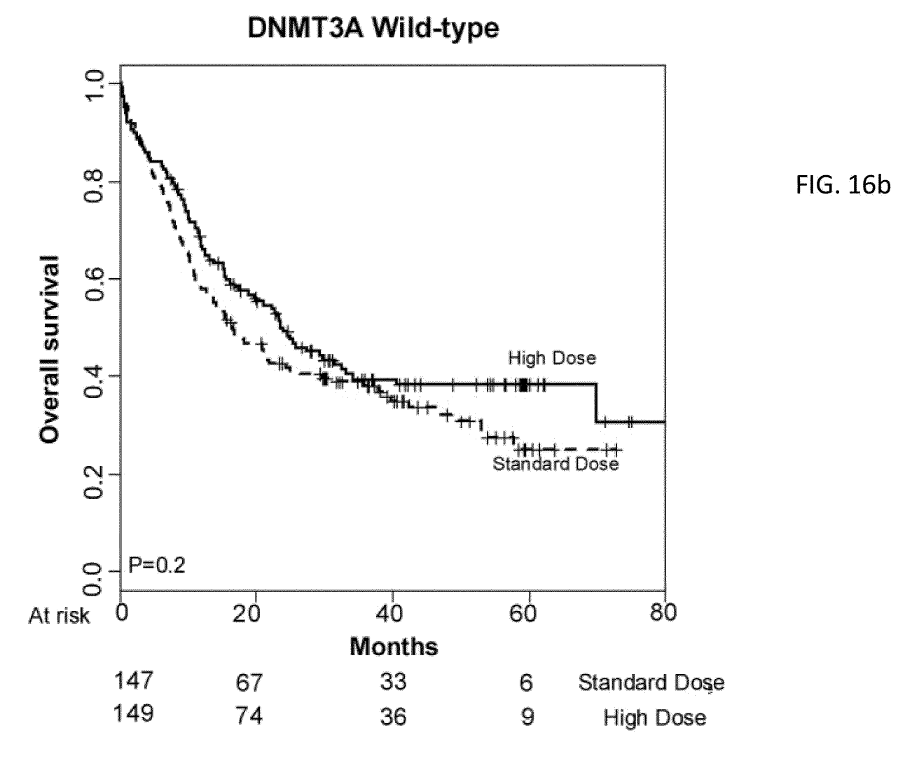

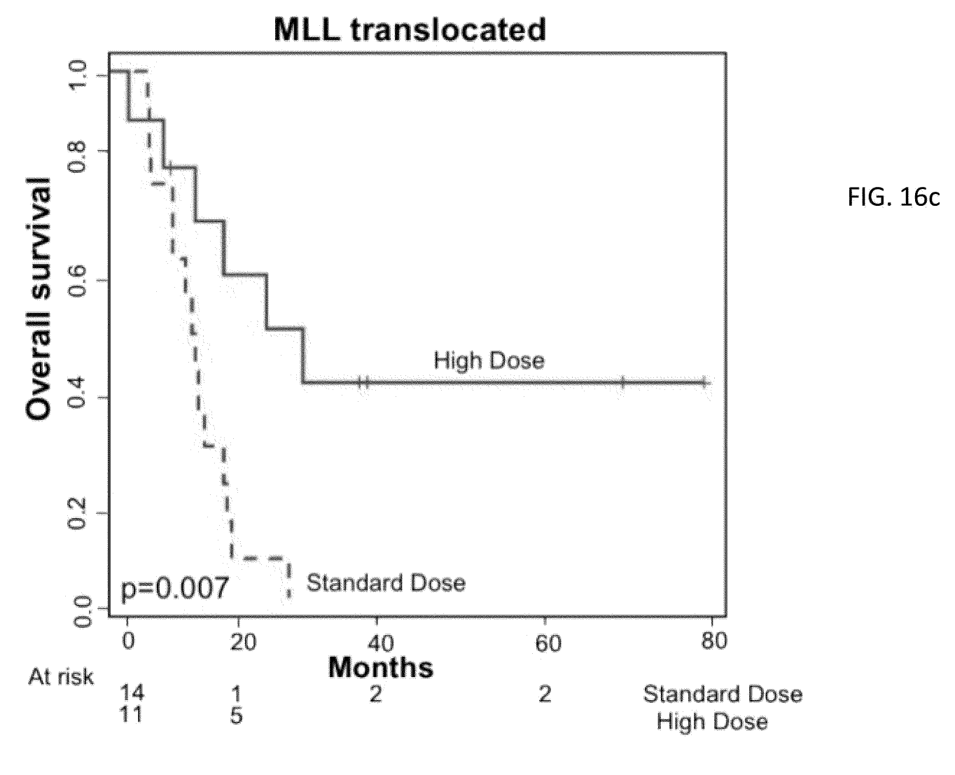

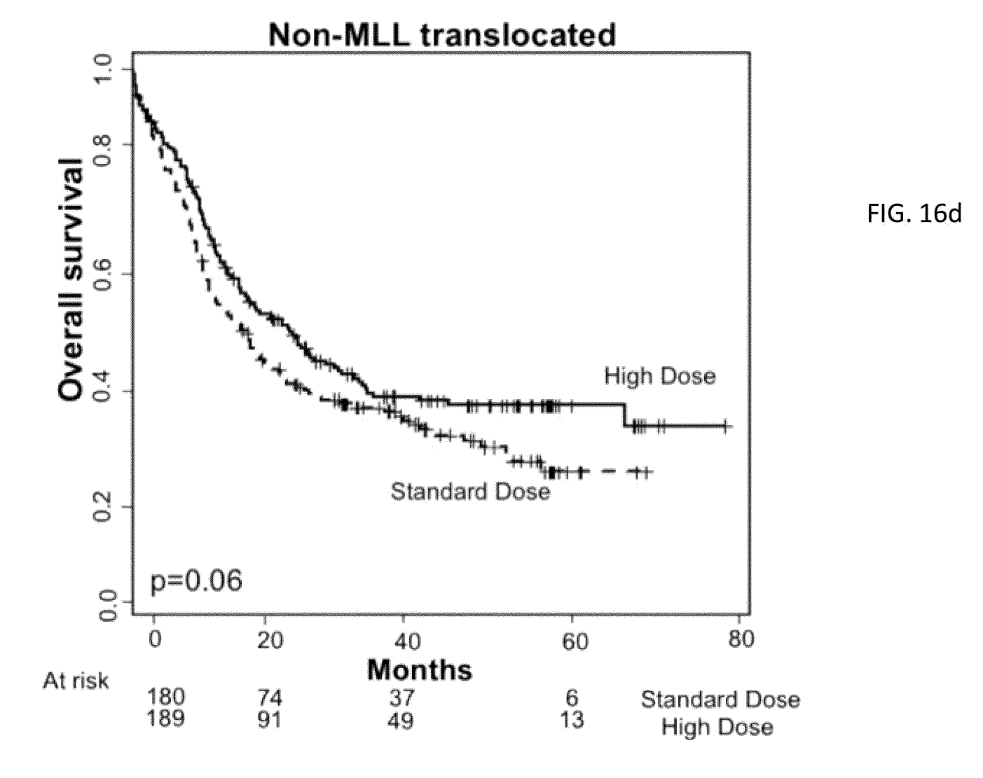

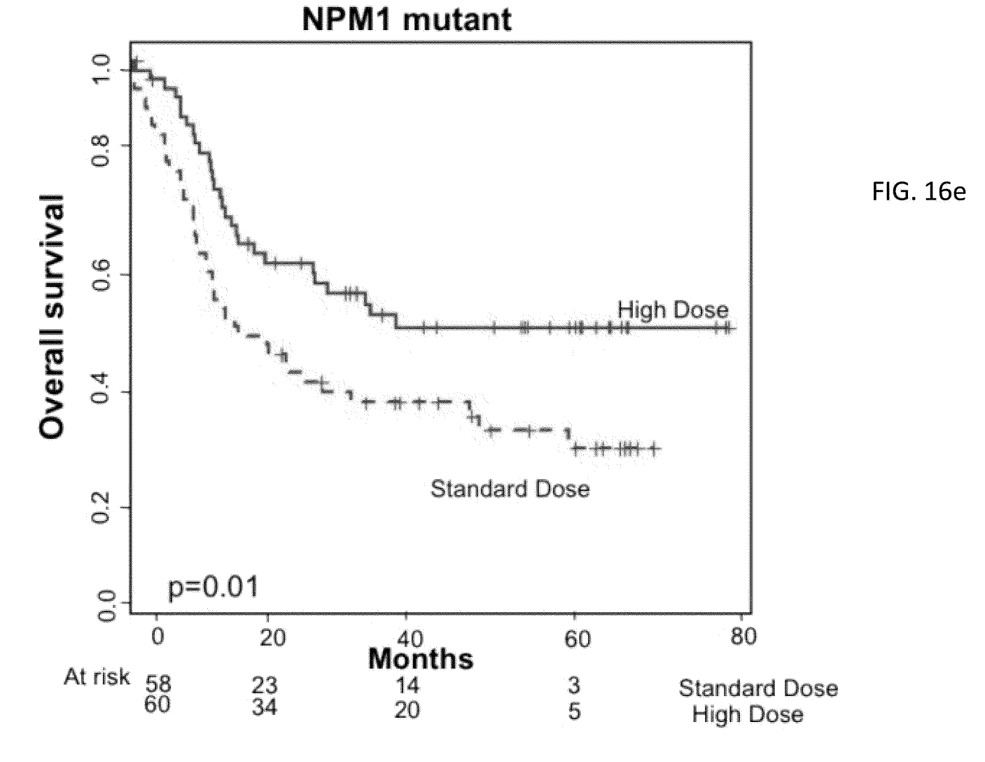

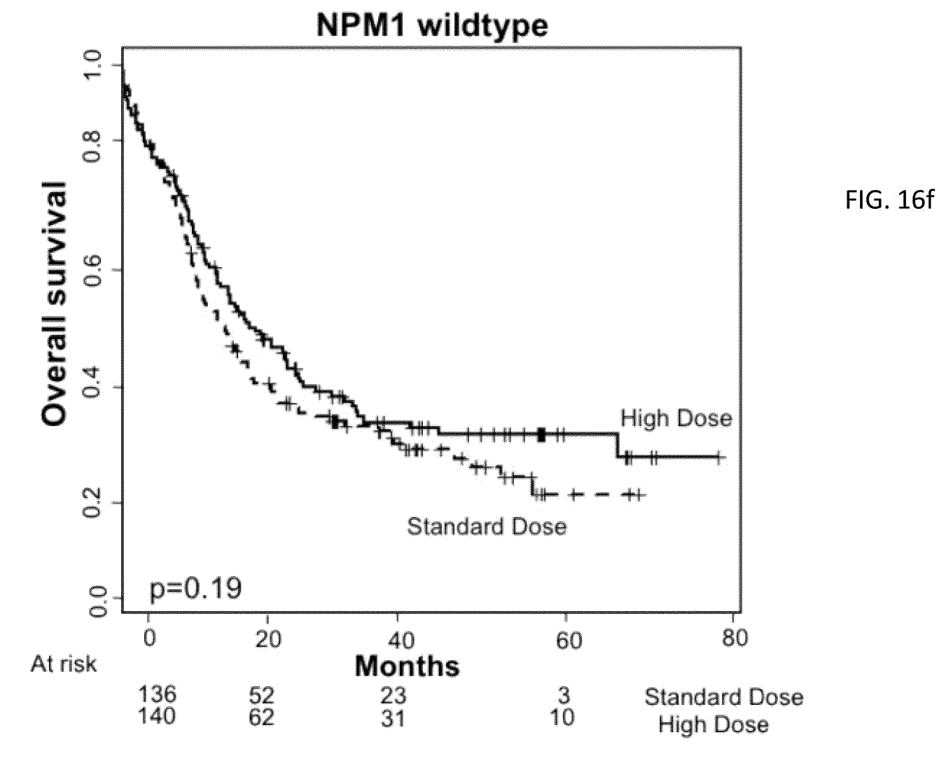

Applicants then assessed the effects of DNMT3A mutational status on outcome according to treatment arm, and found that high-dose daunorubicin was associated with improved survival in DNMT3A mutant patients (FIG. 16A; p=0.04) but not in patients wild-type for DNMT3A (FIG. 16B; p=0.15). In addition to DNMT3A mutations, univariate analysis revealed that dose-intensified induction therapy improved outcome in AML patients with MLL translocations (FIGS. 16C and 11D; p=0.01; p-value adjusted for multiple-testing=0.06) and NPM1 mutations (FIGS. 16E and 11F; p=0.01; p-value adjusted for multiple-testing=0.1; Table 13).

Applicants then separated the patients in our cohort into two groups: patients with mutations in DNMT3A or NPM1 or MLL translocations, and patients wild-type for these 3 genetic abnormalities. Dose-intensive induction therapy was associated with a marked improvement in survival in DNMT3A/NPM1/MLL translocation-positive patients (FIG. 4C; p=0.001) but not in patients wild-type for DNMT3A, NPM1, and MLL translocations (FIG. 4D; p=0.67). This finding was independent of the clinical co-variates of age, WBC count, transplantation status, treatment-related mortality, and chemotherapy resistance (adjusted p=0.008 and p=0.34 for mutant and wild-type patients respectively), suggesting that high-dose anthracycline chemotherapy offers benefit to genetically defined AML subgroups.

All publications, patents, and patent applications mentioned herein are hereby incorporated by reference in their entirety as if each individual publication or patent was specifically and individually indicated to be incorporated by reference. In case of conflict, the present application, including any definitions herein, will control. While several aspects of the present invention have been described and depicted herein, alternative aspects may be effected by those skilled in the art to accomplish the same objectives. Those skilled in the art will recognize, or be able to ascertain using no more than routine experimentation, many equivalents to the specific embodiments of the invention described herein. Accordingly, it is intended by the appended claims to cover all such alternative aspects as fall within the true spirit and scope of the invention.

TABLE 1

| | | Validation | |
|---|---|---|---|
| | Test cohort | cohort | Entire cohort |
| Variable | (N = 398) | (N = 104) | (N = 657) |
| Age | | | |
| Group - no (%) | | | |
| <50 yr | 227 (57.0) | 42 (40.8) | 360 (54.8) |
| ≧50 yr | 171 (43.0) | 61 (59.2) | 297 (45.2) |
| Median - yr | 46.5 | 53 | 48.0 |
| Range - yr | 18-60 | 18-60 | 17-60 |
| Sex - no. (%) | | | |
| Male | 207 (52.0) | 51 (49.5) | 335 (51.0) |
| Female | 191 (48.0) | 52 (50.5) | 322 (49.0) |
| Peripheral blood | | | |
| white-cell count | | | |
| Level - no. (%) | | | |
| <10,000/mm³ | 123 (30.9) | 84 (81.6) | 306 (46.6) |
| ≧10,000/mm³ | 275 (69.1) | 18 (17.5) | 350 (53.3) |
| Missing data | 0 (0) | 1 (1) | 1 (0.2) |
| Median - cells/ | 19.9 | 2.5 | 12.3 |
| mm³ × 1000 | | | |
| Range - cells/ | 1-213 | 1-117 | 1-366 |
| mm³ × 1000 | | | |
| Hemoglobin | | | |
| Level - no. (%) | | | |
| <10 g/dl | 276 (69.3) | 77 (74.8) | 464 (70.6) |
| ≧10 g/dl | 121 (30.4) | 25 (24.3) | 191 (29.1) |
| Missing data | 1 (0.3) | 1 (1) | 2 (0.3) |
| Median - g/dl | 9.2 | 9.2 | 9.2 |
| Range - g/dl | 5-30 | 5-14 | 5-30 |
| Peripheral-blood | | | |
| platelet count | | | |
| Level - no. (%) | | | |
| <50,000/mm3 | 194 (48.7) | 43 (41.7) | 305 (46.4) |
| ≧50,000/mm3 | 204 (51.3) | 59 (57.3) | 351 (53.4) |
| Missing data | 0 (0) | 1 (1) | 1 (0.2) |
| Median - g/dl | 50.0 | 61 | 50.0 |
| Range - g/dl | 1-650 | 6-995 | 1-995 |
| Blasts | | | |
| Peripheral blood | | | |
| Median % | 47.5 | 8 | 31 |
| Range % | 0-98 | 0-99 | 0-99 |
| Bone Marrow | | | |
| Median % | 68.5 | 49 | 64.0 |
| Range % | 3-100 | 17-100 | 3-100 |
| Leukemia | | | |
| Classification - no | | | |
| (%) | | | |
| Not reviewed | 0 (0) | 0 | 21 (3.2) |
| AML Minimally | 20 (5.0) | 5 (4.9) | 29 (4.4) |
| Differentiated | | | |
| AML w/o Maturation | 96 (24.1) | 22 (21.4) | 155 (23.6) |
| AML w/ Maturation | 61 (15.3) | 27 (26.2) | 112 (17.0) |
| Acute myelomonocytic | 52 (13.1) | 7 (6.8) | 63 (9.6) |
| Leukemia | | | |
| Acute monocytic/ | 27 (6.8) | 3 (2.9) | 40 (6.1) |
| monoblastic Leukemia | | | |
| Acute erythroid | 8 (2.0) | 6 (5.8) | 29 (4.4) |
| Leukemia | | | |
| Acute | 0 (0) | 2 (1.9) | 3 (0.5) |
| megakaryoblastic | | | |
| Leukemia | | | |
| Cytogenetic profile - | | | |
| no. (%) | | | |
| Favorable | 67 (16.8) | 10 (9.7) | 89 (13.5) |
| Indeterminate | 85 (21.4) | 22 (21.4) | 176 (26.8) |
| Intermediate | 180 (45.2) | 42 (40.8) | 267 (40.6) |
| Normal karyotype | 163 (41.0) | 42 (40.4) | 244 (37.1) |
| Unfavorable | 65 (16.3) | 29 (28.2) | 122 (18.6) |
| Patients with | 11/398 (2.8) | 4 (3.9) | 22/657 (3.3) |
| secondary AML | | | |
| Survival (days) | | | |
| Median | 535.2 | 650.9 | 621 |

TABLE 2
Genomic DNA primer sequences utilized for comprehensive genetic analysis.
All primer sequences are displayed with Ml3F2/M13R2 tags

| Gene | Ganomic | Forward Orimer Sequence | SEQ ID NO. | Reverse Primer Sequence | SEQ ID NO. |
|---|---|---|---|---|---|
| ASXL1 | chr20:30410194-30410296 | GTAAAACGACGGCCAGTGGTCCTGTCTCAGTCCCTCA | 1 | CAGGAAACAGCTATGACCTCTTAAAGGAAGATGGCCCC | 166 |
| | chr20:30417847-30417930 | GTAAAACGACGGCCAGTCCAGCGGTACCTCATAGCAT | 2 | CAGGAAACAGCTATGACCGCGTTAGGCACAATAGAGGC | 167 |
| | chr20:30420478-30420587 | GTAAAACGACGGCCAGTTGGATTTCGGGTATCACATAA | 3 | CAGGAAACAGCTATGACCtccaagaatcaCTGCACCAA | 168 |
| | chr20:30479591-30479712 | GTAAAACGACGGCCAGTTCCCTCTTTTTCAAAAGCATACA | 4 | CAGGAAACAGCTATGACCACCCATCCATTAAAGGGTCC | 169 |
| | chr20:30479788-30479886 | GTAAAACGACGGCCAGTTTGCTGTCACAGAAGGATGC | 5 | CAGGAAACAGCTATGACCTGTCATCATTCATCCTCCCA | 170 |
| | chr20:30480801-30480895 | GTAAAACGACGGCCAGTAATGATGCTTGGCACAGTGA | 6 | CAGGAAACAGCTATGACCCAGAGCCCAGCACTAGAACC | 171 |
| | chr20:30481364-30481517 | GTAAAACGACGGCCAGTGGTTCTAGTGCTGGGCTCTG | 7 | CAGGAAACAGCTATGACCAAAATAGAGGGCCACCCAAG | 172 |
| | chr20:30482784-30482948 | GTAAAACGACGGCCAGTGCTTTGTGGAGCCTGTTCTC | 8 | CAGGAAACAGCTATGACCAGAAGGATCAAGGGGGAAAA | 173 |
| | chr20:30483046-30483143 | GTAAAACGACGGCCAGTGTCAAATGAAGCGCAACAGA | 9 | CAGGAAACAGCTATGACCGGAGACATGCAACACCACAC | 174 |
| | chr20:30484343-30484449 | GTAAAACGACGGCCAGTCAAGGAGTTGCTTGGTCTCA | 10 | CAGGAAACAGCTATGACCCACGTTCTGCTGCAATGACT | 175 |
| | chr20:30484747-30485127 | GTAAAACGACGGCCAGTCGACAGGAAATGGAGAAGGA | 11 | CAGGAAACAGCTATGACCTTCTGATCCTTGGGTTCCTG | 176 |
| | chr20:30485128-30485381 | GTAAAACGACGGCCAGTAAAAGTGGCTTGTGTGTCCC | 12 | CAGGAAACAGCTATGACCGGCTGTCTCAAGCAAACCTC | 177 |
| | chr20:30485895-30486275 | GTAAAACGACGGCCAGTGAGGTTTGCTTGAGACAGCC | 13 | CAGGAAACAGCTATGACCGAAGGCAGGTCCTCTCTCCT | 178 |
| | chr20:30486276-30486655 | GTAAAACGACGGCCAGTGGACCCTCGCAGACATTAAA | 14 | CAGGAAACAGCTATGACCTGTTCTGCAGGCAATCAGTC | 179 |
| | chr20:30486656-30487035 | GTAAAACGACGGCCAGTGCCATGTCCAGAGCTAGGAG | 15 | CAGGAAACAGCTATGACCTGGCACAGTCCAGAGTGAAG | 180 |
| | chr20:30487036-30487415 | GTAAAACGACGGCCAGTCTTGAAAACCAAGGCTCTCG | 16 | CAGGAAACAGCTATGACCCACAAGTGGGTTAGTGGCCT | 181 |
| | chr20:30487416-30487795 | GTAAAACGACGGCCAGTCAAGGTGAATGGTGACATGC | 17 | CAGGAAACAGCTATGACCCTGGATGGAGGGAGTCAAAA | 182 |
| | chr20:30487796-30488175 | GTAAAACGACGGCCAGTCTGAGTACCAGCCAAGAGCC | 18 | CAGGAAACAGCTATGACCAAGTGACCCACCAGTTCCAG | 183 |
| | chr20:30488176-30488555 | GTAAAACGACGGCCAGTTTTTGACTCCCTCCATCCAG | 19 | CAGGAAACAGCTATGACCACACTGGAGCGAGATGCTTT | 184 |
| | chr20:30488556-30488935 | GTAAAACGACGGCCAGTCTGGAACTGGTGGGTCACTT | 20 | CAGGAAACAGCTATGACCTATACCCAGGAAACCCCTCC | 185 |
| CEBPa | chr19:38483156-38483535 | GTAAAACGACGGCCAGTGCAAGTATCCGAGCAAAACC | 21 | CAGGAAACAGCTATGACCGAGGAGGGGAGAATTCTTGG | 186 |
| | chr19:38483156-38483535 | GTAAAACGACGGCCAGTCCGACGGAGAGTCTCATTTT | 22 | CAGGAAACAGCTATGACCCCTGCTATAGGCTGGGCTTC | 187 |
| | chr19:38483156-38483535 | GTAAAACGACGGCCAGTGGAGAGGCGTGGAACTAGAG | 23 | CAGGAAACAGCTATGACCCTTGGTGCGTCTAAGATGAGG | 188 |
| | chr19:38483536-38483915 | GTAAAACGACGGCCAGTTCATAACTCCGGTCCCTCTG | 24 | CAGGAAACAGCTATGACCCTGGAGCTGACCAGTGACAA | 189 |
| | chr19:38483916-38484295 | GTAAAACGACGGCCAGTCATTTCCAAGGCACAAGGTT | 25 | CAGGAAACAGCTATGACCTGGACAAGAACAGCAACGAG | 190 |
| | chr19:38484296-38484675 | GTAAAACGACGGCCAGTTTGTCACTGGTCAGCTCCAG | 26 | CAGGAAACAGCTATGACCCCTTCAACGACGAGTTCCTG | 181 |
| | chr19:38484296-38484675 | GTAAAACGACGGCCAGTTTGTCACTGGTCAGCTCCAG | 27 | CAGGAAACAGCTATGACCCACCTGCAGTTCCAGATCG | 182 |
| | chr19:38484296-38484675 | GTAAAACGACGGCCAGTCAGGTGCATGGTGGTCTG | 28 | CAGGAAACAGCTATGACCATCGACATCAGCGCCTACAT | 193 |
| | chr19:38484676-38485055 | GTAAAACGACGGCCAGTCTCGTTGCTGTTCTTGTCCA | 29 | CAGGAAACAGCTATGACCCGGGAGAACTCTAACTCCCC | 194 |
| | chr19:38484676-38485055 | GTAAAACGACGGCCAGTCTCGTTGCTGTTCTTGTCCA | 30 | CAGGAAACAGCTATGACCCAGGCTGGAGCCCCTGTA | 195 |
| | chr19:38484676-38485055 | GTAAAACGACGGCCAGTGCTTGGCTTCATCCTCCTC | 31 | CAGGAAACAGCTATGACCTCGGCCGACTTCTACGAG | 196 |
| | chr19:38485056-38485160 | GTAAAACGACGGCCAGTATGTAGGCGCTGATGTCGAT | 32 | CAGGAAACAGCTATGACCCGGGAGAACTCTAACTCCCC | 197 |
| DNMT3a | chr2:25310489-25310793 | GTAAAACGACGGCCAGTCCTCTCTCCCACCTTTCCTC | 33 | CAGGAAACAGCTATGACCCTGAGTGCCGGGTTGTTTAT | |
| | chr2:25312079-25312198 | GTAAAACGACGGCCAGTGGAAAACAAGTCAGGTGGGA | 34 | CAGGAAACAGCTATGACCTGGATCTAAGATTGGCCAGG | 199 |
| | chr2:25313308-25313378 | GTAAAACGACGGCCAGTccacactagctggagaagca | 35 | CAGGAAACAGCTATGACCggggctcttaccctgtgaac | 200 |
| | chr2:25315502-25315588 | GTAAAACGACGGCCAGTcatggcagagcagctagtca | 36 | CAGGAAACAGCTATGACCtgtgtggctcctgagagaga | 201 |
| | chr2:25316674-25316823 | GTAAAACGACGGCCAGTAATACCCAACCCCAGGAGTC | 37 | CAGGAAACAGCTATGACCCTTCCTGTCTGCCTCTGTCC | 202 |
| | chr2:25317012-25317103 | GTAAAACGACGGCCAGTGAAGCCATTAGTGAGCTGGC | 38 | CAGGAAACAGCTATGACCCAACTTGGTCCCGTTCTTGT | 203 |
| | chr2:25317934-25318080 | GTAAAACGACGGCCAGTTTGCCAAAAGTATTGGGAGG | 39 | CAGGAAACAGCTATGACCCCAGTTGGATCCAGAAAGGA | 204 |
| | chr2:25320270-25320355 | GTAAAACGACGGCCAGTaagcttcccctttgggataa | 40 | CAGGAAACAGCTATGACCcagggtgtgtgggtctagga | 205 |
| | chr2:25320527-25320711 | GTAAAACGACGGCCAGTAGGGTCCTAAGCAGTGAGCA | 41 | CAGGAAACAGCTATGACCCGGTCTTTCCATTCCAGGTA | 206 |
| | chr2:25320912-25321025 | GTAAAACGACGGCCAGTaggtgtgctacctggaatgg | 42 | CAGGAAACAGCTATGACCcagggcttaggctctgtgag | 207 |
| | chr2:25321625-25321705 | GTAAAACGACGGCCAGTATCTGGGGACTAAAATGGGG | 43 | CAGGAAACAGCTATGACCCCTGGACTCTTTTCTGGCTG | 208 |
| | chr2:25322392-25322437 | GTAAAACGACGGCCAGTAGCAAAGGTGAAAGGCTGAA | 44 | CAGGAAACAGCTATGACCAGCCCAAGGTCAAGGAGATT | 209 |
| | chr2:25322532-25322682 | GTAAAACGACGGCCAGTTCCCAGGCAACAAACTTACC | 45 | CAGGAAACAGCTATGACCGAACAAGTTGGAGACCAGGC | 210 |
| | chr2:25322992-25323149 | GTAAAACGACGGCCAGTTCTTCTGGAGGAGGAAAGCA | 46 | CAGGAAACAGCTATGACCCCTGTGCCACCCTCACTACT | 211 |
| | chr2:25323423-25323531 | GTAAAACGACGGCCAGTAGTAGTGAGGGTGGCACAGG | 47 | CAGGAAACAGCTATGACCCTCCTCTTTGCATCGGGTAA | 212 |
| | chr2:25323963-25324122 | GTAAAACGACGGCCAGTCTTACACTTGCAAGCACCCA | 48 | CAGGAAACAGCTATGACCGCCTCGTGACCACTGTGTAA | 213 |
| | chr2:25324409-25324625 | GTAAAACGACGGCCAGTCATCCACCAAGACACAATGC | 49 | CAGGAAACAGCTATGACCCTGTCACTGTTCCGGGTTTT | 214 |
| | chr2:25326029-25326097 | GTAAAACGACGGCCAGTTCTTCTCCACAATTCCCCTG | 50 | CAGGAAACAGCTATGACCAGGGCCGTGTTTCCTAGATT | 215 |
| | chr2:25328566-25328684 | GTAAAACGACGGCCAGTCACTCTTTTCAAACCCGGAG | 51 | CAGGAAACAGCTATGACCgcgcTAATCTCTTCCAGAGC | 216 |
| | chr2:25351313-25351460 | GTAAAACGACGGCCAGTactgaggcccatcacttctg | 52 | CAGGAAACAGCTATGACCcattgtgtttgaggcgagtg | 217 |
| | chr2:25351872-25351916 | GTAAAACGACGGCCAGTCTTCCCACAGAGGGATGTGT | 53 | CAGGAAACAGCTATGACCgaaCAGCTAAACGGCCAGAG | 218 |
| | chr2:25358585-25358964 | GTAAAACGACGGCCAGTTACAATCACCCAGCCCTCTC | 54 | CAGGAAACAGCTATGACCAGCGGTCAATGATCCAAAAC | 219 |
| | chr2:25358965-25359084 | GTAAAACGACGGCCAGTAGCCAAGTCCCTGACTCTCA | 55 | CAGGAAACAGCTATGACCAGCGGTCAATGATCCAAAAC | 220 |
| | chr2:25376511-25376616 | GTAAAACGACGGCCAGTTTGAAGAATGGGGTACCTGC | 56 | CAGGAAACAGCTATGACCGGTGGGGGCATATTACACAG | 221 |
| | chr2:25390285-25390534 | GTAAAACGACGGCCAGTtgcggtcatgcaCTCAGTAT | 57 | CAGGAAACAGCTATGACCGATCCTCTTCTCTCCCCCAC | 222 |
| EZH2 | chr7:148135407-148135731 | GTAAAACGACGGCCAGTcttccacatattcacaggcagt | 59 | CAGGAAACAGCTATGACCcttcagcaggctttgttgtg | 223 |
| | chr7:148137095-148137180 | GTAAAACGACGGCCAGTGCGGCATGATATGAGAAGGT | 59 | CAGGAAACAGCTATGACCCGCAAGGGTAACAAAATTCG | 224 |
| | chr7:148137334-148137415 | GTAAAACGACGGCCAGTtggtgtcagtgagcatgaaga | 60 | CAGGAAACAGCTATGACCttttagattttgtggtggatgc | 225 |
| | chr7:148138357-148138439 | GTAAAACGACGGCCAGTCACAAGAGGTGAGGTGAGCA | 61 | CAGGAAACAGCTATGACCGTGACCCTTTTTGTTGCGTT | 226 |
| | chr7:148139649-148139745 | GTAAAACGACGGCCAGTAGCATGCAAATCCACAAACA | 62 | CAGGAAACAGCTATGACCGTGTGCCCAATTACTGCCTT | 227 |
| | chr7:148141983-148142162 | GTAAAACGACGGCCAGTTTTGCCCCAGCTAAATCATC | 63 | CAGGAAACAGCTATGACCgtacagcccttgccacgtaT | 228 |
| | chr7:148142938-148143064 | GTAAAACGACGGCCAGTCCTGCCTCACACACACAGAC | 64 | CAGGAAACAGCTATGACCCTTGGGGGTGGGAGAGTATT | 229 |
| | chr7:148143530-148143571 | GTAAAACGACGGCCAGTCGGCTACATCTCAGTCCCAT | 65 | CAGGAAACAGCTATGACCATTTGTAGCTTCCCGCAGAA | 230 |
| | chr7:148144708-148144803 | GTAAAACGACGGCCAGTCCAACAACAGCCCTTAGGAA | 66 | CAGGAAACAGCTATGACCCCCAGCATCTAGCAGTGTCA | 231 |
| | chr7:148145246-148145416 | GTAAAACGACGGCCAGTTGACACTGCTAGATGCTGGG | 67 | CAGGAAACAGCTATGACCGCCGATTGGATTTGAGTTGT | 232 |
| | chr7:148145901-148146142 | GTAAAACGACGGCCAGTACAACTCAAATCCAATCGGC | 68 | CAGGAAACAGCTATGACCTGCCCTGATGTTGACATTTT | 233 |
| | chr7:148147620-148147712 | GTAAAACGACGGCCAGTGAGAGGGGCTTGGGATCTAC | 69 | CAGGAAACAGCTATGACCTGCGCATCAGTTTTACTTGC | 234 |
| | chr7:148154478-148154657 | GTAAAACGACGGCCAGTTCAGAGCAATCCTCAAGCAA | 70 | CAGGAAACAGCTATGACCTTCTTGATAACACCATGCACAA | 235 |
| | chr7:148155188-148155291 | GTAAAACGACGGCCAGTAAGTGTAGTGGCTCATCCGC | 71 | CAGGAAACAGCTATGACCttctgcttcccagtgctctT | 236 |
| | chr7:148156764-148156905 | GTAAAACGACGGCCAGTccaccctacctggccATAAT | 72 | CAGGAAACAGCTATGACCTGCTTCCTTTGCCTAACACC | 237 |
| | chr7:148157752-148157873 | GTAAAACGACGGCCAGTGAGCCCCTATATGCCACAGA | 73 | CAGGAAACAGCTATGACCTGCTTATTGGTGAGAGGGGT | 238 |
| | chr7:148160658-148160775 | GTAAAACGACGGCCAGTctgtcttgattcaccttgacaat | 74 | CAGGAAACAGCTATGACCggctacagcttaaggttgtcct | 239 |
| | chr7:148174494-148174623 | GTAAAACGACGGCCAGTGGTCAATGATTTCCTCCCAA | 75 | CAGGAAACAGCTATGACCATGGCAATCGTTTCCTGTTC | 240 |
| | chr7:148175206-148175330 | CAGGAAACAGCTATGACCATGGCAATCGTTTCCTGTTC | 76 | CAGGAAACAGCTATGACCgcagcacaaatgagcacct | 241 |
| FLT3 | chr13:27490603-27490726 | GTAAAACGACGGCCAGTCCTGAAGCTGCAGAAAAACC | 77 | CAGGAAACAGCTATGACCTCCATCACCGGTACCTCCTA | 242 |
| | chr13:27490603-27490726 | GTAAAACGACGGCCAGTGTTGACACCCCAATCCACTC | 78 | CAGGAAACAGCTATGACCGTGACCGGCTCCTCAGATAA | 243 |
| | chr13:27506218-27506351 | GTAAAACGACGGCCAGTTTTCCAAAAGCACCTGATCC | 79 | CAGGAAACAGCTATGACCTCATTGTCGTTTTAACCCTGC | 244 |
| HRAS | chr11:523765-523944 | GTAAAACGACGGCCAGTGATCTGCTCCCTGAGAGGTG | 80 | CAGGAAACAGCTATGACCAGAGGCTGGCTGTGTGAACT | 245 |
| | chr11:523765-523944 | GTAAAACGACGGCCAGTCTCCCTGGTACCTCTCATGC | 81 | CAGGAAACAGCTATGACCGTGGGTTTGCCCTTCAGAT | 246 |
| IDH1 | chr2:208821337-208821629 | GTAAAACGACGGCCAGTTGTGTTGAGATGGACGCCTA | 82 | CAGGAAACAGCTATGACCGGTGTACTCAGAGCCTTCGC | 247 |
| IDH2 | chr15:88432822-88432983 | GTAAAACGACGGCCAGTCTGCCTCTTTGTGGCCTAAG | 83 | CAGGAAACAGCTATGACCATTCTGGTTGAAAGATGGCG | 248 |
| JAK2 | chr9:5063697-5063785 | GTAAAACGACGGCCAGTGGGTTTCCTCAGAACGTTGA | 84 | CAGGAAACAGCTATGACCCTGACACCTAGCTGTGATCCTG | 249 |
| KIT | chr4:55284506-55284621 | GTAAAACGACGGCCAGTTTCTGCCCTTTGAACTTGCT | 85 | CAGGAAACAGCTATGACCAAAGCCACATGGCTAGAAAA | 250 |
| | chr4:55288338-55288465 | GTAAAACGACGGCCAGTCCACACCCTGTTCACTCCTT | 86 | CAGGAAACAGCTATGACCTGGCAAACCTATCAAAAGGG | 251 |
| | chr4:55293992-55294115 | GTAAAACGACGGCCAGTTGTGAACATCATTCAAGGCG | 87 | CAGGAAACAGCTATGACCTGTTCAGCATACCATGCAAA | 252 |
| KRas | chr12:25271434-25271613 | GTAAAACGACGGCCAGTTGCATGGCATTAGCAAAGAC | 88 | CAGGAAACAGCTATGACCGGTGCTTAGTGGCCATTTGT | 253 |
| | chr12:25289474-25289596 | GTAAAACGACGGCCAGTCCAAGGAAAGTAAAGTTCCCA | 89 | CAGGAAACAGCTATGACCCGTCTGCAGTCAACTGGAAT | 254 |
| NPM1 | chr5:170770135-170770493 | GTAAAACGACGGCCAGTCTCGGGAGATGAAGTTGGAA | 90 | CAGGAAACAGCTATGACCactccagcctaggggaAAAA | 255 |
| NRas | chr1:115057943-115058122 | GTAAAACGACGGCCAGTGTGGTAACCTCATTTCCCCA | 91 | CAGGAAACAGCTATGACCGGGACAAACCAGATAGGCAG | 256 |
| | chr1:115060193-115060321 | GTAAAACGACGGCCAGTCAGGTTTTAGAAACTTCAGCAGC | 92 | CAGGAAACAGCTATGACCATTAATCCGGTGTTTTTGCG | 257 |
| PHF6 | chrX:133339267-133339451 | GTAAAACGACGGCCAGTggggcttagagtggcttaattt | 93 | CAGGAAACAGCTATGACCgtctctgttgctgccggtat | 258 |
| | chrX:133339700-133339802 | GTAAAACGACGGCCAGTTCTGAAAACCAGAAGGTGGC | 94 | CAGGAAACAGCTATGACCGGATTTTGCTGGCTCAGAGA | 259 |
| | chrX:133355196-133355330 | GTAAAACGACGGCCAGTACCAATTTGTTTTCCTTGACAGA | 95 | CAGGAAACAGCTATGACCCGAGCAGTACACTTCACCCA | 260 |
| | chrX:133355604-133355648 | GTAAAACGACGGCCAGTACCACTGTGCATTGCATGAT | 96 | CAGGAAACAGCTATGACCTGAAAAGTGGCTGAAACGTG | 261 |
| | chrX:133375183-133375353 | GTAAAACGACGGCCAGTCTGAAACATTGGGTGGCTTT | 97 | CAGGAAACAGCTATGACCTTGGGCTTTAGATCACAGGG | 262 |
| | chrX:133375518-133375662 | GTAAAACGACGGCCAGTATGAACATGAACTGGAGCCC | 98 | CAGGAAACAGCTATGACCTTGGGCTTTAGATCACAGGG | 263 |
| | chrX:133376711-133376987 | GTAAAACGACGGCCAGTTTAATCTTGGCTCCACACTGG | 99 | CAGGAAACAGCTATGACCGCTTGCAAATGCCTTGAAAT | 264 |
| | chrX:133378864-133379244 | GTAAAACGACGGCCAGTtttcttgaaatacggcttacga | 100 | CAGGAAACAGCTATGACCccggcccagtgtatgtagtt | 265 |
| | chrX:133386896-133387276 | GTAAAACGACGGCCAGTCCCATGTTTTAAATGGGCAC | 101 | CAGGAAACAGCTATGACCATGATGCTTGAGGGGAACAC | 266 |
| PTEN | chr10:89614098-89614406 | GTAAAACGACGGCCAGTatcagctaagccaagtcc | 102 | CAGGAAACAGCTATGACCgcaacctgaccagggttaaa | 267 |
| | chr10:89643761-89643846 | GTAAAACGACGGCCAGTCTCCAGCTATAGTGGGGAAA | 103 | CAGGAAACAGCTATGACCCTGTATCCCCCTGAAGTCCA | 268 |
| | chr10:89675249-89675294 | GTAAAACGACGGCCAGTCCATAGAAGGGGTATTTGTTGG | 104 | CAGGAAACAGCTATGACCTGCCAACAATGTTTTACCTCA | 269 |
| | chr10:89680782-89680826 | GTAAAACGACGGCCAGTAAAGATTCAGGCAATGTTTGTT | 105 | CAGGAAACAGCTATGACCTCTCACTCGATAATCTGGATGAC | 270 |
| | chr10:89682749-89682988 | GTAAAACGACGGCCAGTGGAATCCAGTGTTTCTTTTAAATACC | 106 | CAGGAAACAGCTATGACCGAAACCCAAAATCTGTTTTCCA | 271 |
| | chr10:89701854-89701996 | GTAAAACGACGGCCAGTGGCTACGACCCAGTTACCAT | 107 | CAGGAAACAGCTATGACCTAAAACCCATTGCTTTTGGC | 272 |
| | chr10:89707589-89707756 | GTAAAACGACGGCCAGTTGCTTGAGATCAAGATTGCAG | 108 | CAGGAAACAGCTATGACCGCCATAAGGCCTTTTCCTTC | 273 |
| | chr10:89710630-89710855 | GTAAAACGACGGCCAGTGCAACAGATAACTCAGATTGCC | 109 | CAGGAAACAGCTATGACCTTTTGACGCTGTGTACATTGG | 274 |
| | chr10:89715023-89715403 | GTAAAACGACGGCCAGTTGTTCATCTGCAAAATGGAAT | 110 | CAGGAAACAGCTATGACCTAAAACGGGAAAGTGCCATC | 275 |
| RUNX1 | chr21:35086148-35086527 | GTAAAACGACGGCCAGTCTTCCTGTTTGCTTTCCAGC | 111 | CAGGAAACAGCTATGACCCACGCGCTACCACACCTAC | 276 |
| | chr21:35086528-35086777 | GTAAAACGACGGCCAGTACCACGTCGCTCTGGTTC | 112 | CAGGAAACAGCTATGACCATCCTCGTCCTCTTGGGAGT | 277 |
| | chr21:35093467-35093629 | GTAAAACGACGGCCAGTAAGAAAATCAGTGCATGGGC | 113 | CAGGAAACAGCTATGACCACCCTGGTACATAGGCCACA | 278 |
| | chr21:35115824-35115863 | GTAAAACGACGGCCAGTTGTTACGACGGTTTGCAGAG | 114 | CAGGAAACAGCTATGACCGGAAGGGAAGGGAAATCTTG | 279 |
| | chr21:35128576-35128768 | GTAAAACGACGGCCAGTAGTTGGTCTGGGAAGGTGTG | 115 | CAGGAAACAGCTATGACCGGAAAGACAAGAAAAGCCCC | 280 |
| | chr21:35153640-35153745 | GTAAAACGACGGCCAGTGCAACTTTTTGGCTTTACGG | 116 | CAGGAAACAGCTATGACCGGTAACTTGTGCTGAAGGGC | 281 |
| | chr21:35174723-35174880 | GTAAAACGACGGCCAGTCCGAGTTTCTAGGGATTCCA | 117 | CAGGAAACAGCTATGACCCATTGCTATTCCTCTGCAACC | 282 |
| | chr21:35181009-35181389 | GTAAAACGACGGCCAGTAGAAAGCTGAGACGAGTGCC | 118 | CAGGAAACAGCTATGACCGCAGAACCAGAACGTTTTCC | 283 |
| | chr21:35187091-35187130 | GTAAAACGACGGCCAGTGGAATCAGCAGAAACAGCCT | 119 | CAGGAAACAGCTATGACCAACCACGTGCATAAGGAACA | 284 |
| | chr21:35343008-35343388 | GTAAAACGACGGCCAGTGGTGAAACAAGCTGCCATTT | 120 | CAGGAAACAGCTATGACCTTTGGGCCTCATAAACAACC | 285 |
| TET2 | chr4:106374502-106374882 | GTAAAACGACGGCCAGTCACCCTTGTTCTCCATGACC | 121 | CAGGAAACAGCTATGACCTGGTTGACTGCTTTCACCTG | 286 |
| | chr4:106374883-106375262 | GTAAAACGACGGCCAGTAAATGGAGACACCAAGTGGC | 122 | CAGGAAACAGCTATGACCGAGGTATGCGATGGGTGAGT | 287 |
| | chr4:106375263-106375642 | GTAAAACGACGGCCAGTATGAGCAGGAGGGGAAAAGT | 123 | CAGGAAACAGCTATGACCTGGTGTGGTAGTGGCAGAAA | 288 |
| | chr4:106375643-106376022 | GTAAAACGACGGCCAGTACTCACCCATCGCATACCTC | 124 | CAGGAAACAGCTATGACCAGATAGTGCTGTGTTGGGGG | 289 |
| | chr4:106376023-106376402 | GTAAAACGACGGCCAGTTTCCACAGGTTCCTCAGCTT | 125 | CAGGAAACAGCTATGACCGAGAAGTGCACCTGGTGTGA | 290 |
| | chr4:106376783-106377162 | GTAAAACGACGGCCAGTAAGGCAAGCTTACACCCAGA | 126 | CAGGAAACAGCTATGACCGGTTCCACCTTAATTGGCCT | 291 |
| | chr4:106377163-106377542 | GTAAAACGACGGCCAGTAATGTCCAAATGGGACTGGA | 127 | CAGGAAACAGCTATGACCACTGGCCCTGACATTTCAAC | 292 |
| | chr4:106377543-106377922 | GTAAAACGACGGCCAGTCCCCAGAAGGACACTCAAAA | 128 | CAGGAAACAGCTATGACCCAAATTGCTGCCAGACTCAA | 293 |
| | chr4:106377923-106378302 | GTAAAACGACGGCCAGTACTTGATAGCCACACCCCAG | 129 | CAGGAAACAGCTATGACCTTCCCCCAACTCATGAAGAC | 294 |
| | chr4:106381723-106382102 | GTAAAACGACGGCCAGTtgcacaaaaggtagaatgcaa | 130 | CAGGAAACAGCTATGACCacgtgggatttcacacaaca | 295 |
| | chr4:106383436-106383533 | GTAAAACGACGGCCAGTTTTCCCATTTTCACCCACAT | 131 | CAGGAAACAGCTATGACCACCCAATTCTCAGGGTCAGA | 296 |
| | chr4:106384175-106384384 | GTAAAACGACGGCCAGTAGGGTCAAAGCCCACTTTTT | 132 | CAGGAAACAGCTATGACCTGAGGCCATGTGGTTACAGA | 297 |
| | chr4:106400224-106400375 | GTAAAACGACGGCCAGTGTGTGGTTATGCCACAGCTT | 133 | CAGGAAACAGCTATGACCCCAAAGAGGAAGTTTTTGTTGC | 298 |
| | chr4:106402364-106402454 | GTAAAACGACGGCCAGTACCATACGGCTTAATTCCCC | 134 | CAGGAAACAGCTATGACCTGTTACAATTGCTGCCAATGA | 299 |
| | chr4:106410215-106410353 | GTAAAACGACGGCCAGTTGTCATTCCATTTTGTTTCTGG | 135 | CAGGAAACAGCTATGACCCTGCTAAGCTGTCCTCAGCC | 300 |
| | chr4:106413169-106413524 | GTAAAACGACGGCCAGTTCTGGATCAACTAGGCCACC | 136 | CAGGAAACAGCTATGACCGGGGGCAAAACCAAAATAAT | 301 |
| | chr4:106415653-106416033 | GTAAAACGACGGCCAGTTCAAGCAGAGGCATGTTCAG | 137 | CAGGAAACAGCTATGACCTATTTCCAAACCTTGGCTGG | 302 |
| | chr4:106416034-106416413 | GTAAAACGACGGCCAGTAATCCCATGAACCCTTACCC | 138 | CAGGAAACAGCTATGACCACCAGACCTCATCGTTGTCC | 303 |
| | chr4:106416414-106416793 | GTAAAACGACGGCCAGTATCAGTGGACAACTGCTCCC | 139 | CAGGAAACAGCTATGACCATGAAACGCAGGTAAGTGGG | 304 |
| | chr4:106416794-106417173 | GTAAAACGACGGCCAGTATTGGCACTAGTCCAGGGTG | 140 | CAGGAAACAGCTATGACCACTGTGACCTTTCCCCACTG | 305 |
| TP53 | chr17:7505821-7506057 | GTAAAACGACGGCCAGTCGGAACTCCTGAGCTGAAAG | 141 | CAGGAAACAGCTATGACCGCAGGAGAGTTGCTTGAACC | 306 |
| | chr17:7510128-7510287 | GTAAAACGACGGCCAGTGTGCTGTGTGCTGGGATTAC | 142 | CAGGAAACAGCTATGACCGTGCCAGGAGCTGTTCTAGG | 307 |
| | chr17:7513585-7513733 | GTAAAACGACGGCCAGTCCACAACAAAACACCAGTGC | 143 | CAGGAAACAGCTATGACCAAAGCATTGGTCAGGGAAAA | 308 |
| | chr17:7514651-7514758 | GTAAAACGACGGCCAGTTCAACCGGAGGAAGACTAAAAA | 144 | CAGGAAACAGCTATGACCATCAGCCAAGATTGCACCAT | 309 |
| | chr17:7517249-7517309 | GTAAAACGACGGCCAGTaagcaggctaggctaagctatg | 145 | CAGGAAACAGCTATGACCaaggaccagaccagctttca | 310 |
| | chr17:7517577-7517651 | GTAAAACGACGGCCAGTTGTCTTTGAGGCATCACTGC | 146 | CAGGAAACAGCTATGACCGCGCACAGAGGAAGAGAATC | 311 |
| | chr17:7517743-7517880 | GTAAAACGACGGCCAGTGTGGTTTCTTCTTTGGCTGG | 147 | CAGGAAACAGCTATGACCCAAGGGTGGTTGGGAGTAGA | 312 |
| | chr17:7518223-7518333 | GTAAAACGACGGCCAGTtggaagaaatcggtaagaggtg | 148 | CAGGAAACAGCTATGACCctgcttgccacaggtctcc | 313 |
| | chr17:7518901-7519014 | GTAAAACGACGGCCAGTTTGCACATCTCATGGGGTTA | 149 | CAGGAAACAGCTATGACCAGTCACAGCACATGACGGAG | 314 |
| | chr17:7519095-7519475 | GTAAAACGACGGCCAGTTTACCTGCAATTGGGGCATT | 150 | CAGGAAACAGCTATGACCGCAGGCTAGGCTAAGCTATGATG | 315 |
| | chr17:7520036-7520315 | GTAAAACGACGGCCAGTGCCAAAGGGTGAAGAGGAAT | 151 | CAGGAAACAGCTATGACCGTAAGGACAAGGGTTGGGCT | 316 |
| | chr17:7520424-7520446 | GTAAAACGACGGCCAGTTCATCTGGACCTGGGTCTTC | 152 | CAGGAAACAGCTATGACCCCCCTCTGAGTCAGGAAACA | 317 |
| | chr11:7520563-7520665 | GTAAAACGACGGCCAGTAGCCCAACCCTTGTCCTTAC | 153 | CAGGAAACAGCTATGACCCAGCCATTCTTTTCCTGCTC | 318 |
| WT1 | chr11:32367041-32367301 | GTAAAACGACGGCCAGTGGGGACATGATCAGCTATGG | 154 | CAGGAAACAGCTATGACCTCCTTAAAGCCCCAAGAGGT | 319 |
| | chr11:32370093-32370186 | CAGGAAACAGCTATGACCGCCACGCACTATTCCTTCTC | 155 | GTAAAACGACGGCCAGTGGGAAATCTAAGGGTGAGGC | 320 |
| | chr11:32370787-32370877 | CAGGAAACAGCTATGACCTGTGGGGTGTTTCCTTTTCT | 156 | GTAAAACGACGGCCAGTGTTGGGGATCATCCTACCCT | 321 |
| | chr11:32374378-32374529 | CAGGAAACAGCTATGACCTAGCAGTGTGAGAGCCTGGA | 157 | GTAAAACGACGGCCAGTGGAGTGTGAATGGGAGTGGT | 322 |
| | chr11:32378069-32378166 | CAGGAAACAGCTATGACCTAAGGAACTAAAGGGCCGGT | 158 | GTAAAACGACGGCCAGTCCATCATTCCCTCCTGATTG | 323 |
| | chr11:32394611-32394662 | CAGGAAACAGCTATGACCGAATAAGAAGAGGTGGGGGC | 159 | GTAAAACGACGGCCAGTGGCTTTTCACTGGATTCTGG | 324 |
| | chr11:32395698-32395776 | CAGGAAACAGCTATGACCACCAACTAGGGGAAGGAGGA | 160 | GTAAAACGACGGCCAGTCTGTGCAGAGATCAGTGGGA | 325 |
| | chr11:32406077-32406180 | GTAAAACGACGGCCAGTCAGAGACCAGGGAGATCAGC | 161 | GTAAAACGACGGCCAGTGACTGCTAGGGGAATGCAAA | 326 |
| | chr11:32406618-32406741 | GTAAAACGACGGCCAGTTGCCATTGGGGTAATGATTT | 162 | CAGGAAACAGCTATGACCCAAGGTCACATCCAGGGACT | 327 |
| | chr11:32408651-32408935 | GTAAAACGACGGCCAGTAGTGAAGGCCGAATTTCTGA | 163 | CAGGAAACAGCTATGACCTCCAAGGCCTGTACAAGGAG | 328 |
| | chr11:32412821-32413201 | GTAAAACGACGGCCAGTGGTAAGAGCTGCGGTCAAAA | 164 | CAGGAAACAGCTATGACCCTACAGCAGCCAGAGCAGC | 329 |
| | chr11:32413202-32413581 | GTAAAACGACGGCCAGTGGCTCCTGTTTGATGAAGGA | 165 | CAGGAAACAGCTATGACCGTAAGGAGTTCAAGGCAGCG | 330 |

TABLE 3

| Gene | p-value |
|---|---|
| DNMT3A | 0.17 |
| IDH1 | 0.24 |
| IDH2 | 0.59 |
| IDH2R140Q | 0.61 |
| IDH2R172K | 0.13 |
| TET2 | 0.92 |
| ASXL1 | 0.16 |
| FLT3 | 0.6 |
| NPM1 | 0.23 |
| PHF6 | 0.09 |
| KIT | 0.24 |
| CEBPA | 0.23 |
| WT1 | 0.68 |
| KRas | 0.45 |
| NRas | 0.49 |
| P53 | 0.85 |
| PTEN | 0.95 |
| RUNX1 | 0.09 |
| CBF | 0.67 |
| Del(5q) | 0.66 |
| EVI | 0.9 |
| MLL-PTD | 0.04 |
| Split MLL | 0.21 |
| Monosomy 7 | 0.97 |
| t(6;9) | 0.36 |
| Trisomy 8 | 0.89 |
| AML1-ETO | 0.08 |

TABLE 4

| | Overall | Favorable | Intermediate | Unfavorable |
|---|---|---|---|---|
| Gene | Frequency (%) | Risk | Risk | Risk |
| FLT3 (ITD, | 37 (30, 7) | 8 (3, 5) | 52 (42, 7)* | 36 (35, 1) |
| TKD)¹ | | | | |
| NPM1 | 29 | 4 | 49* | 12 |
| DNMT3A | 23 | 4 | 33* | 15 |
| NRAS | 10 | 12 | 5 | 2 |
| CEBPA | 9 | 5 | 12 | 5 |
| TET2 | 8 | 5 | 8 | 10 |
| WT1 | 8 | 1 | 12* | 5 |
| IDH2 | 8 | 3 | 9 | 9 |
| IDH1 | 7 | 3 | 9 | 3 |
| KIT | 6 | 28* | 1 | 0 |
| RUNX1 | 5 | 3 | 6 | 6 |
| MLL-PTD² | 5 | 0 | 5 | 8 |
| ASXL1 | 3 | 0 | 4 | 2 |
| PHF6 | 3 | 1 | 2 | 3 |
| KRAS | 2 | 7 | 5 | 3 |
| PTEN | 2 | 1 | 2 | 1 |
| TP53 | 2 | 0 | 1 | 6 |
| HRAS | 0 | 0 | 0 | 0 |
| EZH2 | 0 | 0 | 0 | 0 |

¹ITD—internal tandem duplication; TKD—tyrosine kinase domain mutation.
²PTD—partial tandem duplication.
*denotes mutations which were significantly enriched in a specific cytogenetic risk group compared to the entire cohort (p < 0.01 for all).

TABLE 5

| | DNMT3a | IDH1 | IDH2 | TET2 | ASXL1 | FLT3 | NPM1 | CEBPA | WT1 | KRas | NRas | PHF6 |
|---|---|---|---|---|---|---|---|---|---|---|---|---|
| DNMT3a | | 3.3% | 1.5% | 1.5% | 0% | 13.3% | 14.3% | 1.75% | 0.75% | 0.75% | 2.5% | 0% |
| | | (13/398) | (6/398) | (6/398) | (0/398) | (53/398) | (57/398) | (7/398) | (3/398) | (3/398) | (10/398) | (0/398) |
| IDH1 | 3.3% | | 0% | 0% | 0.25% | 1% | 1.5% | 0.25% | 0% | 0.25% | 0.75% | 0.5% |
| | (13/398) | | (0/398) | (0/398) | (1/398) | (4/398) | (6/398) | (1/398) | (0/398) | (1/398) | (3/398) | (2/398) |
| IDH2 | 1.5% | 0% | | 0% | 0.5% | 2% | 2% | 0% | 0% | 0% | 0.75% | 0% |
| | (6/398) | (0/398) | | (0/398) | (2/398) | (8/398) | (8/398) | (0/398) | (0/398) | (0/398) | (3/398) | (0/398) |
| TET2 | 1.5% | 0% | 0% | | 0.75% | 3% | 1.5% | 0.5% | 0.5% | 0% | 1% | 0.25% |
| | (6/398) | (0/398) | (0/398) | | (3/398) | (12/398) | (6/398) | (2/398) | (2/398) | (0/398) | (4/398) | (1/398) |
| ASXL1 | 0% | 0.25% | 0.5% | 0.75% | | 0% | 0.25% | 0.5% | 0% | 0% | 0.25% | 0.25% |
| | (0/398) | (1/398) | (2/398) | (3/398) | | (0/398) | (1/398) | (2/398) | (0/398) | (0/398) | (1/398) | (1/398) |
| FLT3 | 13.3% | 1% | 2% | 3% | 0% | | 6.8% | 3.5% | 5% | 0.25% | 0.5% | 1% |
| | (53/398) | (4/398) | (8/398) | (12/398) | (0/398) | | (27/398) | (14/398) | (20/398) | (1/398) | (2/398) | (4/398) |
| NPM1 | 14.3% | 1.5% | 2% | 1.5% | 0.25% | 6.8% | | 0.5% | 0.25% | 0.5% | 1.3% | 0% |
| | (57/398) | (6/398) | (8/398 | (6/398) | (1/398) | (27/398) | | (2/398) | (1/398) | (2/398) | (5/398) | (0/398) |
| CEBPA | 1.75% | 0.25% | 0% | 0.5% | 0.5% | 3.5% | 0.5% | | 1.3% | 0% | 0.5% | 0.5% |
| | (7/398) | (1/398) | (0/398) | (2/398) | (2/398) | (14/398) | (2/398) | | (5/398) | (0/398) | (2/398) | (2/398) |
| WT1 | 0.75% | 0% | 0% | 0.5% | 0% | 5% | 0.25% | 1.3% | | 0% | 0.75% | 0% |
| | (3/398) | (0/398) | (0/398) | (2/398) | (0/398) | (20/398) | (1/398) | (5/398) | | (0/398) | (3/398) | (0/398) |
| KRas | 0.75% | 0.25% | 0% | 0% | 0% | 0.25% | 0.5% | 0% | 0% | | 0% | 0% |
| | (3/398) | (1/398) | (0/398) | (0/398) | (0/398) | (1/398) | (2/398) | (0/398) | (0/398) | | (0/398) | (0/398) |
| NRas | 2.5% | 0.75% | 0.75% | 1% | 0.25% | 0.5% | 1.3% | 0.5% | 0.75% | 0% | | 0% |
| | (10/398) | (3/398) | (3/398) | (4/398) | (1/398) | (2/398) | (5/398) | (2/398) | (3/398) | (0/398) | | (0/398) |
| PHF6 | 0% | 0.5% | 0% | 0.25% | 0.25% | 1% | 0% | 0.5% | 0% | 0% | 0% | |
| | (0/398) | (2/398) | (0/398) | (1/398) | (1/398) | (4/398) | (0/398) | (2/398) | (0/398) | (0/398) | (0/398) | |
| KIT | 0.5% | 0.25% | 0% | 0% | 0% | 0% | 0.25% | 0.5% | 0% | 0% | 0.25% | 0% |
| | (2/398) | (1/398) | (0/398) | (0/398) | (0/398) | (0/398) | (1/398) | (2/398) | (0/398) | (0/398) | (1/398) | (0/398) |
| TP53 | 0.25% | 0% | 0% | 0.25% | 0% | 0.25% | 0% | 0% | 0% | 0% | 0% | 0% |
| | (1/398) | (0/398) | (0/398) | (1/398) | (0/398) | (1/398) | (0/398) | (0/398) | (0/398) | (0/398) | (0/398) | (0/398) |
| PTEN | 0.75% | 0.5% | 0% | 0% | 0% | 0.5% | 0.5% | 0% | 0% | 0% | 0.5% | 0% |
| | (3/398) | (2/398) | (0/398) | (0/398) | (0/398) | (2/398) | (2/398) | (0/398) | (0/398) | (0/398) | (2/398) | (0/398) |
| RUNX1 | 0.75% | 0.25% | 0.75% | 0.25% | 1% | 1.5% | 0.5% | 0% | 0.75% | 0.25% | 0.5% | 0% |
| | (3/398) | (1/398) | (3/398) | (1/398) | (4/398) | (6/398) | (2/398) | (0/398) | (3/398 | (1/398) | (2/398) | (0/398) |
| CBF | 0.25% | 0.25% | 0% | 1.3% | 1.3% | 1.5% | 0% | 1% | 1% | 0.5% | 3% | 0.25% |
| | (1/398) | (1/398) | (0/398) | (5/398) | (5/398) | (6/398) | (0/398) | (4/398) | (4/398) | (2/398) | (12/398) | (1/398) |
| Del (5q) | 0% | 0% | 0% | 0.25% | 0% | 0.25% | 0% | 0% | 0% | 0% | 0% | 0.25% |
| | (0/398) | (0/398) | (0/398) | (1/398) | (0/398) | (1/398) | (0/398) | (0/398) | (0/398) | (0/398) | (0/398) | (1/398) |
| EVI1 | 0% | 0% | 0% | 0.25% | 0.25% | 0.25% | 0% | 0% | 0% | 0% | 0.25% | 0.25% |
| | (0/398) | (0/398) | (0/398) | (1/398) | (1/398) | (1/398) | (0/398) | (0/398) | (0/398) | (0/398) | (1/398) | (1/398) |
| MLL-PTD | 1% | 0.5% | 0.75% | 0% | 0.5% | 2.5% | 0% | 0.5% | 0.5% | 0% | 0% | 0.25% |
| | (4/398) | (2/398) | (3/398) | (0/398) | (2/398) | (10/398) | (0/398) | (2/398) | (2/398) | (0/398) | (0/398) | (1/398) |
| Split MLL | 0.25% | 0.25% | 0.5% | 0% | 0.25% | 0.5% | 0% | 0% | 0% | 0.25% | 0.75% | 0% |
| | (1/398) | (1/398) | (2/398) | (0/398) | (1/398) | (2/398) | (0/398) | (0/398) | (0/398) | (1/398) | (3/398) | (0/398) |
| Monosomy (7/7q) | 0.25% | 0.25% | 0.25% | 0.25% | 0% | 0% | 0% | 0.25% | 0% | 0% | 0% | 0% |
| | (1/398) | (1/398) | (1/398) | (1/398) | (0/398) | (0/398) | (0/398) | (1/398) | (0/398) | (0/398) | (0/398) | (0/398) |
| t(6; 9) | 0% | 0% | 0% | 0% | 0.25% | 0.25% | 0% | 0% | 0.25% | 0% | 0% | 0% |
| | (0/398) | (0/398) | (0/398) | (0/398) | (1/398) | (1/398) | (0/398) | (0/398) | (1/398) | (0/398) | (0/398) | (0/398) |
| Tri(8) | 1.5% | 0.5% | 0% | 0.25% | 0.25% | 2.26% | 0.25% | 0.25% | 0% | 0% | 0% | 0% |
| | (6/398) | (2/398) | (0/398) | (1/398) | (1/398) | (9/398) | (1/398) | (1/398) | (0/398) | (0/398) | (0/398) | (0/398) |
| AML1-ETO | 0% | 0% | 0% | 0% | 0% | 0% | 0% | 0% | 0% | 0% | 0% | 0% |
| | (0/398) | (0/398) | (0/398) | (0/398) | (0/398) | (0/398) | (0/398) | (0/398) | (0/398) | (0/398) | (0/398) | (0/398) |

| | | | | | | Del | | | Split | Monosomy | | | |
|---|---|---|---|---|---|---|---|---|---|---|---|---|---|
| | KIT | TP53 | PTEN | RUNX1 | CBF | (5q) | EVI1 | MLL- PTD | MLL | (7/7q) | t(6; 9) | Tri(8) | AML1-ETO |
| DNMT3a | 0.5% | 0.25% | 0.75% | 0.75% | 0.25% | 0% | 0% | 1% | 0.25% | 0.25% | 0% | 1.5% | 0% |
| | (2/398) | (1/398) | (3/398) | (3/398) | (1/398) | (0/398) | (0/398) | (4/398) | (1/398) | (1/398) | (0/398) | (6/398) | (0/398) |
| IDH1 | 0.25% | 0% | 0.5% | 0.25% | 0.25% | 0% | 0% | 0.5% | 0.25% | 0.25% | 0% | 0.5% | 0% |
| | (1/398) | (0/398) | (2/398) | (1/398) | (1/398) | (0/398) | (0/398) | (2/398) | (1/398) | (1/398) | (0/398) | (2/398) | (0/398) |
| IDH2 | 0% | 0% | 0% | 0.75% | 0% | 0% | 0% | 0.75% | 0.5% | 0.25% | 0% | 0% | 0% |
| | (0/398) | (0/398) | (0/398) | (3/398) | (0/398) | (0/398) | (0/398) | (3/398) | (2/398) | (1/398) | (0/398) | (0/398) | (0/398) |
| TET2 | 0% | 0.25% | 0% | 0.25% | 1.3% | 0.25% | 0.25% | 0% | 0% | 0.25% | 0% | 0.25% | 0% |
| | (0/398) | (1/398) | (0/398) | (1/398) | (5/398) | (1/398) | (1/398) | (0/398) | (0/398) | (1/398) | (0/398) | (1/398) | (0/398) |
| ASXL1 | 0% | 0% | 0% | 1% | 1.3% | 0% | 0.25% | 0.5% | 0.25% | 0% | 0.25% | 0.25% | 0% |
| | (0/398) | (0/398) | (0/398) | (4/398) | (5/398) | (0/398) | (1/398) | (2/398) | (1/398) | (0/398) | (1/398) | (1/398) | (0/398) |
| FLT3 | 0% | 0.25% | 0.5% | 1.5% | 1.5% | 0.25% | 0.25% | 2.5% | 0.5% | 0% | 0.25% | 2.26% | 0% |
| | (0/398) | (1/398) | (2/398) | (6/398) | (6/398) | (1/398) | (1/398) | (10/398) | (2/398) | (0/398) | (1/398) | (9/398) | (0/398) |
| NPM1 | 0.25% | 0% | 0.5% | 0.5% | 0% | 0% | 0% | 0% | 0% | 0% | 0% | 0.25% | 0% |
| | (1/398) | (0/398) | (2/398) | (2/398) | (0/398) | (0/398) | (0/398) | (0/398) | (0/398) | (0/398) | (0/398) | (1/398) | (0/398) |
| CEBPA | 0.5% | 0% | 0% | 0% | 1% | 0% | 0% | 0.5% | 0% | 0.25% | 0% | 0.25% | 0% |
| | (2/398) | (0/398) | (0/398) | (0/398) | (4/398) | (0/398) | (0/398) | (2/398) | (0/398) | (1/398) | (0/398) | (1/398) | (0/398) |
| WT1 | 0% | 0% | 0% | 0.75% | 1% | 0% | 0% | 0.5% | 0% | 0% | 0.25% | 0% | 0% |
| | (0/398) | (0/398) | (0/398) | (3/398) | (4/398) | (0/398) | (0/398) | (2/398) | (0/398) | (0/398) | (1/398) | (0/398) | (0/398) |
| KRas | 0% | 0% | 0% | 0.25% | 0.5% | 0% | 0% | 0% | 0.25% | 0% | 0% | 0% | 0% |
| | (0/398) | (0/398) | (0/398) | (1/398) | (2/398) | (0/398) | (0/398) | (0/398) | (1/398) | (0/398) | (0/398) | (0/398) | (0/398) |
| NRas | 0.25% | 0% | 0.5% | 0.5% | 3% | 0% | 0.25% | 0% | 0.75% | 0% | 0% | 0% | 0% |
| | (1/398) | (0/398) | (2/398) | (2/398) | (12/398) | (0/398) | (1/398) | (0/398) | (3/398) | (0/398) | (0/398) | (0/398) | (0/398) |
| PHF6 | 0% | 0% | 0% | 0% | 0.25% | 0.25% | 0.25% | 0.25% | 0% | 0% | 0% | 0% | 0% |
| | (0/398) | (0/398) | (0/398) | (0/398) | (1/398) | (1/398) | (1/398) | (1/398) | (0/398) | (0/398) | (0/398) | (0/398) | (0/398) |
| KIT | | 0% | 0% | 0% | 5.3% | 0% | 0% | 0% | 0% | 0% | 0% | 0% | 0% |
| | | (0/398) | (0/398) | (0/398) | (21/398) | (0/398) | (0/398) | (0/398) | (0/398) | (0/398) | (0/398) | (0/398) | (0/398) |
| TP53 | 0% | | 0.25% | 0.25% | 0% | 0.25% | 0% | 0.25% | 0% | 0% | 0% | 0% | 0% |
| | (0/398) | | (1/398) | (1/398) | (0/398) | (1/398) | (0/398) | (1/398) | (0/398) | (0/398) | (0/398) | (0/398) | (0/398) |
| PTEN | 0% | 0.25% | | 0% | 0.25% | 0% | 0% | 0% | 0% | 0% | 0% | 0% | 0% |
| | (0/398) | (1/398) | | (0/398) | (1/395) | (0/398) | (0/398) | (0/398) | (0/398) | (0/398) | (0/398) | (0/398) | (0/398) |
| RUNX1 | 0% | 0.25% | 0% | | 0.5% | 0.75% | 0% | 1% | 0% | 0.25% | 0% | 0% | 0% |
| | (0/398) | (1/398) | (0/398) | | (2/398) | (3/398) | (0/398) | (4/398) | (0/398) | (1/398) | (0/398) | (0/398) | (0/398) |
| CBF | 5.3% | 0% | 0.25% | 0.5% | | 0% | 0% | 0% | 0% | 0% | 0% | 0% | 0.25% |
| | (21/398) | (0/398) | (1/398) | (2/398) | | (0/398) | (0/398) | (0/398) | (0/398) | (0/398) | (0/398) | (0/398) | (1/398) |
| Del (5q) | 0% | 0.25% | 0% | 0.75% | 0% | | 0% | 1% | 0% | 0% | 0% | 0% | 0% |
| | (0/398) | (1/398) | (0/398) | (3/398) | (0/398) | | (0/398) | (4/398) | (0/398) | (0/398) | (0/398) | (0/398) | (0/398) |
| EVI1 | 0% | 0% | 0% | 0% | 0% | 0% | | 0% | 0% | 0% | 0% | 0% | 0% |
| | (0/398) | (0/398) | (0/398) | (0/398) | (0/398) | (0/398) | | (0/398) | (0/398) | (0/398) | (0/398) | (0/398) | (0/398) |
| MLL-PTD | 0% | 0.25% | 0% | 1% | 0% | 1% | 0% | | 0.5% | 0.25% | 0% | 0.25% | 0% |
| | (0/398) | (1/398) | (0/398) | (4/398) | (0/398) | (4/398) | (0/398) | | (2/398) | (1/398) | (0/398) | (1/398) | (0/398) |
| Split MLL | 0% | 0% | 0% | 0% | 0% | 0% | 0% | 0% | | 0% | 0% | 0% | 0% |
| | (0/398) | (0/398) | (0/398) | (0/398) | (0/398) | (0/398) | (0/398) | (0/398) | | (0/398) | (0/398) | (0/398) | (0/398) |
| Monosomy (7/7q) | 0% | 0% | 0% | 0.25% | 0% | 0% | 0% | 0.25% | 0% | | 0% | 0% | 0% |
| | (0/398) | (0/398) | (0/398) | (1/398) | (0/398) | (0/398) | (0/398) | (1/398) | (0/398) | | (0/398) | (0/398) | (0/398) |
| t(6; 9) | 0% | 0% | 0% | 0% | 0% | 0% | 0% | 0% | 0% | 0% | | 0% | 0% |
| | (0/398) | (0/398) | (0/398) | (0/398) | (0/398) | (0/398) | (0/398) | (0/398) | (0/398) | (0/398) | | (0/398) | (0/398) |
| Tri(8) | 0% | 0% | 0% | 0% | 0% | 0% | 0% | 0.25% | 0% | 0% | 0% | | 0% |
| | (0/398) | (0/398) | (0/398) | (0/398) | (0/398) | (0/398) | (0/398) | (1/398) | (0/398) | (0/398) | (0/398) | | (0/398) |
| AML1-ETO | 0% | 0% | 0% | 0% | 0.25% | 0% | 0% | 0% | 0% | 0% | 0% | 0% | |
| | (0/398) | (0/398) | (0/398) | (0/398) | (1/398) | (0/398) | (0/398) | (0/398) | (0/398) | (0/398) | (0/398) | (0/398) | |

TABLE 6

| | Abnormality #1 | Abnormality #2 | M/M² | WT/M³ | M/W⁴ | WT/WT⁵ |
|---|---|---|---|---|---|---|
| 1 | DNMT3A | IDH | 19 | 32 | 70 | 262 |
| 2 | DNMT3A | IDH1 | 13 | 9 | 76 | 286 |
| 3 | DNMT3A | IDH2 | 6 | 23 | 83 | 272 |
| 4 | DNMT3A | IDH2_R140Q | 3 | 18 | 86 | 277 |
| 5 | DNMT3A | IDH2_R172K | 3 | 5 | 86 | 290 |
| 6 | DNMT3A | TET2 | 6 | 26 | 83 | 266 |
| 7 | DNMT3A | ASXL1 | 0 | 10 | 88 | 285 |
| 8 | DNMT3A | FLT3 | 52 | 92 | 37 | 204 |
| 9 | DNMT3A | NPM1 | 57 | 57 | 32 | 239 |
| 10 | DNMT3A | PHF6 | 0 | 9 | 88 | 284 |
| 11 | DNMT3A | KIT | 2 | 21 | 87 | 275 |
| 12 | DNMT3A | CEBPa | 6 | 26 | 82 | 267 |
| 13 | DNMT3A | WT1 | 3 | 26 | 86 | 264 |
| 14 | DNMT3A | KRAS | 2 | 6 | 87 | 288 |
| 15 | DNMT3A | NRAS | 10 | 28 | 79 | 267 |
| 16 | DNMT3A | TP53 | 1 | 7 | 86 | 283 |
| 17 | DNMT3A | PTEN | 3 | 2 | 86 | 293 |
| 18 | DNMT3A | RUNX1 | 3 | 16 | 85 | 267 |
| 19 | DNMT3A | CBF | 1 | 71 | 88 | 225 |
| 20 | DNMT3A | del5q | 1 | 5 | 88 | 291 |
| 21 | DNMT3A | EVI1pos | 0 | 5 | 89 | 291 |
| 22 | DNMT3A | MLLPTD or split | 4 | 13 | 85 | 283 |
| | | MLLPTD | | | | |
| 23 | DNMT3A | splitMLLPTD or | 1 | 21 | 88 | 275 |
| | | split MLL | | | | |
| 24 | DNMT3A | MLLPTD or split | 5 | 32 | 84 | 264 |
| | | MLL | | | | |
| 25 | DNMT3A | Monosomy7 | 1 | 2 | 88 | 294 |
| 26 | DNMT3A | t(6; 9) | 0 | 2 | 89 | 294 |
| 27 | DNMT3A | trisomy 8 | 6 | 9 | 83 | 287 |
| 28 | DNMT3A | AML1ETO | 0 | 1 | 89 | 295 |
| 29 | DNMT3A_R882 | IDH | 13 | 38 | 50 | 282 |
| 30 | DNMT3A_R882 | IDH1 | 9 | 13 | 54 | 308 |
| 31 | DNMT3A_R882 | IDH2 | 4 | 25 | 59 | 296 |
| 32 | DNMT3A_R882 | IDH2_R140Q | 2 | 19 | 61 | 302 |
| 33 | DNMT3A_R882 | IDH2_R172K | 2 | 6 | 61 | 315 |
| 34 | DNMT3A_R882 | IDH1_IDH2_R172K | 11 | 19 | 52 | 302 |
| 35 | DNMT3A_R882 | TET2 | 4 | 28 | 59 | 290 |
| 36 | DNMT3A_R882 | ASXL1 | 0 | 10 | 62 | 311 |
| 37 | DNMT3A_R882 | FLT3 | 41 | 103 | 22 | 219 |
| 38 | DNMT3A_R882 | NPM1 | 43 | 71 | 20 | 251 |
| 39 | DNMT3A_R882 | PHF6 | 0 | 9 | 62 | 310 |
| 40 | DNMT3A_R882 | KIT | 2 | 21 | 61 | 301 |
| 41 | DNMT3A_R882 | CEBPa | 4 | 28 | 58 | 291 |
| 42 | DNMT3A_R882 | WT1 | 0 | 29 | 63 | 287 |
| 43 | DNMT3A_R882 | KRAS | 2 | 6 | 61 | 314 |
| 44 | DNMT3A_R882 | NRAS | 5 | 33 | 58 | 288 |
| 45 | DNMT3A_R882 | TP53 | 1 | 7 | 60 | 309 |
| 46 | DNMT3A_R882 | PTEN | 2 | 3 | 61 | 318 |
| 47 | DNMT3A_R882 | RUNX1 | 2 | 17 | 61 | 291 |
| 48 | DNMT3A_R882 | CBF | 0 | 72 | 63 | 250 |
| 49 | DNMT3A_R882 | del5q | 1 | 5 | 62 | 317 |
| 50 | DNMT3A_R882 | EVI1pos | 0 | 5 | 63 | 317 |
| 51 | DNMT3A_R882 | MLLPTD or split | 3 | 14 | 60 | 308 |
| | | MLLPTD | | | | |
| 52 | DNMT3A_R882 | splitMLLPTD or | 0 | 22 | 63 | 300 |
| | | split MLL | | | | |
| 53 | DNMT3A_R882 | MLLPTD or split | 3 | 34 | 60 | 288 |
| | | MLL | | | | |
| 54 | DNMT3A_R882 | Monosomy7 | 0 | 3 | 63 | 319 |
| 55 | DNMT3A_R882 | t(6: 9) | 0 | 2 | 63 | 320 |
| 56 | DNMT3A_R882 | trisomy 8 | 5 | 10 | 58 | 312 |
| 57 | DNMT3A_R882 | AML1ETO | 0 | 1 | 63 | 321 |
| 58 | DNMT3A_other | IDH | 6 | 45 | 22 | 310 |
| 59 | DNMT3A_other | IDH1 | 4 | 18 | 24 | 338 |
| 60 | DNMT3A_other | IDH2 | 2 | 27 | 26 | 329 |
| 61 | DNMT3A_other | IDH2_R140Q | 1 | 20 | 27 | 336 |
| 62 | DNMT3A_other | IDH2_R172K | 1 | 7 | 27 | 349 |
| 63 | DNMT3A_other | IDH1_IDH2_R172K | 5 | 25 | 23 | 331 |
| 64 | DNMT3A_other | TET2 | 2 | 30 | 26 | 323 |
| 65 | DNMT3A_other | ASXL1 | 0 | 10 | 28 | 345 |
| 66 | DNMT3A_other | FLT3 | 12 | 132 | 16 | 225 |
| 67 | DNMT3A_other | NPM1 | 15 | 99 | 13 | 258 |
| 68 | DNMT3A_other | PHF6 | 0 | 9 | 28 | 344 |
| 69 | DNMT3A_other | KIT | 0 | 23 | 28 | 334 |
| 70 | DNMT3A_other | CEBPa | 2 | 30 | 26 | 323 |
| 71 | DNMT3A_other | WT1 | 3 | 26 | 25 | 325 |
| 72 | DNMT3A_other | KRAS | 0 | 8 | 28 | 347 |
| 73 | DNMT3A_other | NRAS | 6 | 32 | 22 | 324 |
| 74 | DNMT3A_other | TP53 | 0 | 8 | 28 | 341 |
| 75 | DNMT3A_other | PTEN | 1 | 4 | 27 | 352 |
| 76 | DNMT3A_other | RUNX1 | 2 | 17 | 25 | 327 |
| 77 | DNMT3A_other | CBF | 1 | 71 | 27 | 286 |
| 78 | DNMT3A_other | del5q | 1 | 5 | 27 | 352 |
| 79 | DNMT3A_other | EVI1pos | 0 | 5 | 28 | 352 |
| 80 | DNMT3A_other | MLLPTD or split | 1 | 16 | 27 | 341 |
| | | MLLPTD | | | | |
| 81 | DNMT3A_other | splitMLLPTD or | 1 | 21 | 27 | 336 |
| | | split MLL | | | | |
| 82 | DNMT3A_other | MLLPTD or split | 2 | 35 | 26 | 322 |
| | | MLL | | | | |
| 83 | DNMT3A_other | Monosomy7 | 1 | 2 | 27 | 355 |
| 84 | DNMT3A_other | t(6; 9) | 0 | 2 | 28 | 355 |
| 85 | DNMT3A_other | trisomy 8 | 1 | 14 | 27 | 343 |
| 86 | DNMT3A_other | AML1ETO | 0 | 1 | 28 | 356 |
| 87 | IDH | TET2 | 0 | 33 | 56 | 301 |
| 88 | IDH | ASXL1 | 3 | 7 | 54 | 329 |
| 89 | IDH | FLT3 | 13 | 133 | 44 | 205 |
| 90 | IDH | NPM1 | 31 | 87 | 26 | 251 |
| 91 | IDH | PHF6 | 2 | 7 | 54 | 328 |
| 92 | IDH | KIT | 1 | 22 | 56 | 316 |
| 93 | IDH | CEBPa | 1 | 33 | 56 | 302 |
| 94 | IDH | WT1 | 0 | 30 | 56 | 303 |
| 95 | IDH | KRAS | 1 | 7 | 56 | 329 |
| 96 | IDH | NRAS | 6 | 34 | 51 | 303 |
| 97 | IDH | TP53 | 0 | 8 | 57 | 323 |
| 98 | IDH | PTEN | 2 | 4 | 55 | 333 |
| 99 | IDH | RUNX1 | 4 | 16 | 52 | 308 |
| 100 | IDH | CBF | 1 | 71 | 56 | 267 |
| 101 | IDH | del5q | 0 | 6 | 57 | 332 |
| 102 | IDH | EVI1pos | 0 | 5 | 57 | 333 |
| 103 | IDH | MLLPTD or split | 5 | 13 | 52 | 325 |
| | | MLLPTD | | | | |
| 104 | IDH | splitMLLPTD or | 2 | 19 | 55 | 319 |
| | | split MLL | | | | |
| 105 | IDH | MLLPTD or split | 6 | 31 | 51 | 307 |
| | | MLL | | | | |
| 106 | IDH | Monosomy7 | 2 | 2 | 55 | 336 |
| 107 | IDH | t(6; 9) | 0 | 2 | 57 | 336 |
| 108 | IDH | trisomy 8 | 2 | 13 | 55 | 325 |
| 109 | IDH | AML1ETO | 0 | 1 | 57 | 337 |
| 110 | IDH1 | IDH2 | 0 | 33 | 24 | 338 |
| 111 | IDH1 | IDH2_R140Q | 0 | 24 | 24 | 347 |
| 112 | IDH1 | IDH2_R172K | 0 | 9 | 24 | 362 |
| 113 | IDH1 | TET2 | 0 | 33 | 24 | 334 |
| 114 | IDH1 | ASXL1 | 1 | 9 | 23 | 361 |
| 115 | IDH1 | FLT3 | 4 | 142 | 20 | 230 |
| 116 | IDH1 | NPM1 | 14 | 104 | 10 | 268 |
| 117 | IDH1 | PHF6 | 2 | 7 | 21 | 362 |
| 118 | IDH1 | KIT | 1 | 22 | 23 | 350 |
| 119 | IDH1 | CEBPa | 1 | 33 | 23 | 336 |
| 120 | IDH1 | WT1 | 0 | 30 | 23 | 337 |
| 121 | IDH1 | KRAS | 1 | 7 | 23 | 363 |
| 122 | IDH1 | NRAS | 3 | 37 | 21 | 334 |
| 123 | IDH1 | TP53 | 0 | 8 | 24 | 356 |
| 124 | IDH1 | PTEN | 2 | 4 | 22 | 367 |
| 125 | IDH1 | RUNX1 | 1 | 19 | 22 | 339 |
| 126 | IDH1 | CBF | 1 | 71 | 23 | 301 |
| 127 | IDH1 | del5q | 0 | 6 | 24 | 366 |
| 128 | IDH1 | EVI1pos | 0 | 5 | 24 | 367 |
| 129 | IDH1 | MLLPTD or split | 2 | 16 | 22 | 356 |
| | | MLLPTD | | | | |
| 130 | IDH1 | splitMLLPTD or | 0 | 21 | 24 | 351 |
| | | split MLL | | | | |
| 131 | IDH1 | MLLPTD or split | 2 | 35 | 22 | 337 |
| | | MLL | | | | |
| 132 | IDH1 | Monosomy7 | 1 | 3 | 23 | 369 |
| 133 | IDH1 | t(6; 9) | 0 | 2 | 24 | 370 |
| 134 | IDH1 | trisomy 8 | 2 | 13 | 22 | 359 |
| 135 | IDH1 | AML1ETO | 0 | 1 | 24 | 371 |
| 136 | IDH2 | ASXL1 | 2 | 8 | 31 | 353 |
| 137 | IDH2 | FLT3 | 9 | 138 | 24 | 225 |
| 138 | IDH2 | NPM1 | 17 | 101 | 16 | 262 |
| 139 | IDH2 | PHF6 | 0 | 9 | 33 | 350 |
| 140 | IDH2 | KIT | 0 | 23 | 33 | 340 |
| 141 | IDH2 | CEBPa | 0 | 34 | 33 | 325 |
| 142 | IDH2 | WT1 | 0 | 30 | 33 | 327 |
| 143 | IDH2 | KRAS | 0 | 8 | 33 | 353 |
| 144 | IDH2 | NRAS | 3 | 37 | 30 | 325 |
| 145 | IDH2 | TP53 | 0 | 8 | 33 | 348 |
| 146 | IDH2 | PTEN | 0 | 6 | 33 | 356 |
| 147 | IDH2 | RUNX1 | 3 | 17 | 30 | 331 |
| 148 | IDH2 | CBF | 0 | 72 | 33 | 291 |
| 149 | IDH2 | del5q | 0 | 6 | 33 | 357 |
| 150 | IDH2 | EVI1pos | 0 | 5 | 33 | 358 |
| 151 | IDH2 | MLLPTD or split | 3 | 15 | 30 | 348 |
| | | MLLPTD | | | | |
| 152 | IDH2 | splitMLLPTD or | 2 | 20 | 31 | 343 |
| | | split MLL | | | | |
| 153 | IDH2 | MLLPTD or split | 4 | 34 | 29 | 329 |
| | | MLL | | | | |
| 154 | IDH2 | Monosomy7 | 1 | 3 | 32 | 360 |
| 155 | IDH2 | t(6; 9) | 0 | 2 | 33 | 361 |
| 156 | IDH2 | Trisomy 8 | 0 | 15 | 33 | 348 |
| 157 | IDH2 | AML1ETO | 0 | 1 | 33 | 362 |
| 158 | IDH2_R140Q | IDH2_R172K | 0 | 9 | 24 | 363 |
| 159 | IDH2_R140Q | TET2 | 0 | 33 | 23 | 335 |
| 160 | IDH2_R140Q | ASXL1 | 1 | 9 | 23 | 361 |
| 161 | IDH2_R140Q | FLT3 | 8 | 139 | 16 | 233 |
| 162 | IDH2_R140Q | NPM1 | 16 | 102 | 8 | 270 |
| 163 | IDH2_R140Q | PHF6 | 0 | 9 | 24 | 359 |
| 164 | IDH2_R140Q | KIT | 0 | 23 | 24 | 349 |
| 165 | IDH2_R140Q | CEBPa | 0 | 34 | 24 | 334 |
| 166 | IDH2_R140Q | WT1 | 0 | 30 | 24 | 336 |
| 167 | IDH2_R140Q | KRAS | 0 | 8 | 24 | 362 |
| 168 | IDH2_R140Q | NRAS | 3 | 37 | 21 | 334 |
| 169 | IDH2_R140Q | TP53 | 0 | 8 | 24 | 357 |
| 170 | IDH2_R140Q | PTEN | 0 | 6 | 24 | 365 |
| 171 | IDH2_R140Q | RUNX1 | 2 | 18 | 22 | 339 |
| 172 | IDH2_R140Q | CBF | 0 | 72 | 24 | 300 |
| 173 | IDH2_R140Q | del5q | 0 | 6 | 24 | 366 |
| 174 | IDH2_R140Q | EVI1pos | 0 | 5 | 24 | 367 |
| 175 | IDH2_R140Q | MLLPTD or split | 1 | 17 | 23 | 355 |
| | | MLLPTD | | | | |
| 176 | IDH2_R140Q | splitMLLPTD or | 2 | 20 | 22 | 352 |
| | | split MLL | | | | |
| 177 | IDH2_R140Q | MLLPTD or split | 2 | 36 | 22 | 336 |
| | | MLL | | | | |
| 178 | IDH2_R140Q | Monosomy7 | 1 | 3 | 23 | 369 |
| 179 | IDH2_R140Q | t(6; 9) | 0 | 2 | 24 | 370 |
| 180 | IDH2_R140Q | trisomy 8 | 0 | 15 | 24 | 357 |
| 181 | IDH2_R140Q | AML1ETO | 0 | 1 | 24 | 371 |
| 182 | IDH2_R172K | TET2 | 0 | 33 | 9 | 349 |
| 183 | IDH2_R172K | ASXL1 | 1 | 9 | 8 | 376 |
| 184 | IDH2_R172K | FLT3 | 1 | 146 | 8 | 241 |
| 185 | IDH2_R172K | NPM1 | 1 | 117 | 8 | 270 |
| 186 | IDH2_R172K | PHF6 | 0 | 9 | 9 | 374 |
| 187 | IDH2_R172K | KIT | 0 | 23 | 9 | 364 |
| 188 | IDH2_R172K | CEBPa | 0 | 34 | 9 | 349 |
| 189 | IDH2_R172K | WT1 | 0 | 30 | 9 | 351 |
| 190 | IDH2_R172K | KRAS | 0 | 8 | 9 | 377 |
| 191 | IDH2_R172K | NRAS | 0 | 40 | 9 | 346 |
| 192 | IDH2_R172K | TP53 | 0 | 8 | 9 | 372 |
| 193 | IDH2_R172K | PTEN | 0 | 6 | 9 | 380 |
| 194 | IDH2_R172K | RUNX1 | 1 | 19 | 8 | 353 |
| 195 | IDH2_R172K | CBF | 0 | 72 | 9 | 315 |
| 196 | IDH2_R172K | del5q | 0 | 6 | 9 | 381 |
| 197 | IDH2_R172K | EVI1pos | 0 | 6 | 9 | 382 |
| 198 | IDH2_R172K | MLLPTD or split | 2 | 16 | 7 | 371 |
| | | MLLPTD | | | | |
| 199 | IDH2_R172K | splitMLLPTD or | 0 | 22 | 9 | 365 |
| | | split MLL | | | | |
| 200 | IDH2 R172K | MLLPTD or split | 2 | 36 | 7 | 351 |
| | | MLL | | | | |
| 201 | IDH2_R172K | Monosomy7 | 0 | 4 | 9 | 383 |
| 202 | IDH2_R172K | t(6; 9) | 0 | 2 | 9 | 385 |
| 203 | IDH2_R172K | Trisomy 8 | 0 | 15 | 9 | 372 |
| 204 | IDH2_R172K | AML1ETO | 0 | 1 | 9 | 386 |
| 205 | TET2 | ASXL1 | 4 | 6 | 29 | 351 |
| 206 | TET2 | FLT3 | 12 | 134 | 21 | 225 |
| 207 | TET2 | NPM1 | 10 | 106 | 23 | 253 |
| 208 | TET2 | PHF6 | 2 | 7 | 31 | 348 |
| 209 | TET2 | KIT | 1 | 22 | 32 | 337 |
| 210 | TET2 | CEBPa | 2 | 31 | 30 | 325 |
| 211 | TET2 | WT1 | 3 | 27 | 30 | 326 |
| 212 | TET2 | KRAS | 0 | 8 | 33 | 349 |
| 213 | TET2 | NRAS | 4 | 34 | 29 | 325 |
| 214 | TET2 | TP53 | 1 | 7 | 32 | 344 |
| 215 | TET2 | PTEN | 1 | 5 | 32 | 353 |
| 216 | TET2 | RUNX1 | 3 | 15 | 29 | 330 |
| 217 | TET2 | CBF | 4 | 67 | 29 | 292 |
| 218 | TET2 | del5q | 0 | 6 | 33 | 353 |
| 219 | TET2 | EVI1pos | 1 | 4 | 32 | 355 |
| 220 | TET2 | MLLPTD or split | 0 | 18 | 33 | 341 |
| | | MLLPTD | | | | |
| 221 | TET2 | splitMLLPTD or | 1 | 21 | 32 | 338 |
| | | split MLL | | | | |
| 222 | TET2 | MLLPTD or split | 1 | 37 | 32 | 322 |
| | | MLL | | | | |
| 223 | TET2 | Monosomy7 | 1 | 2 | 32 | 357 |
| 224 | TET2 | t(6; 9) | 0 | 2 | 33 | 357 |
| 225 | TET2 | Trisomy 8 | 1 | 14 | 32 | 345 |
| 226 | TET2 | AML1ETO | 0 | 1 | 33 | 358 |
| 227 | ASXL1 | FLT3 | 0 | 146 | 10 | 239 |
| 228 | ASXL1 | NPM1 | 1 | 117 | 9 | 268 |
| 229 | ASXL1 | PHF6 | 1 | 8 | 9 | 373 |
| 230 | ASXL1 | KIT | 0 | 22 | 10 | 363 |
| 231 | ASXL1 | CEBPa | 2 | 32 | 8 | 349 |
| 232 | ASXL1 | WT1 | 0 | 30 | 10 | 349 |
| 233 | ASXL1 | KRAS | 0 | 8 | 10 | 375 |
| 234 | ASXL1 | NRAS | 1 | 38 | 9 | 346 |
| 235 | ASXL1 | TP53 | 0 | 8 | 9 | 370 |
| 236 | ASXL1 | PTEN | 0 | 6 | 10 | 378 |
| 237 | ASXL1 | RUNX1 | 5 | 15 | 4 | 356 |
| 238 | ASXL1 | CBF | 0 | 71 | 10 | 314 |
| 239 | ASXL1 | del5q | 0 | 6 | 10 | 379 |
| 240 | ASXL1 | EVI1pos | 0 | 5 | 10 | 380 |
| 241 | ASXL1 | MLLPTD or split | 0 | 17 | 10 | 368 |
| | | MLLPTD | | | | |
| 242 | ASXL1 | splitMLLPTD or | 0 | 22 | 10 | 363 |
| | | split MLL | | | | |
| 243 | ASXL1 | MLLPTD or split | 0 | 37 | 10 | 348 |
| | | MLL | | | | |
| 244 | ASXL1 | Monosomy7 | 0 | 4 | 10 | 381 |
| 245 | ASXL1 | t(6; 9) | 0 | 2 | 10 | 383 |
| 246 | ASXL1 | Trisomy 8 | 0 | 15 | 10 | 370 |
| 247 | ASXL1 | AML1ETO | 0 | 1 | 10 | 384 |
| 248 | FLT3 | NPM1 | 63 | 55 | 84 | 195 |
| 249 | FLT3 | PHF6 | 3 | 6 | 143 | 241 |
| 250 | FLT3 | KIT | 0 | 23 | 147 | 227 |
| 251 | FLT3 | CEBPa | 13 | 21 | 131 | 228 |
| 252 | FLT3 | WT1 | 18 | 12 | 127 | 234 |
| 253 | FLT3 | KRAS | 1 | 7 | 146 | 241 |
| 254 | FLT3 | NRAS | 3 | 37 | 144 | 212 |
| 255 | FLT3 | TP53 | 1 | 7 | 144 | 237 |
| 256 | FLT3 | PTEN | 2 | 4 | 144 | 246 |
| 257 | FLT3 | RUNX1 | 6 | 14 | 139 | 223 |
| 258 | FLT3 | CBF | 6 | 66 | 141 | 184 |
| 259 | FLT3 | del5q | 1 | 5 | 146 | 245 |
| 260 | FLT3 | EVI1pos | 1 | 4 | 146 | 246 |
| 261 | FLT3 | MLLPTD or split | 10 | 8 | 137 | 242 |
| | | MLLPTD | | | | |
| 262 | FLT3 | splitMLLPTD or | 2 | 20 | 145 | 230 |
| | | split MLL | | | | |
| 263 | FLT3 | MLLPTD or split | 11 | 27 | 136 | 223 |
| | | MLL | | | | |
| 264 | FLT3 | Monosomy7 | 0 | 4 | 147 | 246 |
| 265 | FLT3 | t(6; 9) | 1 | 1 | 146 | 249 |
| 266 | FLT3 | Trisomy 8 | 9 | 6 | 138 | 244 |
| 267 | FLT3 | AML1ETO | 0 | 1 | 147 | 249 |
| 268 | NPM1 | PHF6 | 0 | 9 | 118 | 266 |
| 269 | NPM1 | KIT | 2 | 21 | 116 | 258 |
| 270 | NPM1 | CEBPa | 3 | 31 | 113 | 246 |
| 271 | NPM1 | WT1 | 6 | 24 | 111 | 250 |
| 272 | NPM1 | KRAS | 3 | 5 | 115 | 272 |
| 273 | NPM1 | NRAS | 14 | 26 | 103 | 253 |
| 274 | NPM1 | TP53 | 1 | 7 | 115 | 266 |
| 275 | NPM1 | PTEN | 3 | 3 | 115 | 275 |
| 276 | NPM1 | RUNX1 | 4 | 16 | 114 | 248 |
| 277 | NPM1 | CBF | 0 | 72 | 118 | 207 |
| 278 | NPM1 | del5q | 0 | 6 | 118 | 273 |
| 279 | NPM1 | EVI1pos | 0 | 5 | 118 | 274 |
| 280 | NPM1 | MLLPTD or split | 0 | 18 | 118 | 261 |
| | | MLLPTD | | | | |
| 281 | NPM1 | splitMLLPTD or | 0 | 22 | 118 | 257 |
| | | split MLL | | | | |
| 282 | NPM1 | MLLPTD or split | 0 | 38 | 118 | 241 |
| | | MLL | | | | |
| 283 | NPM1 | Monosomy7 | 0 | 4 | 118 | 275 |
| 284 | NPM1 | t(6; 9) | 0 | 2 | 118 | 277 |
| 285 | NPM1 | Trisomy 8 | 2 | 13 | 116 | 268 |
| 286 | NPM1 | AML1ETO | 0 | 1 | 118 | 278 |
| 287 | PHF6 | KIT | 0 | 23 | 9 | 361 |
| 288 | PHF6 | CEBPa | 2 | 32 | 7 | 348 |
| 289 | PHF6 | WT1 | 0 | 30 | 9 | 348 |
| 290 | PHF6 | KRAS | 0 | 8 | 9 | 374 |
| 291 | PHF6 | NRAS | 0 | 39 | 9 | 344 |
| 292 | PHF6 | TP53 | 0 | 8 | 9 | 368 |
| 293 | PHF6 | PTEN | 0 | 6 | 9 | 377 |
| 294 | PHF6 | RUNX1 | 1 | 19 | 8 | 350 |
| 295 | PHF6 | CBF | 1 | 70 | 8 | 314 |
| 296 | PHF6 | del5q | 1 | 5 | 8 | 379 |
| 297 | PHF6 | EVI1pos | 1 | 4 | 8 | 380 |
| 298 | PHF6 | MLLPTD or split | 1 | 17 | 8 | 367 |
| | | MLLPTD | | | | |
| 299 | PHF6 | splitMLLPTD or | 0 | 22 | 9 | 362 |
| | | split MLL | | | | |
| 300 | PHF6 | MLLPTD or split | 1 | 37 | 8 | 347 |
| | | MLL | | | | |
| 301 | PHF6 | Monosomy7 | 0 | 4 | 9 | 380 |
| 302 | PHF6 | t(6; 9) | 0 | 2 | 9 | 382 |
| 303 | PHF6 | Trisomy 8 | 1 | 13 | 8 | 371 |
| 304 | PHF6 | AML1ETO | 0 | 1 | 9 | 383 |
| 305 | KIT | CEBPa | 2 | 32 | 21 | 338 |
| 306 | KIT | WT1 | 0 | 30 | 22 | 339 |
| 307 | KIT | KRAS | 0 | 8 | 22 | 365 |
| 308 | KIT | NRAS | 2 | 38 | 21 | 335 |
| 309 | KIT | TP53 | 0 | 8 | 23 | 356 |
| 310 | KIT | PTEN | 0 | 6 | 23 | 367 |
| 311 | KIT | RUNX1 | 0 | 20 | 22 | 340 |
| 312 | KIT | CBF | 21 | 51 | 2 | 323 |
| 313 | KIT | del5q | 0 | 6 | 23 | 368 |
| 314 | KIT | EVI1pos | 0 | 5 | 23 | 369 |
| 315 | KIT | MLLPTD or split | 0 | 18 | 23 | 356 |
| | | MLLPTD | | | | |
| 316 | KIT | splitMLLPTD or | 0 | 22 | 23 | 352 |
| | | split MLL | | | | |
| 317 | KIT | MLLPTD or split | 0 | 38 | 23 | 336 |
| | | MLL | | | | |
| 318 | KIT | Monosomy7 | 0 | 4 | 23 | 370 |
| 319 | KIT | t(6; 9) | 0 | 2 | 23 | 372 |
| 320 | KIT | Trisomy 8 | 0 | 15 | 23 | 359 |
| 321 | KIT | AML1ETO | 0 | 1 | 23 | 373 |
| 322 | CEBPa | WT1 | 4 | 26 | 28 | 329 |
| 323 | CEBPa | KRAS | 0 | 8 | 34 | 349 |
| 324 | CEBPa | NRAS | 2 | 38 | 32 | 320 |
| 325 | CEBPa | TP53 | 0 | 8 | 34 | 343 |
| 326 | CEBPa | PTEN | 0 | 6 | 34 | 352 |
| 327 | CEBPs | RUNX1 | 0 | 20 | 33 | 326 |
| 328 | CEBPa | CBF | 4 | 68 | 30 | 291 |
| 329 | CEBPa | del5q | 0 | 6 | 34 | 353 |
| 330 | CEBPa | EVI1pos | 1 | 4 | 33 | 355 |
| 331 | CEBPa | MLLPTD or split | 2 | 16 | 32 | 343 |
| | | MLLPTD | | | | |
| 332 | CEBPa | splitMLLPTD or | 0 | 21 | 34 | 336 |
| | | split MLL | | | | |
| 333 | CEBPa | MLLPTD or split | 2 | 35 | 32 | 324 |
| | | MLL | | | | |
| 334 | CEBPa | Monosomy7 | 0 | 3 | 34 | 356 |
| 335 | CEBPa | t(6; 9) | 0 | 2 | 34 | 357 |
| 336 | CEBPa | Trisomy 8 | 1 | 14 | 33 | 345 |
| 337 | CEBPa | AML1ETO | 0 | 1 | 34 | 358 |
| 338 | WT1 | KRAS | 0 | 8 | 30 | 351 |
| 339 | WT1 | NRAS | 3 | 37 | 27 | 323 |
| 340 | WT1 | TP53 | 0 | 8 | 30 | 345 |
| 341 | WT1 | PTEN | 0 | 6 | 30 | 354 |
| 342 | WT1 | RUNX1 | 3 | 17 | 26 | 330 |
| 343 | WT1 | CBF | 1 | 69 | 29 | 292 |
| 344 | WT1 | del5q | 0 | 6 | 30 | 355 |
| 345 | WT1 | EVI1pos | 0 | 4 | 30 | 357 |
| 346 | WT1 | MLLPTD or split | 2 | 16 | 28 | 345 |
| | | MLLPTD | | | | |
| 347 | WT1 | splitMLLPTD or | 0 | 22 | 30 | 339 |
| | | split MLL | | | | |
| 348 | WT1 | MLLPTD or split | 2 | 36 | 28 | 325 |
| | | MLL | | | | |
| 349 | WT1 | Monosomy7 | 0 | 4 | 30 | 357 |
| 350 | WT1 | t(6; 9) | 1 | 1 | 29 | 360 |
| 351 | WT1 | Trisomy 8 | 1 | 14 | 29 | 347 |
| 352 | WT1 | AML1ETO | 0 | 1 | 30 | 360 |
| 353 | KRAS | NRAS | 0 | 40 | 8 | 346 |
| 354 | KRAS | TP53 | 0 | 8 | 8 | 371 |
| 355 | KRAS | PTEN | 0 | 6 | 8 | 380 |
| 356 | KRAS | RUNX1 | 1 | 19 | 7 | 353 |
| 357 | KRAS | CBF | 2 | 68 | 6 | 319 |
| 358 | KRAS | del5q | 0 | 6 | 8 | 381 |
| 359 | KRAS | EVI1pos | 0 | 5 | 8 | 382 |
| 360 | KRAS | MLLPTD or split | 0 | 18 | 8 | 369 |
| | | MLLPTD | | | | |
| 361 | KRAS | splitMLLPTD or | 1 | 21 | 7 | 366 |
| | | split MLL | | | | |
| 362 | KRAS | MLLPTD or split | 1 | 37 | 7 | 350 |
| | | MLL | | | | |
| 363 | KRAS | Monosomy7 | 0 | 4 | 8 | 383 |
| 364 | KRAS | t(6; 9) | 0 | 2 | 8 | 385 |
| 365 | KRAS | Trisomy 8 | 0 | 15 | 8 | 372 |
| 366 | KRAS | AML1ETO | 0 | 1 | 8 | 386 |
| 367 | NRAS | TP53 | 0 | 8 | 39 | 341 |
| 368 | NRAS | PTEN | 2 | 4 | 38 | 351 |
| 369 | NRAS | RUNX1 | 2 | 18 | 35 | 326 |
| 370 | NRAS | CBF | 12 | 60 | 28 | 296 |
| 371 | NRAS | del5q | 0 | 6 | 40 | 350 |
| 372 | NRAS | EVI1pos | 1 | 4 | 39 | 352 |
| 373 | NRAS | MLLPTD or split | 0 | 18 | 40 | 338 |
| | | MLLPTD | | | | |
| 374 | NRAS | splitMLLPTD or | 2 | 20 | 38 | 336 |
| | | split MLL | | | | |
| 375 | NRAS | MLLPTD or split | 2 | 36 | 38 | 320 |
| | | MLL | | | | |
| 376 | NRAS | Monosomy7 | 0 | 4 | 40 | 352 |
| 377 | NRAS | t(6; 9) | 0 | 2 | 40 | 354 |
| 378 | NRAS | Trisomy 8 | 0 | 15 | 40 | 341 |
| 379 | NRAS | AML1ETO | 0 | 1 | 40 | 365 |
| 380 | TP53 | PTEN | 1 | 5 | 7 | 375 |
| 381 | TP53 | RUNX1 | 1 | 19 | 7 | 348 |
| 382 | TP53 | CBF | 0 | 72 | 8 | 309 |
| 383 | TP53 | del5q | 1 | 5 | 7 | 376 |
| 384 | TP53 | EVI1pos | 0 | 5 | 8 | 376 |
| 385 | TP53 | MLLPTD or split | 0 | 17 | 8 | 364 |
| | | MLLPTD | | | | |
| 386 | TP53 | splitMLLPTD or | 0 | 22 | 8 | 359 |
| | | split MLL | | | | |
| 387 | TP53 | MLLPTD or split | 0 | 37 | 8 | 344 |
| | | MLL | | | | |
| 388 | TP53 | Monosomy7 | 0 | 4 | 8 | 377 |
| 389 | TP53 | t(6; 9) | 0 | 2 | 8 | 379 |
| 390 | TP53 | trisomy 8 | 0 | 15 | 8 | 366 |
| 391 | TP53 | AML1ETO | 0 | 1 | 8 | 380 |
| 392 | PTEN | RUNX1 | 0 | 20 | 6 | 355 |
| 393 | PTEN | CBF | 1 | 71 | 5 | 319 |
| 394 | PTEN | del5q | 0 | 6 | 6 | 384 |
| 395 | PTEN | EVI1pos | 0 | 5 | 6 | 385 |
| 396 | PTEN | MLLPTD or split | 0 | 18 | 6 | 372 |
| | | MLLPTD | | | | |
| 397 | PTEN | splitMLLPTD or | 0 | 22 | 6 | 368 |
| | | split MLL | | | | |
| 398 | PTEN | MLLPTD or split | 0 | 38 | 6 | 352 |
| | | MLL | | | | |
| 399 | PTEN | Monosomy7 | 0 | 4 | 6 | 386 |
| 400 | PTEN | t(6; 9) | 0 | 2 | 6 | 388 |
| 401 | PTEN | trisomy 8 | 0 | 15 | 6 | 375 |
| 402 | PTEN | AML1ETO | 0 | 1 | 6 | 389 |
| 403 | RUNX1 | CBF | 2 | 65 | 18 | 296 |
| 404 | RUNX1 | del5q | 3 | 3 | 17 | 359 |
| 405 | RUNX1 | EVI1pos | 0 | 4 | 20 | 358 |
| 406 | RUNX1 | MLLPTD or split | 3 | 15 | 17 | 347 |
| | | MLLPTD | | | | |
| 407 | RUNX1 | splitMLLPTD or | 0 | 19 | 20 | 343 |
| | | split MLL | | | | |
| 408 | RUNX1 | MLLPTD or split | 3 | 32 | 17 | 330 |
| | | MLL | | | | |
| 409 | RUNX1 | Monosomy7 | 1 | 2 | 19 | 360 |
| 410 | RUNX1 | t(6; 9) | 0 | 2 | 20 | 360 |
| 411 | RUNX1 | trisomy 8 | 0 | 14 | 20 | 348 |
| 412 | RUNX1 | AML1ETO | 0 | 1 | 20 | 361 |
| 413 | CBF | del5q | 0 | 6 | 72 | 319 |
| 414 | CBF | EVI1pos | 0 | 5 | 72 | 320 |
| 415 | CBF | MLLPTD or split | 0 | 18 | 72 | 307 |
| | | MLLPTD | | | | |
| 416 | CBF | splitMLLPTD or | 0 | 22 | 72 | 303 |
| | | split MLL | | | | |
| 417 | CBF | MLLPTD or split | 0 | 38 | 72 | 287 |
| | | MLL | | | | |
| 418 | CBF | Monosomy7 | 0 | 4 | 72 | 321 |
| 419 | CBF | t(6; 9) | 0 | 2 | 72 | 323 |
| 420 | CBF | trisomy 8 | 0 | 15 | 72 | 310 |
| 421 | CBF | AML1ETO | 1 | 0 | 71 | 325 |
| 422 | del5q | EVI1pos | 0 | 5 | 6 | 386 |
| 423 | del5q | MLLPTD or split | 0 | 18 | 6 | 373 |
| | | MLLPTD | | | | |
| 424 | del5q | splitMLLPTD or | 0 | 22 | 6 | 369 |
| | | split MLL | | | | |
| 425 | del5q | MLLPTD or split | 0 | 38 | 6 | 353 |
| | | MLL | | | | |
| 426 | del5q | Monosomy7 | 0 | 4 | 6 | 387 |
| 427 | del5q | t(6; 9) | 0 | 2 | 6 | 389 |
| 428 | del5q | trisomy 8 | 0 | 15 | 6 | 376 |
| 429 | del5q | AML1ETO | 0 | 1 | 6 | 390 |
| 430 | EVI1pos | MLLPTD or split | 0 | 18 | 5 | 374 |
| | | MLLPTD | | | | |
| 431 | EVI1pos | splitMLLPTD or | 0 | 22 | 5 | 370 |
| | | split MLL | | | | |
| 432 | EVI1pos | MLLPTD or split | 0 | 38 | 5 | 354 |
| | | MLL | | | | |
| 433 | EVI1pos | Monosomy7 | 0 | 4 | 5 | 388 |
| 434 | EVI1pos | t(6; 9) | 0 | 2 | 5 | 390 |
| 435 | EVI1pos | trisomy 8 | 0 | 15 | 5 | 377 |
| 436 | EVI1pos | AML1ETO | 0 | 1 | 5 | 391 |
| 437 | MLLPTD or split | Monosomy7 | 1 | 3 | 17 | 376 |
| | MLLPTD | | | | | |
| 438 | MLLPTD or split | t(6; 9) | 0 | 2 | 18 | 377 |
| | MLLPTD | | | | | |
| 439 | MLLPTD or split | trisomy 8 | 0 | 15 | 18 | 364 |
| | MLLPTD | | | | | |
| 440 | MLLPTD or split | AML1ETO | 0 | 1 | 18 | 378 |
| | MLLPTD | | | | | |
| 441 | splitMLLPTD or | Monosomy7 | 0 | 4 | 22 | 371 |
| | split MLL | | | | | |
| 442 | splitMLLPTD or | t(6; 9) | 0 | 2 | 22 | 373 |
| | split MLL | | | | | |
| 443 | splitMLLPTD or | trisomy 8 | 0 | 15 | 22 | 360 |
| | split MLL | | | | | |
| 444 | splitMLLPTD or | AML1ETO | 0 | 1 | 22 | 374 |
| | split MLL | | | | | |
| 445 | MLLPTD or split | Monosomy7 | 1 | 3 | 37 | 356 |
| | MLL | | | | | |
| 446 | MLLPTD or split | t(6; 9) | 0 | 2 | 38 | 357 |
| | MLL | | | | | |
| 447 | MLLPTD or split | trisomy 8 | 0 | 15 | 38 | 344 |
| | MLL | | | | | |
| 448 | MLLPTD or split | AML1ETO | 0 | 1 | 38 | 358 |
| | MLL | | | | | |
| 449 | Monosomy7 | t(6; 9) | 0 | 2 | 4 | 391 |
| 450 | Monosomy7 | trisomy 8 | 0 | 15 | 4 | 378 |
| 451 | Monosomy7 | AML1ETO | 0 | 1 | 4 | 392 |
| 452 | t(6; 9) | trisomy 8 | 0 | 15 | 2 | 380 |
| 453 | t(6; 9) | AML1ETO | 0 | 1 | 2 | 394 |
| 454 | Trisomy 8 | AML1ETO | 0 | 1 | 15 | 381 |

1) Single nucleotide variants which could not be verified as bona fide somatic mutations were censored from analysis, therefore sample number does not add up to 398 in all instances.
²Number of patients mutated for both gene #1 and gene #2.
³Number of patients wildtype for gene #1 but mutant for gene #2.
⁴Number of patients mutated for gene #1 and wildtype for gene #2.
⁵Number of patients wildtype for both genes.

TABLE 7

[symbol]

| Mutated | Mutated | | | % | | | % | | Adjusted |
|---|---|---|---|---|---|---|---|---|---|
| Gene #1 | Gene #2 | M/M² | WT/M³ | M/M⁴ | M/WT⁵ | WT/WT⁶ | M/WT⁷ | p-value⁸ | p-value⁹ |
| ASXL1 | RUNX1 | 5 | 15 | 25.0 | 4 | 356 | 1.1 | <0.001 | <0.001 |
| DNMT3A | NPM1 | 57 | 57 | 50.0 | 32 | 239 | 11.8 | <0.001 | <0.001 |
| DNMT3A | FLT3 | 52 | 92 | 36.1 | 37 | 204 | 15.4 | <0.001 | <0.001 |
| | ITD | | | | | | | | |
| DNMT3A | IDH1 | 13 | 9 | 59.1 | 76 | 286 | 21.0 | <0.001 | 0.008 |
| DNMT3A | IDH1 or | 19 | 32 | 37.3 | 70 | 262 | 21.1 | 0.02 | 0.91 |
| | IDH2 | | | | | | | | |
| FLT3 ITD | NPM1 | 63 | 55 | 53.4 | 84 | 195 | 30.1 | <0.001 | <0.001 |
| FLT3 ITD | WT1 | 18 | 12 | 60.0 | 127 | 234 | 35.2 | 0.01 | 0.94 |
| IDH1 or | NPM1 | 31 | 87 | 26.3 | 26 | 251 | 9.4 | <0.001 | 0.002 |
| IDH2 | | | | | | | | | |
| IDH1 | NPM1 | 14 | 104 | 11.9 | 10 | 268 | 3.6 | 0.004 | 0.38 |
| IDH1 | PTEN | 2 | 4 | 33.3 | 22 | 367 | 5.7 | 0.05 | 0.69 |
| IDH2 | NPM1 | 17 | 101 | 14.4 | 16 | 262 | 5.8 | 0.01 | 0.67 |
| IDH2 | NPM1 | 16 | 102 | 13.6 | 8 | 270 | 2.9 | <0.001 | 0.01 |
| R140Q | | | | | | | | | |
| KIT | CBF | 21 | 51 | 29.2 | 2 | 323 | 0.6 | <0.001 | <0.001 |
| NRAS | CBF | 12 | 60 | 16.7 | 28 | 296 | 8.6 | 0.05 | 0.1 |
| RUNX1 | Del 5q | 3 | 3 | 50.0 | 17 | 359 | 4.5 | 0.002 | 1.0 |
| TET2 | ASXL1 | 4 | 6 | 40.0 | 29 | 351 | 7.6 | 0.006 | 0.03 |

1) Single nucleotide variants which could not be verified as bona fide somatic mutations were censored from analysis, therefore sample number does not sum up to 398 in all instances.
²Number of patients mutated for both gene #1 and gene #2.
³Number of patients wildtype for gene #1 but mutant for gene #2.
⁴Percentage of patients mutant for gene #1 and gene #2 over all patients mutated for either gene.
⁵Number of patients mutated for gene #1 and wildtype for gene #2.
⁶Number of patients wildtype for both genes.
⁷Percentage of patients mutant for either gene over all patients wildtype for either gene.
⁸P-value by Fisher's exact test.
⁹P-value adjusted for multiple comparisons. [symbol]
indicates data missing or illegible when filed

TABLE 8

[symbol]

| Mutated | Mutated | | | % | | | % | | Adjusted |
|---|---|---|---|---|---|---|---|---|---|
| Gene #1 | Gene #2 | M/M² | WT/M³ | M/M⁴ | M/WT⁵ | WT/WT⁶ | M/WT⁷ | p-value⁸ | p-value⁹ |
| ASXL1 | FLT3 | 0 | 146 | 0 | 10 | 239 | 4.0 | 0.02 | 0.94 |
| CBF | MLL | 0 | 38 | 0 | 72 | 287 | 20.1 | <0.001 | 0.99 |
| | abnormalities | | | | | | | | |
| CBF | Split MLL | 0 | 22 | 0 | 72 | 303 | 19.2 | 0.02 | 1.0 |
| CBF | MLL PTD | 0 | 18 | 0 | 72 | 307 | 19.0 | 0.05 | 1.0 |
| DNMT3A | CBF | 1 | 71 | 1.4 | 88 | 225 | 28.1 | <0.001 | 0.11 |
| DNMT3A | Split MLL | 1 | 21 | 4.6 | 88 | 275 | 24.2 | 0.04 | 0.97 |
| DNMT3A | WT1 | 0 | 29 | 0 | 63 | 287 | 18.0 | 0.01 | 0.92 |
| R882 | | | | | | | | | |
| FLT3 | CBF | 6 | 66 | 8.3 | 141 | 184 | 43.4 | <0.001 | 0.02 |
| FLT3 | NRAS | 3 | 37 | 7.5 | 144 | 212 | 40.5 | <0.001 | 0.008 |
| FLT3 | KIT | 0 | 23 | 0 | 147 | 227 | 39.3 | <0.001 | 0.04 |
| FLT3 | Splt MLL | 2 | 20 | 9.1 | 145 | 230 | 38.7 | 0.005 | 0.39 |
| IDH1 or | CBF | 1 | 71 | 1.4 | 56 | 267 | 17.3 | <0.001 | 0.63 |
| IDH2 | | | | | | | | | |
| IDH1 or | TET2 | 0 | 33 | 0 | 56 | 301 | 15.7 | 0.008 | 0.97 |
| IDH2 | | | | | | | | | |
| IDH1 or | WT1 | 0 | 30 | 0 | 56 | 303 | 15.6 | 0.01 | 0.98 |
| IDH2 | | | | | | | | | |
| IDH1 or | FLT3 | 13 | 133 | 8.9 | 44 | 205 | 17.7 | 0.02 | 1.0 |
| IDH2 | | | | | | | | | |
| IDH1 or | CEBPA | 1 | 33 | 2.9 | 56 | 302 | 15.6 | 0.04 | 0.99 |
| IDH2 | | | | | | | | | |
| IDH1 | FLT3 | 4 | 142 | 2.7 | 20 | 230 | 8.0 | 0.04 | 1.0 |
| IDH2 | CBF | 0 | 72 | 0 | 33 | 291 | 10.2 | 0.002 | 0.99 |
| NPM1 | CBF | 0 | 72 | 0 | 118 | 207 | 36.3 | <0.001 | 0.001 |
| NPM1 | MLL | 0 | 38 | 0 | 118 | 241 | 32.9 | <0.001 | 0.02 |
| | abnormalities | | | | | | | | |
| NPM1 | Split MLL | 0 | 22 | 0 | 118 | 257 | 31.5 | <0.001 | 0.59 |
| NPM1 | MLL PTD | 0 | 18 | 0 | 118 | 261 | 31.1 | 0.002 | 0.59 |
| NPM1 | CEBPA | 3 | 31 | 8.2 | 113 | 246 | 31.5 | 0.005 | 0.34 |
| NPM1 | KIT | 2 | 21 | 8.7 | 116 | 258 | 31.0 | 0.03 | 0.99 |
| WT1 | CBF | 1 | 69 | 1.4 | 29 | 292 | 9.0 | 0.03 | 1.0 |

1) Single nucleotide variants which could not be verified as bona fide somatic mutations were censored from analysis, therefore sample number does not sum up to 398 in all instances.
²Number of patients mutated for both gene #1 and gene #2
³Number of patients wildtype for gene #1 but mutant for gene #2
⁴Percentage of patients mutant for gene #1 and gene #2 over all patients mutated for either gene
⁵Number of patients mutated for gene #1 and wildtype for gene #2
⁶Number of patients wildtype for both genes
⁷Percentage of patients mutant for either genes over all patients wildtype for either gene
⁸P-value by Fisher's exact test.
⁹P-value adjusted for multiple comparisons [symbol]
indicates data missing or illegible when filed

TABLE 9

| | | | | | MV |
|---|---|---|---|---|---|
| Gene/ | | | Median | UV | analysis |
| Cytogenetic | Mutational | Number of | Survival | analysis | p- |
| Abnormality | Status | patients | (months) | p-value² | value³ |
| DNMT3A | Mutant | 88 | 14.1 | 0.19 | 0.29 |
| | Wildtype | 296 | 21.3 | | |
| DNMT3A | R882 Mutant | 63 | 14.1 | 0.14 | 0.26 |
| | Wildtype | 321 | 21.3 | | |
| DNMT3A | Non-R882 | 27 | 18.2 | 0.90 | 0.91 |
| | Mutant | | | | |
| | Wildtype | 357 | 18.0 | | |
| IDH1/2 | Mutant for | 56 | 42.4 | 0.009 | 0.001 |
| | IDH1 or | | | | |
| | IDH2 | | | | |
| | Wildtype | 358 | 16.2 | | |
| IDH1 | Mutant | 23 | 38.7 | 0.42 | 0.59 |
| | Wildtype | 372 | 17.0 | | |
| IDH2 | Mutant | 33 | 49.4 | 0.01 | 0.001 |
| | Wildtype | 362 | 16.3 | | |
| IDH2 | R140Q | 24 | — | 0.009 | 0.001 |
| | Mutant | | | | |
| | Wildtype | 371 | 16.6 | | |
| IDH2 | R172K | 9 | 41.3 | 0.58 | 0.46 |
| | Mutant | | | | |
| | Wildtype | 386 | 16.9 | | |
| TET2 | Mutant | 33 | 13.2 | 0.16 | 0.61 |
| | Wildtype | 358 | 18.0 | | |
| ASXL1 | Mutant | 10 | 10.3 | 0.05 | 0.22 |
| | Wildtype | 384 | 17.7 | | |
| FLT3 | Mutant | 148 | 13.8 | 0.006 | 0.003 |
| | Wildtype | 248 | 22.0 | | |
| NPM1 | Mutant | 118 | 22.3 | 0.07 | 0.005 |
| | Wildtype | 278 | 16.5 | | |
| PHF6 | Mutant | 9 | 4.3 | 0.006 | 0.08 |
| | Wildtype | 383 | 17.7 | | |
| KIT | Mutant | 23 | 57.9 | 0.08 | 0.6 |
| | Wildtype | 373 | 16.6 | | |
| CEBPa | Mutant | 34 | 31.7 | 0.05 | 0.03 |
| | Wildtype | 358 | 16.9 | | |
| WT1 | Mutant | 30 | 12.2 | 0.23 | 0.19 |
| | Wildtype | 360 | 17.7 | | |
| KRAS | Mutant | 8 | — | 0.17 | 0.19 |
| | Wildtype | 386 | 16.9 | | |
| NRAS | Mutant | 40 | 21.3 | 0.13 | 0.19 |
| | Wildtype | 355 | 16.9 | | |
| TP53 | Mutant | 8 | 12.4 | 0.14 | 0.83 |
| | Wildtype | 380 | 18.2 | | |
| PTEN | Mutant | 6 | 15.2 | 0.68 | 0.68 |
| | Wildtype | 389 | 17.9 | | |
| RUNX1 | Mutant | 20 | 16.9 | 0.90 | 0.63 |
| | Wildtype | 361 | 16.9 | | |
| CBF | Present | 43 | — | 0.001 | 0.47 |
| translocations | Absent | 353 | 16.2 | | |
| Del 5q | Present | 12 | 7.0 | 0.001 | 0.46 |
| | Absent | 384 | 18.0 | | |
| EVI positive | Present | 8 | 2.8 | <0.001 | 0.02 |
| | Absent | 388 | 18.0 | | |
| MLL PTD | Present | 19 | 12.6 | 0.009 | 0.19 |
| | Absent | 377 | 18.0 | | |
| Split MLL | Present | 25 | 11.7 | 0.05 | 0.44 |
| | Absent | 371 | 18.2 | | |
| Any MLL | Present | 39 | 10.9 | <0.001 | 0.33 |
| abnormalities | Absent | 357 | 19.7 | | |
| Monosomy 7 | Present | 9 | 3.5 | <0.001 | 0.18 |
| | Absent | 387 | 18.0 | | |
| t(6;9) | Present | 2 | 15.8 | 0.42 | 0.81 |
| | Absent | 394 | 17.5 | | |
| Trisomy 8 | Present | 19 | 10.2 | 0.06 | 0.03 |
| | Absent | 377 | 18.0 | | |
| t(8;21) | Present | 29 | 47.1 | 0.02 | 0.37 |
| | Absent | 367 | 16.5 | | |

1) Absence of value under column for overall survival indicates that deaths were not observed.
²Univariate (UV) analysis p-value (calculated by Log-rank test).
³Multivariate (MV) analysis p-value taking into account WBC count, age, transplantation, and cytogenetics.

TABLE 10

| | | | Median | |
|---|---|---|---|---|
| Gene/Cytogenetic | | Number of | Survival | |
| Abnormality | Mutational Status | patients | (months) | p-value² |
| DNMT3A | Mutant | 75 | 14.08 | 0.17 |
| | Wildtype | 151 | 22.83 | |
| DNMT3A | R882 Mutant | 56 | 14.08 | 0.07 |
| | Wildtype | 170 | 22.83 | |
| DNMT3A | Non-R882 Mutant | 21 | 23.52 | 0.57 |
| | Wildtype | 205 | 17.96 | |
| IDH1/2 | Mutant for IDH1 or | 46 | — | 0.001 |
| | IDH2 | | | |
| | Wildtype | 188 | 15.53 | |
| IDH1 | Mutant | 21 | 38.65 | 0.49 |
| | Wildtype | 213 | 17.53 | |
| IDH2 | Mutant | 25 | — | 0.001 |
| | Wildtype | 209 | 16.15 | |
| IDH2 | R140Q Mutant | 18 | — | 0.001 |
| | Wildtype | 216 | 16.91 | |
| IDH2 | R172K Mutant | 7 | 37.96 | 0.44 |
| | Wildtype | 227 | 16.94 | |
| TET2 | Mutant | 17 | 8.82 | 0.008 |
| | Wildtype | 214 | 19.08 | |
| ASXL1 | Mutant | 6 | 24.42 | 0.48 |
| | Wildtype | 227 | 17.66 | |
| FLT3 | Mutant | 120 | 13.52 | 0.001 |
| | Wildtype | 114 | 34.31 | |
| NPM1 | Mutant | 110 | 23.52 | 0.04 |
| | Wildtype | 124 | 16.15 | |
| PHF6 | Mutant | 3 | 2.53 | <0.0001 |
| | Wildtype | 229 | 17.96 | |
| KIT | Mutant | 2 | — | 0.98 |
| | Wildtype | 232 | 17.66 | |
| CEBPa | Mutant | 26 | 31.68 | 0.14 |
| | Wildtype | 207 | 16.91 | |
| WT1 | Mutant | 26 | 10.94 | 0.12 |
| | Wildtype | 205 | 18.26 | |
| KRAS | Mutant | 5 | — | 0.09 |
| | Wildtype | 229 | 17.53 | |
| NRAS | Mutant | 20 | — | 0.10 |
| | Wildtype | 213 | 16.94 | |
| TP53 | Mutant | 2 | — | 0.57 |
| | Wildtype | 229 | 17.89 | |
| PTEN | Mutant | 4 | — | 0.99 |
| | Wildtype | 229 | 17.89 | |
| RUNX1 | Mutant | 13 | 16.91 | 0.54 |
| | Wildtype | 215 | 17.89 | |
| EVI positive | Present | 2 | 1.25 | <0.0001 |
| | Absent | 232 | 17.89 | |
| MLL PTD | Present | 12 | 16.54 | 0.04 |
| | Absent | 222 | 18.26 | |
| Split MLL | Present | 7 | 21.71 | 0.96 |
| | Absent | 227 | 17.77 | |
| Any MLL | Present | 17 | 16.15 | 0.08 |
| abnormalitiy | Absent | 217 | 18.95 | |
| Trisomy 8 | Present | 19 | 10.16 | 0.04 |
| | Absent | 215 | 18.25 | |

1) Absence of value under column for overall survival indicates that deaths were not observed.
²P-value calculated by Log-rank test.

TABLE 11a

| Cytogenetic | | | Test | Validation | |
|---|---|---|---|---|---|
| Classi- | | | cohort | cohort | Overall |
| fication | Mutations | | (% (N)) | (% (N)) | Risk |
| Inversion | Any | | 19.7% | 15.5% | Favor- |
| (16), t(8;21) | | | (71) | (13) | able |
| Normal | FLT3-ITD | NPM1 and | 5.8% | 7.1% | |
| Karyotype or | negative | IDH1/2 mutant | (21) | (6) | |
| Intermediate | FLT3-ITD | ASXL1, MLL- | 35.5% | 27.4% | Inter- |
| Risk | negative | PTD, PHF6 | (129) | (23) | mediate |
| Cytogenetic | | and TET2- | | | |
| Lesions | | wildtype | | | |
| | FLT3-ITD | CEBPA | | | |
| | negative | mutant | | | |
| | or positive | | | | |
| | FLT3-ITD | MLL-PTD, | | | |
| | positive | TET2, and | | | |
| | | DNMT3A | | | |
| | | wild-type, and | | | |
| | | trisomy 8 | | | |
| | | negative | | | |
| | FLT3-ITD | TET2, MLL- | 20.9% | 21.4% | Unfa- |
| | negative | PTD, ASXL1, | (76) | (18) | vorable |
| | | or PHF6 | | | |
| | | mutant | | | |
| | FLT3-ITD | TET2, MLL- | | | |
| | positive | PTD, | | | |
| | | DNMT3A | | | |
| | | mutant or | | | |
| | | trisomy 8 | | | |

| | | | |
|---|---|---|---|
| Unfavorable | Any | 18.2% | 28.6% |
| | | (66) | (24) |

TABLE 12

| | | |
|---|---|---|
| Hazard Ratio | Confidence Interval | p-value |

Test cohort (n = 398)

| | | | |
|---|---|---|---|
| Favorable | Reference | | <0.001 |
| Intermediate | 1.88 | 1.15-3.05 | |
| Unfavorable | 6.16 | 3.83-9.88 | |

Entire cohort (n = 502)

| | | | |
|---|---|---|---|
| Favorable | Reference | | <0.001 |
| Intermediate | 1.83 | 1.18-2.85 | |
| Unfavorable | 5.76 | 3.76-8.82 | |

¹Treatment-related mortality defined as death within 30 days after beginning induction chemotherapy.
²Chemotherapy resistance defined as failure to enter complete remission despite not incurring treatment-related morality, or relapse.

TABLE 13

| Gene/Cytogenetic | | | |
|---|---|---|---|
| Abnormality | Mutational Status | p-value¹ | Adjusted p-value² |
| DNMT3A | Mutant | 0.01 | 0.10 |
| | Wildtype | 0.14 | 0.28 |
| IDH1 | Mutant | 0.62 | — |
| | Wildtype | 0.01 | — |
| IDH2 | Mutant | 0.33 | — |
| | Wildtype | 0.05 | — |
| IDH2 R140Q | R140Q Mutant | 0.15 | 1.0 |
| | Wildtype | 0.05 | 0.22 |
| IDH2 R172K | R172K Mutant | 0.73 | — |
| | Wildtype | 0.02 | — |
| TET2 | Mutant | 0.45 | 1.0 |
| | Wildtype | 0.006 | 0.04 |
| ASXL1 | Mutant | 0.08 | 0.50 |
| | Wildtype | 0.009 | 0.05 |
| FLT3 | Mutant | 0.14 | 0.71 |
| | Wildtype | 0.10 | 0.30 |
| NPM1 | Mutant | 0.01 | 0.11 |
| | Wildtype | 0.20 | 0.20 |
| PHF6 | Mutant | 0.19 | 0.77 |
| | Wildtype | 0.005 | 0.04 |
| KIT | Mutant | 0.12 | — |
| | Wildtype | 0.004 | — |
| CEBPa | Mutant | 0.56 | 0.56 |
| | Wildtype | 0.003 | 0.03 |
| WT1 | Mutant | 0.2 | — |
| | Wildtype | 0.02 | — |
| KRAS | Mutant | 0.62 | — |
| | Wildtype | 0.01 | — |
| NRAS | Mutant | 0.15 | — |
| | Wildtype | 0.04 | — |
| TP53 | Mutant | 0.75 | — |
| | Wildtype | 0.01 | — |
| PTEN | Mutant | 0.78 | — |
| | Wildtype | 0.02 | — |
| RUNX1 | Mutant | 0.47 | — |
| | Wildtype | 0.01 | — |
| EVI positive | Present | 0.90 | — |
| | Absent | 0.03 | — |
| MLL PTD | Present | 0.27 | — |
| | Absent | 0.01 | — |
| Split MLL | Present | 0.007 | 0.07 |
| | Absent | 0.06 | 0.25 |

¹P-value calculated by Log-rank test.
²P-value adjusted for multiple testing by a step-down Holm procedure (see Supplementary Methods), “—” indicates adjusted p-value not performed.

1. A method of predicting survival of a patient with acute myeloid leukemia (AML), said method comprising:
(a) analyzing a genetic sample isolated from the patient for the presence of cytogenetic abnormalities and a mutation in at least one of FLT3, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2 genes; and
(b) (i) predicting poor survival of the patient if a mutation is present in at least one of FLT3, MLL-PTD, ASXL1 and PHF6 genes, or (ii) predicting favorable survival of the patient if a mutation is present in IDH2R140 and/or a mutation is present in CEBPA.

2. The method of claim 1, further comprising,
predicting intermediate survival of the patient with cytogenetically-defined intermediate risk AML if:
(i) no mutation is present in any of FLT3-ITD, TET2, MLL-PTD, DNMT3A, ASXL1 or PHF6 genes,
(ii) a mutation in CEBPA and the FLT3-ITD is present, or
(iii) a mutation is present in FLT3-ITD but trisomy 8 is absent.

3. The method of claim 1, further comprising:
predicting unfavorable survival of the patient with cytogenetically-defined intermediate-risk AML if
(i) a mutation in TET2, ASXL1, or PHF6 or an MLL-PTD is present in a patient without the FLT3-ITD mutation, or
(ii) the patient has a FLT3-ITD mutation and a mutation in TET2, DNMT3A, MLL-PTD or trisomy 8.

4. The method of claim 2, wherein intermediate survival the patient is survival of about 18 months to about 30 months.

5. A method of predicting survival of a patient with acute myeloid leukemia, said method comprising:
(a) assaying a genetic sample from the patient's blood or bone marrow for the presence of a mutation in at least one of genes FLT3, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2 in said sample; and
(b) predicting a poor survival of the patient if a mutation is present in at least one of genes FLT3-ITD, MLL-PTD, ASXL1, PHF6; or predicting a favorable survival of the patient if a mutation is present in CEBPA or a mutation is present in IDH2 at R140.

6. The method of claim 5, wherein amongst patients with cytogenetically-defined intermediate-risk acute myeloid leukemia who have FLT3-ITD mutation, at least one of the following: trisomy 8 or a mutation in TET2, DNMT3A, or the MLL-PTD are associated with an adverse outcome and poor overall survival of the patient.

7. The method of claim 5, wherein amongst patients with cytogenetically-defined intermediate-risk acute myeloid leukemia who have a mutation in FLT3-ITD gene, a mutation in CEBPA gene is associated with improved outcome and overall survival of the patient.

8. The method of claim 5, wherein in a cytogenetically-defined intermediate risk AML patient with both IDH1/IDH2 and NPM1 mutations, the overall survival is improved compared to NPM1-mutant patients wild-type for both IDH1 and IDH2.

9. The method of claim 5, wherein amongst patients with acute myeloid leukemia, IDH2R140 mutations are associated with improved overall survival.

10. The method of claim 1, wherein poor or unfavorable survival (adverse risk) of the patient is survival of less than or equal to about 10 months.

11. The method of claim 1, wherein favorable survival of the patient is survival of about 32 months or more.

12. A method of predicting survival of a patient with acute myeloid leukemia, said method comprising:
(a) assaying a genetic sample from the patient's blood or bone marrow for the presence of a mutation in genes ASXL1 and WT1; and
(b) determining the patient has or will develop primary refractory acute myeloid leukemia if mutated ASXL1 and WT1 genes are detected.

13. A method of determining responsiveness of a patient with acute myeloid leukemia to high dose therapy, said method comprising:
(a) analyzing a genetic sample isolated from the patient for the presence of a mutation in genes DNMT3A, and NPM1, and for the presence of a MLL translocation; and
(b) (i) identifying the patient as one who will respond to high dose therapy if a mutation in DNMT3A or NPM1 or an MLL translocation are present; or
(ii) identifying the patient as one who will not respond to high dose therapy in the absence of mutations in DNMT3A or NPM1 or an MLL translocation.

14. A method of predicting whether a patient suffering from acute myeloid leukemia will respond better to high dose chemotherapy than to standard dose chemotherapy, the method comprising:
(a) obtaining a DNA sample obtained from the patient's blood or bone marrow;
(b) determining the mutational status of genes DNMT3A and NPM1, and the presence of a MLL translocation; and
(c) predicting that the subject will be more responsive to high dose chemotherapy than standard dose chemotherapy where the sample is positive for a mutation in DNMT3A or NPM1 or an MLL translocation; or predicting that the subject will be non-responsive to high dose chemotherapy compared to standard dose chemotherapy where the sample is wild type with no mutations in DNMT3a or NPM1 genes and no translocation in MLL.

15. A method of screening a patient with acute myeloid leukemia for responsiveness to treatment with high dose of Daunorubicin or a pharmaceutically acceptable salt, solvate, or hydrate thereof, comprising: obtaining a genetic sample comprising an acute myeloid leukemic cell from said individual; and assaying the sample and detecting the presence of a mutation in DNMT3A or NPM1 or an MLL translocation; and correlating a finding of a mutation in DNMT3A or NPM1 or an MLL translocation, as compared to wild type controls where there is no mutation, with said acute myeloid leukemia patient being more sensitive to high dose treatment with Daunorubicin or a pharmaceutically acceptable salt, solvate, or hydrate thereof.

16. The method of claim 15, wherein the method further comprises predicting the patient is at a lower risk of relapse of acute myeloid leukemia following chemotherapy if a mutation in DNMT3A or NPM1 or an MLL translocation is detected.

17. A method of determining whether a human has an increased genetic risk for developing or developing a relapse of acute myeloid leukemia, comprising:
(a) analyzing a genetic sample isolated from the human's blood or bone marrow for the presence of a mutation in at least one gene from FLT3, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2; and
(b) determining the individual with cytogenetically-defined intermediate risk AML has an increased genetic risk for developing or developing a relapse of acute myeloid leukemia, relative to a control human with no such gene mutations in said genes, when: (i) a mutation in at least one of TET2, MLL-PTD, ASXL1 and PHF6 genes is detected when the patient has no FLT3-ITD mutation, or (ii) a mutation in at least one of TET2, MLL-PTD, and DNMT3A genes or trisomy 8 is detected when the patient has a FLT3-ITD mutation.

18. A method for preparing a personalized genomics profile for a patient with acute myeloid leukemia, comprising:
(a) subjecting mononuclear cells extracted from a bone marrow aspirate or blood sample from the patient to gene mutational analysis;
(b) assaying the sample and detecting the presence of a cytogenetic abnormality and one or more mutations in a gene selected from the group consisting of FLT3, NPM1, DNMT3A, NRAS, CEBPA, TET2, WT1, IDH1, IDH2, KIT, RUNX1, MLL-PTD, ASXL1, PHF6, KRAS, PTEN, P53, HRAS, and EZH2 in said cells; and
(c) generating a report of the data obtained by the gene mutation analysis, wherein the report comprises a prediction of the likelihood of survival of the patient or a response to therapy.

19. A kit for determining treatment of a patient with AML, the kit comprising means for detecting a mutation in at least one gene selected from the group consisting of ASXL1, DNMT3A, NPM1, PHF6, WT1, TP53, EZH2, CEBPA, TET2, RUNX1, PTEN, FLT3, HRAS, KRAS, NRAS, KIT, IDH1, and IDH2; and instructions for recommended treatment based on the presence of a mutation in one or more of said genes.

20. The kit of claim 31, wherein the instructions for recommended treatment for the patient based on the presence of a DNMT3A or NPM1 mutation or MLL translocation indicate high-dose daunorubicin as the recommended treatment.

21. A method of treating, preventing or managing acute myeloid leukemia in a patient, comprising:
(a) analyzing a genetic sample isolated from the patient for the presence of a mutation in genes DNMT3A, and NPM1, and for the presence of a MLL translocation;
(b) identifying the patient as one who will respond to high dose chemotherapy better than standard dose chemotherapy if a mutation in DNMT3A or NPM1 or a MLL translocation are present; and
(c) administering high dose therapy to the patient.

22. The method of claim 5, wherein the patient is characterized as intermediate-risk on the basis of cytogenetic analysis.

23. The method of claim 14, wherein the therapy comprises the administration of anthracycline.

24. The method of claim 14 or claim 21, wherein administering high dose therapy comprises administering one or more high dose anthracycline antibiotics selected from the group consisting of Daunorubicin, Doxorubicin, Epirubicin, Idarubicin, Mitoxantrone, and Adriamycin.

25. The method of claim 13, wherein the sample is DNA extracted from bone marrow or blood from the patient.

26. The method of claim 13, wherein the genetic sample is DNA isolated from mononuclear cells (MNC) from the patient.

27. The method of claim 21, wherein the high dose administration is Daunorubicin administered at from about 70 mg/m2 to about 140 mg/m2, or Idarubicin administered at from about 10 mg/m2 to about 20 mg/m2.

28-33. (canceled)

34. A method of predicting survival of a patient with acute myeloid leukemia, comprising:
(a) analyzing a sample isolated from the patient for the presence of
(i) a mutation in at least one of FLT3, MLL-PTD, ASXL1, and PHF6 genes, plus optionally one or more of NPM1, DNMT3A, NRAS, CEBPA, TET2, WT1, IDH1, IDH2, KIT, RUNX1, KRAS, PTEN, P53, HRAS, and EZH2 genes; or
(ii) a mutation in IDH2 and/or CEBPA genes, plus optionally one or more of FLT3, MLL-PTD, ASXL1, PHF6, NPM1, DNMT3A, NRAS, TET2, WT1, IDH1, KIT, RUNX1, KRAS, PTEN, P53, HRAS, and EZH2 genes; and
(b) (i) predicting poor survival of the patient if a mutation is present in at least one of FLT3, MLL-PTD, ASXL1 and PHF6 genes, or (ii) predicting favorable survival of the patient if a mutation is present in IDH2R140 and/or a mutation is present in CEBPA.

35. The method of claim 34, further comprising analyzing the sample for the presence of cytogenetic abnormalities.

36. The method of claim 34, further comprising (ii) predicting favorable survival of the patient if the following mutation is present: IDH2R140Q.
